(19) United States
(12) Patent Application Publication
(10) Pub. No.: US 2022/0332816 A1
(43) Pub. Date: Oct. 20, 2022

(54) ANTI-TIGIT ANTIBODIES

(71) Applicant: Single Cell Technology, Inc., San Jose, CA (US)

(72) Inventors: Leyan TANG, San Jose, CA (US); Allison SCHULKINS, San Jose, CA (US); Kimberly THAN, San Jose, CA (US); Chun-Nan CHEN, San Jose, CA (US)

(73) Assignee: Single Cell Technology, Inc., San Jose, CA (US)

(21) Appl. No.: 17/621,597

(22) PCT Filed: Jun. 22, 2020
(86) PCT No.: PCT/US2020/038925

§ 371 Date: Dec. 21, 2021

Related U.S. Application Data
(60) Provisional application No. 62/864,842, filed on Jun. 21, 2019.

(51) Int. Cl. C07K 16/28

(52) U.S. Cl. CPC C07K 16/2803; C07K 2317/565; C07K 2317/21; C07K 2317/24; C07K 2317/92

(57) ABSTRACT

Provided are antibodies that specifically recognize T cell immunoreceptor with Ig and ITIM domains protein (TIGIT).

### CROSS-REFERENCE TO RELATED APPLICATIONS

This application claims priority to U.S. Provisional Application No. 62/864,842 filed 21 Jun. 2019, which is incorporated herein by reference in its entirety for all purposes.

### INCORPORATION OF SEQUENCE LISTING

The instant application contains a Sequence Listing which has been submitted via EFS-Web and is hereby incorporated by reference in its entirety. The ASCII copy, created on Jun. 22, 2020, is named SCT0010-401-PC_Sequence_Listing_ST25.txt, and is 849,000 bytes in size.

### BACKGROUND

TIGIT (T cell immunoreceptor with Ig and ITIM domains) is an immune receptor present on some T cells and natural killer cells (Yu et al., 2009) and is a co-inhibitory receptor protein also known as WUCAM (Boles et al., 2009) or Vstm3 (Levin et al., 2011). TIGIT was discovered in genomic searches for proteins specifically expressed on T cells, and has an immunoglobulin variable domain, a transmembrane domain, and an immunoreceptor tyrosine-based inhibitory motif (ITIM) and contains signature sequence elements of the PVR protein family. It is known to interact with poliovirus receptor (PVR; CD155) and with nectin2 (CD112) (Stengel et al., 2012). Although PVR may interact with the co-activating receptor DNAM-1 (CD226) to enhance tumor killing, the high affinity TIGIT/PVR interaction would inhibit such killing and may act to prevent killing of normal (self) cells that also express PVR (Stanietsky et al., 2009). The dominance of this inhibitory interaction may be important in suppression of anti-self-immune reactions, but in the tumor context it suppresses tumor eradication.

TIGIT suppresses T cell activation by promoting the generation of mature immunoregulatory dendritic cells (Yu et al., 2009). TIGIT and other such co-inhibitory molecules (e.g. CTLA-4, PD-1, Lag3 and BTLA) may play a role in evasion of immunosurveillance by tumor cells. Experiments have shown that PVR/CD155 is over-expressed on melanoma cells (Gao et al., 2017) and various other tumors. It is possible that the TIGIT/PVR interaction can shield such tumor cells from immune-mediated eradication by inhibiting anti-tumor responses of T and NK cells (Stanietsky et al., 2009; Lozano et al., 2012). Other experiments have identified a TIGIT subset of regulatory T cells (T_{regs}) that selectively suppress Th1 and Th17 responses (Joller et al., 2014), suggesting an alternative mechanism by which an anti-TIGIT antibody may enhance anti-tumor immune response.

TIGIT may act to “turn off” the immune response similarly to other co-inhibitory receptors such as CTLA-4, PD-1 and BTLA. Id. Antibodies targeting CTLA-4 (ipilimumab) and PD-1 (nivolumab, pembrolizumab) have been approved for the treatment of human cancers, validating this therapeutic approach. Antibodies that bind to human TIGIT might also find use in treatment of cancers (Johnston et al., 2014). In mouse models, antibody blockade of both PD-L1 and TIGIT leads to a synergistic enhancement of CD8⁺ T cell mediated tumor rejection (Johnston et al., 2015). Similar results have been obtained in animal models of melanoma. (Gao et al., 2017). Some experiments suggest that TIGIT blockade is effective to enhance anti-tumor CD8⁺ T cell response only in the presence of the co-activating receptor DNAM-1/CD226, which competes with TIGIT for binding to PVR/CD155 (Johnston et al., 2014).

Recent experiments have demonstrated that intratumoral bacteria expressing Fap2 protein may inhibit NK cell mediated tumor killing by binding to TIGIT (Gur et al., 2015), suggesting that eliminating such bacteria, blocking the interaction of TIGIT with Fap2, or blocking the activity of TIGIT generally, may be useful in treatment of cancer, e.g. colorectal cancer (Hampton, 2015).

Thus, the need exists for improved methods of treating cancer and chronic viral infections and medicaments, such as therapeutic monoclonal antibodies. Medicines for use in such improved methods of treatment may comprise antibodies or antibody fragments that specifically bind to TIGIT and reverse or partially reverse the TIGIT-mediated suppression of anti-tumor or anti-viral immune responses.

### SUMMARY

Provided are antibodies that recognize and specifically bind to human TIGIT. In one embodiment, the disclosure provides an antibody that binds to SEQ ID NO:1.

### BRIEF DESCRIPTION OF THE SEQUENCES

TABLE 1

| SEQ ID NO. | Description of sequence |
|---|---|
| 1 | Human TIGIT protein sequence |
| 2 | Human TIGIT His tagged protein sequence |
| 3 | Human TIGIT Human Fc tagged protein sequence |
| 4 | SCT-Ga001 mature heavy chain variable domain protein sequence |
| 5 | SCT-Ga002 mature heavy chain variable domain protein sequence |
| 6 | SCT-Ga003 mature heavy chain variable domain protein sequence |
| 6 | SCT-Ga042 mature heavy chain variable domain protein sequence |
| 7 | SCT-Ga004 mature heavy chain variable domain protein sequence |
| 7 | SCT-Ga068 mature heavy chain variable domain protein sequence |
| 7 | SCT-Ga073 mature heavy chain variable domain protein sequence |
| 7 | SCT-Ga118 mature heavy chain variable domain protein sequence |
| 7 | SCT-Ga125 mature heavy chain variable domain protein sequence |
| 7 | SCT-Ga157 mature heavy chain variable domain protein sequence |
| 7 | SCT-Ga158 mature heavy chain variable domain protein sequence |
| 8 | SCT-Ga005 mature heavy chain variable domain protein sequence |
| 9 | SCT-Ga006 mature heavy chain variable domain protein sequence |
| 10 | SCT-Ga007 mature heavy chain variable domain protein sequence |
| 11 | SCT-Ga008 mature heavy chain variable domain protein sequence |
| 12 | SCT-Ga009 mature heavy chain variable domain protein sequence |
| 13 | SCT-Ga010 mature heavy chain variable domain protein sequence |
| 14 | SCT-Ga011 mature heavy chain variable domain protein sequence |
| 15 | SCT-Ga012 mature heavy chain variable domain protein sequence |
| 16 | SCT-Ga013 mature heavy chain variable domain protein sequence |
| 17 | SCT-Ga014 mature heavy chain variable domain protein sequence |
| 17 | SCT-Ga140 mature heavy chain variable domain protein sequence |
| 17 | SCT-Ga152 mature heavy chain variable domain protein sequence |
| 18 | SCT-Ga015 mature heavy chain variable domain protein sequence |
| 19 | SCT-Ga016 mature heavy chain variable domain protein sequence |
| 20 | SCT-Ga017 mature heavy chain variable domain protein sequence |
| 20 | SCT-Ga022 mature heavy chain variable domain protein sequence |
| 20 | SCT-Ga045 mature heavy chain variable domain protein sequence |
| 20 | SCT-Ga066 mature heavy chain variable domain protein sequence |
| 21 | SCT-Ga018 mature heavy chain variable domain protein sequence |
| 21 | SCT-Ga050 mature heavy chain variable domain protein sequence |
| 22 | SCT-Ga019 mature heavy chain variable domain protein sequence |
| 23 | SCT-Ga020 mature heavy chain variable domain protein sequence |
| 23 | SCT-Ga044 mature heavy chain variable domain protein sequence |
| 23 | SCT-Ga141 mature heavy chain variable domain protein sequence |
| 24 | SCT-Ga021 mature heavy chain variable domain protein sequence |
| 25 | SCT-Ga023 mature heavy chain variable domain protein sequence |
| 26 | SCT-Ga024 mature heavy chain variable domain protein sequence |
| 27 | SCT-Ga025 mature heavy chain variable domain protein sequence |
| 27 | SCT-Ga043 mature heavy chain variable domain protein sequence |
| 27 | SCT-Ga121 mature heavy chain variable domain protein sequence |
| 27 | SCT-Ga133 mature heavy chain variable domain protein sequence |
| 27 | SCT-Ga154 mature heavy chain variable domain protein sequence |
| 28 | SCT-Ga026 mature heavy chain variable domain protein sequence |
| 29 | SCT-Ga027 mature heavy chain variable domain protein sequence |
| 30 | SCT-Ga028 mature heavy chain variable domain protein sequence |
| 31 | SCT-Ga029 mature heavy chain variable domain protein sequence |
| 32 | SCT-Ga030 mature heavy chain variable domain protein sequence |
| 33 | SCT-Ga031 mature heavy chain variable domain protein sequence |
| 33 | SCT-Ga062 mature heavy chain variable domain protein sequence |
| 34 | SCT-Ga032 mature heavy chain variable domain protein sequence |
| 35 | SCT-Ga033 mature heavy chain variable domain protein sequence |
| 36 | SCT-Ga034 mature heavy chain variable domain protein sequence |
| 36 | SCT-Ga083 mature heavy chain variable domain protein sequence |
| 37 | SCT-Ga035 mature heavy chain variable domain protein sequence |
| 38 | SCT-Ga036 mature heavy chain variable domain protein sequence |
| 39 | SCT-Ga037 mature heavy chain variable domain protein sequence |
| 40 | SCT-Ga038 mature heavy chain variable domain protein sequence |
| 41 | SCT-Ga039 mature heavy chain variable domain protein sequence |
| 42 | SCT-Ga040 mature heavy chain variable domain protein sequence |
| 43 | SCT-Ga041 mature heavy chain variable domain protein sequence |
| 44 | SCT-Ga046 mature heavy chain variable domain protein sequence |
| 45 | SCT-Ga047 mature heavy chain variable domain protein sequence |
| 46 | SCT-Ga048 mature heavy chain variable domain protein sequence |
| 47 | SCT-Ga049 mature heavy chain variable domain protein sequence |
| 48 | SCT-Ga051 mature heavy chain variable domain protein sequence |
| 49 | SCT-Ga052 mature heavy chain variable domain protein sequence |
| 50 | SCT-Ga053 mature heavy chain variable domain protein sequence |
| 51 | SCT-Ga054 mature heavy chain variable domain protein sequence |
| 51 | SCT-Ga168 mature heavy chain variable domain protein sequence |
| 51 | SCT-Ga169 mature heavy chain variable domain protein sequence |
| 52 | SCT-Ga055 mature heavy chain variable domain protein sequence |
| 53 | SCT-Ga056 mature heavy chain variable domain protein sequence |
| 54 | SCT-Ga057 mature heavy chain variable domain protein sequence |
| 55 | SCT-Ga058 mature heavy chain variable domain protein sequence |
| 55 | SCT-Ga077 mature heavy chain variable domain protein sequence |
| 56 | SCT-Ga059 mature heavy chain variable domain protein sequence |
| 57 | SCT-Ga060 mature heavy chain variable domain protein sequence |
| 58 | SCT-Ga061 mature heavy chain variable domain protein sequence |
| 58 | SCT-Ga150 mature heavy chain variable domain protein sequence |
| 59 | SCT-Ga063 mature heavy chain variable domain protein sequence |
| 60 | SCT-Ga064 mature heavy chain variable domain protein sequence |
| 61 | SCT-Ga065 mature heavy chain variable domain protein sequence |
| 62 | SCT-Ga067 mature heavy chain variable domain protein sequence |
| 62 | SCT-Ga069 mature heavy chain variable domain protein sequence |
| 62 | SCT-Ga071 mature heavy chain variable domain protein sequence |
| 62 | SCT-Ga080 mature heavy chain variable domain protein sequence |
| 62 | SCT-Ga110 mature heavy chain variable domain protein sequence |
| 63 | SCT-Ga070 mature heavy chain variable domain protein sequence |
| 64 | SCT-Ga072 mature heavy chain variable domain protein sequence |
| 65 | SCT-Ga074 mature heavy chain variable domain protein sequence |
| 66 | SCT-Ga075 mature heavy chain variable domain protein sequence |
| 67 | SCT-Ga076 mature heavy chain variable domain protein sequence |
| 67 | SCT-Ga094 mature heavy chain variable domain protein sequence |
| 67 | SCT-Ga166 mature heavy chain variable domain protein sequence |
| 68 | SCT-Ga078 mature heavy chain variable domain protein sequence |
| 69 | SCT-Ga079 mature heavy chain variable domain protein sequence |
| 70 | SCT-Ga081 mature heavy chain variable domain protein sequence |
| 71 | SCT-Ga082 mature heavy chain variable domain protein sequence |
| 72 | SCT-Ga084 mature heavy chain variable domain protein sequence |
| 73 | SCT-Ga085 mature heavy chain variable domain protein sequence |
| 74 | SCT-Ga086 mature heavy chain variable domain protein sequence |
| 75 | SCT-Ga087 mature heavy chain variable domain protein sequence |
| 75 | SCT-Ga088 mature heavy chain variable domain protein sequence |
| 76 | SCT-Ga089 mature heavy chain variable domain protein sequence |
| 77 | SCT-Ga090 mature heavy chain variable domain protein sequence |
| 78 | SCT-Ga091 mature heavy chain variable domain protein sequence |
| 79 | SCT-Ga092 mature heavy chain variable domain protein sequence |
| 79 | SCT-Ga100 mature heavy chain variable domain protein sequence |
| 80 | SCT-Ga093 mature heavy chain variable domain protein sequence |
| 81 | SCT-Ga095 mature heavy chain variable domain protein sequence |
| 82 | SCT-Ga096 mature heavy chain variable domain protein sequence |
| 83 | SCT-Ga097 mature heavy chain variable domain protein sequence |
| 84 | SCT-Ga098 mature heavy chain variable domain protein sequence |
| 84 | SCT-Ga113 mature heavy chain variable domain protein sequence |
| 84 | SCT-Ga123 mature heavy chain variable domain protein sequence |
| 84 | SCT-Ga146 mature heavy chain variable domain protein sequence |
| 84 | SCT-Ga162 mature heavy chain variable domain protein sequence |
| 85 | SCT-Ga099 mature heavy chain variable domain protein sequence |
| 86 | SCT-Ga101 mature heavy chain variable domain protein sequence |
| 87 | SCT-Ga102 mature heavy chain variable domain protein sequence |
| 88 | SCT-Ga103 mature heavy chain variable domain protein sequence |
| 89 | SCT-Ga104 mature heavy chain variable domain protein sequence |
| 90 | SCT-Ga105 mature heavy chain variable domain protein sequence |
| 91 | SCT-Ga106 mature heavy chain variable domain protein sequence |
| 92 | SCT-Ga107 mature heavy chain variable domain protein sequence |
| 93 | SCT-Ga108 mature heavy chain variable domain protein sequence |
| 93 | SCT-Ga149 mature heavy chain variable domain protein sequence |
| 94 | SCT-Ga109 mature heavy chain variable domain protein sequence |
| 95 | SCT-Ga111 mature heavy chain variable domain protein sequence |
| 96 | SCT-Ga112 mature heavy chain variable domain protein sequence |
| 97 | SCT-Ga114 mature heavy chain variable domain protein sequence |
| 98 | SCT-Ga115 mature heavy chain variable domain protein sequence |
| 99 | SCT-Ga116 mature heavy chain variable domain protein sequence |
| 100 | SCT-Ga117 mature heavy chain variable domain protein sequence |
| 101 | SCT-Ga119 mature heavy chain variable domain protein sequence |
| 102 | SCT-Ga120 mature heavy chain variable domain protein sequence |
| 102 | SCT-Ga171 mature heavy chain variable domain protein sequence |
| 103 | SCT-Ga122 mature heavy chain variable domain protein sequence |
| 104 | SCT-Ga124 mature heavy chain variable domain protein sequence |
| 105 | SCT-Ga126 mature heavy chain variable domain protein sequence |
| 106 | SCT-Ga127 mature heavy chain variable domain protein sequence |
| 107 | SCT-Ga128 mature heavy chain variable domain protein sequence |
| 108 | SCT-Ga129 mature heavy chain variable domain protein sequence |
| 109 | SCT-Ga130 mature heavy chain variable domain protein sequence |
| 110 | SCT-Ga131 mature heavy chain variable domain protein sequence |
| 111 | SCT-Ga132 mature heavy chain variable domain protein sequence |
| 112 | SCT-Ga134 mature heavy chain variable domain protein sequence |
| 113 | SCT-Ga135 mature heavy chain variable domain protein sequence |
| 114 | SCT-Ga136 mature heavy chain variable domain protein sequence |
| 115 | SCT-Ga137 mature heavy chain variable domain protein sequence |
| 116 | SCT-Ga138 mature heavy chain variable domain protein sequence |
| 117 | SCT-Ga139 mature heavy chain variable domain protein sequence |
| 118 | SCT-Ga142 mature heavy chain variable domain protein sequence |
| 119 | SCT-Ga143 mature heavy chain variable domain protein sequence |
| 120 | SCT-Ga144 mature heavy chain variable domain protein sequence |
| 121 | SCT-Ga145 mature heavy chain variable domain protein sequence |
| 122 | SCT-Ga147 mature heavy chain variable domain protein sequence |
| 123 | SCT-Ga148 mature heavy chain variable domain protein sequence |
| 124 | SCT-Ga151 mature heavy chain variable domain protein sequence |
| 125 | SCT-Ga153 mature heavy chain variable domain protein sequence |
| 126 | SCT-Ga155 mature heavy chain variable domain protein sequence |
| 127 | SCT-Ga156 mature heavy chain variable domain protein sequence |
| 128 | SCT-Ga159 mature heavy chain variable domain protein sequence |
| 129 | SCT-Ga160 mature heavy chain variable domain protein sequence |
| 130 | SCT-Ga161 mature heavy chain variable domain protein sequence |
| 131 | SCT-Ga163 mature heavy chain variable domain protein sequence |
| 132 | SCT-Ga164 mature heavy chain variable domain protein sequence |
| 133 | SCT-Ga165 mature heavy chain variable domain protein sequence |
| 134 | SCT-Ga167 mature heavy chain variable domain protein sequence |
| 135 | SCT-Ga170 mature heavy chain variable domain protein sequence |
| 136 | SCT-Ga172 mature heavy chain variable domain protein sequence |
| 137 | SCT-Ga173 mature heavy chain variable domain protein sequence |
| 138 | SCT-Ga174 mature heavy chain variable domain protein sequence |
| 139 | SCT-Ga175 mature heavy chain variable domain protein sequence |
| 140 | SCT-Ga176 mature heavy chain variable domain protein sequence |
| 140 | SCT-Ga233 mature heavy chain variable domain protein sequence |
| 141 | SCT-Ga177 mature heavy chain variable domain protein sequence |
| 142 | SCT-Ga178 mature heavy chain variable domain protein sequence |
| 143 | SCT-Ga179 mature heavy chain variable domain protein sequence |
| 144 | SCT-Ga180 mature heavy chain variable domain protein sequence |
| 145 | SCT-Ga181 mature heavy chain variable domain protein sequence |
| 146 | SCT-Ga182 mature heavy chain variable domain protein sequence |
| 147 | SCT-Ga183 mature heavy chain variable domain protein sequence |
| 148 | SCT-Ga184 mature heavy chain variable domain protein sequence |
| 149 | SCT-Ga185 mature heavy chain variable domain protein sequence |
| 150 | SCT-Ga186 mature heavy chain variable domain protein sequence |
| 151 | SCT-Ga187 mature heavy chain variable domain protein sequence |
| 151 | SCT-Ga273 mature heavy chain variable domain protein sequence |
| 152 | SCT-Ga188 mature heavy chain variable domain protein sequence |
| 153 | SCT-Ga189 mature heavy chain variable domain protein sequence |
| 154 | SCT-Ga190 mature heavy chain variable domain protein sequence |
| 155 | SCT-Ga191 mature heavy chain variable domain protein sequence |
| 156 | SCT-Ga192 mature heavy chain variable domain protein sequence |
| 157 | SCT-Ga193 mature heavy chain variable domain protein sequence |
| 158 | SCT-Ga194 mature heavy chain variable domain protein sequence |
| 159 | SCT-Ga195 mature heavy chain variable domain protein sequence |
| 160 | SCT-Ga196 mature heavy chain variable domain protein sequence |
| 161 | SCT-Ga197 mature heavy chain variable domain protein sequence |
| 162 | SCT-Ga198 mature heavy chain variable domain protein sequence |
| 163 | SCT-Ga199 mature heavy chain variable domain protein sequence |
| 163 | SCT-Ga279 mature heavy chain variable domain protein sequence |
| 164 | SCT-Ga200 mature heavy chain variable domain protein sequence |
| 164 | SCT-Ga285 mature heavy chain variable domain protein sequence |
| 165 | SCT-Ga201 mature heavy chain variable domain protein sequence |
| 166 | SCT-Ga202 mature heavy chain variable domain protein sequence |
| 167 | SCT-Ga203 mature heavy chain variable domain protein sequence |
| 168 | SCT-Ga204 mature heavy chain variable domain protein sequence |
| 169 | SCT-Ga205 mature heavy chain variable domain protein sequence |
| 170 | SCT-Ga206 mature heavy chain variable domain protein sequence |
| 170 | SCT-Ga239 mature heavy chain variable domain protein sequence |
| 171 | SCT-Ga207 mature heavy chain variable domain protein sequence |
| 171 | SCT-Ga236 mature heavy chain variable domain protein sequence |
| 171 | SCT-Ga253 mature heavy chain variable domain protein sequence |
| 172 | SCT-Ga208 mature heavy chain variable domain protein sequence |
| 173 | SCT-Ga209 mature heavy chain variable domain protein sequence |
| 173 | SCT-Ga306 mature heavy chain variable domain protein sequence |
| 173 | SCT-Ga367 mature heavy chain variable domain protein sequence |
| 174 | SCT-Ga210 mature heavy chain variable domain protein sequence |
| 175 | SCT-Ga211 mature heavy chain variable domain protein sequence |
| 176 | SCT-Ga212 mature heavy chain variable domain protein sequence |
| 176 | SCT-Ga373 mature heavy chain variable domain protein sequence |
| 177 | SCT-Ga213 mature heavy chain variable domain protein sequence |
| 177 | SCT-Ga232 mature heavy chain variable domain protein sequence |
| 177 | SCT-Ga335 mature heavy chain variable domain protein sequence |
| 177 | SCT-Ga358 mature heavy chain variable domain protein sequence |
| 177 | SCT-Ga363 mature heavy chain variable domain protein sequence |
| 178 | SCT-Ga214 mature heavy chain variable domain protein sequence |
| 179 | SCT-Ga215 mature heavy chain variable domain protein sequence |
| 180 | SCT-Ga216 mature heavy chain variable domain protein sequence |
| 180 | SCT-Ga353 mature heavy chain variable domain protein sequence |
| 181 | SCT-Ga217 mature heavy chain variable domain protein sequence |
| 181 | SCT-Ga223 mature heavy chain variable domain protein sequence |
| 182 | SCT-Ga218 mature heavy chain variable domain protein sequence |
| 182 | SCT-Ga257 mature heavy chain variable domain protein sequence |
| 182 | SCT-Ga296 mature heavy chain variable domain protein sequence |
| 183 | SCT-Ga219 mature heavy chain variable domain protein sequence |
| 183 | SCT-Ga266 mature heavy chain variable domain protein sequence |
| 183 | SCT-Ga336 mature heavy chain variable domain protein sequence |
| 184 | SCT-Ga220 mature heavy chain variable domain protein sequence |
| 185 | SCT-Ga221 mature heavy chain variable domain protein sequence |
| 186 | SCT-Ga222 mature heavy chain variable domain protein sequence |
| 187 | SCT-Ga224 mature heavy chain variable domain protein sequence |
| 188 | SCT-Ga225 mature heavy chain variable domain protein sequence |
| 189 | SCT-Ga226 mature heavy chain variable domain protein sequence |
| 189 | SCT-Ga312 mature heavy chain variable domain protein sequence |
| 189 | SCT-Ga324 mature heavy chain variable domain protein sequence |
| 190 | SCT-Ga227 mature heavy chain variable domain protein sequence |
| 191 | SCT-Ga228 mature heavy chain variable domain protein sequence |
| 192 | SCT-Ga229 mature heavy chain variable domain protein sequence |
| 192 | SCT-Ga288 mature heavy chain variable domain protein sequence |
| 193 | SCT-Ga230 mature heavy chain variable domain protein sequence |
| 194 | SCT-Ga231 mature heavy chain variable domain protein sequence |
| 195 | SCT-Ga234 mature heavy chain variable domain protein sequence |
| 196 | SCT-Ga235 mature heavy chain variable domain protein sequence |
| 197 | SCT-Ga237 mature heavy chain variable domain protein sequence |
| 198 | SCT-Ga238 mature heavy chain variable domain protein sequence |
| 198 | SCT-Ga305 mature heavy chain variable domain protein sequence |
| 199 | SCT-Ga240 mature heavy chain variable domain protein sequence |
| 200 | SCT-Ga241 mature heavy chain variable domain protein sequence |
| 201 | SCT-Ga242 mature heavy chain variable domain protein sequence |
| 201 | SCT-Ga259 mature heavy chain variable domain protein sequence |
| 201 | SCT-Ga276 mature heavy chain variable domain protein sequence |
| 201 | SCT-Ga287 mature heavy chain variable domain protein sequence |
| 201 | SCT-Ga329 mature heavy chain variable domain protein sequence |
| 201 | SCT-Ga332 mature heavy chain variable domain protein sequence |
| 202 | SCT-Ga243 mature heavy chain variable domain protein sequence |
| 203 | SCT-Ga244 mature heavy chain variable domain protein sequence |
| 204 | SCT-Ga245 mature heavy chain variable domain protein sequence |
| 205 | SCT-Ga246 mature heavy chain variable domain protein sequence |
| 206 | SCT-Ga247 mature heavy chain variable domain protein sequence |
| 207 | SCT-Ga248 mature heavy chain variable domain protein sequence |
| 208 | SCT-Ga249 mature heavy chain variable domain protein sequence |
| 209 | SCT-Ga250 mature heavy chain variable domain protein sequence |
| 210 | SCT-Ga251 mature heavy chain variable domain protein sequence |
| 210 | SCT-Ga290 mature heavy chain variable domain protein sequence |
| 211 | SCT-Ga252 mature heavy chain variable domain protein sequence |
| 212 | SCT-Ga254 mature heavy chain variable domain protein sequence |
| 213 | SCT-Ga255 mature heavy chain variable domain protein sequence |
| 214 | SCT-Ga256 mature heavy chain variable domain protein sequence |
| 215 | SCT-Ga258 mature heavy chain variable domain protein sequence |
| 216 | SCT-Ga260 mature heavy chain variable domain protein sequence |
| 217 | SCT-Ga261 mature heavy chain variable domain protein sequence |
| 218 | SCT-Ga262 mature heavy chain variable domain protein sequence |
| 219 | SCT-Ga263 mature heavy chain variable domain protein sequence |
| 220 | SCT-Ga264 mature heavy chain variable domain protein sequence |
| 220 | SCT-Ga322 mature heavy chain variable domain protein sequence |
| 221 | SCT-Ga265 mature heavy chain variable domain protein sequence |
| 222 | SCT-Ga267 mature heavy chain variable domain protein sequence |
| 223 | SCT-Ga268 mature heavy chain variable domain protein sequence |
| 224 | SCT-Ga269 mature heavy chain variable domain protein sequence |
| 224 | SCT-Ga351 mature heavy chain variable domain protein sequence |
| 225 | SCT-Ga270 mature heavy chain variable domain protein sequence |
| 226 | SCT-Ga271 mature heavy chain variable domain protein sequence |
| 227 | SCT-Ga272 mature heavy chain variable domain protein sequence |
| 228 | SCT-Ga274 mature heavy chain variable domain protein sequence |
| 229 | SCT-Ga275 mature heavy chain variable domain protein sequence |
| 230 | SCT-Ga277 mature heavy chain variable domain protein sequence |
| 230 | SCT-Ga300 mature heavy chain variable domain protein sequence |
| 231 | SCT-Ga278 mature heavy chain variable domain protein sequence |
| 232 | SCT-Ga280 mature heavy chain variable domain protein sequence |
| 233 | SCT-Ga281 mature heavy chain variable domain protein sequence |
| 234 | SCT-Ga282 mature heavy chain variable domain protein sequence |
| 235 | SCT-Ga283 mature heavy chain variable domain protein sequence |
| 236 | SCT-Ga284 mature heavy chain variable domain protein sequence |
| 237 | SCT-Ga286 mature heavy chain variable domain protein sequence |
| 238 | SCT-Ga289 mature heavy chain variable domain protein sequence |
| 238 | SCT-Ga357 mature heavy chain variable domain protein sequence |
| 239 | SCT-Ga291 mature heavy chain variable domain protein sequence |
| 240 | SCT-Ga292 mature heavy chain variable domain protein sequence |
| 241 | SCT-Ga293 mature heavy chain variable domain protein sequence |
| 242 | SCT-Ga294 mature heavy chain variable domain protein sequence |
| 243 | SCT-Ga295 mature heavy chain variable domain protein sequence |
| 244 | SCT-Ga297 mature heavy chain variable domain protein sequence |
| 245 | SCT-Ga298 mature heavy chain variable domain protein sequence |
| 246 | SCT-Ga299 mature heavy chain variable domain protein sequence |
| 246 | SCT-Ga302 mature heavy chain variable domain protein sequence |
| 247 | SCT-Ga301 mature heavy chain variable domain protein sequence |
| 248 | SCT-Ga303 mature heavy chain variable domain protein sequence |
| 249 | SCT-Ga304 mature heavy chain variable domain protein sequence |
| 250 | SCT-Ga307 mature heavy chain variable domain protein sequence |
| 251 | SCT-Ga308 mature heavy chain variable domain protein sequence |
| 252 | SCT-Ga309 mature heavy chain variable domain protein sequence |
| 253 | SCT-Ga310 mature heavy chain variable domain protein sequence |
| 254 | SCT-Ga311 mature heavy chain variable domain protein sequence |
| 255 | SCT-Ga313 mature heavy chain variable domain protein sequence |
| 256 | SCT-Ga314 mature heavy chain variable domain protein sequence |
| 257 | SCT-Ga315 mature heavy chain variable domain protein sequence |
| 257 | SCT-Ga331 mature heavy chain variable domain protein sequence |
| 258 | SCT-Ga316 mature heavy chain variable domain protein sequence |
| 259 | SCT-Ga317 mature heavy chain variable domain protein sequence |
| 260 | SCT-Ga318 mature heavy chain variable domain protein sequence |
| 261 | SCT-Ga319 mature heavy chain variable domain protein sequence |
| 262 | SCT-Ga320 mature heavy chain variable domain protein sequence |
| 263 | SCT-Ga321 mature heavy chain variable domain protein sequence |
| 264 | SCT-Ga323 mature heavy chain variable domain protein sequence |
| 265 | SCT-Ga325 mature heavy chain variable domain protein sequence |
| 266 | SCT-Ga326 mature heavy chain variable domain protein sequence |
| 267 | SCT-Ga327 mature heavy chain variable domain protein sequence |
| 267 | SCT-Ga375 mature heavy chain variable domain protein sequence |
| 268 | SCT-Ga328 mature heavy chain variable domain protein sequence |
| 269 | SCT-Ga330 mature heavy chain variable domain protein sequence |
| 270 | SCT-Ga333 mature heavy chain variable domain protein sequence |
| 271 | SCT-Ga334 mature heavy chain variable domain protein sequence |
| 272 | SCT-Ga337 mature heavy chain variable domain protein sequence |
| 273 | SCT-Ga338 mature heavy chain variable domain protein sequence |
| 274 | SCT-Ga339 mature heavy chain variable domain protein sequence |
| 275 | SCT-Ga340 mature heavy chain variable domain protein sequence |
| 276 | SCT-Ga341 mature heavy chain variable domain protein sequence |
| 277 | SCT-Ga342 mature heavy chain variable domain protein sequence |
| 278 | SCT-Ga343 mature heavy chain variable domain protein sequence |
| 279 | SCT-Ga344 mature heavy chain variable domain protein sequence |
| 280 | SCT-Ga345 mature heavy chain variable domain protein sequence |
| 281 | SCT-Ga346 mature heavy chain variable domain protein sequence |
| 282 | SCT-Ga347 mature heavy chain variable domain protein sequence |
| 283 | SCT-Ga348 mature heavy chain variable domain protein sequence |
| 284 | SCT-Ga349 mature heavy chain variable domain protein sequence |
| 285 | SCT-Ga350 mature heavy chain variable domain protein sequence |
| 286 | SCT-Ga352 mature heavy chain variable domain protein sequence |
| 287 | SCT-Ga354 mature heavy chain variable domain protein sequence |
| 288 | SCT-Ga355 mature heavy chain variable domain protein sequence |
| 289 | SCT-Ga356 mature heavy chain variable domain protein sequence |
| 290 | SCT-Ga359 mature heavy chain variable domain protein sequence |
| 291 | SCT-Ga360 mature heavy chain variable domain protein sequence |
| 292 | SCT-Ga361 mature heavy chain variable domain protein sequence |
| 293 | SCT-Ga362 mature heavy chain variable domain protein sequence |
| 294 | SCT-Ga364 mature heavy chain variable domain protein sequence |
| 295 | SCT-Ga365 mature heavy chain variable domain protein sequence |
| 296 | SCT-Ga366 mature heavy chain variable domain protein sequence |
| 297 | SCT-Ga368 mature heavy chain variable domain protein sequence |
| 298 | SCT-Ga369 mature heavy chain variable domain protein sequence |
| 299 | SCT-Ga370 mature heavy chain variable domain protein sequence |
| 300 | SCT-Ga371 mature heavy chain variable domain protein sequence |
| 301 | SCT-Ga372 mature heavy chain variable domain protein sequence |
| 302 | SCT-Ga374 mature heavy chain variable domain protein sequence |
| 303 | SCT-Ga376 mature heavy chain variable domain protein sequence |
| 304 | SCT-Ga377 mature heavy chain variable domain protein sequence |
| 305 | SCT-Ga378 mature heavy chain variable domain protein sequence |
| 306 | SCT-Ga379 mature heavy chain variable domain protein sequence |
| 307 | SCT-Ga380 mature heavy chain variable domain protein sequence |
| 308 | SCT-Ga381 mature heavy chain variable domain protein sequence |
| 309 | SCT-Ga382 mature heavy chain variable domain protein sequence |
| 310 | SCT-Ga383 mature heavy chain variable domain protein sequence |
| 311 | SCT-Ga384 mature heavy chain variable domain protein sequence |
| 311 | SCT-Ga390 mature heavy chain variable domain protein sequence |
| 312 | SCT-Ga385 mature heavy chain variable domain protein sequence |
| 313 | SCT-Ga386 mature heavy chain variable domain protein sequence |
| 313 | SCT-Ga402 mature heavy chain variable domain protein sequence |
| 314 | SCT-Ga387 mature heavy chain variable domain protein sequence |
| 315 | SCT-Ga388 mature heavy chain variable domain protein sequence |
| 315 | SCT-Ga398 mature heavy chain variable domain protein sequence |
| 316 | SCT-Ga389 mature heavy chain variable domain protein sequence |
| 317 | SCT-Ga391 mature heavy chain variable domain protein sequence |
| 318 | SCT-Ga392 mature heavy chain variable domain protein sequence |
| 319 | SCT-Ga393 mature heavy chain variable domain protein sequence |
| 319 | SCT-Ga438 mature heavy chain variable domain protein sequence |
| 320 | SCT-Ga394 mature heavy chain variable domain protein sequence |
| 321 | SCT-Ga395 mature heavy chain variable domain protein sequence |
| 322 | SCT-Ga396 mature heavy chain variable domain protein sequence |
| 323 | SCT-Ga397 mature heavy chain variable domain protein sequence |
| 324 | SCT-Ga399 mature heavy chain variable domain protein sequence |
| 325 | SCT-Ga400 mature heavy chain variable domain protein sequence |
| 326 | SCT-Ga401 mature heavy chain variable domain protein sequence |
| 327 | SCT-Ga403 mature heavy chain variable domain protein sequence |
| 328 | SCT-Ga404 mature heavy chain variable domain protein sequence |
| 329 | SCT-Ga405 mature heavy chain variable domain protein sequence |
| 330 | SCT-Ga406 mature heavy chain variable domain protein sequence |
| 331 | SCT-Ga407 mature heavy chain variable domain protein sequence |
| 332 | SCT-Ga408 mature heavy chain variable domain protein sequence |
| 333 | SCT-Ga409 mature heavy chain variable domain protein sequence |
| 334 | SCT-Ga410 mature heavy chain variable domain protein sequence |
| 335 | SCT-Ga411 mature heavy chain variable domain protein sequence |
| 336 | SCT-Ga412 mature heavy chain variable domain protein sequence |
| 337 | SCT-Ga413 mature heavy chain variable domain protein sequence |
| 338 | SCT-Ga414 mature heavy chain variable domain protein sequence |
| 339 | SCT-Ga415 mature heavy chain variable domain protein sequence |
| 340 | SCT-Ga416 mature heavy chain variable domain protein sequence |
| 341 | SCT-Ga417 mature heavy chain variable domain protein sequence |
| 342 | SCT-Ga418 mature heavy chain variable domain protein sequence |
| 343 | SCT-Ga419 mature heavy chain variable domain protein sequence |
| 344 | SCT-Ga420 mature heavy chain variable domain protein sequence |
| 345 | SCT-Ga421 mature heavy chain variable domain protein sequence |
| 346 | SCT-Ga422 mature heavy chain variable domain protein sequence |
| 347 | SCT-Ga423 mature heavy chain variable domain protein sequence |
| 348 | SCT-Ga424 mature heavy chain variable domain protein sequence |
| 349 | SCT-Ga425 mature heavy chain variable domain protein sequence |
| 350 | SCT-Ga426 mature heavy chain variable domain protein sequence |
| 351 | SCT-Ga427 mature heavy chain variable domain protein sequence |
| 352 | SCT-Ga428 mature heavy chain variable domain protein sequence |
| 353 | SCT-Ga429 mature heavy chain variable domain protein sequence |
| 354 | SCT-Ga430 mature heavy chain variable domain protein sequence |
| 355 | SCT-Ga431 mature heavy chain variable domain protein sequence |
| 356 | SCT-Ga432 mature heavy chain variable domain protein sequence |
| 357 | SCT-Ga433 mature heavy chain variable domain protein sequence |
| 358 | SCT-Ga434 mature heavy chain variable domain protein sequence |
| 359 | SCT-Ga435 mature heavy chain variable domain protein sequence |
| 360 | SCT-Ga436 mature heavy chain variable domain protein sequence |
| 361 | SCT-Ga437 mature heavy chain variable domain protein sequence |
| 362 | SCT-Ga439 mature heavy chain variable domain protein sequence |
| 363 | SCT-Ga440 mature heavy chain variable domain protein sequence |
| 364 | SCT-Ga441 mature heavy chain variable domain protein sequence |
| 365 | SCT-Ga442 mature heavy chain variable domain protein sequence |
| 366 | SCT-Ga443 mature heavy chain variable domain protein sequence |
| 367 | SCT-Ga444 mature heavy chain variable domain protein sequence |
| 368 | SCT-Ga445 mature heavy chain variable domain protein sequence |
| 369 | SCT-Ga446 mature heavy chain variable domain protein sequence |
| 370 | SCT-Ga447 mature heavy chain variable domain protein sequence |
| 371 | SCT-Ga448 mature heavy chain variable domain protein sequence |
| 372 | SCT-Ga449 mature heavy chain variable domain protein sequence |
| 373 | SCT-Ga450 mature heavy chain variable domain protein sequence |
| 374 | SCT-Ga451 mature heavy chain variable domain protein sequence |
| 375 | SCT-Ga452 mature heavy chain variable domain protein sequence |
| 376 | SCT-Ga453 mature heavy chain variable domain protein sequence |
| 377 | SCT-Ga454 mature heavy chain variable domain protein sequence |
| 378 | SCT-Ga455 mature heavy chain variable domain protein sequence |
| 379 | SCT-Ga456 mature heavy chain variable domain protein sequence |
| 380 | SCT-Ga001 mature light chain variable domain protein sequence |
| 380 | SCT-Ga038 mature light chain variable domain protein sequence |
| 381 | SCT-Ga002 mature light chain variable domain protein sequence |
| 381 | SCT-Ga003 mature light chain variable domain protein sequence |
| 381 | SCT-Ga005 mature light chain variable domain protein sequence |
| 381 | SCT-Ga006 mature light chain variable domain protein sequence |
| 381 | SCT-Ga008 mature light chain variable domain protein sequence |
| 381 | SCT-Ga011 mature light chain variable domain protein sequence |
| 381 | SCT-Ga013 mature light chain variable domain protein sequence |
| 381 | SCT-Ga014 mature light chain variable domain protein sequence |
| 381 | SCT-Ga018 mature light chain variable domain protein sequence |
| 381 | SCT-Ga020 mature light chain variable domain protein sequence |
| 381 | SCT-Ga022 mature light chain variable domain protein sequence |
| 381 | SCT-Ga023 mature light chain variable domain protein sequence |
| 381 | SCT-Ga025 mature light chain variable domain protein sequence |
| 381 | SCT-Ga030 mature light chain variable domain protein sequence |
| 381 | SCT-Ga032 mature light chain variable domain protein sequence |
| 381 | SCT-Ga047 mature light chain variable domain protein sequence |
| 381 | SCT-Ga058 mature light chain variable domain protein sequence |
| 381 | SCT-Ga060 mature light chain variable domain protein sequence |
| 381 | SCT-Ga061 mature light chain variable domain protein sequence |
| 381 | SCT-Ga064 mature light chain variable domain protein sequence |
| 381 | SCT-Ga070 mature light chain variable domain protein sequence |
| 381 | SCT-Ga072 mature light chain variable domain protein sequence |
| 381 | SCT-Ga078 mature light chain variable domain protein sequence |
| 381 | SCT-Ga079 mature light chain variable domain protein sequence |
| 381 | SCT-Ga080 mature light chain variable domain protein sequence |
| 381 | SCT-Ga081 mature light chain variable domain protein sequence |
| 381 | SCT-Ga082 mature light chain variable domain protein sequence |
| 381 | SCT-Ga083 mature light chain variable domain protein sequence |
| 381 | SCT-Ga085 mature light chain variable domain protein sequence |
| 381 | SCT-Ga086 mature light chain variable domain protein sequence |
| 381 | SCT-Ga090 mature light chain variable domain protein sequence |
| 381 | SCT-Ga091 mature light chain variable domain protein sequence |
| 381 | SCT-Ga094 mature light chain variable domain protein sequence |
| 381 | SCT-Ga097 mature light chain variable domain protein sequence |
| 381 | SCT-Ga098 mature light chain variable domain protein sequence |
| 381 | SCT-Ga107 mature light chain variable domain protein sequence |
| 381 | SCT-Ga108 mature light chain variable domain protein sequence |
| 381 | SCT-Ga109 mature light chain variable domain protein sequence |
| 381 | SCT-Ga114 mature light chain variable domain protein sequence |
| 381 | SCT-Ga116 mature light chain variable domain protein sequence |
| 381 | SCT-Ga117 mature light chain variable domain protein sequence |
| 381 | SCT-Ga118 mature light chain variable domain protein sequence |
| 381 | SCT-Ga119 mature light chain variable domain protein sequence |
| 381 | SCT-Ga128 mature light chain variable domain protein sequence |
| 381 | SCT-Ga129 mature light chain variable domain protein sequence |
| 381 | SCT-Ga130 mature light chain variable domain protein sequence |
| 381 | SCT-Ga138 mature light chain variable domain protein sequence |
| 381 | SCT-Ga144 mature light chain variable domain protein sequence |
| 381 | SCT-Ga145 mature light chain variable domain protein sequence |
| 381 | SCT-Ga151 mature light chain variable domain protein sequence |
| 381 | SCT-Ga153 mature light chain variable domain protein sequence |
| 381 | SCT-Ga155 mature light chain variable domain protein sequence |
| 381 | SCT-Ga160 mature light chain variable domain protein sequence |
| 381 | SCT-Ga163 mature light chain variable domain protein sequence |
| 381 | SCT-Ga164 mature light chain variable domain protein sequence |
| 381 | SCT-Ga165 mature light chain variable domain protein sequence |
| 381 | SCT-Ga168 mature light chain variable domain protein sequence |
| 381 | SCT-Ga170 mature light chain variable domain protein sequence |
| 381 | SCT-Ga171 mature light chain variable domain protein sequence |
| 381 | SCT-Ga172 mature light chain variable domain protein sequence |
| 381 | SCT-Ga437 mature light chain variable domain protein sequence |
| 381 | SCT-Ga449 mature light chain variable domain protein sequence |
| 382 | SCT-Ga004 mature light chain variable domain protein sequence |
| 382 | SCT-Ga044 mature light chain variable domain protein sequence |
| 382 | SCT-Ga050 mature light chain variable domain protein sequence |
| 382 | SCT-Ga124 mature light chain variable domain protein sequence |
| 382 | SCT-Ga166 mature light chain variable domain protein sequence |
| 383 | SCT-Ga007 mature light chain variable domain protein sequence |
| 384 | SCT-Ga009 mature light chain variable domain protein sequence |
| 385 | SCT-Ga010 mature light chain variable domain protein sequence |
| 385 | SCT-Ga068 mature light chain variable domain protein sequence |
| 385 | SCT-Ga084 mature light chain variable domain protein sequence |
| 385 | SCT-Ga089 mature light chain variable domain protein sequence |
| 385 | SCT-Ga123 mature light chain variable domain protein sequence |
| 385 | SCT-Ga135 mature light chain variable domain protein sequence |
| 385 | SCT-Ga139 mature light chain variable domain protein sequence |
| 386 | SCT-Ga012 mature light chain variable domain protein sequence |
| 387 | SCT-Ga015 mature light chain variable domain protein sequence |
| 388 | SCT-Ga016 mature light chain variable domain protein sequence |
| 389 | SCT-Ga017 mature light chain variable domain protein sequence |
| 389 | SCT-Ga150 mature light chain variable domain protein sequence |
| 390 | SCT-Ga019 mature light chain variable domain protein sequence |
| 391 | SCT-Ga021 mature light chain variable domain protein sequence |
| 392 | SCT-Ga024 mature light chain variable domain protein sequence |
| 393 | SCT-Ga026 mature light chain variable domain protein sequence |
| 394 | SCT-Ga027 mature light chain variable domain protein sequence |
| 395 | SCT-Ga028 mature light chain variable domain protein sequence |
| 396 | SCT-Ga029 mature light chain variable domain protein sequence |
| 396 | SCT-Ga167 mature light chain variable domain protein sequence |
| 397 | SCT-Ga031 mature light chain variable domain protein sequence |
| 398 | SCT-Ga033 mature light chain variable domain protein sequence |
| 398 | SCT-Ga040 mature light chain variable domain protein sequence |
| 398 | SCT-Ga046 mature light chain variable domain protein sequence |
| 398 | SCT-Ga111 mature light chain variable domain protein sequence |
| 398 | SCT-Ga143 mature light chain variable domain protein sequence |
| 398 | SCT-Ga158 mature light chain variable domain protein sequence |
| 398 | SCT-Ga162 mature light chain variable domain protein sequence |
| 398 | SCT-Ga173 mature light chain variable domain protein sequence |
| 399 | SCT-Ga034 mature light chain variable domain protein sequence |
| 399 | SCT-Ga048 mature light chain variable domain protein sequence |
| 400 | SCT-Ga035 mature light chain variable domain protein sequence |
| 401 | SCT-Ga036 mature light chain variable domain protein sequence |
| 401 | SCT-Ga067 mature light chain variable domain protein sequence |
| 402 | SCT-Ga037 mature light chain variable domain protein sequence |
| 403 | SCT-Ga039 mature light chain variable domain protein sequence |
| 404 | SCT-Ga041 mature light chain variable domain protein sequence |
| 404 | SCT-Ga126 mature light chain variable domain protein sequence |
| 405 | SCT-Ga042 mature light chain variable domain protein sequence |
| 406 | SCT-Ga043 mature light chain variable domain protein sequence |
| 406 | SCT-Ga045 mature light chain variable domain protein sequence |
| 406 | SCT-Ga148 mature light chain variable domain protein sequence |
| 407 | SCT-Ga049 mature light chain variable domain protein sequence |
| 408 | SCT-Ga051 mature light chain variable domain protein sequence |
| 409 | SCT-Ga052 mature light chain variable domain protein sequence |
| 410 | SCT-Ga053 mature light chain variable domain protein sequence |
| 410 | SCT-Ga127 mature light chain variable domain protein sequence |
| 411 | SCT-Ga054 mature light chain variable domain protein sequence |
| 412 | SCT-Ga055 mature light chain variable domain protein sequence |
| 413 | SCT-Ga056 mature light chain variable domain protein sequence |
| 414 | SCT-Ga057 mature light chain variable domain protein sequence |
| 415 | SCT-Ga059 mature light chain variable domain protein sequence |
| 416 | SCT-Ga062 mature light chain variable domain protein sequence |
| 417 | SCT-Ga063 mature light chain variable domain protein sequence |
| 418 | SCT-Ga065 mature light chain variable domain protein sequence |
| 419 | SCT-Ga066 mature light chain variable domain protein sequence |
| 420 | SCT-Ga069 mature light chain variable domain protein sequence |
| 421 | SCT-Ga071 mature light chain variable domain protein sequence |
| 422 | SCT-Ga073 mature light chain variable domain protein sequence |
| 423 | SCT-Ga074 mature light chain variable domain protein sequence |
| 424 | SCT-Ga075 mature light chain variable domain protein sequence |
| 425 | SCT-Ga076 mature light chain variable domain protein sequence |
| 426 | SCT-Ga077 mature light chain variable domain protein sequence |
| 427 | SCT-Ga087 mature light chain variable domain protein sequence |
| 428 | SCT-Ga088 mature light chain variable domain protein sequence |
| 429 | SCT-Ga092 mature light chain variable domain protein sequence |
| 430 | SCT-Ga093 mature light chain variable domain protein sequence |
| 431 | SCT-Ga095 mature light chain variable domain protein sequence |
| 432 | SCT-Ga096 mature light chain variable domain protein sequence |
| 433 | SCT-Ga099 mature light chain variable domain protein sequence |
| 434 | SCT-Ga100 mature light chain variable domain protein sequence |
| 435 | SCT-Ga101 mature light chain variable domain protein sequence |
| 436 | SCT-Ga102 mature light chain variable domain protein sequence |
| 437 | SCT-Ga103 mature light chain variable domain protein sequence |
| 438 | SCT-Ga104 mature light chain variable domain protein sequence |
| 439 | SCT-Ga105 mature light chain variable domain protein sequence |
| 440 | SCT-Ga106 mature light chain variable domain protein sequence |
| 441 | SCT-Ga110 mature light chain variable domain protein sequence |
| 442 | SCT-Ga112 mature light chain variable domain protein sequence |
| 443 | SCT-Ga113 mature light chain variable domain protein sequence |
| 444 | SCT-Ga115 mature light chain variable domain protein sequence |
| 445 | SCT-Ga120 mature light chain variable domain protein sequence |
| 445 | SCT-Ga154 mature light chain variable domain protein sequence |
| 446 | SCT-Ga121 mature light chain variable domain protein sequence |
| 447 | SCT-Ga122 mature light chain variable domain protein sequence |
| 448 | SCT-Ga125 mature light chain variable domain protein sequence |
| 449 | SCT-Ga131 mature light chain variable domain protein sequence |
| 450 | SCT-Ga132 mature light chain variable domain protein sequence |
| 451 | SCT-Ga133 mature light chain variable domain protein sequence |
| 451 | SCT-Ga149 mature light chain variable domain protein sequence |
| 451 | SCT-Ga152 mature light chain variable domain protein sequence |
| 452 | SCT-Ga134 mature light chain variable domain protein sequence |
| 453 | SCT-Ga136 mature light chain variable domain protein sequence |
| 454 | SCT-Ga137 mature light chain variable domain protein sequence |
| 455 | SCT-Ga140 mature light chain variable domain protein sequence |
| 456 | SCT-Ga141 mature light chain variable domain protein sequence |
| 456 | SCT-Ga142 mature light chain variable domain protein sequence |
| 457 | SCT-Ga146 mature light chain variable domain protein sequence |
| 458 | SCT-Ga147 mature light chain variable domain protein sequence |
| 459 | SCT-Ga156 mature light chain variable domain protein sequence |
| 460 | SCT-Ga157 mature light chain variable domain protein sequence |
| 461 | SCT-Ga159 mature light chain variable domain protein sequence |
| 462 | SCT-Ga161 mature light chain variable domain protein sequence |
| 462 | SCT-Ga174 mature light chain variable domain protein sequence |
| 463 | SCT-Ga169 mature light chain variable domain protein sequence |
| 464 | SCT-Ga175 mature light chain variable domain protein sequence |
| 465 | SCT-Ga176 mature light chain variable domain protein sequence |
| 466 | SCT-Ga177 mature light chain variable domain protein sequence |
| 466 | SCT-Ga281 mature light chain variable domain protein sequence |
| 466 | SCT-Ga342 mature light chain variable domain protein sequence |
| 466 | SCT-Ga370 mature light chain variable domain protein sequence |
| 467 | SCT-Ga178 mature light chain variable domain protein sequence |
| 468 | SCT-Ga179 mature light chain variable domain protein sequence |
| 469 | SCT-Ga180 mature light chain variable domain protein sequence |
| 469 | SCT-Ga222 mature light chain variable domain protein sequence |
| 469 | SCT-Ga267 mature light chain variable domain protein sequence |
| 469 | SCT-Ga336 mature light chain variable domain protein sequence |
| 470 | SCT-Ga181 mature light chain variable domain protein sequence |
| 471 | SCT-Ga182 mature light chain variable domain protein sequence |
| 472 | SCT-Ga183 mature light chain variable domain protein sequence |
| 473 | SCT-Ga184 mature light chain variable domain protein sequence |
| 474 | SCT-Ga185 mature light chain variable domain protein sequence |
| 474 | SCT-Ga202 mature light chain variable domain protein sequence |
| 475 | SCT-Ga186 mature light chain variable domain protein sequence |
| 476 | SCT-Ga187 mature light chain variable domain protein sequence |
| 477 | SCT-Ga188 mature light chain variable domain protein sequence |
| 478 | SCT-Ga189 mature light chain variable domain protein sequence |
| 479 | SCT-Ga190 mature light chain variable domain protein sequence |
| 479 | SCT-Ga214 mature light chain variable domain protein sequence |
| 480 | SCT-Ga191 mature light chain variable domain protein sequence |
| 481 | SCT-Ga192 mature light chain variable domain protein sequence |
| 482 | SCT-Ga193 mature light chain variable domain protein sequence |
| 482 | SCT-Ga262 mature light chain variable domain protein sequence |
| 482 | SCT-Ga303 mature light chain variable domain protein sequence |
| 482 | SCT-Ga313 mature light chain variable domain protein sequence |
| 482 | SCT-Ga346 mature light chain variable domain protein sequence |
| 482 | SCT-Ga401 mature light chain variable domain protein sequence |
| 482 | SCT-Ga431 mature light chain variable domain protein sequence |
| 482 | SCT-Ga440 mature light chain variable domain protein sequence |
| 482 | SCT-Ga453 mature light chain variable domain protein sequence |
| 483 | SCT-Ga194 mature light chain variable domain protein sequence |
| 483 | SCT-Ga421 mature light chain variable domain protein sequence |
| 484 | SCT-Ga195 mature light chain variable domain protein sequence |
| 485 | SCT-Ga196 mature light chain variable domain protein sequence |
| 486 | SCT-Ga197 mature light chain variable domain protein sequence |
| 486 | SCT-Ga211 mature light chain variable domain protein sequence |
| 487 | SCT-Ga198 mature light chain variable domain protein sequence |
| 488 | SCT-Ga199 mature light chain variable domain protein sequence |
| 489 | SCT-Ga200 mature light chain variable domain protein sequence |
| 490 | SCT-Ga201 mature light chain variable domain protein sequence |
| 491 | SCT-Ga203 mature light chain variable domain protein sequence |
| 492 | SCT-Ga204 mature light chain variable domain protein sequence |
| 493 | SCT-Ga205 mature light chain variable domain protein sequence |
| 494 | SCT-Ga206 mature light chain variable domain protein sequence |
| 495 | SCT-Ga207 mature light chain variable domain protein sequence |
| 496 | SCT-Ga208 mature light chain variable domain protein sequence |
| 497 | SCT-Ga209 mature light chain variable domain protein sequence |
| 498 | SCT-Ga210 mature light chain variable domain protein sequence |
| 499 | SCT-Ga212 mature light chain variable domain protein sequence |
| 500 | SCT-Ga213 mature light chain variable domain protein sequence |
| 501 | SCT-Ga215 mature light chain variable domain protein sequence |
| 502 | SCT-Ga216 mature light chain variable domain protein sequence |
| 503 | SCT-Ga217 mature light chain variable domain protein sequence |
| 504 | SCT-Ga218 mature light chain variable domain protein sequence |
| 505 | SCT-Ga219 mature light chain variable domain protein sequence |
| 506 | SCT-Ga220 mature light chain variable domain protein sequence |
| 506 | SCT-Ga331 mature light chain variable domain protein sequence |
| 507 | SCT-Ga221 mature light chain variable domain protein sequence |
| 508 | SCT-Ga223 mature light chain variable domain protein sequence |
| 509 | SCT-Ga224 mature light chain variable domain protein sequence |
| 510 | SCT-Ga225 mature light chain variable domain protein sequence |
| 511 | SCT-Ga226 mature light chain variable domain protein sequence |
| 512 | SCT-Ga227 mature light chain variable domain protein sequence |
| 512 | SCT-Ga365 mature light chain variable domain protein sequence |
| 513 | SCT-Ga228 mature light chain variable domain protein sequence |
| 514 | SCT-Ga229 mature light chain variable domain protein sequence |
| 515 | SCT-Ga230 mature light chain variable domain protein sequence |
| 515 | SCT-Ga258 mature light chain variable domain protein sequence |
| 515 | SCT-Ga329 mature light chain variable domain protein sequence |
| 516 | SCT-Ga231 mature light chain variable domain protein sequence |
| 517 | SCT-Ga232 mature light chain variable domain protein sequence |
| 517 | SCT-Ga292 mature light chain variable domain protein sequence |
| 518 | SCT-Ga233 mature light chain variable domain protein sequence |
| 519 | SCT-Ga234 mature light chain variable domain protein sequence |
| 520 | SCT-Ga235 mature light chain variable domain protein sequence |
| 521 | SCT-Ga236 mature light chain variable domain protein sequence |
| 522 | SCT-Ga237 mature light chain variable domain protein sequence |
| 523 | SCT-Ga238 mature light chain variable domain protein sequence |
| 524 | SCT-Ga239 mature light chain variable domain protein sequence |
| 525 | SCT-Ga240 mature light chain variable domain protein sequence |
| 526 | SCT-Ga241 mature light chain variable domain protein sequence |
| 527 | SCT-Ga242 mature light chain variable domain protein sequence |
| 527 | SCT-Ga260 mature light chain variable domain protein sequence |
| 528 | SCT-Ga243 mature light chain variable domain protein sequence |
| 529 | SCT-Ga244 mature light chain variable domain protein sequence |
| 529 | SCT-Ga272 mature light chain variable domain protein sequence |
| 529 | SCT-Ga362 mature light chain variable domain protein sequence |
| 530 | SCT-Ga245 mature light chain variable domain protein sequence |
| 531 | SCT-Ga246 mature light chain variable domain protein sequence |
| 531 | SCT-Ga309 mature light chain variable domain protein sequence |
| 531 | SCT-Ga375 mature light chain variable domain protein sequence |
| 532 | SCT-Ga247 mature light chain variable domain protein sequence |
| 533 | SCT-Ga248 mature light chain variable domain protein sequence |
| 534 | SCT-Ga249 mature light chain variable domain protein sequence |
| 535 | SCT-Ga250 mature light chain variable domain protein sequence |
| 536 | SCT-Ga251 mature light chain variable domain protein sequence |
| 537 | SCT-Ga252 mature light chain variable domain protein sequence |
| 538 | SCT-Ga253 mature light chain variable domain protein sequence |
| 539 | SCT-Ga254 mature light chain variable domain protein sequence |
| 540 | SCT-Ga255 mature light chain variable domain protein sequence |
| 540 | SCT-Ga301 mature light chain variable domain protein sequence |
| 541 | SCT-Ga256 mature light chain variable domain protein sequence |
| 542 | SCT-Ga257 mature light chain variable domain protein sequence |
| 543 | SCT-Ga259 mature light chain variable domain protein sequence |
| 544 | SCT-Ga261 mature light chain variable domain protein sequence |
| 545 | SCT-Ga263 mature light chain variable domain protein sequence |
| 546 | SCT-Ga264 mature light chain variable domain protein sequence |
| 547 | SCT-Ga265 mature light chain variable domain protein sequence |
| 548 | SCT-Ga266 mature light chain variable domain protein sequence |
| 549 | SCT-Ga268 mature light chain variable domain protein sequence |
| 550 | SCT-Ga269 mature light chain variable domain protein sequence |
| 551 | SCT-Ga270 mature light chain variable domain protein sequence |
| 552 | SCT-Ga271 mature light chain variable domain protein sequence |
| 552 | SCT-Ga276 mature light chain variable domain protein sequence |
| 552 | SCT-Ga334 mature light chain variable domain protein sequence |
| 552 | SCT-Ga384 mature light chain variable domain protein sequence |
| 552 | SCT-Ga394 mature light chain variable domain protein sequence |
| 552 | SCT-Ga414 mature light chain variable domain protein sequence |
| 553 | SCT-Ga273 mature light chain variable domain protein sequence |
| 554 | SCT-Ga274 mature light chain variable domain protein sequence |
| 555 | SCT-Ga275 mature light chain variable domain protein sequence |
| 556 | SCT-Ga277 mature light chain variable domain protein sequence |
| 557 | SCT-Ga278 mature light chain variable domain protein sequence |
| 558 | SCT-Ga279 mature light chain variable domain protein sequence |
| 559 | SCT-Ga280 mature light chain variable domain protein sequence |
| 560 | SCT-Ga282 mature light chain variable domain protein sequence |
| 561 | SCT-Ga283 mature light chain variable domain protein sequence |
| 562 | SCT-Ga284 mature light chain variable domain protein sequence |
| 563 | SCT-Ga285 mature light chain variable domain protein sequence |
| 564 | SCT-Ga286 mature light chain variable domain protein sequence |
| 565 | SCT-Ga287 mature light chain variable domain protein sequence |
| 566 | SCT-Ga288 mature light chain variable domain protein sequence |
| 567 | SCT-Ga289 mature light chain variable domain protein sequence |
| 568 | SCT-Ga290 mature light chain variable domain protein sequence |
| 569 | SCT-Ga291 mature light chain variable domain protein sequence |
| 570 | SCT-Ga293 mature light chain variable domain protein sequence |
| 571 | SCT-Ga294 mature light chain variable domain protein sequence |
| 572 | SCT-Ga295 mature light chain variable domain protein sequence |
| 572 | SCT-Ga360 mature light chain variable domain protein sequence |
| 572 | SCT-Ga406 mature light chain variable domain protein sequence |
| 572 | SCT-Ga419 mature light chain variable domain protein sequence |
| 572 | SCT-Ga424 mature light chain variable domain protein sequence |
| 572 | SCT-Ga435 mature light chain variable domain protein sequence |
| 572 | SCT-Ga455 mature light chain variable domain protein sequence |
| 573 | SCT-Ga296 mature light chain variable domain protein sequence |
| 574 | SCT-Ga297 mature light chain variable domain protein sequence |
| 575 | SCT-Ga298 mature light chain variable domain protein sequence |
| 576 | SCT-Ga299 mature light chain variable domain protein sequence |
| 577 | SCT-Ga300 mature light chain variable domain protein sequence |
| 578 | SCT-Ga302 mature light chain variable domain protein sequence |
| 579 | SCT-Ga304 mature light chain variable domain protein sequence |
| 580 | SCT-Ga305 mature light chain variable domain protein sequence |
| 580 | SCT-Ga372 mature light chain variable domain protein sequence |
| 581 | SCT-Ga306 mature light chain variable domain protein sequence |
| 582 | SCT-Ga307 mature light chain variable domain protein sequence |
| 583 | SCT-Ga308 mature light chain variable domain protein sequence |
| 584 | SCT-Ga310 mature light chain variable domain protein sequence |
| 585 | SCT-Ga311 mature light chain variable domain protein sequence |
| 586 | SCT-Ga312 mature light chain variable domain protein sequence |
| 587 | SCT-Ga314 mature light chain variable domain protein sequence |
| 588 | SCT-Ga315 mature light chain variable domain protein sequence |
| 589 | SCT-Ga316 mature light chain variable domain protein sequence |
| 590 | SCT-Ga317 mature light chain variable domain protein sequence |
| 591 | SCT-Ga318 mature light chain variable domain protein sequence |
| 592 | SCT-Ga319 mature light chain variable domain protein sequence |
| 593 | SCT-Ga320 mature light chain variable domain protein sequence |
| 594 | SCT-Ga321 mature light chain variable domain protein sequence |
| 595 | SCT-Ga322 mature light chain variable domain protein sequence |
| 596 | SCT-Ga323 mature light chain variable domain protein sequence |
| 596 | SCT-Ga324 mature light chain variable domain protein sequence |
| 596 | SCT-Ga339 mature light chain variable domain protein sequence |
| 596 | SCT-Ga380 mature light chain variable domain protein sequence |
| 597 | SCT-Ga325 mature light chain variable domain protein sequence |
| 598 | SCT-Ga326 mature light chain variable domain protein sequence |
| 599 | SCT-Ga327 mature light chain variable domain protein sequence |
| 600 | SCT-Ga328 mature light chain variable domain protein sequence |
| 601 | SCT-Ga330 mature light chain variable domain protein sequence |
| 602 | SCT-Ga332 mature light chain variable domain protein sequence |
| 603 | SCT-Ga333 mature light chain variable domain protein sequence |
| 604 | SCT-Ga335 mature light chain variable domain protein sequence |
| 605 | SCT-Ga337 mature light chain variable domain protein sequence |
| 606 | SCT-Ga338 mature light chain variable domain protein sequence |
| 607 | SCT-Ga340 mature light chain variable domain protein sequence |
| 608 | SCT-Ga341 mature light chain variable domain protein sequence |
| 608 | SCT-Ga351 mature light chain variable domain protein sequence |
| 609 | SCT-Ga343 mature light chain variable domain protein sequence |
| 610 | SCT-Ga344 mature light chain variable domain protein sequence |
| 611 | SCT-Ga345 mature light chain variable domain protein sequence |
| 612 | SCT-Ga347 mature light chain variable domain protein sequence |
| 612 | SCT-Ga377 mature light chain variable domain protein sequence |
| 613 | SCT-Ga348 mature light chain variable domain protein sequence |
| 614 | SCT-Ga349 mature light chain variable domain protein sequence |
| 615 | SCT-Ga350 mature light chain variable domain protein sequence |
| 616 | SCT-Ga352 mature light chain variable domain protein sequence |
| 617 | SCT-Ga353 mature light chain variable domain protein sequence |
| 618 | SCT-Ga354 mature light chain variable domain protein sequence |
| 619 | SCT-Ga355 mature light chain variable domain protein sequence |
| 620 | SCT-Ga356 mature light chain variable domain protein sequence |
| 621 | SCT-Ga357 mature light chain variable domain protein sequence |
| 622 | SCT-Ga358 mature light chain variable domain protein sequence |
| 623 | SCT-Ga359 mature light chain variable domain protein sequence |
| 624 | SCT-Ga361 mature light chain variable domain protein sequence |
| 625 | SCT-Ga363 mature light chain variable domain protein sequence |
| 626 | SCT-Ga364 mature light chain variable domain protein sequence |
| 627 | SCT-Ga366 mature light chain variable domain protein sequence |
| 628 | SCT-Ga367 mature light chain variable domain protein sequence |
| 629 | SCT-Ga368 mature light chain variable domain protein sequence |
| 630 | SCT-Ga369 mature light chain variable domain protein sequence |
| 631 | SCT-Ga371 mature light chain variable domain protein sequence |
| 632 | SCT-Ga373 mature light chain variable domain protein sequence |
| 633 | SCT-Ga374 mature light chain variable domain protein sequence |
| 634 | SCT-Ga376 mature light chain variable domain protein sequence |
| 635 | SCT-Ga378 mature light chain variable domain protein sequence |
| 636 | SCT-Ga379 mature light chain variable domain protein sequence |
| 637 | SCT-Ga381 mature light chain variable domain protein sequence |
| 638 | SCT-Ga382 mature light chain variable domain protein sequence |
| 639 | SCT-Ga383 mature light chain variable domain protein sequence |
| 640 | SCT-Ga385 mature light chain variable domain protein sequence |
| 641 | SCT-Ga386 mature light chain variable domain protein sequence |
| 642 | SCT-Ga387 mature light chain variable domain protein sequence |
| 643 | SCT-Ga388 mature light chain variable domain protein sequence |
| 643 | SCT-Ga413 mature light chain variable domain protein sequence |
| 643 | SCT-Ga426 mature light chain variable domain protein sequence |
| 644 | SCT-Ga389 mature light chain variable domain protein sequence |
| 645 | SCT-Ga390 mature light chain variable domain protein sequence |
| 645 | SCT-Ga391 mature light chain variable domain protein sequence |
| 645 | SCT-Ga402 mature light chain variable domain protein sequence |
| 645 | SCT-Ga417 mature light chain variable domain protein sequence |
| 645 | SCT-Ga441 mature light chain variable domain protein sequence |
| 645 | SCT-Ga447 mature light chain variable domain protein sequence |
| 646 | SCT-Ga392 mature light chain variable domain protein sequence |
| 647 | SCT-Ga393 mature light chain variable domain protein sequence |
| 648 | SCT-Ga395 mature light chain variable domain protein sequence |
| 649 | SCT-Ga396 mature light chain variable domain protein sequence |
| 650 | SCT-Ga397 mature light chain variable domain protein sequence |
| 651 | SCT-Ga398 mature light chain variable domain protein sequence |
| 652 | SCT-Ga399 mature light chain variable domain protein sequence |
| 652 | SCT-Ga408 mature light chain variable domain protein sequence |
| 653 | SCT-Ga400 mature light chain variable domain protein sequence |
| 654 | SCT-Ga403 mature light chain variable domain protein sequence |
| 655 | SCT-Ga404 mature light chain variable domain protein sequence |
| 656 | SCT-Ga405 mature light chain variable domain protein sequence |
| 657 | SCT-Ga407 mature light chain variable domain protein sequence |
| 658 | SCT-Ga409 mature light chain variable domain protein sequence |
| 658 | SCT-Ga434 mature light chain variable domain protein sequence |
| 659 | SCT-Ga410 mature light chain variable domain protein sequence |
| 660 | SCT-Ga411 mature light chain variable domain protein sequence |
| 661 | SCT-Ga412 mature light chain variable domain protein sequence |
| 662 | SCT-Ga415 mature light chain variable domain protein sequence |
| 663 | SCT-Ga416 mature light chain variable domain protein sequence |
| 664 | SCT-Ga418 mature light chain variable domain protein sequence |
| 665 | SCT-Ga420 mature light chain variable domain protein sequence |
| 666 | SCT-Ga422 mature light chain variable domain protein sequence |
| 667 | SCT-Ga423 mature light chain variable domain protein sequence |
| 667 | SCT-Ga456 mature light chain variable domain protein sequence |
| 668 | SCT-Ga425 mature light chain variable domain protein sequence |
| 669 | SCT-Ga427 mature light chain variable domain protein sequence |
| 669 | SCT-Ga428 mature light chain variable domain protein sequence |
| 670 | SCT-Ga429 mature light chain variable domain protein sequence |
| 671 | SCT-Ga430 mature light chain variable domain protein sequence |
| 672 | SCT-Ga432 mature light chain variable domain protein sequence |
| 673 | SCT-Ga433 mature light chain variable domain protein sequence |
| 674 | SCT-Ga436 mature light chain variable domain protein sequence |
| 675 | SCT-Ga438 mature light chain variable domain protein sequence |
| 676 | SCT-Ga439 mature light chain variable domain protein sequence |
| 677 | SCT-Ga442 mature light chain variable domain protein sequence |
| 678 | SCT-Ga443 mature light chain variable domain protein sequence |
| 679 | SCT-Ga444 mature light chain variable domain protein sequence |
| 680 | SCT-Ga445 mature light chain variable domain protein sequence |
| 681 | SCT-Ga446 mature light chain variable domain protein sequence |
| 682 | SCT-Ga448 mature light chain variable domain protein sequence |
| 683 | SCT-Ga450 mature light chain variable domain protein sequence |
| 684 | SCT-Ga451 mature light chain variable domain protein sequence |
| 685 | SCT-Ga452 mature light chain variable domain protein sequence |
| 686 | SCT-Ga454 mature light chain variable domain protein sequence |
| 1366 | SCT-Ga457 mature heavy chain variable domain protein sequence |
| 1366 | SCT-Ga458 mature heavy chain variable domain protein sequence |
| 1367 | SCT-Ga457 mature light chain variable domain protein sequence |
| 1368 | SCT-Ga458 mature light chain variable domain protein sequence |
| 1369 | SCT-Ga459 mature heavy chain variable domain protein sequence |
| 1369 | SCT-Ga460 mature heavy chain variable domain protein sequence |
| 1370 | SCT-Ga459 mature light chain variable domain protein sequence |
| 1370 | SCT-Ga461 mature light chain variable domain protein sequence |
| 1371 | SCT-Ga460 mature light chain variable domain protein sequence |
| 1371 | SCT-Ga462 mature light chain variable domain protein sequence |
| 1372 | SCT-Ga461 mature heavy chain variable domain protein sequence |
| 1372 | SCT-Ga462 mature heavy chain variable domain protein sequence |
| 1373 | SCT-Ga463 mature heavy chain variable domain protein sequence |
| 1373 | SCT-Ga464 mature heavy chain variable domain protein sequence |
| 1374 | SCT-Ga463 mature light chain variable domain protein sequence |
| 1374 | SCT-Ga465 mature light chain variable domain protein sequence |
| 1374 | SCT-Ga493 mature light chain variable domain protein sequence |
| 1374 | SCT-Ga495 mature light chain variable domain protein sequence |
| 1375 | SCT-Ga464 mature light chain variable domain protein sequence |
| 1375 | SCT-Ga466 mature light chain variable domain protein sequence |
| 1376 | SCT-Ga465 mature heavy chain variable domain protein sequence |
| 1376 | SCT-Ga466 mature heavy chain variable domain protein sequence |
| 1377 | SCT-Ga467 mature heavy chain variable domain protein sequence |
| 1377 | SCT-Ga468 mature heavy chain variable domain protein sequence |
| 1378 | SCT-Ga467 mature light chain variable domain protein sequence |
| 1378 | SCT-Ga469 mature light chain variable domain protein sequence |
| 1379 | SCT-Ga468 mature light chain variable domain protein sequence |
| 1379 | SCT-Ga470 mature light chain variable domain protein sequence |
| 1380 | SCT-Ga469 mature heavy chain variable domain protein sequence |
| 1380 | SCT-Ga470 mature heavy chain variable domain protein sequence |
| 1381 | SCT-Ga471 mature heavy chain variable domain protein sequence |
| 1381 | SCT-Ga472 mature heavy chain variable domain protein sequence |
| 1382 | SCT-Ga471 mature light chain variable domain protein sequence |
| 1382 | SCT-Ga473 mature light chain variable domain protein sequence |
| 1382 | SCT-Ga490 mature light chain variable domain protein sequence |
| 1382 | SCT-Ga491 mature light chain variable domain protein sequence |
| 1382 | SCT-Ga492 mature light chain variable domain protein sequence |
| 1383 | SCT-Ga472 mature light chain variable domain protein sequence |
| 1383 | SCT-Ga474 mature light chain variable domain protein sequence |
| 1384 | SCT-Ga473 mature heavy chain variable domain protein sequence |
| 1384 | SCT-Ga474 mature heavy chain variable domain protein sequence |
| 1385 | SCT-Ga475 mature heavy chain variable domain protein sequence |
| 1385 | SCT-Ga476 mature heavy chain variable domain protein sequence |
| 1386 | SCT-Ga475 mature light chain variable domain protein sequence |
| 1386 | SCT-Ga477 mature light chain variable domain protein sequence |
| 1386 | SCT-Ga484 mature light chain variable domain protein sequence |
| 1386 | SCT-Ga485 mature light chain variable domain protein sequence |
| 1386 | SCT-Ga486 mature light chain variable domain protein sequence |
| 1387 | SCT-Ga476 mature light chain variable domain protein sequence |
| 1387 | SCT-Ga478 mature light chain variable domain protein sequence |
| 1388 | SCT-Ga477 mature heavy chain variable domain protein sequence |
| 1388 | SCT-Ga478 mature heavy chain variable domain protein sequence |
| 1389 | SCT-Ga479 mature heavy chain variable domain protein sequence |
| 1390 | SCT-Ga479 mature light chain variable domain protein sequence |
| 1391 | SCT-Ga480 mature heavy chain variable domain protein sequence |
| 1391 | SCT-Ga481 mature heavy chain variable domain protein sequence |
| 1392 | SCT-Ga480 mature light chain variable domain protein sequence |
| 1392 | SCT-Ga482 mature light chain variable domain protein sequence |
| 1393 | SCT-Ga481 mature light chain variable domain protein sequence |
| 1393 | SCT-Ga483 mature light chain variable domain protein sequence |
| 1394 | SCT-Ga482 mature heavy chain variable domain protein sequence |
| 1394 | SCT-Ga483 mature heavy chain variable domain protein sequence |
| 1395 | SCT-Ga484 mature heavy chain variable domain protein sequence |
| 1396 | SCT-Ga485 mature heavy chain variable domain protein sequence |
| 1397 | SCT-Ga486 mature heavy chain variable domain protein sequence |
| 1398 | SCT-Ga487 mature heavy chain variable domain protein sequence |
| 1398 | SCT-Ga490 mature heavy chain variable domain protein sequence |
| 1399 | SCT-Ga487 mature light chain variable domain protein sequence |
| 1399 | SCT-Ga488 mature light chain variable domain protein sequence |
| 1399 | SCT-Ga489 mature light chain variable domain protein sequence |
| 1400 | SCT-Ga488 mature heavy chain variable domain protein sequence |
| 1400 | SCT-Ga491 mature heavy chain variable domain protein sequence |
| 1401 | SCT-Ga489 mature heavy chain variable domain protein sequence |
| 1401 | SCT-Ga492 mature heavy chain variable domain protein sequence |
| 1402 | SCT-Ga493 mature heavy chain variable domain protein sequence |
| 1402 | SCT-Ga494 mature heavy chain variable domain protein sequence |
| 1403 | SCT-Ga494 mature light chain variable domain protein sequence |
| 1403 | SCT-Ga496 mature light chain variable domain protein sequence |
| 1404 | SCT-Ga495 mature heavy chain variable domain protein sequence |
| 1404 | SCT-Ga496 mature heavy chain variable domain protein sequence |
| 1405 | SCT-Ga497 mature heavy chain variable domain protein sequence |
| 1405 | SCT-Ga498 mature heavy chain variable domain protein sequence |
| 1406 | SCT-Ga497 mature light chain variable domain protein sequence |
| 1407 | SCT-Ga498 mature light chain variable domain protein sequence |

### BRIEF DESCRIPTION OF THE DRAWINGS

FIG. 1. shows interaction analysis of human TIGIT with select subset of anti-TIGIT antibodies by ELISA.

FIG. 2. shows dose-dependent blockade of anti-human TIGIT antibody using Promega's TIGIT blockade bioassay against a benchmark antibody, MK-7684.

FIG. 3. shows combination assay pairing with anti-PD-1 antibody using a Promega's kit. PM: Promega. The anti-PD-1 antibody (Cat. No. J1201) and anti-TIGIT antibody (Cat. No. J2051) were provided by Promega. Every plot shows a synergistic effect between anti-PD-1 antibody and anti-TIGIT antibody. FIG. 3E shows SCT-Ga461 and SCT-Ga462, both of which are humanized antibodies, have activity comparable to that of their murine counterpart (SCT-Ga433).

### DETAILED DESCRIPTION

The disclosure provides antibodies that bind to TIGIT. In one embodiment, the disclosure provides an isolated antibody that binds to SEQ ID NO:1.

### Antibodies

The disclosure provides antibodies that bind specifically to SEQ ID NO:1. The term “antibody” as used herein, includes both full-length immunoglobulins and antibody fragments that bind to the same antigens. The antibodies can be, e.g., a monoclonal, polyclonal, chimeric, humanized, or single chain antibody. As used herein, the terms “antigen binding fragment,” “fragment,” and “antibody fragment” are used interchangeably to refer to any fragment that comprises a portion of a full-length antibody, generally at least the antigen binding portion or the variable region thereof. Examples of antibody fragments include, but are not limited to, diabodies, single-chain antibody molecules, multi-specific antibodies, Fab, Fab′, F(ab′)2, Fv or scFv.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

The terms “treating,” “treatment,” and the like, as used herein, mean ameliorating a disease, so as to reduce, ameliorate, or eliminate its cause, its progression, its severity, or one or more of its symptoms, or otherwise beneficially alter the disease in a subject. Reference to “treating,” or “treatment” of a patient is intended to include prophylaxis. Treatment may also be preemptive in nature, i.e., it may include prevention of disease in a subject exposed to or at risk for the disease. Prevention of a disease may involve complete protection from disease, for example as in the case of prevention of infection with a pathogen or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The terms “subject” and “patient” are used interchangeably herein to mean all mammals including humans Examples of subjects include, but are not limited to, humans, monkeys, dogs, cats, horses, cows, goats, sheep, pigs, and rabbits. In one embodiment, the subject or patient is a human.

In certain embodiments, the cancer treated with the anti-TIGIT antibody molecule, alone or in combination, includes but is not limited to, a solid tumor, a hematological cancer (e.g., leukemia, lymphoma, myeloma), and a metastatic lesion thereof. In one embodiment, the cancer is a solid tumor. Examples of solid tumors include malignancies, e.g., sarcomas and carcinomas (e.g., adenocarcinomas), of the various organ systems, such as those affecting lung, breast, lymphoid, gastrointestinal or colorectal, genitals and genitourinary tract (e.g., renal, urothelial, bladder cells), pharynx, CNS (e.g., brain, neural or glial cells), skin (e.g., melanoma), head and neck (e.g., head and neck squamous cell carcinoma (HNCC)), and pancreas. For example, melanoma, colon cancers, gastric cancer, rectal cancer, renal-cell carcinoma, breast cancer (e.g., a breast cancer that does not express one, two or all of estrogen receptor, progesterone receptor, or Her2/neu, e.g., a triple negative breast cancer), liver cancer, a lung cancer (e.g., a non-small cell lung cancer (NSCLC) (e.g., a NSCLC with squamous and/or non-squamous histology) or small cell lung cancer), prostate cancer, cancer of head or neck (e.g., HPV+ squamous cell carcinoma), cancer of the small intestine and cancer of the esophagus. Examples of hematological cancer include, but is not limited to, leukemia (e.g., a myeloid leukemia, lymphoid leukemia, or chronic lymphocytic leukemia (CLL)), lymphoma (e.g., Hodgkin lymphoma (HL), non-Hodgkin lymphoma (NHL), Diffuse large B-cell lymphoma (DLBCL), T-cell lymphoma, or mantle cell lymphoma (MCL)), and myeloma, e.g., multiple myeloma. The cancer may be at an early, intermediate, late stage or metastatic cancer. For discovering and developing antibody therapeutics to TIGIT generally known in the art, please see U.S. Pat. Application No. 2018/0066055, which is incorporated in its entirety by reference.

The provided antibodies disclosed herein can be used to treat, prevent and/or diagnose cancerous or malignant disorders (e.g., cancers such as melanoma, e.g., advanced stage melanoma; pancreatic cancer, e.g., advanced pancreatic cancer; solid tumors; breast cancer, e.g., metastatic breast carcinoma; renal cell carcinoma, e.g., advanced or metastatic renal cell carcinoma (MRCC) or clear cell renal cell carcinoma).

The provided antibodies may be used to diagnose, treat, or monitor TIGIT-expressing cancer and its progression, regression, or stability; to determine whether or not a patient should be treated for cancer; or to determine whether or not a subject is afflicted with TIGIT-expressing cancer and thus may be amenable to treatment with a TIGIT-specific anti-cancer therapeutic. In some embodiments, the antibodies or fragments thereof described herein may be used for various in vitro molecular-biology applications such as, for example, enzyme-linked immunosorbent assays (ELISA), Western blots, immunohistochemistry, immunocytochemistry, flow cytometry and fluorescence-activated cell sorting (FACS), immunoprecipitation, and/or enzyme-linked immunospot assays (ELISPOT). In some embodiments, the antibodies or fragments thereof may be packaged in kits with or without additional reagents known to those of skill in the art for practicing any of the molecular biology techniques disclosed above.

The disclosure provides the antibodies SCT-Ga001, SCT-Ga002, SCT-Ga003, SCT-Ga004, SCT-Ga005, SCT-Ga006, SCT-Ga007, SCT-Ga008, SCT-Ga009, SCT-Ga010, SCT-Ga011, SCT-Ga012, SCT-Ga013, SCT-Ga014, SCT-Ga015, SCT-Ga016, SCT-Ga017, SCT-Ga018, SCT-Ga019, SCT-Ga020, SCT-Ga021, SCT-Ga022, SCT-Ga023, SCT-Ga024, SCT-Ga025, SCT-Ga026, SCT-Ga027, SCT-Ga028, SCT-Ga029, SCT-Ga030, SCT-Ga031, SCT-Ga032, SCT-Ga033, SCT-Ga034, SCT-Ga035, SCT-Ga036, SCT-Ga037, SCT-Ga038, SCT-Ga039, SCT-Ga040, SCT-Ga041, SCT-Ga042, SCT-Ga043, SCT-Ga044, SCT-Ga045, SCT-Ga046, SCT-Ga047, SCT-Ga048, SCT-Ga049, SCT-Ga050, SCT-Ga051, SCT-Ga052, SCT-Ga053, SCT-Ga054, SCT-Ga055, SCT-Ga056, SCT-Ga057, SCT-Ga058, SCT-Ga059, SCT-Ga060, SCT-Ga061, SCT-Ga062, SCT-Ga063, SCT-Ga064, SCT-Ga065, SCT-Ga066, SCT-Ga067, SCT-Ga068, SCT-Ga069, SCT-Ga070, SCT-Ga071, SCT-Ga072, SCT-Ga073, SCT-Ga074, SCT-Ga075, SCT-Ga076, SCT-Ga077, SCT-Ga078, SCT-Ga079, SCT-Ga080, SCT-Ga081, SCT-Ga082, SCT-Ga083, SCT-Ga084, SCT-Ga085, SCT-Ga086, SCT-Ga087, SCT-Ga088, SCT-Ga089, SCT-Ga090, SCT-Ga091, SCT-Ga092, SCT-Ga093, SCT-Ga094, SCT-Ga095, SCT-Ga096, SCT-Ga097, SCT-Ga098, SCT-Ga099, SCT-Ga100, SCT-Ga101, SCT-Ga102, SCT-Ga103, SCT-Ga104, SCT-Ga105, SCT-Ga106, SCT-Ga107, SCT-Ga108, SCT-Ga109, SCT-Ga110, SCT-Ga111, SCT-Ga112, SCT-Ga113, SCT-Ga114, SCT-Ga115, SCT-Ga116, SCT-Ga117, SCT-Ga118, SCT-Ga119, SCT-Ga120, SCT-Ga121, SCT-Ga122, SCT-Ga123, SCT-Ga124, SCT-Ga125, SCT-Ga126, SCT-Ga127, SCT-Ga128, SCT-Ga129, SCT-Ga130, SCT-Ga131, SCT-Ga132, SCT-Ga133, SCT-Ga134, SCT-Ga135, SCT-Ga136, SCT-Ga137, SCT-Ga138, SCT-Ga139, SCT-Ga140, SCT-Ga141, SCT-Ga142, SCT-Ga143, SCT-Ga144, SCT-Ga145, SCT-Ga146, SCT-Ga147, SCT-Ga148, SCT-Ga149, SCT-Ga150, SCT-Ga151, SCT-Ga152, SCT-Ga153, SCT-Ga154, SCT-Ga155, SCT-Ga156, SCT-Ga157, SCT-Ga158, SCT-Ga159, SCT-Ga160, SCT-Ga161, SCT-Ga162, SCT-Ga163, SCT-Ga164, SCT-Ga165, SCT-Ga166, SCT-Ga167, SCT-Ga168, SCT-Ga169, SCT-Ga170, SCT-Ga171, SCT-Ga172, SCT-Ga173, SCT-Ga174, SCT-Ga175, SCT-Ga176, SCT-Ga177, SCT-Ga178, SCT-Ga179, SCT-Ga180, SCT-Ga181, SCT-Ga182, SCT-Ga183, SCT-Ga184, SCT-Ga185, SCT-Ga186, SCT-Ga187, SCT-Ga188, SCT-Ga189, SCT-Ga190, SCT-Ga191, SCT-Ga192, SCT-Ga193, SCT-Ga194, SCT-Ga195, SCT-Ga196, SCT-Ga197, SCT-Ga198, SCT-Ga199, SCT-Ga200, SCT-Ga201, SCT-Ga202, SCT-Ga203, SCT-Ga204, SCT-Ga205, SCT-Ga206, SCT-Ga207, SCT-Ga208, SCT-Ga209, SCT-Ga210, SCT-Ga211, SCT-Ga212, SCT-Ga213, SCT-Ga214, SCT-Ga215, SCT-Ga216, SCT-Ga217, SCT-Ga218, SCT-Ga219, SCT-Ga220, SCT-Ga221, SCT-Ga222, SCT-Ga223, SCT-Ga224, SCT-Ga225, SCT-Ga226, SCT-Ga227, SCT-Ga228, SCT-Ga229, SCT-Ga230, SCT-Ga231, SCT-Ga232, SCT-Ga233, SCT-Ga234, SCT-Ga235, SCT-Ga236, SCT-Ga237, SCT-Ga238, SCT-Ga239, SCT-Ga240, SCT-Ga241, SCT-Ga242, SCT-Ga243, SCT-Ga244, SCT-Ga245, SCT-Ga246, SCT-Ga247, SCT-Ga248, SCT-Ga249, SCT-Ga250, SCT-Ga251, SCT-Ga252, SCT-Ga253, SCT-Ga254, SCT-Ga255, SCT-Ga256, SCT-Ga257, SCT-Ga258, SCT-Ga259, SCT-Ga260, SCT-Ga261, SCT-Ga262, SCT-Ga263, SCT-Ga264, SCT-Ga265, SCT-Ga266, SCT-Ga267, SCT-Ga268, SCT-Ga269, SCT-Ga270, SCT-Ga271, SCT-Ga272, SCT-Ga273, SCT-Ga274, SCT-Ga275, SCT-Ga276, SCT-Ga277, SCT-Ga278, SCT-Ga279, SCT-Ga280, SCT-Ga281, SCT-Ga282, SCT-Ga283, SCT-Ga284, SCT-Ga285, SCT-Ga286, SCT-Ga287, SCT-Ga288, SCT-Ga289, SCT-Ga290, SCT-Ga291, SCT-Ga292, SCT-Ga293, SCT-Ga294, SCT-Ga295, SCT-Ga296, SCT-Ga297, SCT-Ga298, SCT-Ga299, SCT-Ga300, SCT-Ga301, SCT-Ga302, SCT-Ga303, SCT-Ga304, SCT-Ga305, SCT-Ga306, SCT-Ga307, SCT-Ga308, SCT-Ga309, SCT-Ga310, SCT-Ga311, SCT-Ga312, SCT-Ga313, SCT-Ga314, SCT-Ga315, SCT-Ga316, SCT-Ga317, SCT-Ga318, SCT-Ga319, SCT-Ga320, SCT-Ga321, SCT-Ga322, SCT-Ga323, SCT-Ga324, SCT-Ga325, SCT-Ga326, SCT-Ga327, SCT-Ga328, SCT-Ga329, SCT-Ga330, SCT-Ga331, SCT-Ga332, SCT-Ga333, SCT-Ga334, SCT-Ga335, SCT-Ga336, SCT-Ga337, SCT-Ga338, SCT-Ga339, SCT-Ga340, SCT-Ga341, SCT-Ga342, SCT-Ga343, SCT-Ga344, SCT-Ga345, SCT-Ga346, SCT-Ga347, SCT-Ga348, SCT-Ga349, SCT-Ga350, SCT-Ga351, SCT-Ga352, SCT-Ga353, SCT-Ga354, SCT-Ga355, SCT-Ga356, SCT-Ga357, SCT-Ga358, SCT-Ga359, SCT-Ga360, SCT-Ga361, SCT-Ga362, SCT-Ga363, SCT-Ga364, SCT-Ga365, SCT-Ga366, SCT-Ga367, SCT-Ga368, SCT-Ga369, SCT-Ga370, SCT-Ga371, SCT-Ga372, SCT-Ga373, SCT-Ga374, SCT-Ga375, SCT-Ga376, SCT-Ga377, SCT-Ga378, SCT-Ga379, SCT-Ga380, SCT-Ga381, SCT-Ga382, SCT-Ga383, SCT-Ga384, SCT-Ga385, SCT-Ga386, SCT-Ga387, SCT-Ga388, SCT-Ga389, SCT-Ga390, SCT-Ga391, SCT-Ga392, SCT-Ga393, SCT-Ga394, SCT-Ga395, SCT-Ga396, SCT-Ga397, SCT-Ga398, SCT-Ga399, SCT-Ga400, SCT-Ga401, SCT-Ga402, SCT-Ga403, SCT-Ga404, SCT-Ga405, SCT-Ga406, SCT-Ga407, SCT-Ga408, SCT-Ga409, SCT-Ga410, SCT-Ga411, SCT-Ga412, SCT-Ga413, SCT-Ga414, SCT-Ga415, SCT-Ga416, SCT-Ga417, SCT-Ga418, SCT-Ga419, SCT-Ga420, SCT-Ga421, SCT-Ga422, SCT-Ga423, SCT-Ga424, SCT-Ga425, SCT-Ga426, SCT-Ga427, SCT-Ga428, SCT-Ga429, SCT-Ga430, SCT-Ga431, SCT-Ga432, SCT-Ga433, SCT-Ga434, SCT-Ga435, SCT-Ga436, SCT-Ga437, SCT-Ga438, SCT-Ga439, SCT-Ga440, SCT-Ga441, SCT-Ga442, SCT-Ga443, SCT-Ga444, SCT-Ga445, SCT-Ga446, SCT-Ga447, SCT-Ga448, SCT-Ga449, SCT-Ga450, SCT-Ga451, SCT-Ga452, SCT-Ga453, SCT-Ga454, SCT-Ga455, and SCT-Ga456. Each of these is a murine monoclonal antibody. The disclosure also provides the antibodies SCT-Ga457, SCT-Ga458, SCT-Ga459, SCT-Ga460, SCT-Ga461, SCT-Ga462, SCT-Ga463, SCT-Ga464, SCT-Ga465, SCT-Ga466, SCT-Ga467, SCT-Ga468, SCT-Ga469, SCT-Ga470, SCT-Ga471, SCT-Ga472, SCT-Ga473, SCT-Ga474, SCT-Ga475, SCT-Ga476, SCT-Ga477, SCT-Ga478, SCT-Ga479, SCT-Ga480, SCT-Ga481, SCT-Ga482, SCT-Ga483, SCT-Ga484, SCT-Ga485, SCT-Ga486, SCT-Ga487, SCT-Ga488, SCT-Ga489, SCT-Ga490, SCT-Ga491, SCT-Ga492, SCT-Ga493, SCT-Ga494, SCT-Ga495, SCT-Ga496, SCT-Ga497, and SCT-Ga498. Each of these is a humanized monoclonal antibody.

Additionally, recombinant anti-TIGIT antibodies, such as chimeric and humanized monoclonal antibodies, comprising both human and non-human portions, which can be made using standard recombinant DNA techniques, are within the scope of the disclosure. Such chimeric and humanized monoclonal antibodies can be produced by recombinant DNA techniques such as, for example, the methods described in U.S. Pat. No. 7,112,421; Better et al. (1988) Science 240:1041-1043; or Liu et al. (1987) Proc. Natl. Acad. Sci. USA 84:3439-3443.

### Antibody Variable Domain Sequence

The antibodies of the disclosure may comprise the heavy chain variable domain sequences of SEQ ID NO:4, SEQ ID NO:5, SEQ ID NO:6, SEQ ID NO:6, SEQ ID NO:7, SEQ ID NO:7, SEQ ID NO:7, SEQ ID NO:7, SEQ ID NO:7, SEQ ID NO:7, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:17, SEQ ID NO:17, SEQ ID NO:18, SEQ ID NO:19, SEQ ID NO:20, SEQ ID NO:20, SEQ ID NO:20, SEQ ID NO:20, SEQ ID NO:21, SEQ ID NO:21, SEQ ID NO:22, SEQ ID NO:23, SEQ ID NO:23, SEQ ID NO:23, SEQ ID NO:24, SEQ ID NO:25, SEQ ID NO:26, SEQ ID NO:27, SEQ ID NO:27, SEQ ID NO:27, SEQ ID NO:27, SEQ ID NO:27, SEQ ID NO:28, SEQ ID NO:29, SEQ ID NO:30, SEQ ID NO:31, SEQ ID NO:32, SEQ ID NO:33, SEQ ID NO:33, SEQ ID NO:34, SEQ ID NO:35, SEQ ID NO:36, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:51, SEQ ID NO:51, SEQ ID NO:52, SEQ ID NO:53, SEQ ID NO:54, SEQ ID NO:55, SEQ ID NO:55, SEQ ID NO:56, SEQ ID NO:57, SEQ ID NO:58, SEQ ID NO:58, SEQ ID NO:59, SEQ ID NO:60, SEQ ID NO:61, SEQ ID NO:62, SEQ ID NO:62, SEQ ID NO:62, SEQ ID NO:62, SEQ ID NO:62, SEQ ID NO:63, SEQ ID NO:64, SEQ ID NO:65, SEQ ID NO:66, SEQ ID NO:67, SEQ ID NO:67, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:75, SEQ ID NO:76, SEQ ID NO:77, SEQ ID NO:78, SEQ ID NO:79, SEQ ID NO:79, SEQ ID NO:80, SEQ ID NO:81, SEQ ID NO:82, SEQ ID NO:83, SEQ ID NO:84, SEQ ID NO:84, SEQ ID NO:84, SEQ ID NO:84, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:163, SEQ ID NO:164, SEQ ID NO:164, SEQ ID NO:165, SEQ ID NO:166, SEQ ID NO:167, SEQ ID NO:168, SEQ ID NO:169, SEQ ID NO:170, SEQ ID NO:170, SEQ ID NO:171, SEQ ID NO:171, SEQ ID NO:171, SEQ ID NO:172, SEQ ID NO:173, SEQ ID NO:173, SEQ ID NO:173, SEQ ID NO:174, SEQ ID NO:175, SEQ ID NO:176, SEQ ID NO:176, SEQ ID NO:177, SEQ ID NO:177, SEQ ID NO:177, SEQ ID NO:177, SEQ ID NO:177, SEQ ID NO:178, SEQ ID NO:179, SEQ ID NO:180, SEQ ID NO:180, SEQ ID NO:181, SEQ ID NO:181, SEQ ID NO:182, SEQ ID NO:182, SEQ ID NO:182, SEQ ID NO:183, SEQ ID NO:183, SEQ ID NO:183, SEQ ID NO:184, SEQ ID NO:185, SEQ ID NO:186, SEQ ID NO:187, SEQ ID NO:188, SEQ ID NO:189, SEQ ID NO:189, SEQ ID NO:189, SEQ ID NO:190, SEQ ID NO:191, SEQ ID NO:192, SEQ ID NO:192, SEQ ID NO:193, SEQ ID NO:194, SEQ ID NO:195, SEQ ID NO:196, SEQ ID NO:197, SEQ ID NO:198, SEQ ID NO:198, SEQ ID NO:199, SEQ ID NO:200, SEQ ID NO:201, SEQ ID NO:201, SEQ ID NO:201, SEQ ID NO:201, SEQ ID NO:201, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:220, SEQ ID NO:221, SEQ ID NO:222, SEQ ID NO:223, SEQ ID NO:224, SEQ ID NO:224, SEQ ID NO:225, SEQ ID NO:226, SEQ ID NO:227, SEQ ID NO:228, SEQ ID NO:229, SEQ ID NO:230, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:311, SEQ ID NO:312, SEQ ID NO:313, SEQ ID NO:313, SEQ ID NO:314, SEQ ID NO:315, SEQ ID NO:315, SEQ ID NO:316, SEQ ID NO:317, SEQ ID NO:318, SEQ ID NO:319, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:330, SEQ ID NO:331, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:1366, SEQ ID NO:1369, SEQ ID NO:1372, SEQ ID NO:1373, SEQ ID NO:1376, SEQ ID NO:1377, SEQ ID NO:1380, SEQ ID NO:1381, SEQ ID NO:1384, SEQ ID NO:1385, SEQ ID NO:1388, SEQ ID NO:1389, SEQ ID NO:1391, SEQ ID NO:1394, SEQ ID NO:1395, SEQ ID NO:1396, SEQ ID NO:1397, SEQ ID NO:1398, SEQ ID NO:1400, SEQ ID NO:1401, SEQ ID NO:1402, SEQ ID NO:1404, or SEQ ID NO:1405. The heavy chain variable domain sequences may consist essentially of SEQ ID NO:4, SEQ ID NO:5, SEQ ID NO:6, SEQ ID NO:6, SEQ ID NO:7, SEQ ID NO:7, SEQ ID NO:7, SEQ ID NO:7, SEQ ID NO:7, SEQ ID NO:7, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:17, SEQ ID NO:17, SEQ ID NO:18, SEQ ID NO:19, SEQ ID NO:20, SEQ ID NO:20, SEQ ID NO:20, SEQ ID NO:20, SEQ ID NO:21, SEQ ID NO:21, SEQ ID NO:22, SEQ ID NO:23, SEQ ID NO:23, SEQ ID NO:23, SEQ ID NO:24, SEQ ID NO:25, SEQ ID NO:26, SEQ ID NO:27, SEQ ID NO:27, SEQ ID NO:27, SEQ ID NO:27, SEQ ID NO:27, SEQ ID NO:28, SEQ ID NO:29, SEQ ID NO:30, SEQ ID NO:31, SEQ ID NO:32, SEQ ID NO:33, SEQ ID NO:33, SEQ ID NO:34, SEQ ID NO:35, SEQ ID NO:36, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:51, SEQ ID NO:51, SEQ ID NO:52, SEQ ID NO:53, SEQ ID NO:54, SEQ ID NO:55, SEQ ID NO:55, SEQ ID NO:56, SEQ ID NO:57, SEQ ID NO:58, SEQ ID NO:58, SEQ ID NO:59, SEQ ID NO:60, SEQ ID NO:61, SEQ ID NO:62, SEQ ID NO:62, SEQ ID NO:62, SEQ ID NO:62, SEQ ID NO:62, SEQ ID NO:63, SEQ ID NO:64, SEQ ID NO:65, SEQ ID NO:66, SEQ ID NO:67, SEQ ID NO:67, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:75, SEQ ID NO:76, SEQ ID NO:77, SEQ ID NO:78, SEQ ID NO:79, SEQ ID NO:79, SEQ ID NO:80, SEQ ID NO:81, SEQ ID NO:82, SEQ ID NO:83, SEQ ID NO:84, SEQ ID NO:84, SEQ ID NO:84, SEQ ID NO:84, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:163, SEQ ID NO:164, SEQ ID NO:164, SEQ ID NO:165, SEQ ID NO:166, SEQ ID NO:167, SEQ ID NO:168, SEQ ID NO:169, SEQ ID NO:170, SEQ ID NO:170, SEQ ID NO:171, SEQ ID NO:171, SEQ ID NO:171, SEQ ID NO:172, SEQ ID NO:173, SEQ ID NO:173, SEQ ID NO:173, SEQ ID NO:174, SEQ ID NO:175, SEQ ID NO:176, SEQ ID NO:176, SEQ ID NO:177, SEQ ID NO:177, SEQ ID NO:177, SEQ ID NO:177, SEQ ID NO:177, SEQ ID NO:178, SEQ ID NO:179, SEQ ID NO:180, SEQ ID NO:180, SEQ ID NO:181, SEQ ID NO:181, SEQ ID NO:182, SEQ ID NO:182, SEQ ID NO:182, SEQ ID NO:183, SEQ ID NO:183, SEQ ID NO:183, SEQ ID NO:184, SEQ ID NO:185, SEQ ID NO:186, SEQ ID NO:187, SEQ ID NO:188, SEQ ID NO:189, SEQ ID NO:189, SEQ ID NO:189, SEQ ID NO:190, SEQ ID NO:191, SEQ ID NO:192, SEQ ID NO:192, SEQ ID NO:193, SEQ ID NO:194, SEQ ID NO:195, SEQ ID NO:196, SEQ ID NO:197, SEQ ID NO:198, SEQ ID NO:198, SEQ ID NO:199, SEQ ID NO:200, SEQ ID NO:201, SEQ ID NO:201, SEQ ID NO:201, SEQ ID NO:201, SEQ ID NO:201, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:220, SEQ ID NO:221, SEQ ID NO:222, SEQ ID NO:223, SEQ ID NO:224, SEQ ID NO:224, SEQ ID NO:225, SEQ ID NO:226, SEQ ID NO:227, SEQ ID NO:228, SEQ ID NO:229, SEQ ID NO:230, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:311, SEQ ID NO:312, SEQ ID NO:313, SEQ ID NO:313, SEQ ID NO:314, SEQ ID NO:315, SEQ ID NO:315, SEQ ID NO:316, SEQ ID NO:317, SEQ ID NO:318, SEQ ID NO:319, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:330, SEQ ID NO:331, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:1366, SEQ ID NO:1369, SEQ ID NO:1372, SEQ ID NO:1373, SEQ ID NO:1376, SEQ ID NO:1377, SEQ ID NO:1380, SEQ ID NO:1381, SEQ ID NO:1384, SEQ ID NO:1385, SEQ ID NO:1388, SEQ ID NO:1389, SEQ ID NO:1391, SEQ ID NO:1394, SEQ ID NO:1395, SEQ ID NO:1396, SEQ ID NO:1397, SEQ ID NO:1398, SEQ ID NO:1400, SEQ ID NO:1401, SEQ ID NO:1402, SEQ ID NO:1404, or SEQ ID NO:1405.

The antibodies of the disclosure may comprise the light chain variable domain sequences of SEQ ID NO:380, SEQ ID NO:380, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:382, SEQ ID NO:382, SEQ ID NO:382, SEQ ID NO:382, SEQ ID NO:382, SEQ ID NO:383, SEQ ID NO:384, SEQ ID NO:385, SEQ ID NO:385, SEQ ID NO:385, SEQ ID NO:385, SEQ ID NO:385, SEQ ID NO:385, SEQ ID NO:385, SEQ ID NO:386, SEQ ID NO:387, SEQ ID NO:388, SEQ ID NO:389, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:396, SEQ ID NO:397, SEQ ID NO:398, SEQ ID NO:398, SEQ ID NO:398, SEQ ID NO:398, SEQ ID NO:398, SEQ ID NO:398, SEQ ID NO:398, SEQ ID NO:398, SEQ ID NO:399, SEQ ID NO:399, SEQ ID NO:400, SEQ ID NO:401, SEQ ID NO:401, SEQ ID NO:402, SEQ ID NO:403, SEQ ID NO:404, SEQ ID NO:404, SEQ ID NO:405, SEQ ID NO:406, SEQ ID NO:406, SEQ ID NO:406, SEQ ID NO:407, SEQ ID NO:408, SEQ ID NO:409, SEQ ID NO:410, SEQ ID NO:410, SEQ ID NO:411, SEQ ID NO:412, SEQ ID NO:413, SEQ ID NO:414, SEQ ID NO:415, SEQ ID NO:416, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:445, SEQ ID NO:446, SEQ ID NO:447, SEQ ID NO:448, SEQ ID NO:449, SEQ ID NO:450, SEQ ID NO:451, SEQ ID NO:451, SEQ ID NO:451, SEQ ID NO:452, SEQ ID NO:453, SEQ ID NO:454, SEQ ID NO:455, SEQ ID NO:456, SEQ ID NO:456, SEQ ID NO:457, SEQ ID NO:458, SEQ ID NO:459, SEQ ID NO:460, SEQ ID NO:461, SEQ ID NO:462, SEQ ID NO:462, SEQ ID NO:463, SEQ ID NO:464, SEQ ID NO:465, SEQ ID NO:466, SEQ ID NO:466, SEQ ID NO:466, SEQ ID NO:466, SEQ ID NO:467, SEQ ID NO:468, SEQ ID NO:469, SEQ ID NO:469, SEQ ID NO:469, SEQ ID NO:469, SEQ ID NO:470, SEQ ID NO:471, SEQ ID NO:472, SEQ ID NO:473, SEQ ID NO:474, SEQ ID NO:474, SEQ ID NO:475, SEQ ID NO:476, SEQ ID NO:477, SEQ ID NO:478, SEQ ID NO:479, SEQ ID NO:479, SEQ ID NO:480, SEQ ID NO:481, SEQ ID NO:482, SEQ ID NO:482, SEQ ID NO:482, SEQ ID NO:482, SEQ ID NO:482, SEQ ID NO:482, SEQ ID NO:482, SEQ ID NO:482, SEQ ID NO:482, SEQ ID NO:483, SEQ ID NO:483, SEQ ID NO:484, SEQ ID NO:485, SEQ ID NO:486, SEQ ID NO:486, SEQ ID NO:487, SEQ ID NO:488, SEQ ID NO:489, SEQ ID NO:490, SEQ ID NO:491, SEQ ID NO:492, SEQ ID NO:493, SEQ ID NO:494, SEQ ID NO:495, SEQ ID NO:496, SEQ ID NO:497, SEQ ID NO:498, SEQ ID NO:499, SEQ ID NO:500, SEQ ID NO:501, SEQ ID NO:502, SEQ ID NO:503, SEQ ID NO:504, SEQ ID NO:505, SEQ ID NO:506, SEQ ID NO:506, SEQ ID NO:507, SEQ ID NO:508, SEQ ID NO:509, SEQ ID NO:510, SEQ ID NO:511, SEQ ID NO:512, SEQ ID NO:512, SEQ ID NO:513, SEQ ID NO:514, SEQ ID NO:515, SEQ ID NO:515, SEQ ID NO:515, SEQ ID NO:516, SEQ ID NO:517, SEQ ID NO:517, SEQ ID NO:518, SEQ ID NO:519, SEQ ID NO:520, SEQ ID NO:521, SEQ ID NO:522, SEQ ID NO:523, SEQ ID NO:524, SEQ ID NO:525, SEQ ID NO:526, SEQ ID NO:527, SEQ ID NO:527, SEQ ID NO:528, SEQ ID NO:529, SEQ ID NO:529, SEQ ID NO:529, SEQ ID NO:530, SEQ ID NO:531, SEQ ID NO:531, SEQ ID NO:531, SEQ ID NO:532, SEQ ID NO:533, SEQ ID NO:534, SEQ ID NO:535, SEQ ID NO:536, SEQ ID NO:537, SEQ ID NO:538, SEQ ID NO:539, SEQ ID NO:540, SEQ ID NO:540, SEQ ID NO:541, SEQ ID NO:542, SEQ ID NO:543, SEQ ID NO:544, SEQ ID NO:545, SEQ ID NO:546, SEQ ID NO:547, SEQ ID NO:548, SEQ ID NO:549, SEQ ID NO:550, SEQ ID NO:551, SEQ ID NO:552, SEQ ID NO:552, SEQ ID NO:552, SEQ ID NO:552, SEQ ID NO:552, SEQ ID NO:552, SEQ ID NO:553, SEQ ID NO:554, SEQ ID NO:555, SEQ ID NO:556, SEQ ID NO:557, SEQ ID NO:558, SEQ ID NO:559, SEQ ID NO:560, SEQ ID NO:561, SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, SEQ ID NO:571, SEQ ID NO:572, SEQ ID NO:572, SEQ ID NO:572, SEQ ID NO:572, SEQ ID NO:572, SEQ ID NO:572, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:580, SEQ ID NO:581, SEQ ID NO:582, SEQ ID NO:583, SEQ ID NO:584, SEQ ID NO:585, SEQ ID NO:586, SEQ ID NO:587, SEQ ID NO:588, SEQ ID NO:589, SEQ ID NO:590, SEQ ID NO:591, SEQ ID NO:592, SEQ ID NO:593, SEQ ID NO:594, SEQ ID NO:595, SEQ ID NO:596, SEQ ID NO:596, SEQ ID NO:596, SEQ ID NO:596, SEQ ID NO:597, SEQ ID NO:598, SEQ ID NO:599, SEQ ID NO:600, SEQ ID NO:601, SEQ ID NO:602, SEQ ID NO:603, SEQ ID NO:604, SEQ ID NO:605, SEQ ID NO:606, SEQ ID NO:607, SEQ ID NO:608, SEQ ID NO:608, SEQ ID NO:609, SEQ ID NO:610, SEQ ID NO:611, SEQ ID NO:612, SEQ ID NO:612, SEQ ID NO:613, SEQ ID NO:614, SEQ ID NO:615, SEQ ID NO:616, SEQ ID NO:617, SEQ ID NO:618, SEQ ID NO:619, SEQ ID NO:620, SEQ ID NO:621, SEQ ID NO:622, SEQ ID NO:623, SEQ ID NO:624, SEQ ID NO:625, SEQ ID NO:626, SEQ ID NO:627, SEQ ID NO:628, SEQ ID NO:629, SEQ ID NO:630, SEQ ID NO:631, SEQ ID NO:632, SEQ ID NO:633, SEQ ID NO:634, SEQ ID NO:635, SEQ ID NO:636, SEQ ID NO:637, SEQ ID NO:638, SEQ ID NO:639, SEQ ID NO:640, SEQ ID NO:641, SEQ ID NO:642, SEQ ID NO:643, SEQ ID NO:643, SEQ ID NO:643, SEQ ID NO:644, SEQ ID NO:645, SEQ ID NO:645, SEQ ID NO:645, SEQ ID NO:645, SEQ ID NO:645, SEQ ID NO:645, SEQ ID NO:646, SEQ ID NO:647, SEQ ID NO:648, SEQ ID NO:649, SEQ ID NO:650, SEQ ID NO:651, SEQ ID NO:652, SEQ ID NO:652, SEQ ID NO:653, SEQ ID NO:654, SEQ ID NO:655, SEQ ID NO:656, SEQ ID NO:657, SEQ ID NO:658, SEQ ID NO:658, SEQ ID NO:659, SEQ ID NO:660, SEQ ID NO:661, SEQ ID NO:662, SEQ ID NO:663, SEQ ID NO:664, SEQ ID NO:665, SEQ ID NO:666, SEQ ID NO:667, SEQ ID NO:667, SEQ ID NO:668, SEQ ID NO:669, SEQ ID NO:669, SEQ ID NO:670, SEQ ID NO:671, SEQ ID NO:672, SEQ ID NO:673, SEQ ID NO:674, SEQ ID NO:675, SEQ ID NO:676, SEQ ID NO:677, SEQ ID NO:678, SEQ ID NO:679, SEQ ID NO:680, SEQ ID NO:681, SEQ ID NO:682, SEQ ID NO:683, SEQ ID NO:684, SEQ ID NO:685, SEQ ID NO:686, SEQ ID NO:1367, SEQ ID NO:1368, SEQ ID NO:1370, SEQ ID NO:1371, SEQ ID NO:1374, SEQ ID NO:1375, SEQ ID NO:1378, SEQ ID NO:1379, SEQ ID NO:1382, SEQ ID NO:1383, SEQ ID NO:1386, SEQ ID NO:1387, SEQ ID NO:1390, SEQ ID NO:1392, SEQ ID NO:1393, SEQ ID NO:1399, SEQ ID NO:1403, SEQ ID NO:1406, or SEQ ID NO:1407. The light chain variable domain sequences may consist essentially of SEQ ID NO:380, SEQ ID NO:380, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:382, SEQ ID NO:382, SEQ ID NO:382, SEQ ID NO:382, SEQ ID NO:382, SEQ ID NO:383, SEQ ID NO:384, SEQ ID NO:385, SEQ ID NO:385, SEQ ID NO:385, SEQ ID NO:385, SEQ ID NO:385, SEQ ID NO:385, SEQ ID NO:385, SEQ ID NO:386, SEQ ID NO:387, SEQ ID NO:388, SEQ ID NO:389, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:396, SEQ ID NO:397, SEQ ID NO:398, SEQ ID NO:398, SEQ ID NO:398, SEQ ID NO:398, SEQ ID NO:398, SEQ ID NO:398, SEQ ID NO:398, SEQ ID NO:398, SEQ ID NO:399, SEQ ID NO:399, SEQ ID NO:400, SEQ ID NO:401, SEQ ID NO:401, SEQ ID NO:402, SEQ ID NO:403, SEQ ID NO:404, SEQ ID NO:404, SEQ ID NO:405, SEQ ID NO:406, SEQ ID NO:406, SEQ ID NO:406, SEQ ID NO:407, SEQ ID NO:408, SEQ ID NO:409, SEQ ID NO:410, SEQ ID NO:410, SEQ ID NO:411, SEQ ID NO:412, SEQ ID NO:413, SEQ ID NO:414, SEQ ID NO:415, SEQ ID NO:416, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:445, SEQ ID NO:446, SEQ ID NO:447, SEQ ID NO:448, SEQ ID NO:449, SEQ ID NO:450, SEQ ID NO:451, SEQ ID NO:451, SEQ ID NO:451, SEQ ID NO:452, SEQ ID NO:453, SEQ ID NO:454, SEQ ID NO:455, SEQ ID NO:456, SEQ ID NO:456, SEQ ID NO:457, SEQ ID NO:458, SEQ ID NO:459, SEQ ID NO:460, SEQ ID NO:461, SEQ ID NO:462, SEQ ID NO:462, SEQ ID NO:463, SEQ ID NO:464, SEQ ID NO:465, SEQ ID NO:466, SEQ ID NO:466, SEQ ID NO:466, SEQ ID NO:466, SEQ ID NO:467, SEQ ID NO:468, SEQ ID NO:469, SEQ ID NO:469, SEQ ID NO:469, SEQ ID NO:469, SEQ ID NO:470, SEQ ID NO:471, SEQ ID NO:472, SEQ ID NO:473, SEQ ID NO:474, SEQ ID NO:474, SEQ ID NO:475, SEQ ID NO:476, SEQ ID NO:477, SEQ ID NO:478, SEQ ID NO:479, SEQ ID NO:479, SEQ ID NO:480, SEQ ID NO:481, SEQ ID NO:482, SEQ ID NO:482, SEQ ID NO:482, SEQ ID NO:482, SEQ ID NO:482, SEQ ID NO:482, SEQ ID NO:482, SEQ ID NO:482, SEQ ID NO:482, SEQ ID NO:483, SEQ ID NO:483, SEQ ID NO:484, SEQ ID NO:485, SEQ ID NO:486, SEQ ID NO:486, SEQ ID NO:487, SEQ ID NO:488, SEQ ID NO:489, SEQ ID NO:490, SEQ ID NO:491, SEQ ID NO:492, SEQ ID NO:493, SEQ ID NO:494, SEQ ID NO:495, SEQ ID NO:496, SEQ ID NO:497, SEQ ID NO:498, SEQ ID NO:499, SEQ ID NO:500, SEQ ID NO:501, SEQ ID NO:502, SEQ ID NO:503, SEQ ID NO:504, SEQ ID NO:505, SEQ ID NO:506, SEQ ID NO:506, SEQ ID NO:507, SEQ ID NO:508, SEQ ID NO:509, SEQ ID NO:510, SEQ ID NO:511, SEQ ID NO:512, SEQ ID NO:512, SEQ ID NO:513, SEQ ID NO:514, SEQ ID NO:515, SEQ ID NO:515, SEQ ID NO:515, SEQ ID NO:516, SEQ ID NO:517, SEQ ID NO:517, SEQ ID NO:518, SEQ ID NO:519, SEQ ID NO:520, SEQ ID NO:521, SEQ ID NO:522, SEQ ID NO:523, SEQ ID NO:524, SEQ ID NO:525, SEQ ID NO:526, SEQ ID NO:527, SEQ ID NO:527, SEQ ID NO:528, SEQ ID NO:529, SEQ ID NO:529, SEQ ID NO:529, SEQ ID NO:530, SEQ ID NO:531, SEQ ID NO:531, SEQ ID NO:531, SEQ ID NO:532, SEQ ID NO:533, SEQ ID NO:534, SEQ ID NO:535, SEQ ID NO:536, SEQ ID NO:537, SEQ ID NO:538, SEQ ID NO:539, SEQ ID NO:540, SEQ ID NO:540, SEQ ID NO:541, SEQ ID NO:542, SEQ ID NO:543, SEQ ID NO:544, SEQ ID NO:545, SEQ ID NO:546, SEQ ID NO:547, SEQ ID NO:548, SEQ ID NO:549, SEQ ID NO:550, SEQ ID NO:551, SEQ ID NO:552, SEQ ID NO:552, SEQ ID NO:552, SEQ ID NO:552, SEQ ID NO:552, SEQ ID NO:552, SEQ ID NO:553, SEQ ID NO:554, SEQ ID NO:555, SEQ ID NO:556, SEQ ID NO:557, SEQ ID NO:558, SEQ ID NO:559, SEQ ID NO:560, SEQ ID NO:561, SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, SEQ ID NO:571, SEQ ID NO:572, SEQ ID NO:572, SEQ ID NO:572, SEQ ID NO:572, SEQ ID NO:572, SEQ ID NO:572, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:580, SEQ ID NO:581, SEQ ID NO:582, SEQ ID NO:583, SEQ ID NO:584, SEQ ID NO:585, SEQ ID NO:586, SEQ ID NO:587, SEQ ID NO:588, SEQ ID NO:589, SEQ ID NO:590, SEQ ID NO:591, SEQ ID NO:592, SEQ ID NO:593, SEQ ID NO:594, SEQ ID NO:595, SEQ ID NO:596, SEQ ID NO:596, SEQ ID NO:596, SEQ ID NO:596, SEQ ID NO:597, SEQ ID NO:598, SEQ ID NO:599, SEQ ID NO:600, SEQ ID NO:601, SEQ ID NO:602, SEQ ID NO:603, SEQ ID NO:604, SEQ ID NO:605, SEQ ID NO:606, SEQ ID NO:607, SEQ ID NO:608, SEQ ID NO:608, SEQ ID NO:609, SEQ ID NO:610, SEQ ID NO:611, SEQ ID NO:612, SEQ ID NO:612, SEQ ID NO:613, SEQ ID NO:614, SEQ ID NO:615, SEQ ID NO:616, SEQ ID NO:617, SEQ ID NO:618, SEQ ID NO:619, SEQ ID NO:620, SEQ ID NO:621, SEQ ID NO:622, SEQ ID NO:623, SEQ ID NO:624, SEQ ID NO:625, SEQ ID NO:626, SEQ ID NO:627, SEQ ID NO:628, SEQ ID NO:629, SEQ ID NO:630, SEQ ID NO:631, SEQ ID NO:632, SEQ ID NO:633, SEQ ID NO:634, SEQ ID NO:635, SEQ ID NO:636, SEQ ID NO:637, SEQ ID NO:638, SEQ ID NO:639, SEQ ID NO:640, SEQ ID NO:641, SEQ ID NO:642, SEQ ID NO:643, SEQ ID NO:643, SEQ ID NO:643, SEQ ID NO:644, SEQ ID NO:645, SEQ ID NO:645, SEQ ID NO:645, SEQ ID NO:645, SEQ ID NO:645, SEQ ID NO:645, SEQ ID NO:646, SEQ ID NO:647, SEQ ID NO:648, SEQ ID NO:649, SEQ ID NO:650, SEQ ID NO:651, SEQ ID NO:652, SEQ ID NO:652, SEQ ID NO:653, SEQ ID NO:654, SEQ ID NO:655, SEQ ID NO:656, SEQ ID NO:657, SEQ ID NO:658, SEQ ID NO:658, SEQ ID NO:659, SEQ ID NO:660, SEQ ID NO:661, SEQ ID NO:662, SEQ ID NO:663, SEQ ID NO:664, SEQ ID NO:665, SEQ ID NO:666, SEQ ID NO:667, SEQ ID NO:667, SEQ ID NO:668, SEQ ID NO:669, SEQ ID NO:669, SEQ ID NO:670, SEQ ID NO:671, SEQ ID NO:672, SEQ ID NO:673, SEQ ID NO:674, SEQ ID NO:675, SEQ ID NO:676, SEQ ID NO:677, SEQ ID NO:678, SEQ ID NO:679, SEQ ID NO:680, SEQ ID NO:681, SEQ ID NO:682, SEQ ID NO:683, SEQ ID NO:684, SEQ ID NO:685, SEQ ID NO:686, SEQ ID NO:1367, SEQ ID NO:1368, SEQ ID NO:1370, SEQ ID NO:1371, SEQ ID NO:1374, SEQ ID NO:1375, SEQ ID NO:1378, SEQ ID NO:1379, SEQ ID NO:1382, SEQ ID NO:1383, SEQ ID NO:1386, SEQ ID NO:1387, SEQ ID NO:1390, SEQ ID NO:1392, SEQ ID NO:1393, SEQ ID NO:1399, SEQ ID NO:1403, SEQ ID NO:1406, or SEQ ID NO:1407.

The disclosure also provides a variable domain sequence comprising a sequence that is at least 80%, at least 85%, at least 90%, or at least 95% identical to a sequence selected from SEQ ID NO:4, SEQ ID NO:5, SEQ ID NO:6, SEQ ID NO:6, SEQ ID NO:7, SEQ ID NO:7, SEQ ID NO:7, SEQ ID NO:7, SEQ ID NO:7, SEQ ID NO:7, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:17, SEQ ID NO:17, SEQ ID NO:18, SEQ ID NO:19, SEQ ID NO:20, SEQ ID NO:20, SEQ ID NO:20, SEQ ID NO:20, SEQ ID NO:21, SEQ ID NO:21, SEQ ID NO:22, SEQ ID NO:23, SEQ ID NO:23, SEQ ID NO:23, SEQ ID NO:24, SEQ ID NO:25, SEQ ID NO:26, SEQ ID NO:27, SEQ ID NO:27, SEQ ID NO:27, SEQ ID NO:27, SEQ ID NO:27, SEQ ID NO:28, SEQ ID NO:29, SEQ ID NO:30, SEQ ID NO:31, SEQ ID NO:32, SEQ ID NO:33, SEQ ID NO:33, SEQ ID NO:34, SEQ ID NO:35, SEQ ID NO:36, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:51, SEQ ID NO:51, SEQ ID NO:52, SEQ ID NO:53, SEQ ID NO:54, SEQ ID NO:55, SEQ ID NO:55, SEQ ID NO:56, SEQ ID NO:57, SEQ ID NO:58, SEQ ID NO:58, SEQ ID NO:59, SEQ ID NO:60, SEQ ID NO:61, SEQ ID NO:62, SEQ ID NO:62, SEQ ID NO:62, SEQ ID NO:62, SEQ ID NO:62, SEQ ID NO:63, SEQ ID NO:64, SEQ ID NO:65, SEQ ID NO:66, SEQ ID NO:67, SEQ ID NO:67, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:75, SEQ ID NO:76, SEQ ID NO:77, SEQ ID NO:78, SEQ ID NO:79, SEQ ID NO:79, SEQ ID NO:80, SEQ ID NO:81, SEQ ID NO:82, SEQ ID NO:83, SEQ ID NO:84, SEQ ID NO:84, SEQ ID NO:84, SEQ ID NO:84, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:163, SEQ ID NO:164, SEQ ID NO:164, SEQ ID NO:165, SEQ ID NO:166, SEQ ID NO:167, SEQ ID NO:168, SEQ ID NO:169, SEQ ID NO:170, SEQ ID NO:170, SEQ ID NO:171, SEQ ID NO:171, SEQ ID NO:171, SEQ ID NO:172, SEQ ID NO:173, SEQ ID NO:173, SEQ ID NO:173, SEQ ID NO:174, SEQ ID NO:175, SEQ ID NO:176, SEQ ID NO:176, SEQ ID NO:177, SEQ ID NO:177, SEQ ID NO:177, SEQ ID NO:177, SEQ ID NO:177, SEQ ID NO:178, SEQ ID NO:179, SEQ ID NO:180, SEQ ID NO:180, SEQ ID NO:181, SEQ ID NO:181, SEQ ID NO:182, SEQ ID NO:182, SEQ ID NO:182, SEQ ID NO:183, SEQ ID NO:183, SEQ ID NO:183, SEQ ID NO:184, SEQ ID NO:185, SEQ ID NO:186, SEQ ID NO:187, SEQ ID NO:188, SEQ ID NO:189, SEQ ID NO:189, SEQ ID NO:189, SEQ ID NO:190, SEQ ID NO:191, SEQ ID NO:192, SEQ ID NO:192, SEQ ID NO:193, SEQ ID NO:194, SEQ ID NO:195, SEQ ID NO:196, SEQ ID NO:197, SEQ ID NO:198, SEQ ID NO:198, SEQ ID NO:199, SEQ ID NO:200, SEQ ID NO:201, SEQ ID NO:201, SEQ ID NO:201, SEQ ID NO:201, SEQ ID NO:201, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:220, SEQ ID NO:221, SEQ ID NO:222, SEQ ID NO:223, SEQ ID NO:224, SEQ ID NO:224, SEQ ID NO:225, SEQ ID NO:226, SEQ ID NO:227, SEQ ID NO:228, SEQ ID NO:229, SEQ ID NO:230, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:311, SEQ ID NO:312, SEQ ID NO:313, SEQ ID NO:313, SEQ ID NO:314, SEQ ID NO:315, SEQ ID NO:315, SEQ ID NO:316, SEQ ID NO:317, SEQ ID NO:318, SEQ ID NO:319, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:330, SEQ ID NO:331, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:1366, SEQ ID NO:1369, SEQ ID NO:1372, SEQ ID NO:1373, SEQ ID NO:1376, SEQ ID NO:1377, SEQ ID NO:1380, SEQ ID NO:1381, SEQ ID NO:1384, SEQ ID NO:1385, SEQ ID NO:1388, SEQ ID NO:1389, SEQ ID NO:1391, SEQ ID NO:1394, SEQ ID NO:1395, SEQ ID NO:1396, SEQ ID NO:1397, SEQ ID NO:1398, SEQ ID NO:1400, SEQ ID NO:1401, SEQ ID NO:1402, SEQ ID NO:1404, or SEQ ID NO:1405. The disclosure also provides a variable domain sequence comprising a sequence that is at least 80%, at least 85%, at least 90%, or at least 95% identical to a sequence selected from SEQ ID NO:380, SEQ ID NO:380, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:381, SEQ ID NO:382, SEQ ID NO:382, SEQ ID NO:382, SEQ ID NO:382, SEQ ID NO:382, SEQ ID NO:383, SEQ ID NO:384, SEQ ID NO:385, SEQ ID NO:385, SEQ ID NO:385, SEQ ID NO:385, SEQ ID NO:385, SEQ ID NO:385, SEQ ID NO:385, SEQ ID NO:386, SEQ ID NO:387, SEQ ID NO:388, SEQ ID NO:389, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:396, SEQ ID NO:397, SEQ ID NO:398, SEQ ID NO:398, SEQ ID NO:398, SEQ ID NO:398, SEQ ID NO:398, SEQ ID NO:398, SEQ ID NO:398, SEQ ID NO:398, SEQ ID NO:399, SEQ ID NO:399, SEQ ID NO:400, SEQ ID NO:401, SEQ ID NO:401, SEQ ID NO:402, SEQ ID NO:403, SEQ ID NO:404, SEQ ID NO:404, SEQ ID NO:405, SEQ ID NO:406, SEQ ID NO:406, SEQ ID NO:406, SEQ ID NO:407, SEQ ID NO:408, SEQ ID NO:409, SEQ ID NO:410, SEQ ID NO:410, SEQ ID NO:411, SEQ ID NO:412, SEQ ID NO:413, SEQ ID NO:414, SEQ ID NO:415, SEQ ID NO:416, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:445, SEQ ID NO:446, SEQ ID NO:447, SEQ ID NO:448, SEQ ID NO:449, SEQ ID NO:450, SEQ ID NO:451, SEQ ID NO:451, SEQ ID NO:451, SEQ ID NO:452, SEQ ID NO:453, SEQ ID NO:454, SEQ ID NO:455, SEQ ID NO:456, SEQ ID NO:456, SEQ ID NO:457, SEQ ID NO:458, SEQ ID NO:459, SEQ ID NO:460, SEQ ID NO:461, SEQ ID NO:462, SEQ ID NO:462, SEQ ID NO:463, SEQ ID NO:464, SEQ ID NO:465, SEQ ID NO:466, SEQ ID NO:466, SEQ ID NO:466, SEQ ID NO:466, SEQ ID NO:467, SEQ ID NO:468, SEQ ID NO:469, SEQ ID NO:469, SEQ ID NO:469, SEQ ID NO:469, SEQ ID NO:470, SEQ ID NO:471, SEQ ID NO:472, SEQ ID NO:473, SEQ ID NO:474, SEQ ID NO:474, SEQ ID NO:475, SEQ ID NO:476, SEQ ID NO:477, SEQ ID NO:478, SEQ ID NO:479, SEQ ID NO:479, SEQ ID NO:480, SEQ ID NO:481, SEQ ID NO:482, SEQ ID NO:482, SEQ ID NO:482, SEQ ID NO:482, SEQ ID NO:482, SEQ ID NO:482, SEQ ID NO:482, SEQ ID NO:482, SEQ ID NO:482, SEQ ID NO:483, SEQ ID NO:483, SEQ ID NO:484, SEQ ID NO:485, SEQ ID NO:486, SEQ ID NO:486, SEQ ID NO:487, SEQ ID NO:488, SEQ ID NO:489, SEQ ID NO:490, SEQ ID NO:491, SEQ ID NO:492, SEQ ID NO:493, SEQ ID NO:494, SEQ ID NO:495, SEQ ID NO:496, SEQ ID NO:497, SEQ ID NO:498, SEQ ID NO:499, SEQ ID NO:500, SEQ ID NO:501, SEQ ID NO:502, SEQ ID NO:503, SEQ ID NO:504, SEQ ID NO:505, SEQ ID NO:506, SEQ ID NO:506, SEQ ID NO:507, SEQ ID NO:508, SEQ ID NO:509, SEQ ID NO:510, SEQ ID NO:511, SEQ ID NO:512, SEQ ID NO:512, SEQ ID NO:513, SEQ ID NO:514, SEQ ID NO:515, SEQ ID NO:515, SEQ ID NO:515, SEQ ID NO:516, SEQ ID NO:517, SEQ ID NO:517, SEQ ID NO:518, SEQ ID NO:519, SEQ ID NO:520, SEQ ID NO:521, SEQ ID NO:522, SEQ ID NO:523, SEQ ID NO:524, SEQ ID NO:525, SEQ ID NO:526, SEQ ID NO:527, SEQ ID NO:527, SEQ ID NO:528, SEQ ID NO:529, SEQ ID NO:529, SEQ ID NO:529, SEQ ID NO:530, SEQ ID NO:531, SEQ ID NO:531, SEQ ID NO:531, SEQ ID NO:532, SEQ ID NO:533, SEQ ID NO:534, SEQ ID NO:535, SEQ ID NO:536, SEQ ID NO:537, SEQ ID NO:538, SEQ ID NO:539, SEQ ID NO:540, SEQ ID NO:540, SEQ ID NO:541, SEQ ID NO:542, SEQ ID NO:543, SEQ ID NO:544, SEQ ID NO:545, SEQ ID NO:546, SEQ ID NO:547, SEQ ID NO:548, SEQ ID NO:549, SEQ ID NO:550, SEQ ID NO:551, SEQ ID NO:552, SEQ ID NO:552, SEQ ID NO:552, SEQ ID NO:552, SEQ ID NO:552, SEQ ID NO:552, SEQ ID NO:553, SEQ ID NO:554, SEQ ID NO:555, SEQ ID NO:556, SEQ ID NO:557, SEQ ID NO:558, SEQ ID NO:559, SEQ ID NO:560, SEQ ID NO:561, SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, SEQ ID NO:571, SEQ ID NO:572, SEQ ID NO:572, SEQ ID NO:572, SEQ ID NO:572, SEQ ID NO:572, SEQ ID NO:572, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:580, SEQ ID NO:581, SEQ ID NO:582, SEQ ID NO:583, SEQ ID NO:584, SEQ ID NO:585, SEQ ID NO:586, SEQ ID NO:587, SEQ ID NO:588, SEQ ID NO:589, SEQ ID NO:590, SEQ ID NO:591, SEQ ID NO:592, SEQ ID NO:593, SEQ ID NO:594, SEQ ID NO:595, SEQ ID NO:596, SEQ ID NO:596, SEQ ID NO:596, SEQ ID NO:596, SEQ ID NO:597, SEQ ID NO:598, SEQ ID NO:599, SEQ ID NO:600, SEQ ID NO:601, SEQ ID NO:602, SEQ ID NO:603, SEQ ID NO:604, SEQ ID NO:605, SEQ ID NO:606, SEQ ID NO:607, SEQ ID NO:608, SEQ ID NO:608, SEQ ID NO:609, SEQ ID NO:610, SEQ ID NO:611, SEQ ID NO:612, SEQ ID NO:612, SEQ ID NO:613, SEQ ID NO:614, SEQ ID NO:615, SEQ ID NO:616, SEQ ID NO:617, SEQ ID NO:618, SEQ ID NO:619, SEQ ID NO:620, SEQ ID NO:621, SEQ ID NO:622, SEQ ID NO:623, SEQ ID NO:624, SEQ ID NO:625, SEQ ID NO:626, SEQ ID NO:627, SEQ ID NO:628, SEQ ID NO:629, SEQ ID NO:630, SEQ ID NO:631, SEQ ID NO:632, SEQ ID NO:633, SEQ ID NO:634, SEQ ID NO:635, SEQ ID NO:636, SEQ ID NO:637, SEQ ID NO:638, SEQ ID NO:639, SEQ ID NO:640, SEQ ID NO:641, SEQ ID NO:642, SEQ ID NO:643, SEQ ID NO:643, SEQ ID NO:643, SEQ ID NO:644, SEQ ID NO:645, SEQ ID NO:645, SEQ ID NO:645, SEQ ID NO:645, SEQ ID NO:645, SEQ ID NO:645, SEQ ID NO:646, SEQ ID NO:647, SEQ ID NO:648, SEQ ID NO:649, SEQ ID NO:650, SEQ ID NO:651, SEQ ID NO:652, SEQ ID NO:652, SEQ ID NO:653, SEQ ID NO:654, SEQ ID NO:655, SEQ ID NO:656, SEQ ID NO:657, SEQ ID NO:658, SEQ ID NO:658, SEQ ID NO:659, SEQ ID NO:660, SEQ ID NO:661, SEQ ID NO:662, SEQ ID NO:663, SEQ ID NO:664, SEQ ID NO:665, SEQ ID NO:666, SEQ ID NO:667, SEQ ID NO:667, SEQ ID NO:668, SEQ ID NO:669, SEQ ID NO:669, SEQ ID NO:670, SEQ ID NO:671, SEQ ID NO:672, SEQ ID NO:673, SEQ ID NO:674, SEQ ID NO:675, SEQ ID NO:676, SEQ ID NO:677, SEQ ID NO:678, SEQ ID NO:679, SEQ ID NO:680, SEQ ID NO:681, SEQ ID NO:682, SEQ ID NO:683, SEQ ID NO:684, SEQ ID NO:685, SEQ ID NO:686, SEQ ID NO:1367, SEQ ID NO:1368, SEQ ID NO:1370, SEQ ID NO:1371, SEQ ID NO:1374, SEQ ID NO:1375, SEQ ID NO:1378, SEQ ID NO:1379, SEQ ID NO:1382, SEQ ID NO:1383, SEQ ID NO:1386, SEQ ID NO:1387, SEQ ID NO:1390, SEQ ID NO:1392, SEQ ID NO:1393, SEQ ID NO:1399, SEQ ID NO:1403, SEQ ID NO:1406, or SEQ ID NO:1407. The disclosure also provides antibodies comprising a heavy chain variable domain sequence that is at least 80%, at least 85%, at least 90%, or at least 95% identical to SEQ ID NO:4 and a light chain variable domain sequence that is at least 80%, at least 85%, at least 90%, or at least 95% identical to SEQ ID NO:380. The disclosure also provides antibodies comprising a heavy chain variable domain sequence that is at least 80%, at least 85%, at least 90%, or at least 95% identical to SEQ ID NO:5 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:6 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:7 and a light chain variable domain sequence that is at least 80%, at least 85%, at least 90%, or at least 95% identical to SEQ ID NO:382. The disclosure also provides antibodies comprising a heavy chain variable domain sequence that is at least 80%, at least 85%, at least 90%, or at least 95% identical to SEQ ID NO:8 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:9 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:10 and a light chain variable domain sequence that is at least 80%, at least 85%, at least 90%, or at least 95% identical to SEQ ID NO:383. The disclosure also provides antibodies comprising a heavy chain variable domain sequence that is at least 80%, at least 85%, at least 90%, or at least 95% identical to SEQ ID NO:11 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:12 and a light chain variable domain sequence that is at least 80%, at least 85%, at least 90%, or at least 95% identical to SEQ ID NO:384. The disclosure also provides antibodies comprising a heavy chain variable domain sequence that is at least 80%, at least 85%, at least 90%, or at least 95% identical to SEQ ID NO:13 and a light chain variable domain sequence that is at least 80%, at least 85%, at least 90%, or at least 95% identical to SEQ ID NO:385. The disclosure also provides antibodies comprising a heavy chain variable domain sequence that is at least 80%, at least 85%, at least 90%, or at least 95% identical to SEQ ID NO:14 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:15 and a light chain variable domain sequence that is at least 80%, at least 85%, at least 90%, or at least 95% identical to SEQ ID NO:386. The disclosure also provides antibodies comprising a heavy chain variable domain sequence that is at least 80%, at least 85%, at least 90%, or at least 95% identical to SEQ ID NO:16 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:17 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:18 and a light chain variable domain sequence that is at least 80%, at least 85%, at least 90%, or at least 95% identical to SEQ ID NO:387. The disclosure also provides antibodies comprising a heavy chain variable domain sequence that is at least 80%, at least 85%, at least 90%, or at least 95% identical to SEQ ID NO:19 and a light chain variable domain sequence that is at least 80%, at least 85%, at least 90%, or at least 95% identical to SEQ ID NO:388. The disclosure also provides antibodies comprising a heavy chain variable domain sequence that is at least 80%, at least 85%, at least 90%, or at least 95% identical to SEQ ID NO:20 and a light chain variable domain sequence that is at least 80%, at least 85%, at least 90%, or at least 95% identical to SEQ ID NO:389. The disclosure also provides antibodies comprising a heavy chain variable domain sequence that is at least 80%, at least 85%, at least 90%, or at least 95% identical to SEQ ID NO:21 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:22 and a light chain variable domain sequence that is at least 80%, at least 85%, at least 90%, or at least 95% identical to SEQ ID NO:390. The disclosure also provides antibodies comprising a heavy chain variable domain sequence that is at least 80%, at least 85%, at least 90%, or at least 95% identical to SEQ ID NO:23 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:24 and a light chain variable domain sequence that is at least 80%, at least 85%, at least 90%, or at least 95% identical to SEQ ID NO:391. The disclosure also provides antibodies comprising a heavy chain variable domain sequence that is at least 80%, at least 85%, at least 90%, or at least 95% identical to SEQ ID NO:20 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:25 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:26 and a light chain variable domain sequence that is at least 80%, at least 85%, at least 90%, or at least 95% identical to SEQ ID NO:392. The disclosure also provides antibodies comprising a heavy chain variable domain sequence that is at least 80%, at least 85%, at least 90%, or at least 95% identical to SEQ ID NO:27 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:28 and a light chain variable domain sequence that is at least 80%, at least 85%, at least 90%, or at least 95% identical to SEQ ID NO:393. The disclosure also provides antibodies comprising a heavy chain variable domain sequence that is at least 80%, at least 85%, at least 90%, or at least 95% identical to SEQ ID NO:29 and a light chain variable domain sequence that is at least 80%, at least 85%, at least 90%, or at least 95% identical to SEQ ID NO:394. The disclosure also provides antibodies comprising a heavy chain variable domain sequence that is at least 80%, at least 85%, at least 90%, or at least 95% identical to SEQ ID NO:30 and a light chain variable domain sequence that is at least 80%, at least 85%, at least 90%, or at least 95% identical to SEQ ID NO:395. The disclosure also provides antibodies comprising a heavy chain variable domain sequence that is at least 80%, at least 85%, at least 90%, or at least 95% identical to SEQ ID NO:31 and a light chain variable domain sequence that is at least 80%, at least 85%, at least 90%, or at least 95% identical to SEQ ID NO:396. The disclosure also provides antibodies comprising a heavy chain variable domain sequence that is at least 80%, at least 85%, at least 90%, or at least 95% identical to SEQ ID NO:32 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:33 and a light chain variable domain sequence that is at least 80%, at least 85%, at least 90%, or at least 95% identical to SEQ ID NO:397. The disclosure also provides antibodies comprising a heavy chain variable domain sequence that is at least 80%, at least 85%, at least 90%, or at least 95% identical to SEQ ID NO:34 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:35 and a light chain variable domain sequence that is at least 80%, at least 85%, at least 90%, or at least 95% identical to SEQ ID NO:398. The disclosure also provides antibodies comprising a heavy chain variable domain sequence that is at least 80%, at least 85%, at least 90%, or at least 95% identical to SEQ ID NO:36 and a light chain variable domain sequence that is at least 80%, at least 85%, at least 90%, or at least 95% identical to SEQ ID NO:399. The disclosure also provides antibodies comprising a heavy chain variable domain sequence that is at least 80%, at least 85%, at least 90%, or at least 95% identical to SEQ ID NO:37 and a light chain variable domain sequence that is at least 80%, at least 85%, at least 90%, or at least 95% identical to SEQ ID NO:400. The disclosure also provides antibodies comprising a heavy chain variable domain sequence that is at least 80%, at least 85%, at least 90%, or at least 95% identical to SEQ ID NO:38 and a light chain variable domain sequence that is at least 80%, at least 85%, at least 90%, or at least 95% identical to SEQ ID NO:401. The disclosure also provides antibodies comprising a heavy chain variable domain sequence that is at least 80%, at least 85%, at least 90%, or at least 95% identical to SEQ ID NO:39 and a light chain variable domain sequence that is at least 80%, at least 85%, at least 90%, or at least 95% identical to SEQ ID NO:402. The disclosure also provides antibodies comprising a heavy chain variable domain sequence that is at least 80%, at least 85%, at least 90%, or at least 95% identical to SEQ ID NO:40 and a light chain variable domain sequence that is at least 80%, at least 85%, at least 90%, or at least 95% identical to SEQ ID NO:380. The disclosure also provides antibodies comprising a heavy chain variable domain sequence that is at least 80%, at least 85%, at least 90%, or at least 95% identical to SEQ ID NO:41 and a light chain variable domain sequence that is at least 80%, at least 85%, at least 90%, or at least 95% identical to SEQ ID NO:403. The disclosure also provides antibodies comprising a heavy chain variable domain sequence that is at least 80%, at least 85%, at least 90%, or at least 95% identical to SEQ ID NO:42 and a light chain variable domain sequence that is at least 80%, at least 85%, at least 90%, or at least 95% identical to SEQ ID NO:398. The disclosure also provides antibodies comprising a heavy chain variable domain sequence that is at least 80%, at least 85%, at least 90%, or at least 95% identical to SEQ ID NO:43 and a light chain variable domain sequence that is at least 80%, at least 85%, at least 90%, or at least 95% identical to SEQ ID NO:404. The disclosure also provides antibodies comprising a heavy chain variable domain sequence that is at least 80%, at least 85%, at least 90%, or at least 95% identical to SEQ ID NO:6 and a light chain variable domain sequence that is at least 80%, at least 85%, at least 90%, or at least 95% identical to SEQ ID NO:405. The disclosure also provides antibodies comprising a heavy chain variable domain sequence that is at least 80%, at least 85%, at least 90%, or at least 95% identical to SEQ ID NO:27 and a light chain variable domain sequence that is at least 80%, at least 85%, at least 90%, or at least 95% identical to SEQ ID NO:406. The disclosure also provides antibodies comprising a heavy chain variable domain sequence that is at least 80%, at least 85%, at least 90%, or at least 95% identical to SEQ ID NO:23 and a light chain variable domain sequence that is at least 80%, at least 85%, at least 90%, or at least 95% identical to SEQ ID NO:382. The disclosure also provides antibodies comprising a heavy chain variable domain sequence that is at least 80%, at least 85%, at least 90%, or at least 95% identical to SEQ ID NO:20 and a light chain variable domain sequence that is at least 80%, at least 85%, at least 90%, or at least 95% identical to SEQ ID NO:406. The disclosure also provides antibodies comprising a heavy chain variable domain sequence that is at least 80%, at least 85%, at least 90%, or at least 95% identical to SEQ ID NO:44 and a light chain variable domain sequence that is at least 80%, at least 85%, at least 90%, or at least 95% identical to SEQ ID NO:398. The disclosure also provides antibodies comprising a heavy chain variable domain sequence that is at least 80%, at least 85%, at least 90%, or at least 95% identical to SEQ ID NO:45 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:46 and a light chain variable domain sequence that is at least 80%, at least 85%, at least 90%, or at least 95% identical to SEQ ID NO:399. The disclosure also provides antibodies comprising a heavy chain variable domain sequence that is at least 80%, at least 85%, at least 90%, or at least 95% identical to SEQ ID NO:47 and a light chain variable domain sequence that is at least 80%, at least 85%, at least 90%, or at least 95% identical to SEQ ID NO:407. The disclosure also provides antibodies comprising a heavy chain variable domain sequence that is at least 80%, at least 85%, at least 90%, or at least 95% identical to SEQ ID NO:21 and a light chain variable domain sequence that is at least 80%, at least 85%, at least 90%, or at least 95% identical to SEQ ID NO:382. The disclosure also provides antibodies comprising a heavy chain variable domain sequence that is at least 80%, at least 85%, at least 90%, or at least 95% identical to SEQ ID NO:48 and a light chain variable domain sequence that is at least 80%, at least 85%, at least 90%, or at least 95% identical to SEQ ID NO:408. The disclosure also provides antibodies comprising a heavy chain variable domain sequence that is at least 80%, at least 85%, at least 90%, or at least 95% identical to SEQ ID NO:49 and a light chain variable domain sequence that is at least 80%, at least 85%, at least 90%, or at least 95% identical to SEQ ID NO:409. The disclosure also provides antibodies comprising a heavy chain variable domain sequence that is at least 80%, at least 85%, at least 90%, or at least 95% identical to SEQ ID NO:50 and a light chain variable domain sequence that is at least 80%, at least 85%, at least 90%, or at least 95% identical to SEQ ID NO:410. The disclosure also provides antibodies comprising a heavy chain variable domain sequence that is at least 80%, at least 85%, at least 90%, or at least 95% identical to SEQ ID NO:51 and a light chain variable domain sequence that is at least 80%, at least 85%, at least 90%, or at least 95% identical to SEQ ID NO:411. The disclosure also provides antibodies comprising a heavy chain variable domain sequence that is at least 80%, at least 85%, at least 90%, or at least 95% identical to SEQ ID NO:52 and a light chain variable domain sequence that is at least 80%, at least 85%, at least 90%, or at least 95% identical to SEQ ID NO:412. The disclosure also provides antibodies comprising a heavy chain variable domain sequence that is at least 80%, at least 85%, at least 90%, or at least 95% identical to SEQ ID NO:53 and a light chain variable domain sequence that is at least 80%, at least 85%, at least 90%, or at least 95% identical to SEQ ID NO:413. The disclosure also provides antibodies comprising a heavy chain variable domain sequence that is at least 80%, at least 85%, at least 90%, or at least 95% identical to SEQ ID NO:54 and a light chain variable domain sequence that is at least 80%, at least 85%, at least 90%, or at least 95% identical to SEQ ID NO:414. The disclosure also provides antibodies comprising a heavy chain variable domain sequence that is at least 80%, at least 85%, at least 90%, or at least 95% identical to SEQ ID NO:55 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:56 and a light chain variable domain sequence that is at least 80%, at least 85%, at least 90%, or at least 95% identical to SEQ ID NO:415. The disclosure also provides antibodies comprising a heavy chain variable domain sequence that is at least 80%, at least 85%, at least 90%, or at least 95% identical to SEQ ID NO:57 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:58 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:33 and a light chain variable domain sequence that is at least 80%, at least 85%, at least 90%, or at least 95% identical to SEQ ID NO:416. The disclosure also provides antibodies comprising a heavy chain variable domain sequence that is at least 80%, at least 85%, at least 90%, or at least 95% identical to SEQ ID NO:59 and a light chain variable domain sequence that is at least 80%, at least 85%, at least 90%, or at least 95% identical to SEQ ID NO:417. The disclosure also provides antibodies comprising a heavy chain variable domain sequence that is at least 80%, at least 85%, at least 90%, or at least 95% identical to SEQ ID NO:60 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:61 and a light chain variable domain sequence that is at least 80%, at least 85%, at least 90%, or at least 95% identical to SEQ ID NO:418. The disclosure also provides antibodies comprising a heavy chain variable domain sequence that is at least 80%, at least 85%, at least 90%, or at least 95% identical to SEQ ID NO:20 and a light chain variable domain sequence that is at least 80%, at least 85%, at least 90%, or at least 95% identical to SEQ ID NO:419. The disclosure also provides antibodies comprising a heavy chain variable domain sequence that is at least 80%, at least 85%, at least 90%, or at least 95% identical to SEQ ID NO:62 and a light chain variable domain sequence that is at least 80%, at least 85%, at least 90%, or at least 95% identical to SEQ ID NO:401. The disclosure also provides antibodies comprising a heavy chain variable domain sequence that is at least 80%, at least 85%, at least 90%, or at least 95% identical to SEQ ID NO:7 and a light chain variable domain sequence that is at least 80%, at least 85%, at least 90%, or at least 95% identical to SEQ ID NO:385. The disclosure also provides antibodies comprising a heavy chain variable domain sequence that is at least 80%, at least 85%, at least 90%, or at least 95% identical to SEQ ID NO:62 and a light chain variable domain sequence that is at least 80%, at least 85%, at least 90%, or at least 95% identical to SEQ ID NO:420. The disclosure also provides antibodies comprising a heavy chain variable domain sequence that is at least 80%, at least 85%, at least 90%, or at least 95% identical to SEQ ID NO:63 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:62 and a light chain variable domain sequence that is at least 80%, at least 85%, at least 90%, or at least 95% identical to SEQ ID NO:421. The disclosure also provides antibodies comprising a heavy chain variable domain sequence that is at least 80%, at least 85%, at least 90%, or at least 95% identical to SEQ ID NO:64 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:7 and a light chain variable domain sequence that is at least 80%, at least 85%, at least 90%, or at least 95% identical to SEQ ID NO:422. The disclosure also provides antibodies comprising a heavy chain variable domain sequence that is at least 80%, at least 85%, at least 90%, or at least 95% identical to SEQ ID NO:65 and a light chain variable domain sequence that is at least 80%, at least 85%, at least 90%, or at least 95% identical to SEQ ID NO:423. The disclosure also provides antibodies comprising a heavy chain variable domain sequence that is at least 80%, at least 85%, at least 90%, or at least 95% identical to SEQ ID NO:66 and a light chain variable domain sequence that is at least 80%, at least 85%, at least 90%, or at least 95% identical to SEQ ID NO:424. The disclosure also provides antibodies comprising a heavy chain variable domain sequence that is at least 80%, at least 85%, at least 90%, or at least 95% identical to SEQ ID NO:67 and a light chain variable domain sequence that is at least 80%, at least 85%, at least 90%, or at least 95% identical to SEQ ID NO:425. The disclosure also provides antibodies comprising a heavy chain variable domain sequence that is at least 80%, at least 85%, at least 90%, or at least 95% identical to SEQ ID NO:55 and a light chain variable domain sequence that is at least 80%, at least 85%, at least 90%, or at least 95% identical to SEQ ID NO:426. The disclosure also provides antibodies comprising a heavy chain variable domain sequence that is at least 80%, at least 85%, at least 90%, or at least 95% identical to SEQ ID NO:68 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:69 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:62 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:70 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:71 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:36 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:72 and a light chain variable domain sequence that is at least 80%, at least 85%, at least 90%, or at least 95% identical to SEQ ID NO:385. The disclosure also provides antibodies comprising a heavy chain variable domain sequence that is at least 80%, at least 85%, at least 90%, or at least 95% identical to SEQ ID NO:73 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:74 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:75 and a light chain variable domain sequence that is at least 80%, at least 85%, at least 90%, or at least 95% identical to SEQ ID NO:427. The disclosure also provides antibodies comprising a heavy chain variable domain sequence that is at least 80%, at least 85%, at least 90%, or at least 95% identical to SEQ ID NO:75 and a light chain variable domain sequence that is at least 80%, at least 85%, at least 90%, or at least 95% identical to SEQ ID NO:428. The disclosure also provides antibodies comprising a heavy chain variable domain sequence that is at least 80%, at least 85%, at least 90%, or at least 95% identical to SEQ ID NO:76 and a light chain variable domain sequence that is at least 80%, at least 85%, at least 90%, or at least 95% identical to SEQ ID NO:385. The disclosure also provides antibodies comprising a heavy chain variable domain sequence that is at least 80%, at least 85%, at least 90%, or at least 95% identical to SEQ ID NO:77 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:78 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:79 and a light chain variable domain sequence that is at least 80%, at least 85%, at least 90%, or at least 95% identical to SEQ ID NO:429. The disclosure also provides antibodies comprising a heavy chain variable domain sequence that is at least 80%, at least 85%, at least 90%, or at least 95% identical to SEQ ID NO:80 and a light chain variable domain sequence that is at least 80%, at least 85%, at least 90%, or at least 95% identical to SEQ ID NO:430. The disclosure also provides antibodies comprising a heavy chain variable domain sequence that is at least 80%, at least 85%, at least 90%, or at least 95% identical to SEQ ID NO:67 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:81 and a light chain variable domain sequence that is at least 80%, at least 85%, at least 90%, or at least 95% identical to SEQ ID NO:431. The disclosure also provides antibodies comprising a heavy chain variable domain sequence that is at least 80%, at least 85%, at least 90%, or at least 95% identical to SEQ ID NO:82 and a light chain variable domain sequence that is at least 80%, at least 85%, at least 90%, or at least 95% identical to SEQ ID NO:432. The disclosure also provides antibodies comprising a heavy chain variable domain sequence that is at least 80%, at least 85%, at least 90%, or at least 95% identical to SEQ ID NO:83 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:84 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:85 and a light chain variable domain sequence that is at least 80%, at least 85%, at least 90%, or at least 95% identical to SEQ ID NO:433. The disclosure also provides antibodies comprising a heavy chain variable domain sequence that is at least 80%, at least 85%, at least 90%, or at least 95% identical to SEQ ID NO:79 and a light chain variable domain sequence that is at least 80%, at least 85%, at least 90%, or at least 95% identical to SEQ ID NO:434. The disclosure also provides antibodies comprising a heavy chain variable domain sequence that is at least 80%, at least 85%, at least 90%, or at least 95% identical to SEQ ID NO:86 and a light chain variable domain sequence that is at least 80%, at least 85%, at least 90%, or at least 95% identical to SEQ ID NO:435. The disclosure also provides antibodies comprising a heavy chain variable domain sequence that is at least 80%, at least 85%, at least 90%, or at least 95% identical to SEQ ID NO:87 and a light chain variable domain sequence that is at least 80%, at least 85%, at least 90%, or at least 95% identical to SEQ ID NO:436. The disclosure also provides antibodies comprising a heavy chain variable domain sequence that is at least 80%, at least 85%, at least 90%, or at least 95% identical to SEQ ID NO:88 and a light chain variable domain sequence that is at least 80%, at least 85%, at least 90%, or at least 95% identical to SEQ ID NO:437. The disclosure also provides antibodies comprising a heavy chain variable domain sequence that is at least 80%, at least 85%, at least 90%, or at least 95% identical to SEQ ID NO:89 and a light chain variable domain sequence that is at least 80%, at least 85%, at least 90%, or at least 95% identical to SEQ ID NO:438. The disclosure also provides antibodies comprising a heavy chain variable domain sequence that is at least 80%, at least 85%, at least 90%, or at least 95% identical to SEQ ID NO:90 and a light chain variable domain sequence that is at least 80%, at least 85%, at least 90%, or at least 95% identical to SEQ ID NO:439. The disclosure also provides antibodies comprising a heavy chain variable domain sequence that is at least 80%, at least 85%, at least 90%, or at least 95% identical to SEQ ID NO:91 and a light chain variable domain sequence that is at least 80%, at least 85%, at least 90%, or at least 95% identical to SEQ ID NO:440. The disclosure also provides antibodies comprising a heavy chain variable domain sequence that is at least 80%, at least 85%, at least 90%, or at least 95% identical to SEQ ID NO:92 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:93 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:94 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:62 and a light chain variable domain sequence that is at least 80%, at least 85%, at least 90%, or at least 95% identical to SEQ ID NO:441. The disclosure also provides antibodies comprising a heavy chain variable domain sequence that is at least 80%, at least 85%, at least 90%, or at least 95% identical to SEQ ID NO:95 and a light chain variable domain sequence that is at least 80%, at least 85%, at least 90%, or at least 95% identical to SEQ ID NO:398. The disclosure also provides antibodies comprising a heavy chain variable domain sequence that is at least 80%, at least 85%, at least 90%, or at least 95% identical to SEQ ID NO:96 and a light chain variable domain sequence that is at least 80%, at least 85%, at least 90%, or at least 95% identical to SEQ ID NO:442. The disclosure also provides antibodies comprising a heavy chain variable domain sequence that is at least 80%, at least 85%, at least 90%, or at least 95% identical to SEQ ID NO:84 and a light chain variable domain sequence that is at least 80%, at least 85%, at least 90%, or at least 95% identical to SEQ ID NO:443. The disclosure also provides antibodies comprising a heavy chain variable domain sequence that is at least 80%, at least 85%, at least 90%, or at least 95% identical to SEQ ID NO:97 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:98 and a light chain variable domain sequence that is at least 80%, at least 85%, at least 90%, or at least 95% identical to SEQ ID NO:444. The disclosure also provides antibodies comprising a heavy chain variable domain sequence that is at least 80%, at least 85%, at least 90%, or at least 95% identical to SEQ ID NO:99 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:100 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:7 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:101 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:102 and a light chain variable domain sequence that is at least 80%, at least 85%, at least 90%, or at least 95% identical to SEQ ID NO:445. The disclosure also provides antibodies comprising a heavy chain variable domain sequence that is at least 80%, at least 85%, at least 90%, or at least 95% identical to SEQ ID NO:27 and a light chain variable domain sequence that is at least 80%, at least 85%, at least 90%, or at least 95% identical to SEQ ID NO:446. The disclosure also provides antibodies comprising a heavy chain variable domain sequence that is at least 80%, at least 85%, at least 90%, or at least 95% identical to SEQ ID NO:103 and a light chain variable domain sequence that is at least 80%, at least 85%, at least 90%, or at least 95% identical to SEQ ID NO:447. The disclosure also provides antibodies comprising a heavy chain variable domain sequence that is at least 80%, at least 85%, at least 90%, or at least 95% identical to SEQ ID NO:84 and a light chain variable domain sequence that is at least 80%, at least 85%, at least 90%, or at least 95% identical to SEQ ID NO:385. The disclosure also provides antibodies comprising a heavy chain variable domain sequence that is at least 80%, at least 85%, at least 90%, or at least 95% identical to SEQ ID NO:104 and a light chain variable domain sequence that is at least 80%, at least 85%, at least 90%, or at least 95% identical to SEQ ID NO:382. The disclosure also provides antibodies comprising a heavy chain variable domain sequence that is at least 80%, at least 85%, at least 90%, or at least 95% identical to SEQ ID NO:7 and a light chain variable domain sequence that is at least 80%, at least 85%, at least 90%, or at least 95% identical to SEQ ID NO:448. The disclosure also provides antibodies comprising a heavy chain variable domain sequence that is at least 80%, at least 85%, at least 90%, or at least 95% identical to SEQ ID NO:105 and a light chain variable domain sequence that is at least 80%, at least 85%, at least 90%, or at least 95% identical to SEQ ID NO:404. The disclosure also provides antibodies comprising a heavy chain variable domain sequence that is at least 80%, at least 85%, at least 90%, or at least 95% identical to SEQ ID NO:106 and a light chain variable domain sequence that is at least 80%, at least 85%, at least 90%, or at least 95% identical to SEQ ID NO:410. The disclosure also provides antibodies comprising a heavy chain variable domain sequence that is at least 80%, at least 85%, at least 90%, or at least 95% identical to SEQ ID NO:107 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:108 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:109 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:110 and a light chain variable domain sequence that is at least 80%, at least 85%, at least 90%, or at least 95% identical to SEQ ID NO:449. The disclosure also provides antibodies comprising a heavy chain variable domain sequence that is at least 80%, at least 85%, at least 90%, or at least 95% identical to SEQ ID NO:111 and a light chain variable domain sequence that is at least 80%, at least 85%, at least 90%, or at least 95% identical to SEQ ID NO:450. The disclosure also provides antibodies comprising a heavy chain variable domain sequence that is at least 80%, at least 85%, at least 90%, or at least 95% identical to SEQ ID NO:27 and a light chain variable domain sequence that is at least 80%, at least 85%, at least 90%, or at least 95% identical to SEQ ID NO:451. The disclosure also provides antibodies comprising a heavy chain variable domain sequence that is at least 80%, at least 85%, at least 90%, or at least 95% identical to SEQ ID NO:112 and a light chain variable domain sequence that is at least 80%, at least 85%, at least 90%, or at least 95% identical to SEQ ID NO:452. The disclosure also provides antibodies comprising a heavy chain variable domain sequence that is at least 80%, at least 85%, at least 90%, or at least 95% identical to SEQ ID NO:113 and a light chain variable domain sequence that is at least 80%, at least 85%, at least 90%, or at least 95% identical to SEQ ID NO:385. The disclosure also provides antibodies comprising a heavy chain variable domain sequence that is at least 80%, at least 85%, at least 90%, or at least 95% identical to SEQ ID NO:114 and a light chain variable domain sequence that is at least 80%, at least 85%, at least 90%, or at least 95% identical to SEQ ID NO:453. The disclosure also provides antibodies comprising a heavy chain variable domain sequence that is at least 80%, at least 85%, at least 90%, or at least 95% identical to SEQ ID NO:115 and a light chain variable domain sequence that is at least 80%, at least 85%, at least 90%, or at least 95% identical to SEQ ID NO:454. The disclosure also provides antibodies comprising a heavy chain variable domain sequence that is at least 80%, at least 85%, at least 90%, or at least 95% identical to SEQ ID NO:116 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:117 and a light chain variable domain sequence that is at least 80%, at least 85%, at least 90%, or at least 95% identical to SEQ ID NO:385. The disclosure also provides antibodies comprising a heavy chain variable domain sequence that is at least 80%, at least 85%, at least 90%, or at least 95% identical to SEQ ID NO:17 and a light chain variable domain sequence that is at least 80%, at least 85%, at least 90%, or at least 95% identical to SEQ ID NO:455. The disclosure also provides antibodies comprising a heavy chain variable domain sequence that is at least 80%, at least 85%, at least 90%, or at least 95% identical to SEQ ID NO:23 and a light chain variable domain sequence that is at least 80%, at least 85%, at least 90%, or at least 95% identical to SEQ ID NO:456. The disclosure also provides antibodies comprising a heavy chain variable domain sequence that is at least 80%, at least 85%, at least 90%, or at least 95% identical to SEQ ID NO:118 and a light chain variable domain sequence that is at least 80%, at least 85%, at least 90%, or at least 95% identical to SEQ ID NO:456. The disclosure also provides antibodies comprising a heavy chain variable domain sequence that is at least 80%, at least 85%, at least 90%, or at least 95% identical to SEQ ID NO:119 and a light chain variable domain sequence that is at least 80%, at least 85%, at least 90%, or at least 95% identical to SEQ ID NO:398. The disclosure also provides antibodies comprising a heavy chain variable domain sequence that is at least 80%, at least 85%, at least 90%, or at least 95% identical to SEQ ID NO:120 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:121 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:84 and a light chain variable domain sequence that is at least 80%, at least 85%, at least 90%, or at least 95% identical to SEQ ID NO:457. The disclosure also provides antibodies comprising a heavy chain variable domain sequence that is at least 80%, at least 85%, at least 90%, or at least 95% identical to SEQ ID NO:122 and a light chain variable domain sequence that is at least 80%, at least 85%, at least 90%, or at least 95% identical to SEQ ID NO:458. The disclosure also provides antibodies comprising a heavy chain variable domain sequence that is at least 80%, at least 85%, at least 90%, or at least 95% identical to SEQ ID NO:123 and a light chain variable domain sequence that is at least 80%, at least 85%, at least 90%, or at least 95% identical to SEQ ID NO:406. The disclosure also provides antibodies comprising a heavy chain variable domain sequence that is at least 80%, at least 85%, at least 90%, or at least 95% identical to SEQ ID NO:93 and a light chain variable domain sequence that is at least 80%, at least 85%, at least 90%, or at least 95% identical to SEQ ID NO:451. The disclosure also provides antibodies comprising a heavy chain variable domain sequence that is at least 80%, at least 85%, at least 90%, or at least 95% identical to SEQ ID NO:58 and a light chain variable domain sequence that is at least 80%, at least 85%, at least 90%, or at least 95% identical to SEQ ID NO:389. The disclosure also provides antibodies comprising a heavy chain variable domain sequence that is at least 80%, at least 85%, at least 90%, or at least 95% identical to SEQ ID NO:124 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:17 and a light chain variable domain sequence that is at least 80%, at least 85%, at least 90%, or at least 95% identical to SEQ ID NO:451. The disclosure also provides antibodies comprising a heavy chain variable domain sequence that is at least 80%, at least 85%, at least 90%, or at least 95% identical to SEQ ID NO:125 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:27 and a light chain variable domain sequence that is at least 80%, at least 85%, at least 90%, or at least 95% identical to SEQ ID NO:445. The disclosure also provides antibodies comprising a heavy chain variable domain sequence that is at least 80%, at least 85%, at least 90%, or at least 95% identical to SEQ ID NO:126 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:127 and a light chain variable domain sequence that is at least 80%, at least 85%, at least 90%, or at least 95% identical to SEQ ID NO:459. The disclosure also provides antibodies comprising a heavy chain variable domain sequence that is at least 80%, at least 85%, at least 90%, or at least 95% identical to SEQ ID NO:7 and a light chain variable domain sequence that is at least 80%, at least 85%, at least 90%, or at least 95% identical to SEQ ID NO:460. The disclosure also provides antibodies comprising a heavy chain variable domain sequence that is at least 80%, at least 85%, at least 90%, or at least 95% identical to SEQ ID NO:7 and a light chain variable domain sequence that is at least 80%, at least 85%, at least 90%, or at least 95% identical to SEQ ID NO:398. The disclosure also provides antibodies comprising a heavy chain variable domain sequence that is at least 80%, at least 85%, at least 90%, or at least 95% identical to SEQ ID NO:128 and a light chain variable domain sequence that is at least 80%, at least 85%, at least 90%, or at least 95% identical to SEQ ID NO:461. The disclosure also provides antibodies comprising a heavy chain variable domain sequence that is at least 80%, at least 85%, at least 90%, or at least 95% identical to SEQ ID NO:129 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:130 and a light chain variable domain sequence that is at least 80%, at least 85%, at least 90%, or at least 95% identical to SEQ ID NO:462. The disclosure also provides antibodies comprising a heavy chain variable domain sequence that is at least 80%, at least 85%, at least 90%, or at least 95% identical to SEQ ID NO:84 and a light chain variable domain sequence that is at least 80%, at least 85%, at least 90%, or at least 95% identical to SEQ ID NO:398. The disclosure also provides antibodies comprising a heavy chain variable domain sequence that is at least 80%, at least 85%, at least 90%, or at least 95% identical to SEQ ID NO:131 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:132 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:133 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:67 and a light chain variable domain sequence that is at least 80%, at least 85%, at least 90%, or at least 95% identical to SEQ ID NO:382. The disclosure also provides antibodies comprising a heavy chain variable domain sequence that is at least 80%, at least 85%, at least 90%, or at least 95% identical to SEQ ID NO:134 and a light chain variable domain sequence that is at least 80%, at least 85%, at least 90%, or at least 95% identical to SEQ ID NO:396. The disclosure also provides antibodies comprising a heavy chain variable domain sequence that is at least 80%, at least 85%, at least 90%, or at least 95% identical to SEQ ID NO:51 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:51 and a light chain variable domain sequence that is at least 80%, at least 85%, at least 90%, or at least 95% identical to SEQ ID NO:463. The disclosure also provides antibodies comprising a heavy chain variable domain sequence that is at least 80%, at least 85%, at least 90%, or at least 95% identical to SEQ ID NO:135 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:102 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:136 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:137 and a light chain variable domain sequence that is at least 80%, at least 85%, at least 90%, or at least 95% identical to SEQ ID NO:398. The disclosure also provides antibodies comprising a heavy chain variable domain sequence that is at least 80%, at least 85%, at least 90%, or at least 95% identical to SEQ ID NO:138 and a light chain variable domain sequence that is at least 80%, at least 85%, at least 90%, or at least 95% identical to SEQ ID NO:462. The disclosure also provides antibodies comprising a heavy chain variable domain sequence that is at least 80%, at least 85%, at least 90%, or at least 95% identical to SEQ ID NO:139 and a light chain variable domain sequence that is at least 80%, at least 85%, at least 90%, or at least 95% identical to SEQ ID NO:464. The disclosure also provides antibodies comprising a heavy chain variable domain sequence that is at least 80%, at least 85%, at least 90%, or at least 95% identical to SEQ ID NO:140 and a light chain variable domain sequence that is at least 80%, at least 85%, at least 90%, or at least 95% identical to SEQ ID NO:465. The disclosure also provides antibodies comprising a heavy chain variable domain sequence that is at least 80%, at least 85%, at least 90%, or at least 95% identical to SEQ ID NO:141 and a light chain variable domain sequence that is at least 80%, at least 85%, at least 90%, or at least 95% identical to SEQ ID NO:466. The disclosure also provides antibodies comprising a heavy chain variable domain sequence that is at least 80%, at least 85%, at least 90%, or at least 95% identical to SEQ ID NO:142 and a light chain variable domain sequence that is at least 80%, at least 85%, at least 90%, or at least 95% identical to SEQ ID NO:467. The disclosure also provides antibodies comprising a heavy chain variable domain sequence that is at least 80%, at least 85%, at least 90%, or at least 95% identical to SEQ ID NO:143 and a light chain variable domain sequence that is at least 80%, at least 85%, at least 90%, or at least 95% identical to SEQ ID NO:468. The disclosure also provides antibodies comprising a heavy chain variable domain sequence that is at least 80%, at least 85%, at least 90%, or at least 95% identical to SEQ ID NO:144 and a light chain variable domain sequence that is at least 80%, at least 85%, at least 90%, or at least 95% identical to SEQ ID NO:469. The disclosure also provides antibodies comprising a heavy chain variable domain sequence that is at least 80%, at least 85%, at least 90%, or at least 95% identical to SEQ ID NO:145 and a light chain variable domain sequence that is at least 80%, at least 85%, at least 90%, or at least 95% identical to SEQ ID NO:470. The disclosure also provides antibodies comprising a heavy chain variable domain sequence that is at least 80%, at least 85%, at least 90%, or at least 95% identical to SEQ ID NO:146 and a light chain variable domain sequence that is at least 80%, at least 85%, at least 90%, or at least 95% identical to SEQ ID NO:471. The disclosure also provides antibodies comprising a heavy chain variable domain sequence that is at least 80%, at least 85%, at least 90%, or at least 95% identical to SEQ ID NO:147 and a light chain variable domain sequence that is at least 80%, at least 85%, at least 90%, or at least 95% identical to SEQ ID NO:472. The disclosure also provides antibodies comprising a heavy chain variable domain sequence that is at least 80%, at least 85%, at least 90%, or at least 95% identical to SEQ ID NO:148 and a light chain variable domain sequence that is at least 80%, at least 85%, at least 90%, or at least 95% identical to SEQ ID NO:473. The disclosure also provides antibodies comprising a heavy chain variable domain sequence that is at least 80%, at least 85%, at least 90%, or at least 95% identical to SEQ ID NO:149 and a light chain variable domain sequence that is at least 80%, at least 85%, at least 90%, or at least 95% identical to SEQ ID NO:474. The disclosure also provides antibodies comprising a heavy chain variable domain sequence that is at least 80%, at least 85%, at least 90%, or at least 95% identical to SEQ ID NO:150 and a light chain variable domain sequence that is at least 80%, at least 85%, at least 90%, or at least 95% identical to SEQ ID NO:475. The disclosure also provides antibodies comprising a heavy chain variable domain sequence that is at least 80%, at least 85%, at least 90%, or at least 95% identical to SEQ ID NO:151 and a light chain variable domain sequence that is at least 80%, at least 85%, at least 90%, or at least 95% identical to SEQ ID NO:476. The disclosure also provides antibodies comprising a heavy chain variable domain sequence that is at least 80%, at least 85%, at least 90%, or at least 95% identical to SEQ ID NO:152 and a light chain variable domain sequence that is at least 80%, at least 85%, at least 90%, or at least 95% identical to SEQ ID NO:477. The disclosure also provides antibodies comprising a heavy chain variable domain sequence that is at least 80%, at least 85%, at least 90%, or at least 95% identical to SEQ ID NO:153 and a light chain variable domain sequence that is at least 80%, at least 85%, at least 90%, or at least 95% identical to SEQ ID NO:478. The disclosure also provides antibodies comprising a heavy chain variable domain sequence that is at least 80%, at least 85%, at least 90%, or at least 95% identical to SEQ ID NO:154 and a light chain variable domain sequence that is at least 80%, at least 85%, at least 90%, or at least 95% identical to SEQ ID NO:479. The disclosure also provides antibodies comprising a heavy chain variable domain sequence that is at least 80%, at least 85%, at least 90%, or at least 95% identical to SEQ ID NO:155 and a light chain variable domain sequence that is at least 80%, at least 85%, at least 90%, or at least 95% identical to SEQ ID NO:480. The disclosure also provides antibodies comprising a heavy chain variable domain sequence that is at least 80%, at least 85%, at least 90%, or at least 95% identical to SEQ ID NO:156 and a light chain variable domain sequence that is at least 80%, at least 85%, at least 90%, or at least 95% identical to SEQ ID NO:481. The disclosure also provides antibodies comprising a heavy chain variable domain sequence that is at least 80%, at least 85%, at least 90%, or at least 95% identical to SEQ ID NO:157 and a light chain variable domain sequence that is at least 80%, at least 85%, at least 90%, or at least 95% identical to SEQ ID NO:482. The disclosure also provides antibodies comprising a heavy chain variable domain sequence that is at least 80%, at least 85%, at least 90%, or at least 95% identical to SEQ ID NO:158 and a light chain variable domain sequence that is at least 80%, at least 85%, at least 90%, or at least 95% identical to SEQ ID NO:483. The disclosure also provides antibodies comprising a heavy chain variable domain sequence that is at least 80%, at least 85%, at least 90%, or at least 95% identical to SEQ ID NO:159 and a light chain variable domain sequence that is at least 80%, at least 85%, at least 90%, or at least 95% identical to SEQ ID NO:484. The disclosure also provides antibodies comprising a heavy chain variable domain sequence that is at least 80%, at least 85%, at least 90%, or at least 95% identical to SEQ ID NO:160 and a light chain variable domain sequence that is at least 80%, at least 85%, at least 90%, or at least 95% identical to SEQ ID NO:485. The disclosure also provides antibodies comprising a heavy chain variable domain sequence that is at least 80%, at least 85%, at least 90%, or at least 95% identical to SEQ ID NO:161 and a light chain variable domain sequence that is at least 80%, at least 85%, at least 90%, or at least 95% identical to SEQ ID NO:486. The disclosure also provides antibodies comprising a heavy chain variable domain sequence that is at least 80%, at least 85%, at least 90%, or at least 95% identical to SEQ ID NO:162 and a light chain variable domain sequence that is at least 80%, at least 85%, at least 90%, or at least 95% identical to SEQ ID NO:487. The disclosure also provides antibodies comprising a heavy chain variable domain sequence that is at least 80%, at least 85%, at least 90%, or at least 95% identical to SEQ ID NO:163 and a light chain variable domain sequence that is at least 80%, at least 85%, at least 90%, or at least 95% identical to SEQ ID NO:488. The disclosure also provides antibodies comprising a heavy chain variable domain sequence that is at least 80%, at least 85%, at least 90%, or at least 95% identical to SEQ ID NO:164 and a light chain variable domain sequence that is at least 80%, at least 85%, at least 90%, or at least 95% identical to SEQ ID NO:489. The disclosure also provides antibodies comprising a heavy chain variable domain sequence that is at least 80%, at least 85%, at least 90%, or at least 95% identical to SEQ ID NO:165 and a light chain variable domain sequence that is at least 80%, at least 85%, at least 90%, or at least 95% identical to SEQ ID NO:490. The disclosure also provides antibodies comprising a heavy chain variable domain sequence that is at least 80%, at least 85%, at least 90%, or at least 95% identical to SEQ ID NO:166 and a light chain variable domain sequence that is at least 80%, at least 85%, at least 90%, or at least 95% identical to SEQ ID NO:474. The disclosure also provides antibodies comprising a heavy chain variable domain sequence that is at least 80%, at least 85%, at least 90%, or at least 95% identical to SEQ ID NO:167 and a light chain variable domain sequence that is at least 80%, at least 85%, at least 90%, or at least 95% identical to SEQ ID NO:491. The disclosure also provides antibodies comprising a heavy chain variable domain sequence that is at least 80%, at least 85%, at least 90%, or at least 95% identical to SEQ ID NO:168 and a light chain variable domain sequence that is at least 80%, at least 85%, at least 90%, or at least 95% identical to SEQ ID NO:492. The disclosure also provides antibodies comprising a heavy chain variable domain sequence that is at least 80%, at least 85%, at least 90%, or at least 95% identical to SEQ ID NO:169 and a light chain variable domain sequence that is at least 80%, at least 85%, at least 90%, or at least 95% identical to SEQ ID NO:493. The disclosure also provides antibodies comprising a heavy chain variable domain sequence that is at least 80%, at least 85%, at least 90%, or at least 95% identical to SEQ ID NO:170 and a light chain variable domain sequence that is at least 80%, at least 85%, at least 90%, or at least 95% identical to SEQ ID NO:494. The disclosure also provides antibodies comprising a heavy chain variable domain sequence that is at least 80%, at least 85%, at least 90%, or at least 95% identical to SEQ ID NO:171 and a light chain variable domain sequence that is at least 80%, at least 85%, at least 90%, or at least 95% identical to SEQ ID NO:495. The disclosure also provides antibodies comprising a heavy chain variable domain sequence that is at least 80%, at least 85%, at least 90%, or at least 95% identical to SEQ ID NO:172 and a light chain variable domain sequence that is at least 80%, at least 85%, at least 90%, or at least 95% identical to SEQ ID NO:496. The disclosure also provides antibodies comprising a heavy chain variable domain sequence that is at least 80%, at least 85%, at least 90%, or at least 95% identical to SEQ ID NO:173 and a light chain variable domain sequence that is at least 80%, at least 85%, at least 90%, or at least 95% identical to SEQ ID NO:497. The disclosure also provides antibodies comprising a heavy chain variable domain sequence that is at least 80%, at least 85%, at least 90%, or at least 95% identical to SEQ ID NO:174 and a light chain variable domain sequence that is at least 80%, at least 85%, at least 90%, or at least 95% identical to SEQ ID NO:498. The disclosure also provides antibodies comprising a heavy chain variable domain sequence that is at least 80%, at least 85%, at least 90%, or at least 95% identical to SEQ ID NO:175 and a light chain variable domain sequence that is at least 80%, at least 85%, at least 90%, or at least 95% identical to SEQ ID NO:486. The disclosure also provides antibodies comprising a heavy chain variable domain sequence that is at least 80%, at least 85%, at least 90%, or at least 95% identical to SEQ ID NO:176 and a light chain variable domain sequence that is at least 80%, at least 85%, at least 90%, or at least 95% identical to SEQ ID NO:499. The disclosure also provides antibodies comprising a heavy chain variable domain sequence that is at least 80%, at least 85%, at least 90%, or at least 95% identical to SEQ ID NO:177 and a light chain variable domain sequence that is at least 80%, at least 85%, at least 90%, or at least 95% identical to SEQ ID NO:500. The disclosure also provides antibodies comprising a heavy chain variable domain sequence that is at least 80%, at least 85%, at least 90%, or at least 95% identical to SEQ ID NO:178 and a light chain variable domain sequence that is at least 80%, at least 85%, at least 90%, or at least 95% identical to SEQ ID NO:479. The disclosure also provides antibodies comprising a heavy chain variable domain sequence that is at least 80%, at least 85%, at least 90%, or at least 95% identical to SEQ ID NO:179 and a light chain variable domain sequence that is at least 80%, at least 85%, at least 90%, or at least 95% identical to SEQ ID NO:501. The disclosure also provides antibodies comprising a heavy chain variable domain sequence that is at least 80%, at least 85%, at least 90%, or at least 95% identical to SEQ ID NO:180 and a light chain variable domain sequence that is at least 80%, at least 85%, at least 90%, or at least 95% identical to SEQ ID NO:502. The disclosure also provides antibodies comprising a heavy chain variable domain sequence that is at least 80%, at least 85%, at least 90%, or at least 95% identical to SEQ ID NO:181 and a light chain variable domain sequence that is at least 80%, at least 85%, at least 90%, or at least 95% identical to SEQ ID NO:503. The disclosure also provides antibodies comprising a heavy chain variable domain sequence that is at least 80%, at least 85%, at least 90%, or at least 95% identical to SEQ ID NO:182 and a light chain variable domain sequence that is at least 80%, at least 85%, at least 90%, or at least 95% identical to SEQ ID NO:504. The disclosure also provides antibodies comprising a heavy chain variable domain sequence that is at least 80%, at least 85%, at least 90%, or at least 95% identical to SEQ ID NO:183 and a light chain variable domain sequence that is at least 80%, at least 85%, at least 90%, or at least 95% identical to SEQ ID NO:505. The disclosure also provides antibodies comprising a heavy chain variable domain sequence that is at least 80%, at least 85%, at least 90%, or at least 95% identical to SEQ ID NO:184 and a light chain variable domain sequence that is at least 80%, at least 85%, at least 90%, or at least 95% identical to SEQ ID NO:506. The disclosure also provides antibodies comprising a heavy chain variable domain sequence that is at least 80%, at least 85%, at least 90%, or at least 95% identical to SEQ ID NO:185 and a light chain variable domain sequence that is at least 80%, at least 85%, at least 90%, or at least 95% identical to SEQ ID NO:507. The disclosure also provides antibodies comprising a heavy chain variable domain sequence that is at least 80%, at least 85%, at least 90%, or at least 95% identical to SEQ ID NO:186 and a light chain variable domain sequence that is at least 80%, at least 85%, at least 90%, or at least 95% identical to SEQ ID NO:469. The disclosure also provides antibodies comprising a heavy chain variable domain sequence that is at least 80%, at least 85%, at least 90%, or at least 95% identical to SEQ ID NO:181 and a light chain variable domain sequence that is at least 80%, at least 85%, at least 90%, or at least 95% identical to SEQ ID NO:508. The disclosure also provides antibodies comprising a heavy chain variable domain sequence that is at least 80%, at least 85%, at least 90%, or at least 95% identical to SEQ ID NO:187 and a light chain variable domain sequence that is at least 80%, at least 85%, at least 90%, or at least 95% identical to SEQ ID NO:509. The disclosure also provides antibodies comprising a heavy chain variable domain sequence that is at least 80%, at least 85%, at least 90%, or at least 95% identical to SEQ ID NO:188 and a light chain variable domain sequence that is at least 80%, at least 85%, at least 90%, or at least 95% identical to SEQ ID NO:510. The disclosure also provides antibodies comprising a heavy chain variable domain sequence that is at least 80%, at least 85%, at least 90%, or at least 95% identical to SEQ ID NO:189 and a light chain variable domain sequence that is at least 80%, at least 85%, at least 90%, or at least 95% identical to SEQ ID NO:511. The disclosure also provides antibodies comprising a heavy chain variable domain sequence that is at least 80%, at least 85%, at least 90%, or at least 95% identical to SEQ ID NO:190 and a light chain variable domain sequence that is at least 80%, at least 85%, at least 90%, or at least 95% identical to SEQ ID NO:512. The disclosure also provides antibodies comprising a heavy chain variable domain sequence that is at least 80%, at least 85%, at least 90%, or at least 95% identical to SEQ ID NO:191 and a light chain variable domain sequence that is at least 80%, at least 85%, at least 90%, or at least 95% identical to SEQ ID NO:513. The disclosure also provides antibodies comprising a heavy chain variable domain sequence that is at least 80%, at least 85%, at least 90%, or at least 95% identical to SEQ ID NO:192 and a light chain variable domain sequence that is at least 80%, at least 85%, at least 90%, or at least 95% identical to SEQ ID NO:514. The disclosure also provides antibodies comprising a heavy chain variable domain sequence that is at least 80%, at least 85%, at least 90%, or at least 95% identical to SEQ ID NO:193 and a light chain variable domain sequence that is at least 80%, at least 85%, at least 90%, or at least 95% identical to SEQ ID NO:515. The disclosure also provides antibodies comprising a heavy chain variable domain sequence that is at least 80%, at least 85%, at least 90%, or at least 95% identical to SEQ ID NO:194 and a light chain variable domain sequence that is at least 80%, at least 85%, at least 90%, or at least 95% identical to SEQ ID NO:516. The disclosure also provides antibodies comprising a heavy chain variable domain sequence that is at least 80%, at least 85%, at least 90%, or at least 95% identical to SEQ ID NO:177 and a light chain variable domain sequence that is at least 80%, at least 85%, at least 90%, or at least 95% identical to SEQ ID NO:517. The disclosure also provides antibodies comprising a heavy chain variable domain sequence that is at least 80%, at least 85%, at least 90%, or at least 95% identical to SEQ ID NO:140 and a light chain variable domain sequence that is at least 80%, at least 85%, at least 90%, or at least 95% identical to SEQ ID NO:518. The disclosure also provides antibodies comprising a heavy chain variable domain sequence that is at least 80%, at least 85%, at least 90%, or at least 95% identical to SEQ ID NO:195 and a light chain variable domain sequence that is at least 80%, at least 85%, at least 90%, or at least 95% identical to SEQ ID NO:519. The disclosure also provides antibodies comprising a heavy chain variable domain sequence that is at least 80%, at least 85%, at least 90%, or at least 95% identical to SEQ ID NO:196 and a light chain variable domain sequence that is at least 80%, at least 85%, at least 90%, or at least 95% identical to SEQ ID NO:520. The disclosure also provides antibodies comprising a heavy chain variable domain sequence that is at least 80%, at least 85%, at least 90%, or at least 95% identical to SEQ ID NO:171 and a light chain variable domain sequence that is at least 80%, at least 85%, at least 90%, or at least 95% identical to SEQ ID NO:521. The disclosure also provides antibodies comprising a heavy chain variable domain sequence that is at least 80%, at least 85%, at least 90%, or at least 95% identical to SEQ ID NO:197 and a light chain variable domain sequence that is at least 80%, at least 85%, at least 90%, or at least 95% identical to SEQ ID NO:522. The disclosure also provides antibodies comprising a heavy chain variable domain sequence that is at least 80%, at least 85%, at least 90%, or at least 95% identical to SEQ ID NO:198 and a light chain variable domain sequence that is at least 80%, at least 85%, at least 90%, or at least 95% identical to SEQ ID NO:523. The disclosure also provides antibodies comprising a heavy chain variable domain sequence that is at least 80%, at least 85%, at least 90%, or at least 95% identical to SEQ ID NO:170 and a light chain variable domain sequence that is at least 80%, at least 85%, at least 90%, or at least 95% identical to SEQ ID NO:524. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:525. The disclosure also provides antibodies comprising a heavy chain variable domain sequence that is at least 80%, at least 85%, at least 90%, or at least 95% identical to SEQ ID NO:200 and a light chain variable domain sequence that is at least 80%, at least 85%, at least 90%, or at least 95% identical to SEQ ID NO:526. The disclosure also provides antibodies comprising a heavy chain variable domain sequence that is at least 80%, at least 85%, at least 90%, or at least 95% identical to SEQ ID NO:201 and a light chain variable domain sequence that is at least 80%, at least 85%, at least 90%, or at least 95% identical to SEQ ID NO:527. The disclosure also provides antibodies comprising a heavy chain variable domain sequence that is at least 80%, at least 85%, at least 90%, or at least 95% identical to SEQ ID NO:202 and a light chain variable domain sequence that is at least 80%, at least 85%, at least 90%, or at least 95% identical to SEQ ID NO:528. The disclosure also provides antibodies comprising a heavy chain variable domain sequence that is at least 80%, at least 85%, at least 90%, or at least 95% identical to SEQ ID NO:203 and a light chain variable domain sequence that is at least 80%, at least 85%, at least 90%, or at least 95% identical to SEQ ID NO:529. The disclosure also provides antibodies comprising a heavy chain variable domain sequence that is at least 80%, at least 85%, at least 90%, or at least 95% identical to SEQ ID NO:204 and a light chain variable domain sequence that is at least 80%, at least 85%, at least 90%, or at least 95% identical to SEQ ID NO:530. The disclosure also provides antibodies comprising a heavy chain variable domain sequence that is at least 80%, at least 85%, at least 90%, or at least 95% identical to SEQ ID NO:205 and a light chain variable domain sequence that is at least 80%, at least 85%, at least 90%, or at least 95% identical to SEQ ID NO:531. The disclosure also provides antibodies comprising a heavy chain variable domain sequence that is at least 80%, at least 85%, at least 90%, or at least 95% identical to SEQ ID NO:206 and a light chain variable domain sequence that is at least 80%, at least 85%, at least 90%, or at least 95% identical to SEQ ID NO:532. The disclosure also provides antibodies comprising a heavy chain variable domain sequence that is at least 80%, at least 85%, at least 90%, or at least 95% identical to SEQ ID NO:207 and a light chain variable domain sequence that is at least 80%, at least 85%, at least 90%, or at least 95% identical to SEQ ID NO:533. The disclosure also provides antibodies comprising a heavy chain variable domain sequence that is at least 80%, at least 85%, at least 90%, or at least 95% identical to SEQ ID NO:208 and a light chain variable domain sequence that is at least 80%, at least 85%, at least 90%, or at least 95% identical to SEQ ID NO:534. The disclosure also provides antibodies comprising a heavy chain variable domain sequence that is at least 80%, at least 85%, at least 90%, or at least 95% identical to SEQ ID NO:209 and a light chain variable domain sequence that is at least 80%, at least 85%, at least 90%, or at least 95% identical to SEQ ID NO:535. The disclosure also provides antibodies comprising a heavy chain variable domain sequence that is at least 80%, at least 85%, at least 90%, or at least 95% identical to SEQ ID NO:210 and a light chain variable domain sequence that is at least 80%, at least 85%, at least 90%, or at least 95% identical to SEQ ID NO:536. The disclosure also provides antibodies comprising a heavy chain variable domain sequence that is at least 80%, at least 85%, at least 90%, or at least 95% identical to SEQ ID NO:211 and a light chain variable domain sequence that is at least 80%, at least 85%, at least 90%, or at least 95% identical to SEQ ID NO:537. The disclosure also provides antibodies comprising a heavy chain variable domain sequence that is at least 80%, at least 85%, at least 90%, or at least 95% identical to SEQ ID NO:171 and a light chain variable domain sequence that is at least 80%, at least 85%, at least 90%, or at least 95% identical to SEQ ID NO:538. The disclosure also provides antibodies comprising a heavy chain variable domain sequence that is at least 80%, at least 85%, at least 90%, or at least 95% identical to SEQ ID NO:212 and a light chain variable domain sequence that is at least 80%, at least 85%, at least 90%, or at least 95% identical to SEQ ID NO:539. The disclosure also provides antibodies comprising a heavy chain variable domain sequence that is at least 80%, at least 85%, at least 90%, or at least 95% identical to SEQ ID NO:213 and a light chain variable domain sequence that is at least 80%, at least 85%, at least 90%, or at least 95% identical to SEQ ID NO:540. The disclosure also provides antibodies comprising a heavy chain variable domain sequence that is at least 80%, at least 85%, at least 90%, or at least 95% identical to SEQ ID NO:214 and a light chain variable domain sequence that is at least 80%, at least 85%, at least 90%, or at least 95% identical to SEQ ID NO:541. The disclosure also provides antibodies comprising a heavy chain variable domain sequence that is at least 80%, at least 85%, at least 90%, or at least 95% identical to SEQ ID NO:182 and a light chain variable domain sequence that is at least 80%, at least 85%, at least 90%, or at least 95% identical to SEQ ID NO:542. The disclosure also provides antibodies comprising a heavy chain variable domain sequence that is at least 80%, at least 85%, at least 90%, or at least 95% identical to SEQ ID NO:215 and a light chain variable domain sequence that is at least 80%, at least 85%, at least 90%, or at least 95% identical to SEQ ID NO:515. The disclosure also provides antibodies comprising a heavy chain variable domain sequence that is at least 80%, at least 85%, at least 90%, or at least 95% identical to SEQ ID NO:201 and a light chain variable domain sequence that is at least 80%, at least 85%, at least 90%, or at least 95% identical to SEQ ID NO:543. The disclosure also provides antibodies comprising a heavy chain variable domain sequence that is at least 80%, at least 85%, at least 90%, or at least 95% identical to SEQ ID NO:216 and a light chain variable domain sequence that is at least 80%, at least 85%, at least 90%, or at least 95% identical to SEQ ID NO:527. The disclosure also provides antibodies comprising a heavy chain variable domain sequence that is at least 80%, at least 85%, at least 90%, or at least 95% identical to SEQ ID NO:217 and a light chain variable domain sequence that is at least 80%, at least 85%, at least 90%, or at least 95% identical to SEQ ID NO:544. The disclosure also provides antibodies comprising a heavy chain variable domain sequence that is at least 80%, at least 85%, at least 90%, or at least 95% identical to SEQ ID NO:218 and a light chain variable domain sequence that is at least 80%, at least 85%, at least 90%, or at least 95% identical to SEQ ID NO:482. The disclosure also provides antibodies comprising a heavy chain variable domain sequence that is at least 80%, at least 85%, at least 90%, or at least 95% identical to SEQ ID NO:219 and a light chain variable domain sequence that is at least 80%, at least 85%, at least 90%, or at least 95% identical to SEQ ID NO:545. The disclosure also provides antibodies comprising a heavy chain variable domain sequence that is at least 80%, at least 85%, at least 90%, or at least 95% identical to SEQ ID NO:220 and a light chain variable domain sequence that is at least 80%, at least 85%, at least 90%, or at least 95% identical to SEQ ID NO:546. The disclosure also provides antibodies comprising a heavy chain variable domain sequence that is at least 80%, at least 85%, at least 90%, or at least 95% identical to SEQ ID NO:221 and a light chain variable domain sequence that is at least 80%, at least 85%, at least 90%, or at least 95% identical to SEQ ID NO:547. The disclosure also provides antibodies comprising a heavy chain variable domain sequence that is at least 80%, at least 85%, at least 90%, or at least 95% identical to SEQ ID NO:183 and a light chain variable domain sequence that is at least 80%, at least 85%, at least 90%, or at least 95% identical to SEQ ID NO:548. The disclosure also provides antibodies comprising a heavy chain variable domain sequence that is at least 80%, at least 85%, at least 90%, or at least 95% identical to SEQ ID NO:222 and a light chain variable domain sequence that is at least 80%, at least 85%, at least 90%, or at least 95% identical to SEQ ID NO:469. The disclosure also provides antibodies comprising a heavy chain variable domain sequence that is at least 80%, at least 85%, at least 90%, or at least 95% identical to SEQ ID NO:223 and a light chain variable domain sequence that is at least 80%, at least 85%, at least 90%, or at least 95% identical to SEQ ID NO:549. The disclosure also provides antibodies comprising a heavy chain variable domain sequence that is at least 80%, at least 85%, at least 90%, or at least 95% identical to SEQ ID NO:224 and a light chain variable domain sequence that is at least 80%, at least 85%, at least 90%, or at least 95% identical to SEQ ID NO:550. The disclosure also provides antibodies comprising a heavy chain variable domain sequence that is at least 80%, at least 85%, at least 90%, or at least 95% identical to SEQ ID NO:225 and a light chain variable domain sequence that is at least 80%, at least 85%, at least 90%, or at least 95% identical to SEQ ID NO:551. The disclosure also provides antibodies comprising a heavy chain variable domain sequence that is at least 80%, at least 85%, at least 90%, or at least 95% identical to SEQ ID NO:226 and a light chain variable domain sequence that is at least 80%, at least 85%, at least 90%, or at least 95% identical to SEQ ID NO:552. The disclosure also provides antibodies comprising a heavy chain variable domain sequence that is at least 80%, at least 85%, at least 90%, or at least 95% identical to SEQ ID NO:227 and a light chain variable domain sequence that is at least 80%, at least 85%, at least 90%, or at least 95% identical to SEQ ID NO:529. The disclosure also provides antibodies comprising a heavy chain variable domain sequence that is at least 80%, at least 85%, at least 90%, or at least 95% identical to SEQ ID NO:151 and a light chain variable domain sequence that is at least 80%, at least 85%, at least 90%, or at least 95% identical to SEQ ID NO:553. The disclosure also provides antibodies comprising a heavy chain variable domain sequence that is at least 80%, at least 85%, at least 90%, or at least 95% identical to SEQ ID NO:228 and a light chain variable domain sequence that is at least 80%, at least 85%, at least 90%, or at least 95% identical to SEQ ID NO:554. The disclosure also provides antibodies comprising a heavy chain variable domain sequence that is at least 80%, at least 85%, at least 90%, or at least 95% identical to SEQ ID NO:229 and a light chain variable domain sequence that is at least 80%, at least 85%, at least 90%, or at least 95% identical to SEQ ID NO:555. The disclosure also provides antibodies comprising a heavy chain variable domain sequence that is at least 80%, at least 85%, at least 90%, or at least 95% identical to SEQ ID NO:201 and a light chain variable domain sequence that is at least 80%, at least 85%, at least 90%, or at least 95% identical to SEQ ID NO:552. The disclosure also provides antibodies comprising a heavy chain variable domain sequence that is at least 80%, at least 85%, at least 90%, or at least 95% identical to SEQ ID NO:230 and a light chain variable domain sequence that is at least 80%, at least 85%, at least 90%, or at least 95% identical to SEQ ID NO:556. The disclosure also provides antibodies comprising a heavy chain variable domain sequence that is at least 80%, at least 85%, at least 90%, or at least 95% identical to SEQ ID NO:231 and a light chain variable domain sequence that is at least 80%, at least 85%, at least 90%, or at least 95% identical to SEQ ID NO:557. The disclosure also provides antibodies comprising a heavy chain variable domain sequence that is at least 80%, at least 85%, at least 90%, or at least 95% identical to SEQ ID NO:163 and a light chain variable domain sequence that is at least 80%, at least 85%, at least 90%, or at least 95% identical to SEQ ID NO:558. The disclosure also provides antibodies comprising a heavy chain variable domain sequence that is at least 80%, at least 85%, at least 90%, or at least 95% identical to SEQ ID NO:232 and a light chain variable domain sequence that is at least 80%, at least 85%, at least 90%, or at least 95% identical to SEQ ID NO:559. The disclosure also provides antibodies comprising a heavy chain variable domain sequence that is at least 80%, at least 85%, at least 90%, or at least 95% identical to SEQ ID NO:233 and a light chain variable domain sequence that is at least 80%, at least 85%, at least 90%, or at least 95% identical to SEQ ID NO:466. The disclosure also provides antibodies comprising a heavy chain variable domain sequence that is at least 80%, at least 85%, at least 90%, or at least 95% identical to SEQ ID NO:234 and a light chain variable domain sequence that is at least 80%, at least 85%, at least 90%, or at least 95% identical to SEQ ID NO:560. The disclosure also provides antibodies comprising a heavy chain variable domain sequence that is at least 80%, at least 85%, at least 90%, or at least 95% identical to SEQ ID NO:235 and a light chain variable domain sequence that is at least 80%, at least 85%, at least 90%, or at least 95% identical to SEQ ID NO:561. The disclosure also provides antibodies comprising a heavy chain variable domain sequence that is at least 80%, at least 85%, at least 90%, or at least 95% identical to SEQ ID NO:236 and a light chain variable domain sequence that is at least 80%, at least 85%, at least 90%, or at least 95% identical to SEQ ID NO:562. The disclosure also provides antibodies comprising a heavy chain variable domain sequence that is at least 80%, at least 85%, at least 90%, or at least 95% identical to SEQ ID NO:164 and a light chain variable domain sequence that is at least 80%, at least 85%, at least 90%, or at least 95% identical to SEQ ID NO:563. The disclosure also provides antibodies comprising a heavy chain variable domain sequence that is at least 80%, at least 85%, at least 90%, or at least 95% identical to SEQ ID NO:237 and a light chain variable domain sequence that is at least 80%, at least 85%, at least 90%, or at least 95% identical to SEQ ID NO:564. The disclosure also provides antibodies comprising a heavy chain variable domain sequence that is at least 80%, at least 85%, at least 90%, or at least 95% identical to SEQ ID NO:201 and a light chain variable domain sequence that is at least 80%, at least 85%, at least 90%, or at least 95% identical to SEQ ID NO:565. The disclosure also provides antibodies comprising a heavy chain variable domain sequence that is at least 80%, at least 85%, at least 90%, or at least 95% identical to SEQ ID NO:192 and a light chain variable domain sequence that is at least 80%, at least 85%, at least 90%, or at least 95% identical to SEQ ID NO:566. The disclosure also provides antibodies comprising a heavy chain variable domain sequence that is at least 80%, at least 85%, at least 90%, or at least 95% identical to SEQ ID NO:238 and a light chain variable domain sequence that is at least 80%, at least 85%, at least 90%, or at least 95% identical to SEQ ID NO:567. The disclosure also provides antibodies comprising a heavy chain variable domain sequence that is at least 80%, at least 85%, at least 90%, or at least 95% identical to SEQ ID NO:210 and a light chain variable domain sequence that is at least 80%, at least 85%, at least 90%, or at least 95% identical to SEQ ID NO:568. The disclosure also provides antibodies comprising a heavy chain variable domain sequence that is at least 80%, at least 85%, at least 90%, or at least 95% identical to SEQ ID NO:239 and a light chain variable domain sequence that is at least 80%, at least 85%, at least 90%, or at least 95% identical to SEQ ID NO:569. The disclosure also provides antibodies comprising a heavy chain variable domain sequence that is at least 80%, at least 85%, at least 90%, or at least 95% identical to SEQ ID NO:240 and a light chain variable domain sequence that is at least 80%, at least 85%, at least 90%, or at least 95% identical to SEQ ID NO:517. The disclosure also provides antibodies comprising a heavy chain variable domain sequence that is at least 80%, at least 85%, at least 90%, or at least 95% identical to SEQ ID NO:241 and a light chain variable domain sequence that is at least 80%, at least 85%, at least 90%, or at least 95% identical to SEQ ID NO:570. The disclosure also provides antibodies comprising a heavy chain variable domain sequence that is at least 80%, at least 85%, at least 90%, or at least 95% identical to SEQ ID NO:242 and a light chain variable domain sequence that is at least 80%, at least 85%, at least 90%, or at least 95% identical to SEQ ID NO:571. The disclosure also provides antibodies comprising a heavy chain variable domain sequence that is at least 80%, at least 85%, at least 90%, or at least 95% identical to SEQ ID NO:243 and a light chain variable domain sequence that is at least 80%, at least 85%, at least 90%, or at least 95% identical to SEQ ID NO:572. The disclosure also provides antibodies comprising a heavy chain variable domain sequence that is at least 80%, at least 85%, at least 90%, or at least 95% identical to SEQ ID NO:182 and a light chain variable domain sequence that is at least 80%, at least 85%, at least 90%, or at least 95% identical to SEQ ID NO:573. The disclosure also provides antibodies comprising a heavy chain variable domain sequence that is at least 80%, at least 85%, at least 90%, or at least 95% identical to SEQ ID NO:244 and a light chain variable domain sequence that is at least 80%, at least 85%, at least 90%, or at least 95% identical to SEQ ID NO:574. The disclosure also provides antibodies comprising a heavy chain variable domain sequence that is at least 80%, at least 85%, at least 90%, or at least 95% identical to SEQ ID NO:245 and a light chain variable domain sequence that is at least 80%, at least 85%, at least 90%, or at least 95% identical to SEQ ID NO:575. The disclosure also provides antibodies comprising a heavy chain variable domain sequence that is at least 80%, at least 85%, at least 90%, or at least 95% identical to SEQ ID NO:246 and a light chain variable domain sequence that is at least 80%, at least 85%, at least 90%, or at least 95% identical to SEQ ID NO:576. The disclosure also provides antibodies comprising a heavy chain variable domain sequence that is at least 80%, at least 85%, at least 90%, or at least 95% identical to SEQ ID NO:230 and a light chain variable domain sequence that is at least 80%, at least 85%, at least 90%, or at least 95% identical to SEQ ID NO:577. The disclosure also provides antibodies comprising a heavy chain variable domain sequence that is at least 80%, at least 85%, at least 90%, or at least 95% identical to SEQ ID NO:247 and a light chain variable domain sequence that is at least 80%, at least 85%, at least 90%, or at least 95% identical to SEQ ID NO:540. The disclosure also provides antibodies comprising a heavy chain variable domain sequence that is at least 80%, at least 85%, at least 90%, or at least 95% identical to SEQ ID NO:246 and a light chain variable domain sequence that is at least 80%, at least 85%, at least 90%, or at least 95% identical to SEQ ID NO:578. The disclosure also provides antibodies comprising a heavy chain variable domain sequence that is at least 80%, at least 85%, at least 90%, or at least 95% identical to SEQ ID NO:248 and a light chain variable domain sequence that is at least 80%, at least 85%, at least 90%, or at least 95% identical to SEQ ID NO:482. The disclosure also provides antibodies comprising a heavy chain variable domain sequence that is at least 80%, at least 85%, at least 90%, or at least 95% identical to SEQ ID NO:249 and a light chain variable domain sequence that is at least 80%, at least 85%, at least 90%, or at least 95% identical to SEQ ID NO:579. The disclosure also provides antibodies comprising a heavy chain variable domain sequence that is at least 80%, at least 85%, at least 90%, or at least 95% identical to SEQ ID NO:198 and a light chain variable domain sequence that is at least 80%, at least 85%, at least 90%, or at least 95% identical to SEQ ID NO:580. The disclosure also provides antibodies comprising a heavy chain variable domain sequence that is at least 80%, at least 85%, at least 90%, or at least 95% identical to SEQ ID NO:173 and a light chain variable domain sequence that is at least 80%, at least 85%, at least 90%, or at least 95% identical to SEQ ID NO:581. The disclosure also provides antibodies comprising a heavy chain variable domain sequence that is at least 80%, at least 85%, at least 90%, or at least 95% identical to SEQ ID NO:250 and a light chain variable domain sequence that is at least 80%, at least 85%, at least 90%, or at least 95% identical to SEQ ID NO:582. The disclosure also provides antibodies comprising a heavy chain variable domain sequence that is at least 80%, at least 85%, at least 90%, or at least 95% identical to SEQ ID NO:251 and a light chain variable domain sequence that is at least 80%, at least 85%, at least 90%, or at least 95% identical to SEQ ID NO:583. The disclosure also provides antibodies comprising a heavy chain variable domain sequence that is at least 80%, at least 85%, at least 90%, or at least 95% identical to SEQ ID NO:252 and a light chain variable domain sequence that is at least 80%, at least 85%, at least 90%, or at least 95% identical to SEQ ID NO:531. The disclosure also provides antibodies comprising a heavy chain variable domain sequence that is at least 80%, at least 85%, at least 90%, or at least 95% identical to SEQ ID NO:253 and a light chain variable domain sequence that is at least 80%, at least 85%, at least 90%, or at least 95% identical to SEQ ID NO:584. The disclosure also provides antibodies comprising a heavy chain variable domain sequence that is at least 80%, at least 85%, at least 90%, or at least 95% identical to SEQ ID NO:254 and a light chain variable domain sequence that is at least 80%, at least 85%, at least 90%, or at least 95% identical to SEQ ID NO:585. The disclosure also provides antibodies comprising a heavy chain variable domain sequence that is at least 80%, at least 85%, at least 90%, or at least 95% identical to SEQ ID NO:189 and a light chain variable domain sequence that is at least 80%, at least 85%, at least 90%, or at least 95% identical to SEQ ID NO:586. The disclosure also provides antibodies comprising a heavy chain variable domain sequence that is at least 80%, at least 85%, at least 90%, or at least 95% identical to SEQ ID NO:255 and a light chain variable domain sequence that is at least 80%, at least 85%, at least 90%, or at least 95% identical to SEQ ID NO:482. The disclosure also provides antibodies comprising a heavy chain variable domain sequence that is at least 80%, at least 85%, at least 90%, or at least 95% identical to SEQ ID NO:256 and a light chain variable domain sequence that is at least 80%, at least 85%, at least 90%, or at least 95% identical to SEQ ID NO:587. The disclosure also provides antibodies comprising a heavy chain variable domain sequence that is at least 80%, at least 85%, at least 90%, or at least 95% identical to SEQ ID NO:257 and a light chain variable domain sequence that is at least 80%, at least 85%, at least 90%, or at least 95% identical to SEQ ID NO:588. The disclosure also provides antibodies comprising a heavy chain variable domain sequence that is at least 80%, at least 85%, at least 90%, or at least 95% identical to SEQ ID NO:258 and a light chain variable domain sequence that is at least 80%, at least 85%, at least 90%, or at least 95% identical to SEQ ID NO:589. The disclosure also provides antibodies comprising a heavy chain variable domain sequence that is at least 80%, at least 85%, at least 90%, or at least 95% identical to SEQ ID NO:259 and a light chain variable domain sequence that is at least 80%, at least 85%, at least 90%, or at least 95% identical to SEQ ID NO:590. The disclosure also provides antibodies comprising a heavy chain variable domain sequence that is at least 80%, at least 85%, at least 90%, or at least 95% identical to SEQ ID NO:260 and a light chain variable domain sequence that is at least 80%, at least 85%, at least 90%, or at least 95% identical to SEQ ID NO:591. The disclosure also provides antibodies comprising a heavy chain variable domain sequence that is at least 80%, at least 85%, at least 90%, or at least 95% identical to SEQ ID NO:261 and a light chain variable domain sequence that is at least 80%, at least 85%, at least 90%, or at least 95% identical to SEQ ID NO:592. The disclosure also provides antibodies comprising a heavy chain variable domain sequence that is at least 80%, at least 85%, at least 90%, or at least 95% identical to SEQ ID NO:262 and a light chain variable domain sequence that is at least 80%, at least 85%, at least 90%, or at least 95% identical to SEQ ID NO:593. The disclosure also provides antibodies comprising a heavy chain variable domain sequence that is at least 80%, at least 85%, at least 90%, or at least 95% identical to SEQ ID NO:263 and a light chain variable domain sequence that is at least 80%, at least 85%, at least 90%, or at least 95% identical to SEQ ID NO:594. The disclosure also provides antibodies comprising a heavy chain variable domain sequence that is at least 80%, at least 85%, at least 90%, or at least 95% identical to SEQ ID NO:220 and a light chain variable domain sequence that is at least 80%, at least 85%, at least 90%, or at least 95% identical to SEQ ID NO:595. The disclosure also provides antibodies comprising a heavy chain variable domain sequence that is at least 80%, at least 85%, at least 90%, or at least 95% identical to SEQ ID NO:264 and a light chain variable domain sequence that is at least 80%, at least 85%, at least 90%, or at least 95% identical to SEQ ID NO:596. The disclosure also provides antibodies comprising a heavy chain variable domain sequence that is at least 80%, at least 85%, at least 90%, or at least 95% identical to SEQ ID NO:189 and a light chain variable domain sequence that is at least 80%, at least 85%, at least 90%, or at least 95% identical to SEQ ID NO:596. The disclosure also provides antibodies comprising a heavy chain variable domain sequence that is at least 80%, at least 85%, at least 90%, or at least 95% identical to SEQ ID NO:265 and a light chain variable domain sequence that is at least 80%, at least 85%, at least 90%, or at least 95% identical to SEQ ID NO:597. The disclosure also provides antibodies comprising a heavy chain variable domain sequence that is at least 80%, at least 85%, at least 90%, or at least 95% identical to SEQ ID NO:266 and a light chain variable domain sequence that is at least 80%, at least 85%, at least 90%, or at least 95% identical to SEQ ID NO:598. The disclosure also provides antibodies comprising a heavy chain variable domain sequence that is at least 80%, at least 85%, at least 90%, or at least 95% identical to SEQ ID NO:267 and a light chain variable domain sequence that is at least 80%, at least 85%, at least 90%, or at least 95% identical to SEQ ID NO:599. The disclosure also provides antibodies comprising a heavy chain variable domain sequence that is at least 80%, at least 85%, at least 90%, or at least 95% identical to SEQ ID NO:268 and a light chain variable domain sequence that is at least 80%, at least 85%, at least 90%, or at least 95% identical to SEQ ID NO:600. The disclosure also provides antibodies comprising a heavy chain variable domain sequence that is at least 80%, at least 85%, at least 90%, or at least 95% identical to SEQ ID NO:201 and a light chain variable domain sequence that is at least 80%, at least 85%, at least 90%, or at least 95% identical to SEQ ID NO:515. The disclosure also provides antibodies comprising a heavy chain variable domain sequence that is at least 80%, at least 85%, at least 90%, or at least 95% identical to SEQ ID NO:269 and a light chain variable domain sequence that is at least 80%, at least 85%, at least 90%, or at least 95% identical to SEQ ID NO:601. The disclosure also provides antibodies comprising a heavy chain variable domain sequence that is at least 80%, at least 85%, at least 90%, or at least 95% identical to SEQ ID NO:257 and a light chain variable domain sequence that is at least 80%, at least 85%, at least 90%, or at least 95% identical to SEQ ID NO:506. The disclosure also provides antibodies comprising a heavy chain variable domain sequence that is at least 80%, at least 85%, at least 90%, or at least 95% identical to SEQ ID NO:201 and a light chain variable domain sequence that is at least 80%, at least 85%, at least 90%, or at least 95% identical to SEQ ID NO:602. The disclosure also provides antibodies comprising a heavy chain variable domain sequence that is at least 80%, at least 85%, at least 90%, or at least 95% identical to SEQ ID NO:270 and a light chain variable domain sequence that is at least 80%, at least 85%, at least 90%, or at least 95% identical to SEQ ID NO:603. The disclosure also provides antibodies comprising a heavy chain variable domain sequence that is at least 80%, at least 85%, at least 90%, or at least 95% identical to SEQ ID NO:271 and a light chain variable domain sequence that is at least 80%, at least 85%, at least 90%, or at least 95% identical to SEQ ID NO:552. The disclosure also provides antibodies comprising a heavy chain variable domain sequence that is at least 80%, at least 85%, at least 90%, or at least 95% identical to SEQ ID NO:177 and a light chain variable domain sequence that is at least 80%, at least 85%, at least 90%, or at least 95% identical to SEQ ID NO:604. The disclosure also provides antibodies comprising a heavy chain variable domain sequence that is at least 80%, at least 85%, at least 90%, or at least 95% identical to SEQ ID NO:183 and a light chain variable domain sequence that is at least 80%, at least 85%, at least 90%, or at least 95% identical to SEQ ID NO:469. The disclosure also provides antibodies comprising a heavy chain variable domain sequence that is at least 80%, at least 85%, at least 90%, or at least 95% identical to SEQ ID NO:272 and a light chain variable domain sequence that is at least 80%, at least 85%, at least 90%, or at least 95% identical to SEQ ID NO:605. The disclosure also provides antibodies comprising a heavy chain variable domain sequence that is at least 80%, at least 85%, at least 90%, or at least 95% identical to SEQ ID NO:273 and a light chain variable domain sequence that is at least 80%, at least 85%, at least 90%, or at least 95% identical to SEQ ID NO:606. The disclosure also provides antibodies comprising a heavy chain variable domain sequence that is at least 80%, at least 85%, at least 90%, or at least 95% identical to SEQ ID NO:274 and a light chain variable domain sequence that is at least 80%, at least 85%, at least 90%, or at least 95% identical to SEQ ID NO:596. The disclosure also provides antibodies comprising a heavy chain variable domain sequence that is at least 80%, at least 85%, at least 90%, or at least 95% identical to SEQ ID NO:275 and a light chain variable domain sequence that is at least 80%, at least 85%, at least 90%, or at least 95% identical to SEQ ID NO:607. The disclosure also provides antibodies comprising a heavy chain variable domain sequence that is at least 80%, at least 85%, at least 90%, or at least 95% identical to SEQ ID NO:276 and a light chain variable domain sequence that is at least 80%, at least 85%, at least 90%, or at least 95% identical to SEQ ID NO:608. The disclosure also provides antibodies comprising a heavy chain variable domain sequence that is at least 80%, at least 85%, at least 90%, or at least 95% identical to SEQ ID NO:277 and a light chain variable domain sequence that is at least 80%, at least 85%, at least 90%, or at least 95% identical to SEQ ID NO:466. The disclosure also provides antibodies comprising a heavy chain variable domain sequence that is at least 80%, at least 85%, at least 90%, or at least 95% identical to SEQ ID NO:278 and a light chain variable domain sequence that is at least 80%, at least 85%, at least 90%, or at least 95% identical to SEQ ID NO:609. The disclosure also provides antibodies comprising a heavy chain variable domain sequence that is at least 80%, at least 85%, at least 90%, or at least 95% identical to SEQ ID NO:279 and a light chain variable domain sequence that is at least 80%, at least 85%, at least 90%, or at least 95% identical to SEQ ID NO:610. The disclosure also provides antibodies comprising a heavy chain variable domain sequence that is at least 80%, at least 85%, at least 90%, or at least 95% identical to SEQ ID NO:280 and a light chain variable domain sequence that is at least 80%, at least 85%, at least 90%, or at least 95% identical to SEQ ID NO:611. The disclosure also provides antibodies comprising a heavy chain variable domain sequence that is at least 80%, at least 85%, at least 90%, or at least 95% identical to SEQ ID NO:281 and a light chain variable domain sequence that is at least 80%, at least 85%, at least 90%, or at least 95% identical to SEQ ID NO:482. The disclosure also provides antibodies comprising a heavy chain variable domain sequence that is at least 80%, at least 85%, at least 90%, or at least 95% identical to SEQ ID NO:282 and a light chain variable domain sequence that is at least 80%, at least 85%, at least 90%, or at least 95% identical to SEQ ID NO:612. The disclosure also provides antibodies comprising a heavy chain variable domain sequence that is at least 80%, at least 85%, at least 90%, or at least 95% identical to SEQ ID NO:283 and a light chain variable domain sequence that is at least 80%, at least 85%, at least 90%, or at least 95% identical to SEQ ID NO:613. The disclosure also provides antibodies comprising a heavy chain variable domain sequence that is at least 80%, at least 85%, at least 90%, or at least 95% identical to SEQ ID NO:284 and a light chain variable domain sequence that is at least 80%, at least 85%, at least 90%, or at least 95% identical to SEQ ID NO:614. The disclosure also provides antibodies comprising a heavy chain variable domain sequence that is at least 80%, at least 85%, at least 90%, or at least 95% identical to SEQ ID NO:285 and a light chain variable domain sequence that is at least 80%, at least 85%, at least 90%, or at least 95% identical to SEQ ID NO:615. The disclosure also provides antibodies comprising a heavy chain variable domain sequence that is at least 80%, at least 85%, at least 90%, or at least 95% identical to SEQ ID NO:224 and a light chain variable domain sequence that is at least 80%, at least 85%, at least 90%, or at least 95% identical to SEQ ID NO:608. The disclosure also provides antibodies comprising a heavy chain variable domain sequence that is at least 80%, at least 85%, at least 90%, or at least 95% identical to SEQ ID NO:286 and a light chain variable domain sequence that is at least 80%, at least 85%, at least 90%, or at least 95% identical to SEQ ID NO:616. The disclosure also provides antibodies comprising a heavy chain variable domain sequence that is at least 80%, at least 85%, at least 90%, or at least 95% identical to SEQ ID NO:180 and a light chain variable domain sequence that is at least 80%, at least 85%, at least 90%, or at least 95% identical to SEQ ID NO:617. The disclosure also provides antibodies comprising a heavy chain variable domain sequence that is at least 80%, at least 85%, at least 90%, or at least 95% identical to SEQ ID NO:287 and a light chain variable domain sequence that is at least 80%, at least 85%, at least 90%, or at least 95% identical to SEQ ID NO:618. The disclosure also provides antibodies comprising a heavy chain variable domain sequence that is at least 80%, at least 85%, at least 90%, or at least 95% identical to SEQ ID NO:288 and a light chain variable domain sequence that is at least 80%, at least 85%, at least 90%, or at least 95% identical to SEQ ID NO:619. The disclosure also provides antibodies comprising a heavy chain variable domain sequence that is at least 80%, at least 85%, at least 90%, or at least 95% identical to SEQ ID NO:289 and a light chain variable domain sequence that is at least 80%, at least 85%, at least 90%, or at least 95% identical to SEQ ID NO:620. The disclosure also provides antibodies comprising a heavy chain variable domain sequence that is at least 80%, at least 85%, at least 90%, or at least 95% identical to SEQ ID NO:238 and a light chain variable domain sequence that is at least 80%, at least 85%, at least 90%, or at least 95% identical to SEQ ID NO:621. The disclosure also provides antibodies comprising a heavy chain variable domain sequence that is at least 80%, at least 85%, at least 90%, or at least 95% identical to SEQ ID NO:177 and a light chain variable domain sequence that is at least 80%, at least 85%, at least 90%, or at least 95% identical to SEQ ID NO:622. The disclosure also provides antibodies comprising a heavy chain variable domain sequence that is at least 80%, at least 85%, at least 90%, or at least 95% identical to SEQ ID NO:290 and a light chain variable domain sequence that is at least 80%, at least 85%, at least 90%, or at least 95% identical to SEQ ID NO:623. The disclosure also provides antibodies comprising a heavy chain variable domain sequence that is at least 80%, at least 85%, at least 90%, or at least 95% identical to SEQ ID NO:291 and a light chain variable domain sequence that is at least 80%, at least 85%, at least 90%, or at least 95% identical to SEQ ID NO:572. The disclosure also provides antibodies comprising a heavy chain variable domain sequence that is at least 80%, at least 85%, at least 90%, or at least 95% identical to SEQ ID NO:292 and a light chain variable domain sequence that is at least 80%, at least 85%, at least 90%, or at least 95% identical to SEQ ID NO:624. The disclosure also provides antibodies comprising a heavy chain variable domain sequence that is at least 80%, at least 85%, at least 90%, or at least 95% identical to SEQ ID NO:293 and a light chain variable domain sequence that is at least 80%, at least 85%, at least 90%, or at least 95% identical to SEQ ID NO:529. The disclosure also provides antibodies comprising a heavy chain variable domain sequence that is at least 80%, at least 85%, at least 90%, or at least 95% identical to SEQ ID NO:177 and a light chain variable domain sequence that is at least 80%, at least 85%, at least 90%, or at least 95% identical to SEQ ID NO:625. The disclosure also provides antibodies comprising a heavy chain variable domain sequence that is at least 80%, at least 85%, at least 90%, or at least 95% identical to SEQ ID NO:294 and a light chain variable domain sequence that is at least 80%, at least 85%, at least 90%, or at least 95% identical to SEQ ID NO:626. The disclosure also provides antibodies comprising a heavy chain variable domain sequence that is at least 80%, at least 85%, at least 90%, or at least 95% identical to SEQ ID NO:295 and a light chain variable domain sequence that is at least 80%, at least 85%, at least 90%, or at least 95% identical to SEQ ID NO:512. The disclosure also provides antibodies comprising a heavy chain variable domain sequence that is at least 80%, at least 85%, at least 90%, or at least 95% identical to SEQ ID NO:296 and a light chain variable domain sequence that is at least 80%, at least 85%, at least 90%, or at least 95% identical to SEQ ID NO:627. The disclosure also provides antibodies comprising a heavy chain variable domain sequence that is at least 80%, at least 85%, at least 90%, or at least 95% identical to SEQ ID NO:173 and a light chain variable domain sequence that is at least 80%, at least 85%, at least 90%, or at least 95% identical to SEQ ID NO:628. The disclosure also provides antibodies comprising a heavy chain variable domain sequence that is at least 80%, at least 85%, at least 90%, or at least 95% identical to SEQ ID NO:297 and a light chain variable domain sequence that is at least 80%, at least 85%, at least 90%, or at least 95% identical to SEQ ID NO:629. The disclosure also provides antibodies comprising a heavy chain variable domain sequence that is at least 80%, at least 85%, at least 90%, or at least 95% identical to SEQ ID NO:298 and a light chain variable domain sequence that is at least 80%, at least 85%, at least 90%, or at least 95% identical to SEQ ID NO:630. The disclosure also provides antibodies comprising a heavy chain variable domain sequence that is at least 80%, at least 85%, at least 90%, or at least 95% identical to SEQ ID NO:299 and a light chain variable domain sequence that is at least 80%, at least 85%, at least 90%, or at least 95% identical to SEQ ID NO:466. The disclosure also provides antibodies comprising a heavy chain variable domain sequence that is at least 80%, at least 85%, at least 90%, or at least 95% identical to SEQ ID NO:300 and a light chain variable domain sequence that is at least 80%, at least 85%, at least 90%, or at least 95% identical to SEQ ID NO:631. The disclosure also provides antibodies comprising a heavy chain variable domain sequence that is at least 80%, at least 85%, at least 90%, or at least 95% identical to SEQ ID NO:301 and a light chain variable domain sequence that is at least 80%, at least 85%, at least 90%, or at least 95% identical to SEQ ID NO:580. The disclosure also provides antibodies comprising a heavy chain variable domain sequence that is at least 80%, at least 85%, at least 90%, or at least 95% identical to SEQ ID NO:176 and a light chain variable domain sequence that is at least 80%, at least 85%, at least 90%, or at least 95% identical to SEQ ID NO:632. The disclosure also provides antibodies comprising a heavy chain variable domain sequence that is at least 80%, at least 85%, at least 90%, or at least 95% identical to SEQ ID NO:302 and a light chain variable domain sequence that is at least 80%, at least 85%, at least 90%, or at least 95% identical to SEQ ID NO:633. The disclosure also provides antibodies comprising a heavy chain variable domain sequence that is at least 80%, at least 85%, at least 90%, or at least 95% identical to SEQ ID NO:267 and a light chain variable domain sequence that is at least 80%, at least 85%, at least 90%, or at least 95% identical to SEQ ID NO:531. The disclosure also provides antibodies comprising a heavy chain variable domain sequence that is at least 80%, at least 85%, at least 90%, or at least 95% identical to SEQ ID NO:303 and a light chain variable domain sequence that is at least 80%, at least 85%, at least 90%, or at least 95% identical to SEQ ID NO:634. The disclosure also provides antibodies comprising a heavy chain variable domain sequence that is at least 80%, at least 85%, at least 90%, or at least 95% identical to SEQ ID NO:304 and a light chain variable domain sequence that is at least 80%, at least 85%, at least 90%, or at least 95% identical to SEQ ID NO:612. The disclosure also provides antibodies comprising a heavy chain variable domain sequence that is at least 80%, at least 85%, at least 90%, or at least 95% identical to SEQ ID NO:305 and a light chain variable domain sequence that is at least 80%, at least 85%, at least 90%, or at least 95% identical to SEQ ID NO:635. The disclosure also provides antibodies comprising a heavy chain variable domain sequence that is at least 80%, at least 85%, at least 90%, or at least 95% identical to SEQ ID NO:306 and a light chain variable domain sequence that is at least 80%, at least 85%, at least 90%, or at least 95% identical to SEQ ID NO:636. The disclosure also provides antibodies comprising a heavy chain variable domain sequence that is at least 80%, at least 85%, at least 90%, or at least 95% identical to SEQ ID NO:307 and a light chain variable domain sequence that is at least 80%, at least 85%, at least 90%, or at least 95% identical to SEQ ID NO:596. The disclosure also provides antibodies comprising a heavy chain variable domain sequence that is at least 80%, at least 85%, at least 90%, or at least 95% identical to SEQ ID NO:308 and a light chain variable domain sequence that is at least 80%, at least 85%, at least 90%, or at least 95% identical to SEQ ID NO:637. The disclosure also provides antibodies comprising a heavy chain variable domain sequence that is at least 80%, at least 85%, at least 90%, or at least 95% identical to SEQ ID NO:309 and a light chain variable domain sequence that is at least 80%, at least 85%, at least 90%, or at least 95% identical to SEQ ID NO:638. The disclosure also provides antibodies comprising a heavy chain variable domain sequence that is at least 80%, at least 85%, at least 90%, or at least 95% identical to SEQ ID NO:310 and a light chain variable domain sequence that is at least 80%, at least 85%, at least 90%, or at least 95% identical to SEQ ID NO:639. The disclosure also provides antibodies comprising a heavy chain variable domain sequence that is at least 80%, at least 85%, at least 90%, or at least 95% identical to SEQ ID NO:311 and a light chain variable domain sequence that is at least 80%, at least 85%, at least 90%, or at least 95% identical to SEQ ID NO:552. The disclosure also provides antibodies comprising a heavy chain variable domain sequence that is at least 80%, at least 85%, at least 90%, or at least 95% identical to SEQ ID NO:312 and a light chain variable domain sequence that is at least 80%, at least 85%, at least 90%, or at least 95% identical to SEQ ID NO:640. The disclosure also provides antibodies comprising a heavy chain variable domain sequence that is at least 80%, at least 85%, at least 90%, or at least 95% identical to SEQ ID NO:313 and a light chain variable domain sequence that is at least 80%, at least 85%, at least 90%, or at least 95% identical to SEQ ID NO:641. The disclosure also provides antibodies comprising a heavy chain variable domain sequence that is at least 80%, at least 85%, at least 90%, or at least 95% identical to SEQ ID NO:314 and a light chain variable domain sequence that is at least 80%, at least 85%, at least 90%, or at least 95% identical to SEQ ID NO:642. The disclosure also provides antibodies comprising a heavy chain variable domain sequence that is at least 80%, at least 85%, at least 90%, or at least 95% identical to SEQ ID NO:315 and a light chain variable domain sequence that is at least 80%, at least 85%, at least 90%, or at least 95% identical to SEQ ID NO:643. The disclosure also provides antibodies comprising a heavy chain variable domain sequence that is at least 80%, at least 85%, at least 90%, or at least 95% identical to SEQ ID NO:316 and a light chain variable domain sequence that is at least 80%, at least 85%, at least 90%, or at least 95% identical to SEQ ID NO:644. The disclosure also provides antibodies comprising a heavy chain variable domain sequence that is at least 80%, at least 85%, at least 90%, or at least 95% identical to SEQ ID NO:311 and a light chain variable domain sequence that is at least 80%, at least 85%, at least 90%, or at least 95% identical to SEQ ID NO:645. The disclosure also provides antibodies comprising a heavy chain variable domain sequence that is at least 80%, at least 85%, at least 90%, or at least 95% identical to SEQ ID NO:317 and a light chain variable domain sequence that is at least 80%, at least 85%, at least 90%, or at least 95% identical to SEQ ID NO:645. The disclosure also provides antibodies comprising a heavy chain variable domain sequence that is at least 80%, at least 85%, at least 90%, or at least 95% identical to SEQ ID NO:318 and a light chain variable domain sequence that is at least 80%, at least 85%, at least 90%, or at least 95% identical to SEQ ID NO:646. The disclosure also provides antibodies comprising a heavy chain variable domain sequence that is at least 80%, at least 85%, at least 90%, or at least 95% identical to SEQ ID NO:319 and a light chain variable domain sequence that is at least 80%, at least 85%, at least 90%, or at least 95% identical to SEQ ID NO:647. The disclosure also provides antibodies comprising a heavy chain variable domain sequence that is at least 80%, at least 85%, at least 90%, or at least 95% identical to SEQ ID NO:320 and a light chain variable domain sequence that is at least 80%, at least 85%, at least 90%, or at least 95% identical to SEQ ID NO:552. The disclosure also provides antibodies comprising a heavy chain variable domain sequence that is at least 80%, at least 85%, at least 90%, or at least 95% identical to SEQ ID NO:321 and a light chain variable domain sequence that is at least 80%, at least 85%, at least 90%, or at least 95% identical to SEQ ID NO:648. The disclosure also provides antibodies comprising a heavy chain variable domain sequence that is at least 80%, at least 85%, at least 90%, or at least 95% identical to SEQ ID NO:322 and a light chain variable domain sequence that is at least 80%, at least 85%, at least 90%, or at least 95% identical to SEQ ID NO:649. The disclosure also provides antibodies comprising a heavy chain variable domain sequence that is at least 80%, at least 85%, at least 90%, or at least 95% identical to SEQ ID NO:323 and a light chain variable domain sequence that is at least 80%, at least 85%, at least 90%, or at least 95% identical to SEQ ID NO:650. The disclosure also provides antibodies comprising a heavy chain variable domain sequence that is at least 80%, at least 85%, at least 90%, or at least 95% identical to SEQ ID NO:315 and a light chain variable domain sequence that is at least 80%, at least 85%, at least 90%, or at least 95% identical to SEQ ID NO:651. The disclosure also provides antibodies comprising a heavy chain variable domain sequence that is at least 80%, at least 85%, at least 90%, or at least 95% identical to SEQ ID NO:324 and a light chain variable domain sequence that is at least 80%, at least 85%, at least 90%, or at least 95% identical to SEQ ID NO:652. The disclosure also provides antibodies comprising a heavy chain variable domain sequence that is at least 80%, at least 85%, at least 90%, or at least 95% identical to SEQ ID NO:325 and a light chain variable domain sequence that is at least 80%, at least 85%, at least 90%, or at least 95% identical to SEQ ID NO:653. The disclosure also provides antibodies comprising a heavy chain variable domain sequence that is at least 80%, at least 85%, at least 90%, or at least 95% identical to SEQ ID NO:326 and a light chain variable domain sequence that is at least 80%, at least 85%, at least 90%, or at least 95% identical to SEQ ID NO:482. The disclosure also provides antibodies comprising a heavy chain variable domain sequence that is at least 80%, at least 85%, at least 90%, or at least 95% identical to SEQ ID NO:313 and a light chain variable domain sequence that is at least 80%, at least 85%, at least 90%, or at least 95% identical to SEQ ID NO:645. The disclosure also provides antibodies comprising a heavy chain variable domain sequence that is at least 80%, at least 85%, at least 90%, or at least 95% identical to SEQ ID NO:327 and a light chain variable domain sequence that is at least 80%, at least 85%, at least 90%, or at least 95% identical to SEQ ID NO:654. The disclosure also provides antibodies comprising a heavy chain variable domain sequence that is at least 80%, at least 85%, at least 90%, or at least 95% identical to SEQ ID NO:328 and a light chain variable domain sequence that is at least 80%, at least 85%, at least 90%, or at least 95% identical to SEQ ID NO:655. The disclosure also provides antibodies comprising a heavy chain variable domain sequence that is at least 80%, at least 85%, at least 90%, or at least 95% identical to SEQ ID NO:329 and a light chain variable domain sequence that is at least 80%, at least 85%, at least 90%, or at least 95% identical to SEQ ID NO:656. The disclosure also provides antibodies comprising a heavy chain variable domain sequence that is at least 80%, at least 85%, at least 90%, or at least 95% identical to SEQ ID NO:330 and a light chain variable domain sequence that is at least 80%, at least 85%, at least 90%, or at least 95% identical to SEQ ID NO:572. The disclosure also provides antibodies comprising a heavy chain variable domain sequence that is at least 80%, at least 85%, at least 90%, or at least 95% identical to SEQ ID NO:331 and a light chain variable domain sequence that is at least 80%, at least 85%, at least 90%, or at least 95% identical to SEQ ID NO:657. The disclosure also provides antibodies comprising a heavy chain variable domain sequence that is at least 80%, at least 85%, at least 90%, or at least 95% identical to SEQ ID NO:332 and a light chain variable domain sequence that is at least 80%, at least 85%, at least 90%, or at least 95% identical to SEQ ID NO:652. The disclosure also provides antibodies comprising a heavy chain variable domain sequence that is at least 80%, at least 85%, at least 90%, or at least 95% identical to SEQ ID NO:333 and a light chain variable domain sequence that is at least 80%, at least 85%, at least 90%, or at least 95% identical to SEQ ID NO:658. The disclosure also provides antibodies comprising a heavy chain variable domain sequence that is at least 80%, at least 85%, at least 90%, or at least 95% identical to SEQ ID NO:334 and a light chain variable domain sequence that is at least 80%, at least 85%, at least 90%, or at least 95% identical to SEQ ID NO:659. The disclosure also provides antibodies comprising a heavy chain variable domain sequence that is at least 80%, at least 85%, at least 90%, or at least 95% identical to SEQ ID NO:335 and a light chain variable domain sequence that is at least 80%, at least 85%, at least 90%, or at least 95% identical to SEQ ID NO:660. The disclosure also provides antibodies comprising a heavy chain variable domain sequence that is at least 80%, at least 85%, at least 90%, or at least 95% identical to SEQ ID NO:336 and a light chain variable domain sequence that is at least 80%, at least 85%, at least 90%, or at least 95% identical to SEQ ID NO:661. The disclosure also provides antibodies comprising a heavy chain variable domain sequence that is at least 80%, at least 85%, at least 90%, or at least 95% identical to SEQ ID NO:337 and a light chain variable domain sequence that is at least 80%, at least 85%, at least 90%, or at least 95% identical to SEQ ID NO:643. The disclosure also provides antibodies comprising a heavy chain variable domain sequence that is at least 80%, at least 85%, at least 90%, or at least 95% identical to SEQ ID NO:338 and a light chain variable domain sequence that is at least 80%, at least 85%, at least 90%, or at least 95% identical to SEQ ID NO:552. The disclosure also provides antibodies comprising a heavy chain variable domain sequence that is at least 80%, at least 85%, at least 90%, or at least 95% identical to SEQ ID NO:339 and a light chain variable domain sequence that is at least 80%, at least 85%, at least 90%, or at least 95% identical to SEQ ID NO:662. The disclosure also provides antibodies comprising a heavy chain variable domain sequence that is at least 80%, at least 85%, at least 90%, or at least 95% identical to SEQ ID NO:340 and a light chain variable domain sequence that is at least 80%, at least 85%, at least 90%, or at least 95% identical to SEQ ID NO:663. The disclosure also provides antibodies comprising a heavy chain variable domain sequence that is at least 80%, at least 85%, at least 90%, or at least 95% identical to SEQ ID NO:341 and a light chain variable domain sequence that is at least 80%, at least 85%, at least 90%, or at least 95% identical to SEQ ID NO:645. The disclosure also provides antibodies comprising a heavy chain variable domain sequence that is at least 80%, at least 85%, at least 90%, or at least 95% identical to SEQ ID NO:342 and a light chain variable domain sequence that is at least 80%, at least 85%, at least 90%, or at least 95% identical to SEQ ID NO:664. The disclosure also provides antibodies comprising a heavy chain variable domain sequence that is at least 80%, at least 85%, at least 90%, or at least 95% identical to SEQ ID NO:343 and a light chain variable domain sequence that is at least 80%, at least 85%, at least 90%, or at least 95% identical to SEQ ID NO:572. The disclosure also provides antibodies comprising a heavy chain variable domain sequence that is at least 80%, at least 85%, at least 90%, or at least 95% identical to SEQ ID NO:344 and a light chain variable domain sequence that is at least 80%, at least 85%, at least 90%, or at least 95% identical to SEQ ID NO:665. The disclosure also provides antibodies comprising a heavy chain variable domain sequence that is at least 80%, at least 85%, at least 90%, or at least 95% identical to SEQ ID NO:345 and a light chain variable domain sequence that is at least 80%, at least 85%, at least 90%, or at least 95% identical to SEQ ID NO:483. The disclosure also provides antibodies comprising a heavy chain variable domain sequence that is at least 80%, at least 85%, at least 90%, or at least 95% identical to SEQ ID NO:346 and a light chain variable domain sequence that is at least 80%, at least 85%, at least 90%, or at least 95% identical to SEQ ID NO:666. The disclosure also provides antibodies comprising a heavy chain variable domain sequence that is at least 80%, at least 85%, at least 90%, or at least 95% identical to SEQ ID NO:347 and a light chain variable domain sequence that is at least 80%, at least 85%, at least 90%, or at least 95% identical to SEQ ID NO:667. The disclosure also provides antibodies comprising a heavy chain variable domain sequence that is at least 80%, at least 85%, at least 90%, or at least 95% identical to SEQ ID NO:348 and a light chain variable domain sequence that is at least 80%, at least 85%, at least 90%, or at least 95% identical to SEQ ID NO:572. The disclosure also provides antibodies comprising a heavy chain variable domain sequence that is at least 80%, at least 85%, at least 90%, or at least 95% identical to SEQ ID NO:349 and a light chain variable domain sequence that is at least 80%, at least 85%, at least 90%, or at least 95% identical to SEQ ID NO:668. The disclosure also provides antibodies comprising a heavy chain variable domain sequence that is at least 80%, at least 85%, at least 90%, or at least 95% identical to SEQ ID NO:350 and a light chain variable domain sequence that is at least 80%, at least 85%, at least 90%, or at least 95% identical to SEQ ID NO:643. The disclosure also provides antibodies comprising a heavy chain variable domain sequence that is at least 80%, at least 85%, at least 90%, or at least 95% identical to SEQ ID NO:351 and a light chain variable domain sequence that is at least 80%, at least 85%, at least 90%, or at least 95% identical to SEQ ID NO:669. The disclosure also provides antibodies comprising a heavy chain variable domain sequence that is at least 80%, at least 85%, at least 90%, or at least 95% identical to SEQ ID NO:352 and a light chain variable domain sequence that is at least 80%, at least 85%, at least 90%, or at least 95% identical to SEQ ID NO:669. The disclosure also provides antibodies comprising a heavy chain variable domain sequence that is at least 80%, at least 85%, at least 90%, or at least 95% identical to SEQ ID NO:353 and a light chain variable domain sequence that is at least 80%, at least 85%, at least 90%, or at least 95% identical to SEQ ID NO:670. The disclosure also provides antibodies comprising a heavy chain variable domain sequence that is at least 80%, at least 85%, at least 90%, or at least 95% identical to SEQ ID NO:354 and a light chain variable domain sequence that is at least 80%, at least 85%, at least 90%, or at least 95% identical to SEQ ID NO:671. The disclosure also provides antibodies comprising a heavy chain variable domain sequence that is at least 80%, at least 85%, at least 90%, or at least 95% identical to SEQ ID NO:355 and a light chain variable domain sequence that is at least 80%, at least 85%, at least 90%, or at least 95% identical to SEQ ID NO:482. The disclosure also provides antibodies comprising a heavy chain variable domain sequence that is at least 80%, at least 85%, at least 90%, or at least 95% identical to SEQ ID NO:356 and a light chain variable domain sequence that is at least 80%, at least 85%, at least 90%, or at least 95% identical to SEQ ID NO:672. The disclosure also provides antibodies comprising a heavy chain variable domain sequence that is at least 80%, at least 85%, at least 90%, or at least 95% identical to SEQ ID NO:357 and a light chain variable domain sequence that is at least 80%, at least 85%, at least 90%, or at least 95% identical to SEQ ID NO:673. The disclosure also provides antibodies comprising a heavy chain variable domain sequence that is at least 80%, at least 85%, at least 90%, or at least 95% identical to SEQ ID NO:358 and a light chain variable domain sequence that is at least 80%, at least 85%, at least 90%, or at least 95% identical to SEQ ID NO:658. The disclosure also provides antibodies comprising a heavy chain variable domain sequence that is at least 80%, at least 85%, at least 90%, or at least 95% identical to SEQ ID NO:359 and a light chain variable domain sequence that is at least 80%, at least 85%, at least 90%, or at least 95% identical to SEQ ID NO:572. The disclosure also provides antibodies comprising a heavy chain variable domain sequence that is at least 80%, at least 85%, at least 90%, or at least 95% identical to SEQ ID NO:360 and a light chain variable domain sequence that is at least 80%, at least 85%, at least 90%, or at least 95% identical to SEQ ID NO:674. The disclosure also provides antibodies comprising a heavy chain variable domain sequence that is at least 80%, at least 85%, at least 90%, or at least 95% identical to SEQ ID NO:361 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:319 and a light chain variable domain sequence that is at least 80%, at least 85%, at least 90%, or at least 95% identical to SEQ ID NO:675. The disclosure also provides antibodies comprising a heavy chain variable domain sequence that is at least 80%, at least 85%, at least 90%, or at least 95% identical to SEQ ID NO:362 and a light chain variable domain sequence that is at least 80%, at least 85%, at least 90%, or at least 95% identical to SEQ ID NO:676. The disclosure also provides antibodies comprising a heavy chain variable domain sequence that is at least 80%, at least 85%, at least 90%, or at least 95% identical to SEQ ID NO:363 and a light chain variable domain sequence that is at least 80%, at least 85%, at least 90%, or at least 95% identical to SEQ ID NO:482. The disclosure also provides antibodies comprising a heavy chain variable domain sequence that is at least 80%, at least 85%, at least 90%, or at least 95% identical to SEQ ID NO:364 and a light chain variable domain sequence that is at least 80%, at least 85%, at least 90%, or at least 95% identical to SEQ ID NO:645. The disclosure also provides antibodies comprising a heavy chain variable domain sequence that is at least 80%, at least 85%, at least 90%, or at least 95% identical to SEQ ID NO:365 and a light chain variable domain sequence that is at least 80%, at least 85%, at least 90%, or at least 95% identical to SEQ ID NO:677. The disclosure also provides antibodies comprising a heavy chain variable domain sequence that is at least 80%, at least 85%, at least 90%, or at least 95% identical to SEQ ID NO:366 and a light chain variable domain sequence that is at least 80%, at least 85%, at least 90%, or at least 95% identical to SEQ ID NO:678. The disclosure also provides antibodies comprising a heavy chain variable domain sequence that is at least 80%, at least 85%, at least 90%, or at least 95% identical to SEQ ID NO:367 and a light chain variable domain sequence that is at least 80%, at least 85%, at least 90%, or at least 95% identical to SEQ ID NO:679. The disclosure also provides antibodies comprising a heavy chain variable domain sequence that is at least 80%, at least 85%, at least 90%, or at least 95% identical to SEQ ID NO:368 and a light chain variable domain sequence that is at least 80%, at least 85%, at least 90%, or at least 95% identical to SEQ ID NO:680. The disclosure also provides antibodies comprising a heavy chain variable domain sequence that is at least 80%, at least 85%, at least 90%, or at least 95% identical to SEQ ID NO:369 and a light chain variable domain sequence that is at least 80%, at least 85%, at least 90%, or at least 95% identical to SEQ ID NO:681. The disclosure also provides antibodies comprising a heavy chain variable domain sequence that is at least 80%, at least 85%, at least 90%, or at least 95% identical to SEQ ID NO:370 and a light chain variable domain sequence that is at least 80%, at least 85%, at least 90%, or at least 95% identical to SEQ ID NO:645. The disclosure also provides antibodies comprising a heavy chain variable domain sequence that is at least 80%, at least 85%, at least 90%, or at least 95% identical to SEQ ID NO:371 and a light chain variable domain sequence that is at least 80%, at least 85%, at least 90%, or at least 95% identical to SEQ ID NO:682. The disclosure also provides antibodies comprising a heavy chain variable domain sequence that is at least 80%, at least 85%, at least 90%, or at least 95% identical to SEQ ID NO:372 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:373 and a light chain variable domain sequence that is at least 80%, at least 85%, at least 90%, or at least 95% identical to SEQ ID NO:683. The disclosure also provides antibodies comprising a heavy chain variable domain sequence that is at least 80%, at least 85%, at least 90%, or at least 95% identical to SEQ ID NO:374 and a light chain variable domain sequence that is at least 80%, at least 85%, at least 90%, or at least 95% identical to SEQ ID NO:684. The disclosure also provides antibodies comprising a heavy chain variable domain sequence that is at least 80%, at least 85%, at least 90%, or at least 95% identical to SEQ ID NO:375 and a light chain variable domain sequence that is at least 80%, at least 85%, at least 90%, or at least 95% identical to SEQ ID NO:685. The disclosure also provides antibodies comprising a heavy chain variable domain sequence that is at least 80%, at least 85%, at least 90%, or at least 95% identical to SEQ ID NO:376 and a light chain variable domain sequence that is at least 80%, at least 85%, at least 90%, or at least 95% identical to SEQ ID NO:482. The disclosure also provides antibodies comprising a heavy chain variable domain sequence that is at least 80%, at least 85%, at least 90%, or at least 95% identical to SEQ ID NO:377 and a light chain variable domain sequence that is at least 80%, at least 85%, at least 90%, or at least 95% identical to SEQ ID NO:686. The disclosure also provides antibodies comprising a heavy chain variable domain sequence that is at least 80%, at least 85%, at least 90%, or at least 95% identical to SEQ ID NO:378 and a light chain variable domain sequence that is at least 80%, at least 85%, at least 90%, or at least 95% identical to SEQ ID NO:572. The disclosure also provides antibodies comprising a heavy chain variable domain sequence that is at least 80%, at least 85%, at least 90%, or at least 95% identical to SEQ ID NO:379 and a light chain variable domain sequence that is at least 80%, at least 85%, at least 90%, or at least 95% identical to SEQ ID NO:667. The disclosure also provides antibodies comprising a heavy chain variable domain sequence that is at least 80%, at least 85%, at least 90%, or at least 95% identical to SEQ ID NO:1366 and a light chain variable domain sequence that is at least 80%, at least 85%, at least 90%, or at least 95% identical to SEQ ID NO:1367. The disclosure also provides antibodies comprising a heavy chain variable domain sequence that is at least 80%, at least 85%, at least 90%, or at least 95% identical to SEQ ID NO:1366 and a light chain variable domain sequence that is at least 80%, at least 85%, at least 90%, or at least 95% identical to SEQ ID NO:1368. The disclosure also provides antibodies comprising a heavy chain variable domain sequence that is at least 80%, at least 85%, at least 90%, or at least 95% identical to SEQ ID NO:1369 and a light chain variable domain sequence that is at least 80%, at least 85%, at least 90%, or at least 95% identical to SEQ ID NO:1370. The disclosure also provides antibodies comprising a heavy chain variable domain sequence that is at least 80%, at least 85%, at least 90%, or at least 95% identical to SEQ ID NO:1369 and a light chain variable domain sequence that is at least 80%, at least 85%, at least 90%, or at least 95% identical to SEQ ID NO:1371. The disclosure also provides antibodies comprising a heavy chain variable domain sequence that is at least 80%, at least 85%, at least 90%, or at least 95% identical to SEQ ID NO:1372 and a light chain variable domain sequence that is at least 80%, at least 85%, at least 90%, or at least 95% identical to SEQ ID NO:1370. The disclosure also provides antibodies comprising a heavy chain variable domain sequence that is at least 80%, at least 85%, at least 90%, or at least 95% identical to SEQ ID NO:1372 and a light chain variable domain sequence that is at least 80%, at least 85%, at least 90%, or at least 95% identical to SEQ ID NO:1371. The disclosure also provides antibodies comprising a heavy chain variable domain sequence that is at least 80%, at least 85%, at least 90%, or at least 95% identical to SEQ ID NO:1373 and a light chain variable domain sequence that is at least 80%, at least 85%, at least 90%, or at least 95% identical to SEQ ID NO:1374. The disclosure also provides antibodies comprising a heavy chain variable domain sequence that is at least 80%, at least 85%, at least 90%, or at least 95% identical to SEQ ID NO:1373 and a light chain variable domain sequence that is at least 80%, at least 85%, at least 90%, or at least 95% identical to SEQ ID NO:1375. The disclosure also provides antibodies comprising a heavy chain variable domain sequence that is at least 80%, at least 85%, at least 90%, or at least 95% identical to SEQ ID NO:1376 and a light chain variable domain sequence that is at least 80%, at least 85%, at least 90%, or at least 95% identical to SEQ ID NO:1374. The disclosure also provides antibodies comprising a heavy chain variable domain sequence that is at least 80%, at least 85%, at least 90%, or at least 95% identical to SEQ ID NO:1376 and a light chain variable domain sequence that is at least 80%, at least 85%, at least 90%, or at least 95% identical to SEQ ID NO:1375. The disclosure also provides antibodies comprising a heavy chain variable domain sequence that is at least 80%, at least 85%, at least 90%, or at least 95% identical to SEQ ID NO:1377 and a light chain variable domain sequence that is at least 80%, at least 85%, at least 90%, or at least 95% identical to SEQ ID NO:1378. The disclosure also provides antibodies comprising a heavy chain variable domain sequence that is at least 80%, at least 85%, at least 90%, or at least 95% identical to SEQ ID NO:1377 and a light chain variable domain sequence that is at least 80%, at least 85%, at least 90%, or at least 95% identical to SEQ ID NO:1379. The disclosure also provides antibodies comprising a heavy chain variable domain sequence that is at least 80%, at least 85%, at least 90%, or at least 95% identical to SEQ ID NO:1380 and a light chain variable domain sequence that is at least 80%, at least 85%, at least 90%, or at least 95% identical to SEQ ID NO:1378. The disclosure also provides antibodies comprising a heavy chain variable domain sequence that is at least 80%, at least 85%, at least 90%, or at least 95% identical to SEQ ID NO:1380 and a light chain variable domain sequence that is at least 80%, at least 85%, at least 90%, or at least 95% identical to SEQ ID NO:1379. The disclosure also provides antibodies comprising a heavy chain variable domain sequence that is at least 80%, at least 85%, at least 90%, or at least 95% identical to SEQ ID NO:1381 and a light chain variable domain sequence that is at least 80%, at least 85%, at least 90%, or at least 95% identical to SEQ ID NO:1382. The disclosure also provides antibodies comprising a heavy chain variable domain sequence that is at least 80%, at least 85%, at least 90%, or at least 95% identical to SEQ ID NO:1381 and a light chain variable domain sequence that is at least 80%, at least 85%, at least 90%, or at least 95% identical to SEQ ID NO:1383. The disclosure also provides antibodies comprising a heavy chain variable domain sequence that is at least 80%, at least 85%, at least 90%, or at least 95% identical to SEQ ID NO:1384 and a light chain variable domain sequence that is at least 80%, at least 85%, at least 90%, or at least 95% identical to SEQ ID NO:1382. The disclosure also provides antibodies comprising a heavy chain variable domain sequence that is at least 80%, at least 85%, at least 90%, or at least 95% identical to SEQ ID NO:1384 and a light chain variable domain sequence that is at least 80%, at least 85%, at least 90%, or at least 95% identical to SEQ ID NO:1383. The disclosure also provides antibodies comprising a heavy chain variable domain sequence that is at least 80%, at least 85%, at least 90%, or at least 95% identical to SEQ ID NO:1385 and a light chain variable domain sequence that is at least 80%, at least 85%, at least 90%, or at least 95% identical to SEQ ID NO:1386. The disclosure also provides antibodies comprising a heavy chain variable domain sequence that is at least 80%, at least 85%, at least 90%, or at least 95% identical to SEQ ID NO:1385 and a light chain variable domain sequence that is at least 80%, at least 85%, at least 90%, or at least 95% identical to SEQ ID NO:1387. The disclosure also provides antibodies comprising a heavy chain variable domain sequence that is at least 80%, at least 85%, at least 90%, or at least 95% identical to SEQ ID NO:1388 and a light chain variable domain sequence that is at least 80%, at least 85%, at least 90%, or at least 95% identical to SEQ ID NO:1386. The disclosure also provides antibodies comprising a heavy chain variable domain sequence that is at least 80%, at least 85%, at least 90%, or at least 95% identical to SEQ ID NO:1388 and a light chain variable domain sequence that is at least 80%, at least 85%, at least 90%, or at least 95% identical to SEQ ID NO:1387. The disclosure also provides antibodies comprising a heavy chain variable domain sequence that is at least 80%, at least 85%, at least 90%, or at least 95% identical to SEQ ID NO:1389 and a light chain variable domain sequence that is at least 80%, at least 85%, at least 90%, or at least 95% identical to SEQ ID NO:1390. The disclosure also provides antibodies comprising a heavy chain variable domain sequence that is at least 80%, at least 85%, at least 90%, or at least 95% identical to SEQ ID NO:1391 and a light chain variable domain sequence that is at least 80%, at least 85%, at least 90%, or at least 95% identical to SEQ ID NO:1392. The disclosure also provides antibodies comprising a heavy chain variable domain sequence that is at least 80%, at least 85%, at least 90%, or at least 95% identical to SEQ ID NO:1391 and a light chain variable domain sequence that is at least 80%, at least 85%, at least 90%, or at least 95% identical to SEQ ID NO:1393. The disclosure also provides antibodies comprising a heavy chain variable domain sequence that is at least 80%, at least 85%, at least 90%, or at least 95% identical to SEQ ID NO:1394 and a light chain variable domain sequence that is at least 80%, at least 85%, at least 90%, or at least 95% identical to SEQ ID NO:1392. The disclosure also provides antibodies comprising a heavy chain variable domain sequence that is at least 80%, at least 85%, at least 90%, or at least 95% identical to SEQ ID NO:1394 and a light chain variable domain sequence that is at least 80%, at least 85%, at least 90%, or at least 95% identical to SEQ ID NO:1393. The disclosure also provides antibodies comprising a heavy chain variable domain sequence that is at least 80%, at least 85%, at least 90%, or at least 95% identical to SEQ ID NO:1395 and a light chain variable domain sequence that is at least 80%, at least 85%, at least 90%, or at least 95% identical to SEQ ID NO:1386. The disclosure also provides antibodies comprising a heavy chain variable domain sequence that is at least 80%, at least 85%, at least 90%, or at least 95% identical to SEQ ID NO:1396 and a light chain variable domain sequence that is at least 80%, at least 85%, at least 90%, or at least 95% identical to SEQ ID NO:1386. The disclosure also provides antibodies comprising a heavy chain variable domain sequence that is at least 80%, at least 85%, at least 90%, or at least 95% identical to SEQ ID NO:1397 and a light chain variable domain sequence that is at least 80%, at least 85%, at least 90%, or at least 95% identical to SEQ ID NO:1386. The disclosure also provides antibodies comprising a heavy chain variable domain sequence that is at least 80%, at least 85%, at least 90%, or at least 95% identical to SEQ ID NO:1398 and a light chain variable domain sequence that is at least 80%, at least 85%, at least 90%, or at least 95% identical to SEQ ID NO:1399. The disclosure also provides antibodies comprising a heavy chain variable domain sequence that is at least 80%, at least 85%, at least 90%, or at least 95% identical to SEQ ID NO:1400 and a light chain variable domain sequence that is at least 80%, at least 85%, at least 90%, or at least 95% identical to SEQ ID NO:1399. The disclosure also provides antibodies comprising a heavy chain variable domain sequence that is at least 80%, at least 85%, at least 90%, or at least 95% identical to SEQ ID NO:1401 and a light chain variable domain sequence that is at least 80%, at least 85%, at least 90%, or at least 95% identical to SEQ ID NO:1399. The disclosure also provides antibodies comprising a heavy chain variable domain sequence that is at least 80%, at least 85%, at least 90%, or at least 95% identical to SEQ ID NO:1398 and a light chain variable domain sequence that is at least 80%, at least 85%, at least 90%, or at least 95% identical to SEQ ID NO:1382. The disclosure also provides antibodies comprising a heavy chain variable domain sequence that is at least 80%, at least 85%, at least 90%, or at least 95% identical to SEQ ID NO:1400 and a light chain variable domain sequence that is at least 80%, at least 85%, at least 90%, or at least 95% identical to SEQ ID NO:1382. The disclosure also provides antibodies comprising a heavy chain variable domain sequence that is at least 80%, at least 85%, at least 90%, or at least 95% identical to SEQ ID NO:1401 and a light chain variable domain sequence that is at least 80%, at least 85%, at least 90%, or at least 95% identical to SEQ ID NO:1382. The disclosure also provides antibodies comprising a heavy chain variable domain sequence that is at least 80%, at least 85%, at least 90%, or at least 95% identical to SEQ ID NO:1402 and a light chain variable domain sequence that is at least 80%, at least 85%, at least 90%, or at least 95% identical to SEQ ID NO:1374. The disclosure also provides antibodies comprising a heavy chain variable domain sequence that is at least 80%, at least 85%, at least 90%, or at least 95% identical to SEQ ID NO:1402 and a light chain variable domain sequence that is at least 80%, at least 85%, at least 90%, or at least 95% identical to SEQ ID NO:1403. The disclosure also provides antibodies comprising a heavy chain variable domain sequence that is at least 80%, at least 85%, at least 90%, or at least 95% identical to SEQ ID NO:1404 and a light chain variable domain sequence that is at least 80%, at least 85%, at least 90%, or at least 95% identical to SEQ ID NO:1374. The disclosure also provides antibodies comprising a heavy chain variable domain sequence that is at least 80%, at least 85%, at least 90%, or at least 95% identical to SEQ ID NO:1404 and a light chain variable domain sequence that is at least 80%, at least 85%, at least 90%, or at least 95% identical to SEQ ID NO:1403. The disclosure also provides antibodies comprising a heavy chain variable domain sequence that is at least 80%, at least 85%, at least 90%, or at least 95% identical to SEQ ID NO:1405 and a light chain variable domain sequence that is at least 80%, at least 85%, at least 90%, or at least 95% identical to SEQ ID NO:1406. The disclosure also provides antibodies comprising a heavy chain variable domain sequence that is at least 80%, at least 85%, at least 90%, or at least 95% identical to SEQ ID NO:1405 and a light chain variable domain sequence that is at least 80%, at least 85%, at least 90%, or at least 95% identical to SEQ ID NO:1407.

Table 1 provides a summary of the TIGIT-specific antibodies described herein:

### Pharmaceutical Compositions

Pharmaceutical compositions comprising the antibodies or antibody fragments of the present disclosure are also contemplated and can be used in the methods disclosed herein. Pharmaceutical compositions can comprise one or more of the antibodies or antibody fragments described herein and a pharmaceutically acceptable carrier or excipient. Although the carrier or excipient may facilitate administration, it should not itself induce the production of antibodies harmful to the subject or individual receiving the composition; nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles, and are known to one of skill in the art.

The antibodies or an antigen binding fragments described herein, or the pharmaceutical compositions disclosed herein, may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Typically, the therapeutic compositions may be prepared as injectables, either as liquid solutions or suspensions. Solid forms suitable for solution in, or suspension in, liquid vehicles prior to injection may also be prepared.

In one embodiment, the antibody, or an antigen binding fragment thereof, or pharmaceutical composition is administered intravenously. In another embodiment, the antibody, or an antigen binding fragment thereof, or pharmaceutical composition is administered by intravenous infusion.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule. Known antibody-based pharmaceuticals provide guidance relating to frequency of administration e.g., whether a pharmaceutical should be delivered daily, weekly, monthly, etc. Frequency and dosage may also depend on the severity of symptoms.

It will be appreciated that the active ingredient in the composition will be an antibody molecule, an antibody fragment or variants and derivatives thereof. As such, it will be susceptible to degradation in the gastrointestinal tract. Thus, if the composition is to be administered by a route using the gastrointestinal tract, the composition will need to contain agents which protect the antibody from degradation, but which release the antibody once it has been absorbed from the gastrointestinal tract.

The methods of the present invention can use an antibody, or an antigen binding fragment thereof, as described above, alone or in combination with other pharmaceutically active compounds, to treat conditions such as those disclosed hereinabove. The additional pharmaceutically active compound(s) can be administered simultaneously (either in the same dosage form or in separate dosage forms) or sequentially. Accordingly, in one embodiment, the present invention comprises methods for treating a condition by administering to the subject a therapeutically effective amount of an antibody, or an antigen binding fragment thereof, of the present invention and one or more additional pharmaceutically active compounds.

In some embodiments, the antibody, or an antigen binding fragment thereof, of the present invention is used in combination with existing TIGIT-related disease therapies.

### EXAMPLES

The following examples put forth so as to provide those of ordinary skill in the art with a complete description of how to make and use the present disclosure, and is not intended to limit the scope of what the inventors regard as their disclosure nor is it intended to represent that the experiment below is all or the only experiment that could be performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Isolation of Murine Anti-Human TIGIT Antibodies

### Immunization, & Single Cell Suspension Generation

Recombinant human TIGIT His tagged, catalog no: TIT-H52H3, ACROBiosystems, Beijing, China, SEQ ID NO:2) was used to immunize young C57/BL6 mice each with 80 μg of the protein in Sigma Adjuvant System® (Sigma-Aldrich, St. Louis, Mo.) over a period of 35 days using a rapid immunization protocol of Antibody Solutions (Sunnyvale, Calif.). The lymph nodes were harvested on day 35. Single cell suspension of the lymph node was generated, and the suspension was filtered through a 70 μm mesh (BD Bioscience) to remove clumps.

### Plasma Cell Isolation, Antibody Capture, and Antigen Interrogation

The filtered lymphocyte suspension was enriched for plasma cells actively secreting IgGs using a kit based on cell surface expression of CD138 (Miltenyi, Auburn, Calif.). Using a method described in U.S. Pat. No. 9,328,172, freshly enriched plasma cells were deposited on a PDMS device to allow a single cell settled in the microwells on the device. Antibody secreted from each plasma cell was captured on a derivatized microscope slide. Antigen-specific antibody secreting cells were identified by interrogating the antibody capture slide with varying concentrations of fluorescently labeled human TIGIT protein tagged with human Fc (ACROBiosystems, Beijing, China, catalog no: TIT-H5254, SEQ ID NO:3). Labeling was done using a kit (AnaSpec, Fremont, Calif., AS-72046, AnaTag™ HiLyte™ Fluor 555 Microscale Protein Labeling Kit *Ultra Convenient*).

mRNA Capture

After antibody capture, the medium was removed, and replaced with lysis buffer followed by prompt closure of the top of the microwells with a custom oligonucleotide microarray (Agilent, Santa Clara, Calif.). This procedure was previously described in U.S. Pat. No. 9,328,172. The custom oligonucleotide microarray is prepared such that each feature contains not only a unique tag specifying its coordinate but also capture probes for all subclasses (1, 2a, 2b, and 3) of murine IgG heavy chain, murine Ig kappa light chain.

cDNA Synthesis, PCR Amplification, and Next Generation Sequencing

Captured mRNA on the custom microarray was further processed to synthesize cDNA of each mRNA incorporating the unique tag originally on each feature. The cDNA is then amplify using a Taq polymerase (Promega, Madison, Wis.) and appropriate set of primers to allow amplification of the following genes: variable domain of IgG heavy chain subclasses and variable domain of Ig kappa light chain. Though now released from cells, these fragments of each gene are now labeled with the unique tag from the custom oligonucleotide microarray manifesting their originating locations. The amplicons were further manipulated to have appropriate sequence attached at both ends to enable sequencing on an Illumina MiSeq instrument using 2×250 bp chemistry at SeqMatic LLC (Fremont, Calif.).

### Bioinformatic Analysis of Images and DNA Sequences

Sequencing reads from MiSeq were processed and the embedded tag in each read was identified and converted into coordinates. The coordinates were plotted to yield a synthetic map of the mRNA recovered. Most of the coordinates form clusters that designate the location of the originating cell for the recovered mRNA sequences. Next, CDR3 motif present in each read with the coordinates was identified and collated according to the clusters that matched the location of an antibody spot visualized by an appropriate fluorescently labeled secondary antibody. Identical or nearly identical CDR3s for a given antibody spot were organized and form consistent pair of V_{H} and V_{L} sequences. The remaining part of V_{H} or V_{L} sequence containing the identified CDR3s was identified and the associated sequencing reads were assembled into full-length cDNA sequences for V_{H} and V_{L}. The pair of full-length cDNA was correlated with the affinity measurements associated with each of the antigen-specific antibody spot.

### Example 2: Molecular Reconstruction, Recombinant Expression, and Humanization of Anti-TIGIT Antibodies

A select subset of paired V_{H} and V_{L} anti-TIGIT antibody sequences was used to synthesize corresponding gene fragments by a service provider according to the known art. The resulting gene fragments were cloned into appropriate plasmid vectors to be in-frame fused with human IgG1 Fc or human kappa constant region, respectively, and transfected into an appropriate mammalian host, such as HEK293, for recombinant expression to produce a chimeric antibody preparation in full-IgG format. The antibody preparations were characterized by measurements at OD280 to assess the amount produced and by gel electrophoresis on PAGE to assess the size of the antibody chains produced. Chimeric or humanized antibodies described herein can be prepared based on the sequence of a murine monoclonal antibody generated as described above. To create a chimeric antibody, the murine variable regions were linked to human constant regions using methods known in the art (see e.g., U.S. Pat. No. 4,816,567 to Cabilly et al.). To create a humanized antibody, the murine CDR regions were inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

### Example 3: Characterization of Recombinant Anti-TIGIT Antibodies

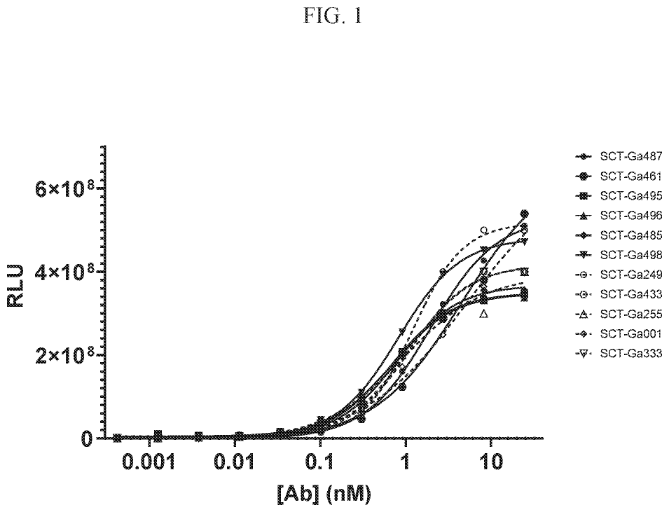

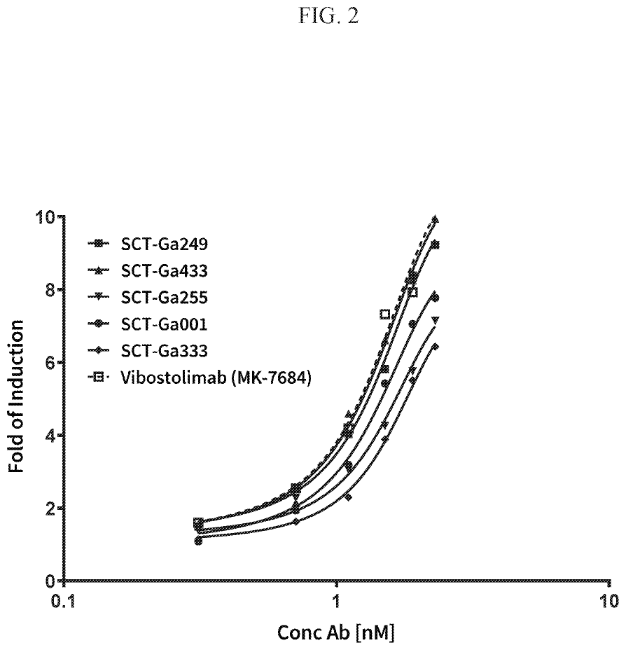

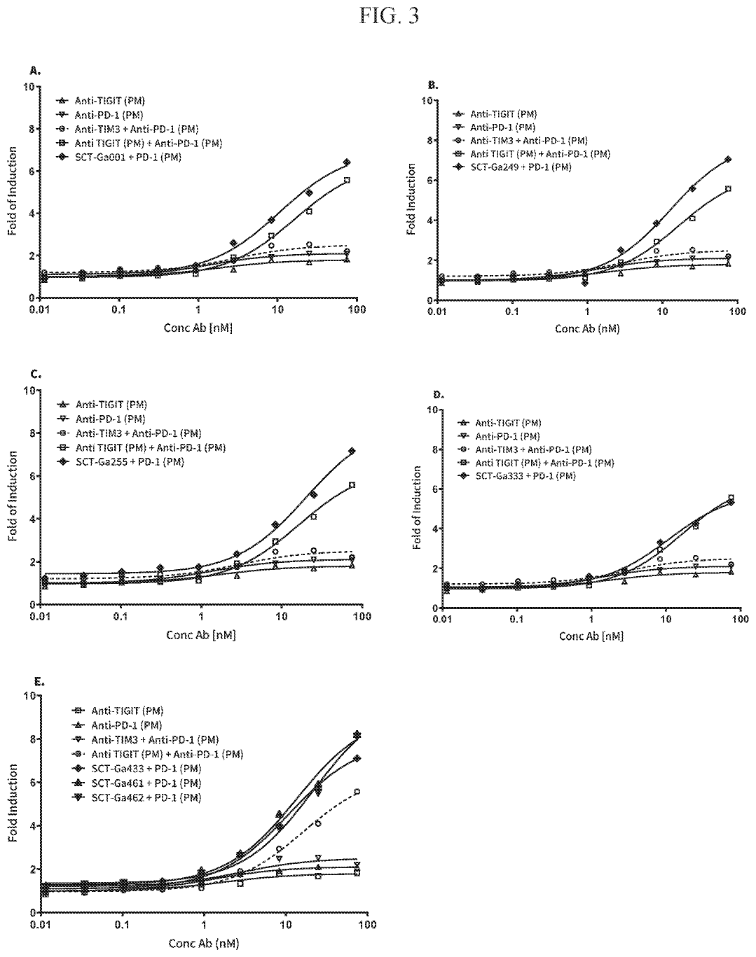

The recombinantly expressed antibodies were used to assess binding activity to TIGIT by conventional ELISA according to the known art. Recombinant human TIGIT was coated onto ELISA plates to detect binding at serially diluted concentrations of the anti-TIGIT antibody preparations (FIG. 1). Each antibody's EC50 value is shown in Table 2. Anti-TIGIT antibodies' blockade activity was assessed using engineered cell lines, kit reagents (TIGIT/CD155 Blockade Bioassay, J2201, Promega, Madison, Wis.), and a luminometer (Promega, Madison, Wis.) and the results were used to compare against benchmark antibodies with known blockade activity (FIG. 2). Their EC50 values are shown in table 3. Similarly, these antibodies were assessed for their ability to synergize with an anti-PD-1 antibody using a PD-1+TIGIT combination bioassay (Cat. No. J2211 Promega, Madison, Wis.). The results are shown in FIG. 3.

Antibodies recovered from the antibody campaign described above are listed herein. The CDR sequences and the V_{H} and V_{L} sequences for the anti-TIGIT antibodies described herein are depicted in Tables 4 and 5, respectively.

TABLE 2

| Antibody | EC50 (nM) |
|---|---|
| Ga001 | 5.03 |
| Ga249 | 1.22 |
| Ga255 | 0.96 |
| Ga333 | 1.08 |
| Ga433 | 1.10 |
| Ga461 | 4.55 |
| Ga487 | 1.96 |
| Ga495 | 0.69 |
| Ga496 | 0.67 |
| Ga485 | 0.79 |
| Ga498 | 0.82 |

TABLE 3

| | Blockade |
|---|---|
| Antibody | EC50 (nM) |
| Ga001 | 26.59 |
| Ga249 | 28.08 |
| Ga255 | 33.35 |
| Ga333 | 42.22 |
| Ga433 | 25.19 |
| MK-7683 | 24.84 |

TABLE 4
CDR sequences of mAbs generated against human TIGIT
(SEQ ID Nos for each listed sequence are provided in parenthesis)

| mAb AA ID | HCDR1 | HCDR2 | HCDR3 | LCDR1 | LCDR2 | LCDR3 |
|---|---|---|---|---|---|---|
| SCT-Ga001 | GYAFTNYL | INPGSGGS | ARTGVYGNFLIFDY | QSLLNSGIQKNY | GAS | QNDHRYPFT |
| | (687) | (688) | (689) | (690) | (691) | (692) |
| SCT-Ga002 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga003 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga004 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga005 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga006 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga007 | GFTFTDHY | IRNKVNGYTT | ARYIPYYDPFDY | QDINSY | RAN | LQYDELYT |
| SCT-Ga008 | (699) | (700) | (701) | (702) | (703) | (704) |
| | GYSITNGNHW | ITSSGRT | SREGGWLPPV | QSLLDSDGKTY | LVS | WQGTHFPYT |
| | (693) | (694) | (705) | (696) | (697) | (698) |
| SCT-Ga009 | GYTFTDYY | INPYNGGT | AREGTD | QSLLDSDGKTY | LVS | WQGTHFPWT |
| | (706) | (707) | (708) | (696) | (697) | (709) |
| SCT-Ga010 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga011 | GYSITNGNHW | ISSSGRI | AREGGWLPPV | QSLLDSDGKTY | LVS | WQGTHFPYT |
| | (693) | (710) | (695) | (696) | (697) | (698) |
| SCT-Ga012 | GFTFSNYG | ISTGGSYT | ARPHYYGSSSGNYFDY | EDIYNR | GAT | QQYWSTPWT |
| | (711) | (712) | (713) | (714) | (715) | (716) |
| SCT-Ga013 | GYSITHGNHW | ISSSGRI | AREGGWLPPV | QSLLDSDGKTY | LVS | WQGTHFPYT |
| | (717) | (710) | (695) | (696) | (697) | (698) |
| SCT-Ga014 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga015 | GYSITNGNHW | IRSSGST | AREGGWLPPV | QSLLDSDGKTY | LLS | WQGTHFPYT |
| | (693) | (718) | (695) | (696) | (719) | (698) |
| SCT-Ga016 | GFSLTSYV | IWTGGGT | ARTRAYYSNWGYFDV | QSVLYSSNQKNY | WAS | HQYLSSYT |
| | (720) | (721) | (722) | (723) | (724) | (725) |
| SCT-Ga017 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga018 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga019 | GYSITSGYY | ISYDGSN | ARGLYYDYDDYFDY | QDINKY | YTS | LQYDNLWT |
| | (727) | (728) | (729) | (730) | (731) | (732) |
| SCT-Ga020 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga021 | GYSITNGNHW | ITSSGRI | AREGGWLPPV | QSLFDSDGKTY | LVS | WQGTHFPYT |
| | (693) | (734) | (695) | (735) | (697) | (698) |
| SCT-Ga022 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga023 | GYSITNGNHW | ISSSGST | AREGGWLPPV | QSLLDSDGKTY | LVS | WQGTHFPYT |
| | (693) | (736) | (695) | (696) | (697) | (698) |
| SCT-Ga024 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga025 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga026 | GYSITNGNHW | ITSSGRT | TREGGWLPPV | QSLLDIDGKTY | LVS | WQGTHFPYT |
| | (693) | (694) | (733) | (737) | (697) | (698) |
| SCT-Ga027 | GYTFTTYG | INTYSGVP | ARYDYDVGFAY | QSLVHSNGNTY | KIS | SQSTHVPA |
| | (738) | (739) | (740) | (741) | (742) | (743) |
| SCT-Ga028 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga029 | GYTFTTYG | INTFSRVP | ARYDYDVGFAY | QSLVHSNGNTY | KVS | SQSTHVPT |
| | (738) | (744) | (740) | (741) | (745) | (746) |
| SCT-Ga030 | GYSITNGNHW | ITSSGRT | SREGGWLPPV | QSLLDSDGKTY | LVS | WQGTHFPYT |
| | (693) | (694) | (705) | (696) | (697) | (698) |
| SCT-Ga031 | GISITTGNYK | IYYSGTI | ARETVLRVFDY | QDVGTA | SAS | QQHYSSPRT |
| | (747) | (748) | (749) | (750) | (751) | (752) |
| SCT-Ga032 | GYSITNGHHW | ISSSGST | AREGGWLPPV | QSLLDSDGKTY | LVS | WQGTHFPYT |
| | (753) | (736) | (695) | (696) | (697) | (698) |
| SCT-Ga033 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga034 | GYSITNGNHW | ISSSGRI | AREGGWLPPV | QSLLDSDGKTY | LVS | WQGTHFPYT |
| | (693) | (710) | (695) | (696) | (697) | (698) |
| SCT-Ga035 | GYSITNGNHW | IRSSGST | AREGGWLPPV | QSLLDSDGKTY | LVS | WQGTHFPYT |
| | (693) | (718) | (695) | (696) | (697) | (698) |
| SCT-Ga036 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga037 | GYTFTDHT | IYPRDGST | ARRVVASGMDY | QDVSTA | SAS | QQHYSTPYT |
| | (754) | (755) | (756) | (757) | (751) | (758) |
| SCT-Ga038 | GYAFTNYL | INPGSGGS | ARTGVYGNFLIFDY | QSLLNSGIQKNY | GAS | QNDHRYPFT |
| | (687) | (688) | (689) | (690) | (691) | (692) |
| SCT-Ga039 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga040 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga041 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga042 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga043 | GYSITNGNHW | IRSSGST | TREGGWLPPV | QSLLDRDGKTY | LVS | WQGTHFPYT |
| | (693) | (718) | (733) | (759) | (697) | (698) |
| SCT-Ga044 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga045 | GYSITNGNHW | ISSSGRT | AREGGWLPPV | QSLLDRDGKTY | LVS | WQGTHFPYT |
| | (693) | (726) | (695) | (759) | (697) | (698) |
| SCT-Ga046 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga047 | GYSITNGNHW | IRSSGST | AREGGWLPPV | QSLLDSDGKTY | LVS | WQGTHFPYT |
| | (693) | (718) | (695) | (696) | (697) | (698) |
| SCT-Ga048 | GYSITSGNHW | ISSSGRI | AREGGWLPPV | QSLLDSDGKTY | LVS | WQGTHFPYT |
| | (760) | (710) | (695) | (696) | (697) | (698) |
| SCT-Ga049 | GISITTGNYK | IYYSGTI | ARETVLRVFDY | QDVSTA | SAS | QQHYSSPRT |
| | (747) | (748) | (749) | (757) | (751) | (752) |
| SCT-Ga050 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga051 | GYTFTDYY | IFPGSGST | ARFTV | QSLLDSDGKTY | LVS | WQGTHFPHT |
| | (706) | (761) | (762) | (696) | (697) | (763) |
| SCT-Ga052 | GYTFTDYY | IYPGSGNT | ARVTYYGGYFDY | QDISNY | YTS | QQGNTLPFT |
| | (706) | (764) | (765) | (766) | (731) | (767) |
| SCT-Ga053 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga054 | GYSITNGNHW | IRSSGST | AREGGWLPPV | QSLLDSDGKTY | LVS | WQGTHFPYT |
| | (693) | (718) | (695) | (696) | (697) | (698) |
| SCT-Ga055 | GYILSGYW | IFPGSGST | ARIRITTVPYFDF | QDVSKA | WAS | QQHYSTPWT |
| | (768) | (761) | (769) | (770) | (724) | (771) |
| SCT-Ga056 | GYTFTSYW | INPSNGVT | ARDYDYFDY | VNIHNY | NAK | QHFWSTPYT |
| | (772) | (773) | (774) | (775) | (776) | (777) |
| SCT-Ga057 | GYSITSAYY | ISYDGTN | GRGRPFVPY | QNVGSN | SAS | QQYNNYPFT |
| | (778) | (779) | (780) | (781) | (751) | (782) |
| SCT-Ga058 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga059 | GYTFTDHT | IYPRDGST | ARRALPYFDC | QDVFTA | SAS | QQHYSTPLT |
| | (754) | (755) | (783) | (784) | (751) | (785) |
| SCT-Ga060 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga061 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga062 | GISITTGNYK | IYYSGTI | ARETVLRVFDY | QDVSTA | SAS | QQHYSSPRT |
| | (747) | (748) | (749) | (757) | (751) | (752) |
| SCT-Ga063 | GYAFTNYL | INPGSGGS | ARTGVYGNFLIFDY | QSLLNSGVQKNY | GAS | QNDHRYPFT |
| | (687) | (688) | (689) | (786) | (691) | (692) |
| SCT-Ga064 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga065 | GYAFTNYL | INPGSGGS | ARTGVYANFLIFDY | QSLLNSGIQKNY | GAS | QNDHRYPFT |
| | (687) | (688) | (787) | (690) | (691) | (692) |
| SCT-Ga066 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga067 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga068 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga069 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga070 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga071 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga072 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga073 | GYSITNGNHW | ITSSGRT | AREGGWLPPV | QSLLDSDGKTY | LLS | WQGTHFPYT |
| | (693) | (694) | (695) | (696) | (719) | (698) |
| SCT-Ga074 | GYTFTSYW | INPSNGGT | ARRTTVLAGPYFDY | QNINVW | KAS | QQGQSYPLT |
| | (772) | (788) | (789) | (790) | (791) | (792) |
| SCT-Ga075 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga076 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga077 | GYSITNGNHW | ITSSGRT | AREGGWLPPV | QSLLDSDGQTY | LVS | WQGTHFPYT |
| | (693) | (694) | (695) | (793) | (697) | (698) |
| SCT-Ga078 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga079 | GYSITNGNHW | IRSSGST | AREGGRLPPV | QSLLDSDGKTY | LVS | WQGTHFPYT |
| | (693) | (718) | (794) | (696) | (697) | (698) |
| SCT-Ga080 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga081 | GYSISNGNHW | ITSSGRT | AREGGWLPPV | QSLLDSDGKTY | LVS | WQGTHFPYT |
| | (795) | (694) | (695) | (696) | (697) | (698) |
| SCT-Ga082 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga083 | GYSITNGNHW | ISSSGRI | AREGGWLPPV | QSLLDSDGKTY | LVS | WQGTHFPYT |
| | (693) | (710) | (695) | (696) | (697) | (698) |
| SCT-Ga084 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga085 | GYSITNGNHW | ISSSGRI | AREGGWLPPV | QSLLDSDGKTY | LVS | WQGTHFPYT |
| | (693) | (710) | (695) | (696) | (697) | (698) |
| SCT-Ga086 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga087 | GFITSRYI | ISGGGGNT | ARWLPSYYAMDY | QDIGSS | ATS | LQYASSPWT |
| | (796) | (797) | (798) | (799) | (800) | (801) |
| SCT-Ga088 | GFITSRYI | ISGGGGNT | ARWLPSYYAMDY | QNINIW | KAS | QQGQSYPYT |
| | (796) | (797) | (798) | (802) | (791) | (803) |
| SCT-Ga089 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga090 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga091 | GYSITNGNHW | IRSSGST | AREGGWLPPV | QSLLDSDGKTY | LVS | WQGTHFPYT |
| | (693) | (718) | (695) | (696) | (697) | (698) |
| SCT-Ga092 | GFITSSYT | ISGGGGNT | ARWLLTYYAMDY | QDIGSS | ATS | LQYVSCPWT |
| | (804) | (797) | (805) | (799) | (800) | (806) |
| SCT-Ga093 | GYTFTDCY | IFPGSDFT | ARYGLGHYFDY | QNINVW | KAS | QQGQSYPLT |
| | (807) | (808) | (809) | (790) | (791) | (792) |
| SCT-Ga094 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga095 | GYTFTAYC | IFPGSGST | ARYGLGHYFDY | QNINVW | KAS | QQGQSYPLT |
| | (810) | (761) | (809) | (790) | (791) | (792) |
| SCT-Ga096 | GYSITNGNHW | ITSSGRT | SREGGWFPPV | QSLLDSDGKTY | LVS | WQGTHFPYT |
| | (693) | (694) | (811) | (696) | (697) | (698) |
| SCT-Ga097 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga098 | GYSITNGNHW | ITSSGRT | TREGGWLPPV | QSLLDSDGKTY | LVS | WQGTHFPYT |
| | (693) | (694) | (733) | (696) | (697) | (698) |
| SCT-Ga099 | GYTFTSYW | INPSNGGT | ARRTTVLAGPYFDY | QNINVW | QAS | QQGQSYPLT |
| | (772) | (788) | (789) | (790) | (812) | (792) |
| SCT-Ga100 | GFTFSSYT | ISGGGGNT | ARWLLTYYAMDY | QNINVW | KAS | QQGQSYPYT |
| | (804) | (797) | (805) | (790) | (791) | (803) |
| SCT-Ga101 | GFTFSSYG | ISSGGSYT | ARHHYYYGSSYIFYFDV | QSVLYSSNQKNY | WAS | HQYLSSLT |
| | (813) | (814) | (815) | (723) | (724) | (816) |
| SCT-Ga102 | GFTFSSYT | ISGRGGNT | ARWLLTYYAMDY | QNINVW | KAS | QQGQSYPYT |
| | (804) | (817) | (805) | (790) | (791) | (803) |
| SCT-Ga103 | GFTFSSYT | ISGRGGNT | VRWLLTYYAMDY | QNINIW | KAS | QQGQSYPYT |
| | (804) | (817) | (818) | (802) | (791) | (803) |
| SCT-Ga104 | GFTFSSYT | ISGGGGNT | ARWLPTYYAMDY | QNINVW | KAS | QQGQSYPYT |
| | (804) | (797) | (819) | (790) | (791) | (803) |
| SCT-Ga105 | GYSFTTYG | INTYSGVP | ARYDYDVGFAY | QSLVHSNGNTY | KIS | SQSTHVPA |
| | (820) | (739) | (740) | (741) | (742) | (743) |
| SCT-Ga106 | GYSITHGNHW | ISSSGRI | AREGGWLPPV | QSLLDSDGKTY | LVS | WQGTHFPYT |
| | (717) | (710) | (695) | (696) | (697) | (698) |
| SCT-Ga107 | GYSITNGNHW | ITSSGRT | TREGGWLPPV | QSLLDSDGKTY | LVS | WQGTHFPYT |
| | (693) | (694) | (733) | (696) | (697) | (698) |
| SCT-Ga108 | GYSITNGNHW | ITSSGRI | AREGGWLPPV | QSLLDSDGKTY | LVS | WQGTHFPYT |
| | (693) | (734) | (695) | (696) | (697) | (698) |
| SCT-Ga109 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga110 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga111 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga112 | GYAFTNYL | INPGSGGS | ARTGVYGNFLIFDY | QSLLNSGIQKNY | GAS | QNDHRYPFT |
| | (687) | (688) | (689) | (690) | (691) | (692) |
| SCT-Ga113 | GYSITNGNHW | ITSSGRT | TREGGWLPPV | QSLLDSDGKTY | LVS | WQGTHFPYT |
| | (693) | (694) | (733) | (696) | (697) | (698) |
| SCT-Ga114 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga115 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga116 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga117 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga118 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga119 | GYSITNGNHW | IRSSGRT | TREGGWLPPV | QSLLDSDGKTY | LVS | WQGTHFPYT |
| | (693) | (821) | (733) | (696) | (697) | (698) |
| SCT-Ga120 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga121 | GYSITNGNHW | IRSSGST | TREGGWLPPV | QSLLDGDGRTY | LVS | WQGTHFPYT |
| | (693) | (718) | (733) | (822) | (697) | (698) |
| SCT-Ga122 | GYSITNGNHW | ITSSGRI | AREGGWLPPV | QSLLDSDGKTY | LVS | WQGTHFPYT |
| | (693) | (734) | (695) | (696) | (697) | (698) |
| SCT-Ga123 | GYSITNGNHW | ITSSGRT | TREGGWLPPV | QSLLDSDGKTY | LVS | WQGTHFPYT |
| | (693) | (694) | (733) | (696) | (697) | (698) |
| SCT-Ga124 | GYSITNGNHW | IRSSGST | SREGGWLPPV | QSLLDSDGKTY | LVS | WQGTHFPYT |
| | (693) | (718) | (705) | (696) | (697) | (698) |
| SCT-Ga125 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga126 | GYSITNGNHW | IRSSGRT | TREGGRLPPV | QSLLDSDGKTY | LVS | WQGTHFPYT |
| | (693) | (821) | (823) | (696) | (697) | (698) |
| SCT-Ga127 | GYSITNGNHW | ITSSGRT | AREGGWFPPV | QSLLDSDGKTY | LVS | WQGTHFPYT |
| | (693) | (694) | (824) | (696) | (697) | (698) |
| SCT-Ga128 | GYSIINGNHW | ITSSGRT | AREGGWLPPV | QSLLDSDGKTY | LVS | WQGTHFPYT |
| | (825) | (694) | (695) | (696) | (697) | (698) |
| SCT-Ga129 | GYSITTGNHW | IRSSGST | TREGGLFPPV | QSLLDSDGKTY | LVS | WQGTHFPYT |
| | (826) | (718) | (827) | (696) | (697) | (698) |
| SCT-Ga130 | GYSITNGNHW | ITSNGRT | AREGGWLPPV | QSLLDSDGKTY | LVS | WQGTHFPYT |
| | (693) | (828) | (695) | (696) | (697) | (698) |
| SCT-Ga131 | GFTFSRYI | ITGGGGNT | ARWLPTYYAMDY | QDIGSS | ATY | LLYVNSPWT |
| | (796) | (829) | (819) | (799) | (830) | (831) |
| SCT-Ga132 | GYSITNGNHW | ITSSGRT | AREGGWLPPV | QSLLDSDGRTY | LVS | WQGTHFPYT |
| | (693) | (694) | (695) | (832) | (697) | (698) |
| SCT-Ga133 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga134 | GYTFTDYY | IFPGSDNT | ARYGLGHYFDY | QNINVW | TTS | QQGQSYPLT |
| | (706) | (833) | (809) | (790) | (834) | (792) |
| SCT-Ga135 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga136 | GYTFTSYW | FYPGGGST | ARSYSNYVRFAY | QDVFTA | SAS | QQHYNTPLT |
| | (772) | (835) | (836) | (784) | (751) | (837) |
| SCT-Ga137 | GHTFTDYY | IFPGSGNT | AGYGLGHYFDN | SSISSSN | GTS | QQWSSYPLT |
| | (838) | (839) | (840) | (841) | (842) | (843) |
| SCT-Ga138 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga139 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga140 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga141 | GYSITNGNHW | IRSSGST | TREGGWLPPV | QSLLDSDGRTY | LVS | WQGTHFPYT |
| | (693) | (718) | (733) | (832) | (697) | (698) |
| SCT-Ga142 | GYSITNGKHW | IRSSGST | TREGGWLPPV | QSLLDSDGRTY | LVS | WQGTHFPYT |
| | (844) | (718) | (733) | (832) | (697) | (698) |
| SCT-Ga143 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga144 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga145 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga146 | GYSITNGNHW | ITSSGRT | TREGGWLPPV | QSLLDSDGKTY | LVS | WQGTHFPYT |
| | (693) | (694) | (733) | (696) | (697) | (698) |
| SCT-Ga147 | GYSITNGNHW | ITSSGRT | TREGGWLPPV | QSLLDGDGKTY | LVS | WQGTHFPYT |
| | (693) | (694) | (733) | (845) | (697) | (698) |
| SCT-Ga148 | GYSITNGNHW | IRSSGST | TREGGWLPPV | QSLLDRDGKTY | LVS | WQGTHFPYT |
| | (693) | (718) | (733) | (759) | (697) | (698) |
| SCT-Ga149 | GYSITNGNHW | ITSSGRI | AREGGWLPPV | QSLLDSDGKTY | LVS | WQGTHFPYT |
| | (693) | (734) | (695) | (696) | (697) | (698) |
| SCT-Ga150 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga151 | GYSITNGNHW | IRSSGGT | SREGGWLPPV | QSLLDSDGKTY | LVS | WQGTHFPYT |
| | (693) | (846) | (705) | (696) | (697) | (698) |
| SCT-Ga152 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga153 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga154 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga155 | GYSITNGNHW | ITSSGRT | SREGGWLPPV | QSLLDSDGKTY | LVS | WQGTHFPYT |
| | (693) | (694) | (705) | (696) | (697) | (698) |
| SCT-Ga156 | GYSITNGHHW | ITSSGRT | AREGGWLPPV | QSLLDSDGKTY | LVS | WQGTHFPYT |
| | (753) | (694) | (695) | (696) | (697) | (698) |
| SCT-Ga157 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga158 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga159 | DYSITNGNHW | ISSSGRI | AREGGLLPPV | QSLLDGDGKTY | LVS | WQGTHFPYT |
| | (847) | (710) | (848) | (845) | (697) | (698) |
| SCT-Ga160 | GYSITNGNHW | ISSSGRI | AREGGWLPPV | QSLLDSDGKTY | LVS | WQGTHFPYT |
| | (693) | (710) | (695) | (696) | (697) | (698) |
| SCT-Ga161 | GYSITNGNHW | ITSSGRT | TREGGWFPPV | QSLLDSDGKTY | LVS | WQGTHFPYT |
| | (693) | (694) | (849) | (696) | (697) | (698) |
| SCT-Ga162 | GYSITNGNHW | ITSSGRT | TREGGWLPPV | QSLLDSDGKTY | LVS | WQGTHFPYT |
| | (693) | (694) | (733) | (696) | (697) | (698) |
| SCT-Ga163 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga164 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga165 | GYSITNGNHW | IRSSGST | AREGGWLPPV | QSLLDSDGKTY | LVS | WQGTHFPYT |
| | (693) | (718) | (695) | (696) | (697) | (698) |
| SCT-Ga166 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga167 | GYTFTTYG | INTYSGVP | ARYDYDVGFAY | QSLVHSNGNTY | KVS | SQSTHVPT |
| | (738) | (739) | (740) | (741) | (745) | (746) |
| SCT-Ga168 | GYSITNGNHW | IRSSGST | AREGGWLPPV | QSLLDSDGKTY | LVS | WQGTHFPYT |
| | (693) | (718) | (695) | (696) | (697) | (698) |
| SCT-Ga169 | GYSITNGNHW | IRSSGST | AREGGWLPPV | QSLLDSDGKTY | LVS | WQGTHFPYT |
| | (693) | (718) | (695) | (696) | (697) | (698) |
| SCT-Ga170 | GYSITNGNHW | IRSSGST | TREGGWLPPV | QSLLDSDGKTY | LVS | WQGTHFPYT |
| | (693) | (718) | (733) | (696) | (697) | (698) |
| SCT-Ga171 | GYSITNGNHW | ITSSGRT | AREGGWLPPV | QSLLDSDGKTY | LVS | WQGTHFPYT |
| | (693) | (694) | (695) | (696) | (697) | (698) |
| SCT-Ga172 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga173 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga174 | GYSITNGNHW | ISSSGRT | AREGGWLPPV | QSLLDSDGKTY | LVS | WQGTHFPYT |
| | (693) | (726) | (695) | (696) | (697) | (698) |
| SCT-Ga175 | GYSFTGYI | IVPYTGGT | ARQLRGYFAMDY | QSLANSYGNTY | GIS | LQGTHQPWT |
| | (850) | (851) | (852) | (853) | (854) | (855) |
| SCT-Ga176 | GFSLTRHG | IWPGGNT | ARSGGYGNFAPYYFDQ | QEISGY | AAS | LQYASYPRT |
| | (856) | (857) | (858) | (859) | (860) | (861) |
| SCT-Ga177 | GYTFTNYG | INTYTGES | ARDSFAY | KNLLHSNGITY | RMS | GQLLELPYT |
| | (862) | (863) | (864) | (865) | (866) | (867) |
| SCT-Ga178 | GYSFTDYN | IDVYYSGT | ARSLLRWYFDV | SSVSSSY | DTS | QQWSSYPLT |
| | (868) | (869) | (870) | (871) | (872) | (843) |
| SCT-Ga179 | GFSLSTSGIG | IWWDDDN | AQSGGITLMDY | QSIVHSDGNTY | KVS | FQSSHVPGT |
| | (873) | (874) | (875) | (876) | (745) | (877) |
| SCT-Ga180 | GYSFTGYT | IVPSNGGT | ARSLRFSFDY | QSLANSYGTTY | GIS | LQGTHQPPT |
| | (878) | (879) | (880) | (881) | (854) | (882) |
| SCT-Ga181 | GYTFTDYS | INTETGVP | ARTDYGNPYWYFDV | SSVSD | ATS | QQWSFNPQT |
| | (883) | (884) | (885) | (886) | (800) | (887) |
| SCT-Ga182 | GYSFTGYT | IIPYNGGT | ARGFRGYFAVDY | QSLTNSYGDTY | GIS | LQGTHQPWT |
| | (878) | (888) | (889) | (890) | (854) | (855) |
| SCT-Ga183 | GYTFTSYW | IDPTNDYS | ARSRGKDWYFDV | QDIGSS | ATS | LQCATSWT |
| | (772) | (891) | (892) | (799) | (800) | (893) |
| SCT-Ga184 | GYTFTSFN | FFPGNGAT | AREGWLSLGY | QNVYTN | SAS | QQHNRYPYT |
| | (894) | (895) | (896) | (897) | (751) | (898) |
| SCT-Ga185 | GYTFSNYW | IFPGGGSA | ARQKYGNYYYFDY | QDISNY | YTS | QQYSKLPYT |
| | (899) | (900) | (901) | (766) | (731) | (902) |
| SCT-Ga186 | GYSFTGYT | FIPYNGGT | ARGLRGFFSMDY | QSLANSYGNTY | GIS | LQGTHQPWT |
| | (878) | (903) | (904) | (853) | (854) | (855) |
| SCT-Ga187 | GYSFTGYT | IIPYNGGT | ARGFRGYFAVDY | QSLTNSYGNTY | GIS | LQGTHQPWT |
| | (878) | (888) | (889) | (905) | (854) | (855) |
| SCT-Ga188 | GFSLTSYG | IWSDGST | ARQIYYDYGWFAY | QNLTNSYGNTY | GIS | LQGTHQPWT |
| | (906) | (907) | (908) | (909) | (854) | (855) |
| SCT-Ga189 | GYSFTDYI | IIPYNGGT | ARGLRGYYAMDY | QSLANSYGSTF | GIS | LQGTHQPWT |
| | (910) | (888) | (911) | (912) | (854) | (855) |
| SCT-Ga190 | GYTFSNYW | ILPGSGSA | ARQKYGNYYYFDY | QGISNY | YTS | QHYSKLPYT |
| | (899) | (913) | (901) | (914) | (731) | (915) |
| SCT-Ga191 | GYTFTDYS | INTETGEP | TRTDYGNPYWYFDV | SSVNY | ATS | QQWSFNPQT |
| | (883) | (916) | (917) | (918) | (800) | (887) |
| SCT-Ga192 | GYSFTGYT | IIPYNGGT | ARGFRGYFAVDY | QSLTNSYGDTY | GIS | LQGTHQPWT |
| | (878) | (888) | (889) | (890) | (854) | (855) |
| SCT-Ga193 | GYSFTGYT | ISPYNGGT | ARSLRFSFDY | QSLANSYGNTY | GIS | LQGTHQPPT |
| | (878) | (919) | (880) | (853) | (854) | (882) |
| SCT-Ga194 | GYSFTGYT | INPYNGGT | ARRNYGYYFDY | QSLANSYGNTY | GIS | LQGTHQPLT |
| | (878) | (707) | (920) | (853) | (854) | (921) |
| SCT-Ga195 | GFDFSRYD | ISSGGSYT | ARQDYRYPYIMDY | QNINVW | KAS | QQGQSYPWT |
| | (922) | (814) | (923) | (790) | (791) | (924) |
| SCT-Ga196 | GFSLSTYGIG | IWWNDNK | ARIGSGKFYFDY | SSVSY | LTS | QQWSSNPPT |
| | (925) | (926) | (927) | (928) | (929) | (930) |
| SCT-Ga197 | GFSLTRHG | IWPGGNT | ARSGGYGNFAPYYFDQ | QSVLYSSNQKNF | WAS | HQYLSSYT |
| | (856) | (857) | (858) | (931) | (724) | (725) |
| SCT-Ga198 | GYTFTNYW | IDPSDSFP | TRGPRGYGDY | QDIKNY | YAT | LHHGESPPT |
| | (932) | (933) | (934) | (935) | (936) | (937) |
| SCT-Ga199 | GYTFTSYW | IFPGSGTS | AREGLYAFDY | QNIRNS | YAS | LQSNSWPYT |
| | (772) | (938) | (939) | (940) | (941) | (942) |
| SCT-Ga200 | GYTFTSYN | IFPGNGFT | TREGWLSLGY | SSVNSTY | RSS | HQWSGYPFT |
| | (943) | (944) | (945) | (946) | (947) | (948) |
| SCT-Ga201 | GYAFTGYY | ITCYNGAT | ARSNYRPWYFDV | QDVSTD | SAS | QQHYSTPYT |
| | (949) | (950) | (951) | (952) | (751) | (758) |
| SCT-Ga202 | GYTFSNYW | IFPGGGSA | ARQKYGNYYYFDY | QDISNY | YTS | QQYSKLPYT |
| | (899) | (900) | (901) | (766) | (731) | (902) |
| SCT-Ga203 | GYTFTSFW | IFPGGGDP | AREGLYAFDY | QSVRNN | YAS | QQSNSWPYT |
| | (953) | (954) | (939) | (955) | (941) | (956) |
| SCT-Ga204 | GYTFSNYW | IFPGSDSS | ARQKYGNFYYFDY | QDISNY | YTS | QQYSKLPYT |
| | (899) | (957) | (958) | (766) | (731) | (902) |
| SCT-Ga205 | GFSLTRHG | IWPGGST | ARSGGYGNFAPYYFDQ | QSVLYSSNQKNY | WAS | HQYLSSYT |
| | (856) | (959) | (858) | (723) | (724) | (725) |
| SCT-Ga206 | GYTFTSYN | IFPGNGFT | TREGWLSLGY | QNVGTN | SAS | QQHNSYPYT |
| | (943) | (944) | (945) | (960) | (751) | (961) |
| SCT-Ga207 | GYTFTSYW | IFPGSGTT | AREGLYAFDY | QSLLNSRTRKNS | WAS | KQSFNLYT |
| | (772) | (962) | (939) | (963) | (724) | (964) |
| SCT-Ga208 | GYTFTSYW | IFPGSGTN | AREGLYAFDY | QSIRNS | YAS | QQSYSWPYT |
| | (772) | (965) | (939) | (966) | (941) | (967) |
| SCT-Ga209 | GYSFTGYT | IFPYNSAT | ARGLRGFFALDY | QSLANSYGNTY | GIS | LQGTHQPWT |
| | (878) | (968) | (969) | (853) | (854) | (855) |
| SCT-Ga210 | GYTFTSYW | IFPGSGTS | AREGLYAFDY | QNIRNS | YAS | LQSNSWPYT |
| | (772) | (938) | (939) | (940) | (941) | (942) |
| SCT-Ga211 | GFSLTRHG | IWPGGNT | ASSGGYGNFAPYYFDQ | QSVLYSSNQKNF | WAS | HQYLSSYT |
| | (856) | (857) | (970) | (931) | (724) | (725) |
| SCT-Ga212 | GFAFSTYD | ISSGGSYT | ARQDYRYPYIMDY | QSISDY | YAS | QDGNTFPPT |
| | (971) | (814) | (923) | (972) | (941) | (973) |
| SCT-Ga213 | GYSFTDYN | IDVYYSGT | ARSSLRWYFDL | SSVSSSY | DTS | QQWSSYPLT |
| | (868) | (869) | (974) | (871) | (872) | (843) |
| SCT-Ga214 | GYTFSNYW | ILPVSGSA | ARQKYGNYYYFDY | QGISNY | YTS | QHYSKLPYT |
| | (899) | (975) | (901) | (914) | (731) | (915) |
| SCT-Ga215 | GYTFSSHN | IFPGNGFT | TREGWLSLGY | QNVVTN | SAS | QQHNSYPYT |
| | (976) | (944) | (945) | (977) | (751) | (961) |
| SCT-Ga216 | GYSFTDYT | IFPYNGAT | ARGLRGFFALDY | QSLANSYGNTY | GIS | LQGTHQPWT |
| | (978) | (979) | (969) | (853) | (854) | (855) |
| SCT-Ga217 | GYSFTGYI | IVPNNGGT | ARSLRFSFDY | QSLANSYGTTY | GIS | LQNTHQPPT |
| | (850) | (980) | (880) | (881) | (854) | (981) |
| SCT-Ga218 | GFSLSTSGIG | IWWDDDT | AQSGGITLMDY | QSIVYSNGNTY | KVS | FQGSHVPFT |
| | (873) | (982) | (875) | (983) | (745) | (984) |
| SCT-Ga219 | GYSFTGYT | IVPYNGGT | ARSLRFSFDY | QSLANSYGTTY | GIS | LQGTHQPPT |
| | (878) | (985) | (880) | (881) | (854) | (882) |
| SCT-Ga220 | GFTFTDYY | IRNKAKGYTT | ARDGGY | DHINNW | GAT | QQYWSTPFT |
| | (986) | (987) | (988) | (989) | (715) | (990) |
| SCT-Ga221 | GFSLSTSGMG | IWWDDVK | ARIAVGQAWFAY | QSVLYSSNQKNY | WAS | HQYLSSLT |
| | (991) | (992) | (993) | (723) | (724) | (816) |
| SCT-Ga222 | GYSFTGYT | IFPYNGGT | ARSLRFSFDY | QSLANSYGTTY | GIS | LQGTHQPPT |
| | (878) | (994) | (880) | (881) | (854) | (882) |
| SCT-Ga223 | GYSFTGYI | IVPNNGGT | ARSLRFSFDY | QSLANSYGTTY | GIS | LQGTHQPPT |
| | (850) | (980) | (880) | (881) | (854) | (882) |
| SCT-Ga224 | GYIFTNYW | IYPGGGYT | ARSRQLASYAMDY | ENIYSY | NAK | QHHYGTPPT |
| | (995) | (996) | (997) | (998) | (776) | (999) |
| SCT-Ga225 | GYSFTGYT | IFPYNSAT | ARGLRGFFALDY | QSLANSYGNTY | GIS | LQGTHQPWT |
| | (878) | (968) | (969) | (853) | (854) | (855) |
| SCT-Ga226 | GFPFNDYY | IRNKANGYTT | ARDGGY | DHINNW | GAT | QQYWSIPFT |
| | (1000) | (1001) | (988) | (989) | (715) | (1002) |
| SCT-Ga227 | GYTFTSYN | IYPGNGYT | AREGGWAMDY | SSVSY | ATS | QQWNSNPPT |
| | (943) | (1003) | (1004) | (928) | (800) | (1005) |
| SCT-Ga228 | GYSFTGYT | INPYNGGT | ARRNYGYYFDY | QSLANSYGDTF | GIS | LQGTHQPLT |
| | (878) | (707) | (920) | (1006) | (854) | (921) |
| SCT-Ga229 | GFSLSTSGIG | IWWDDDN | AQSGGITLMDY | QSIVHSDGNTY | KVS | FQSSHVPGT |
| | (873) | (874) | (875) | (876) | (745) | (877) |
| SCT-Ga230 | GFTFTDYY | IRNKAHGYTT | ARDGGY | DHINNW | GAT | QQYWSTPFT |
| | (986) | (1007) | (988) | (989) | (715) | (990) |
| SCT-Ga231 | DYSFTSYW | IYPGNSAA | SREGGYGLDY | QSIYNN | YAS | QQSNSWPYT |
| | (1008) | (1009) | (1010) | (1011) | (941) | (956) |
| SCT-Ga232 | GYSFTDYN | IDVYYSGT | ARSSLRWYFDL | SSVSSSN | DTS | QQWSSYPLT |
| | (868) | (869) | (974) | (1012) | (872) | (843) |
| SCT-Ga233 | GFSLTRHG | IWPGGNT | ARSGGYGNFAPYYFDQ | QNILYSSNQKNY | WAS | HQYLSSYT |
| | (856) | (857) | (858) | (1013) | (724) | (725) |
| SCT-Ga234 | GYTFSNYW | ILPVSGSS | ARQKYGNYYYFDY | QGISNY | YTS | QHYSKLPYT |
| | (899) | (1014) | (901) | (914) | (731) | (915) |
| SCT-Ga235 | GYTFSNYW | ILPGSGSS | ARQKYGNYYYFDF | QGLSNY | YTS | QQYSKLPYT |
| | (899) | (1015) | (1016) | (1017) | (731) | (902) |
| SCT-Ga236 | GYTFTSYW | IFPGSGTT | AREGLYAFDY | QSIRNS | YAS | QQSNSWPYT |
| | (772) | (962) | (939) | (966) | (941) | (956) |
| SCT-Ga237 | GFTFSTYA | ISSGGTYT | ARHFISTIITHFDY | QSVLYSSNQMNY | WAS | HQYLSSLT |
| | (1018) | (1019) | (1020) | (1021) | (724) | (816) |
| SCT-Ga238 | GYSITSYW | VYPGNSNT | TREGGYGMDY | QTISNN | YSS | QQSYSWPYT |
| | (1022) | (1023) | (1024) | (1025) | (1026) | (967) |
| SCT-Ga239 | GYTFTSYN | IFPGNGFT | TREGWLSLGY | QNVVTN | SAS | QQHNSYPYT |
| | (943) | (944) | (945) | (977) | (751) | (961) |
| SCT-Ga240 | GYTFTSYW | IFPGSGTT | AREGLYAFDY | QNIRNS | YAS | QQSNSWPYT |
| | (772) | (962) | (939) | (940) | (941) | (956) |
| SCT-Ga241 | GYTFTDYS | INTETGEP | TRTNYGNPYWYFDV | SSVNY | ATS | QQWNFNPQT |
| | (883) | (916) | (1027) | (918) | (800) | (1028) |
| SCT-Ga242 | GFTFNDYY | IRNKANGYTT | ARDGGY | DHINNW | GAT | QQYWSIPFT |
| | (1029) | (1001) | (988) | (989) | (715) | (1002) |
| SCT-Ga243 | GYSFIDYI | IIPYNGGT | ARGLRGYYAMDY | QSLTNSYGSTF | GIS | LQGTHQPWT |
| | (1030) | (888) | (911) | (1031) | (854) | (855) |
| SCT-Ga244 | GYTFTSYW | IFPGTGSA | TREGLYPFDY | QNIRNN | YAS | QQSNSWPYT |
| | (772) | (1032) | (1033) | (1034) | (941) | (956) |
| SCT-Ga245 | GFSLTSYG | IWSGGST | AREVRRYFDV | SSVISSY | STS | HQYHRSPLT |
| | (906) | (1035) | (1036) | (1037) | (1038) | (1039) |
| SCT-Ga246 | GFSLSTSGIG | IWWDDDN | AQSGGITLMDY | QSIVHSDGNTY | KVS | FQGSHVPGT |
| | (873) | (874) | (875) | (876) | (745) | (1040) |
| SCT-Ga247 | GYSFTGYT | IIPYNGGS | ARGLRGYFAMDY | QNLTNSYGNTY | GIS | LQGTHQPWT |
| | (878) | (1041) | (1042) | (909) | (854) | (855) |
| SCT-Ga248 | GYTFTSYN | IYPGNGYT | AREGGWAMDY | SSVNY | DTS | QQWNSNPPT |
| | (943) | (1003) | (1004) | (918) | (872) | (1005) |
| SCT-Ga249 | GYTFSNYW | ILPGSGSS | ARQKYGNFYYFDF | QGIGNY | YTS | HQYSKLPYT |
| | (899) | (1015) | (1043) | (1044) | (731) | (1045) |
| SCT-Ga250 | GYTFTNYG | ISTYIGEP | ARDSFAH | KNLLHSNGITY | RVS | GQLLELPYT |
| | (862) | (1046) | (1047) | (865) | (1048) | (867) |
| SCT-Ga251 | GYTFTSYN | IYPGNGYT | AREGGWAMDY | SSVSS | DTS | QQWSSNPST |
| | (943) | (1003) | (1004) | (1049) | (872) | (1050) |
| SCT-Ga252 | GYTFTSFW | IYPGSGTT | TREGLYAFDQ | QSIRSS | YAS | QQSNTWPYT |
| | (953) | (1051) | (1052) | (1053) | (941) | (1054) |
| SCT-Ga253 | GYTFTSYW | IFPGSGTT | AREGLYAFDY | QSIRNS | YAS | QQSNSWPYT |
| | (772) | (962) | (939) | (966) | (941) | (956) |
| SCT-Ga254 | GYTFTIHW | IYPGGGYT | ARDYGYDVYAMDY | QSLLDSDGKTY | LVS | WQGTHFPRT |
| | (1055) | (996) | (1056) | (696) | (697) | (1057) |
| SCT-Ga255 | GFAFSSFD | ISSGGSYT | TRQDYRYPYIMDY | QNINVW | KAS | QQGQSYPWT |
| | (1058) | (814) | (1059) | (790) | (791) | (924) |
| SCT-Ga256 | GYIFTNFW | IYPGGGYT | ARSRQLASYAMDY | ENIYSY | NAK | QHHYGTPPT |
| | (1060) | (996) | (997) | (998) | (776) | (999) |
| SCT-Ga257 | GFSLSTSGIG | IWWDDDT | AQSGGITLMDY | QNIVHSNGNTY | KVS | FQTSHVPGT |
| | (873) | (982) | (875) | (1061) | (745) | (1062) |
| SCT-Ga258 | GFTFTDYY | IRNKAHGYTT | ARDGGY | DHINNW | GAT | QQYWSTPFT |
| | (986) | (1007) | (988) | (989) | (715) | (990) |
| SCT-Ga259 | GFTFNDYY | IRNKANGYTT | ARDGGY | DHINNW | GAT | QQYWSIPFT |
| | (1029) | (1001) | (988) | (989) | (715) | (1002) |
| SCT-Ga260 | GFTFNDYY | IRDKANGYTT | ARDGGY | DHINNW | GAT | QQYWSIPFT |
| | (1029) | (1063) | (988) | (989) | (715) | (1002) |
| SCT-Ga261 | GYSFSDYN | IDVYYLST | ARSLLRWYFDV | SSVTY | DTS | QHWSSYPLT |
| | (1064) | (1065) | (870) | (1066) | (872) | (1067) |
| SCT-Ga262 | GYSFTGYT | IIPYNGGT | ARSLRFSFDY | QSLANSYGNTY | GIS | LQGTHQPPT |
| | (878) | (888) | (880) | (853) | (854) | (882) |
| SCT-Ga263 | GYTFTSYW | IFPGSGTT | TREGLYSFDY | QSIRNN | YVS | QQSISWPYT |
| | (772) | (962) | (1068) | (1069) | (1070) | (1071) |
| SCT-Ga264 | GYTFTSYW | IFPGSGIT | AREGLYAFDY | QSIRNS | YAS | QQTNSWPYT |
| | (772) | (1072) | (939) | (966) | (941) | (1073) |
| SCT-Ga265 | GYTFTSYN | IFPGNGFT | TREGWLSLGY | QNVVTN | SAS | QQHNSYPYT |
| | (943) | (944) | (945) | (977) | (751) | (961) |
| SCT-Ga266 | GYSFTGYT | IVPYNGGT | ARSLRFSFDY | QSLANSYGTTY | GIS | LQGTHQPPT |
| | (878) | (985) | (880) | (881) | (854) | (882) |
| SCT-Ga267 | GYSFTGYT | IVPNNGGT | ARSLRFSFDY | QSLANSYGTTY | GIS | LQGTHQPPT |
| | (878) | (980) | (880) | (881) | (854) | (882) |
| SCT-Ga268 | GYIFTNYG | INSYTGES | ARDSFAY | KNLLHSNGITY | RMS | GQLLELPYT |
| | (1074) | (1075) | (864) | (865) | (866) | (867) |
| SCT-Ga269 | GYTFTSYN | IFPGNGFT | TREGWLSLGY | QNVVTN | SAS | QQHNSYPYT |
| | (943) | (944) | (945) | (977) | (751) | (961) |
| SCT-Ga270 | GYTFTTFW | IFPGRGIT | SRGLGYFDV | QSIGTS | YGS | QQSYRWPYT |
| | (1076) | (1077) | (1078) | (1079) | (1080) | (1081) |
| SCT-Ga271 | GFTFNDYY | IRDKANGYTT | ARDGGY | DHINNW | GAT | QQYWSIPFT |
| | (1029) | (1063) | (988) | (989) | (715) | (1002) |
| SCT-Ga272 | GYTFTSYW | IFPGTGST | TREGLYPFDY | QNIRNN | YAS | QQSNSWPYT |
| | (772) | (1082) | (1033) | (1034) | (941) | (956) |
| SCT-Ga273 | GYSFTGYT | IIPYNGGT | ARGFRGYFAVDY | QSLTNSYGNTF | GIS | LQGTHQPWT |
| | (878) | (888) | (889) | (1083) | (854) | (855) |
| SCT-Ga274 | GYSFTDYN | IDVYYSGT | ARSLLRWYFDV | SSVSSSY | DTS | QQWSSYPLT |
| | (868) | (869) | (870) | (871) | (872) | (843) |
| SCT-Ga275 | GFTFSTYA | ISSGGSYT | ARHFISTIITHFDY | QSVLYSSNQMNY | WAS | HQYLSSLT |
| | (1018) | (814) | (1020) | (1021) | (724) | (816) |
| SCT-Ga276 | GFTFNDYY | IRNKANGYTT | ARDGGY | DHINNW | GAT | QQYWSIPFT |
| | (1029) | (1001) | (988) | (989) | (715) | (1002) |
| SCT-Ga277 | GYTFTSYY | IYPGDGST | TRDGNYGDWFPY | QNIVHSNGNTY | KVS | FQGSHVPYT |
| | (1084) | (1085) | (1086) | (1061) | (745) | (1087) |
| SCT-Ga278 | GYTFSNYW | ILPVSDSA | ARQKYGNYYYFDY | QGISSY | YTS | QHYSKLPYT |
| | (899) | (1088) | (901) | (1089) | (731) | (915) |
| SCT-Ga279 | GYTFTSYW | IFPGSGTS | AREGLYAFDY | ESVDKYGISF | AAS | QQSKEVPWT |
| | (772) | (938) | (939) | (1090) | (860) | (1091) |
| SCT-Ga280 | GYSFTGYT | IVPYNGGT | ARSVRFSFDY | QSLANSYGTTY | EIS | LQGTHQPPT |
| | (878) | (985) | (1092) | (881) | (1093) | (882) |
| SCT-Ga281 | GYTFTNYG | INTYTGES | ARDSFAY | KNLLHSNGITY | RMS | GQLLELPYT |
| | (862) | (863) | (864) | (865) | (866) | (867) |
| SCT-Ga282 | GYSFTGYY | VNPNNGGT | ASSPLLRLRGYAMDD | ESVDSYGKSF | VAS | QQNNEDPWT |
| | (1094) | (1095) | (1096) | (1097) | (1098) | (1099) |
| SCT-Ga283 | GYTFTSFW | IYPGRGIT | SRGLGYFDV | QSIGTS | YGS | QQSYRWPYT |
| | (953) | (1100) | (1078) | (1079) | (1080) | (1081) |
| SCT-Ga284 | GYTFTSYW | IFPGSGTT | SREGLYPFDY | QSIRNN | YAS | QQSNSWPYT |
| | (772) | (962) | (1101) | (1069) | (941) | (956) |
| SCT-Ga285 | GYTFTSYN | IFPGNGFT | TREGWLSLGY | QHVVTN | SAS | QQHNSYPYT |
| | (943) | (944) | (945) | (1102) | (751) | (961) |
| SCT-Ga286 | GFSLTSYD | IWTGGGT | VRSTMITHYAMDY | QNIGTS | YAF | QQSNSWPLT |
| | (1103) | (721) | (1104) | (1105) | (1106) | (1107) |
| SCT-Ga287 | GFTFNDYY | IRNKANGYTT | ARDGGY | DHINNW | GAT | QQYWSIPFT |
| | (1029) | (1001) | (988) | (989) | (715) | (1002) |
| SCT-Ga288 | GFSLSTSGIG | IWWDDDN | AQSGGITLMDY | DHINNW | GAT | QQYWSIPFT |
| | (873) | (874) | (875) | (989) | (715) | (1002) |
| SCT-Ga289 | GYTFTSYW | IYPGSGTT | TREGLYPFDY | QSIRNS | YVF | QQSNSWPYT |
| | (772) | (1051) | (1033) | (966) | (1108) | (956) |
| SCT-Ga290 | GYTFTSYN | IYPGNGYT | AREGGWAMDY | SSVSS | DTS | QQWSSNPST |
| | (943) | (1003) | (1004) | (1049) | (872) | (1050) |
| SCT-Ga291 | GYTFTSYW | IFPGSGIT | AREGLYAFDY | QSIRNS | YAS | QQTNSWPYT |
| | (772) | (1072) | (939) | (966) | (941) | (1073) |
| SCT-Ga292 | GFSFTDYN | VDIYYSGT | ARSSLRWYFDL | SSVSSSN | DTS | QQWSSYPLT |
| | (1109) | (1110) | (974) | (1012) | (872) | (843) |
| SCT-Ga293 | GYTFSNYW | IDPSDSYT | TRPSSYFFDY | QSIRNN | YAS | QQSYSWPYT |
| | (899) | (1111) | (1112) | (1069) | (941) | (967) |
| SCT-Ga294 | GYTFTSYN | IYPGNDGT | AREGWLSLDY | QNVVTN | SAS | QQYKSYPYT |
| | (943) | (1113) | (1114) | (977) | (751) | (1115) |
| SCT-Ga295 | GYSFTGNI | INPYNGGT | ARGFRGYFAMDY | QSLANSYGNTY | GIS | LQGTHQPWT |
| | (1116) | (707) | (1117) | (853) | (854) | (855) |
| SCT-Ga296 | GFSLSTSGIG | IWWDDDT | AQSGGITLMDY | QNIVHSNGNTY | KVS | FQTSHVPGT |
| | (873) | (982) | (875) | (1061) | (745) | (1062) |
| SCT-Ga297 | GYTFTDYN | IYPYNVGT | ARKNPLDY | KNLLHSNGITY | RVS | AQLLELPYT |
| | (1118) | (1119) | (1120) | (865) | (1048) | (1121) |
| SCT-Ga298 | GYTFSNCW | ILPGSGSA | ARQKYGNYYYFDY | QGISNY | YTS | QHYSKLPYT |
| | (1122) | (913) | (901) | (914) | (731) | (915) |
| SCT-Ga299 | GYSFTSYW | FYPGNSAT | SREGGYGMDH | QSIRNN | YSS | QQSNSWPYT |
| | (1123) | (1124) | (1125) | (1069) | (1026) | (956) |
| SCT-Ga300 | GYTFTSYY | IYPGDGST | TRDGNYGDWFPY | QSVSTSRYSY | YAS | QHSWEIPLT |
| | (1084) | (1085) | (1086) | (1126) | (941) | (1127) |
| SCT-Ga301 | GFAFSSFD | ISSGGSYT | ARQDYRYPYIMDY | QNINVW | KAS | QQGQSYPWT |
| | (1058) | (814) | (923) | (790) | (791) | (924) |
| SCT-Ga302 | GYSFTSYW | FYPGNSAT | SREGGYGMDH | QSIRNN | YAS | QQSNSWPYT |
| | (1123) | (1124) | (1125) | (1069) | (941) | (956) |
| SCT-Ga303 | GYSFTGYT | IIPYNGGT | ARSLRFSFDY | QSLANSYGNTY | GIS | LQGTHQPPT |
| | (878) | (888) | (880) | (853) | (854) | (882) |
| SCT-Ga304 | GYTFSNYN | IFPGNGVT | ARGGFGGALDY | SSVSH | DTS | QQWNNNPLT |
| | (1128) | (1129) | (1130) | (1131) | (872) | (1132) |
| SCT-Ga305 | GYSITSYW | VYPGNSNT | TREGGYGMDY | QTISNN | YSS | QQSYSWPYT |
| | (1022) | (1023) | (1024) | (1025) | (1026) | (967) |
| SCT-Ga306 | GYSFTGYT | IFPYNSAT | ARGLRGFFALDY | QSLANSYGNTY | GIS | LQGTHQPWT |
| | (878) | (968) | (969) | (853) | (854) | (855) |
| SCT-Ga307 | GYTFSNYN | IFPGNGVT | ARGGFGGAMDY | SSVSH | DTS | QQWNNNPLT |
| | (1128) | (1129) | (1133) | (1131) | (872) | (1132) |
| SCT-Ga308 | GFSLSTSGIG | IWWDDDN | AQSGGITLMDY | QSIVHSDGNTY | KVS | FQGSHVPGT |
| | (873) | (874) | (875) | (876) | (745) | (1040) |
| SCT-Ga309 | GFSLSTSGIG | IWWDDDN | AQSGGITLMDY | QSIVHSDGNTY | KVS | FQGSHVPGT |
| | (873) | (874) | (875) | (876) | (745) | (1040) |
| SCT-Ga310 | GYTFTNFG | INTYTGEP | ARDSFAH | KNLQHNNGITY | RVS | GQLLELPYT |
| | (1134) | (1135) | (1047) | (1136) | (1048) | (867) |
| SCT-Ga311 | GYTFTSYN | IYPGNGFT | AREGGWAMDY | SSVSH | DTS | QQWNSNPST |
| | (943) | (1137) | (1004) | (1131) | (872) | (1138) |
| SCT-Ga312 | GFPFNDYY | IRNKANGYTT | ARDGGY | DHIHNW | GAT | QQYWSIPFT |
| | (1000) | (1001) | (988) | (1139) | (715) | (1002) |
| SCT-Ga313 | GYSFTGYT | IIPYNGGT | ARSLRFSFDY | QSLANSYGNTY | GIS | LQGTHQPPT |
| | (878) | (888) | (880) | (853) | (854) | (882) |
| SCT-Ga314 | GYTFTSYW | IFPGSGTT | TREGLYAFDY | QSIRNS | YAS | QQSNSWPYT |
| | (772) | (962) | (1140) | (966) | (941) | (956) |
| SCT-Ga315 | GFTFTDYY | IRNKANGYTT | ARDGGY | DHINNW | GAT | QQYWSTPFT |
| | (986) | (1001) | (988) | (989) | (715) | (990) |
| SCT-Ga316 | GYTFTSYW | IYPGGGTT | TREGLYPFDY | QSIRNN | YVS | QQSNSWPYT |
| | (772) | (1141) | (1033) | (1069) | (1070) | (956) |
| SCT-Ga317 | GYSFTSYW | IYPGNSDT | TREGGYGMDY | QSIYNN | FSS | QQSNSWPYT |
| | (1123) | (1142) | (1024) | (1011) | (1143) | (956) |
| SCT-Ga318 | GYTFTSYW | IFPGSGTS | AREGLYAFDY | QNIRNS | YAS | LQSNSWPYT |
| | (772) | (938) | (939) | (940) | (941) | (942) |
| SCT-Ga319 | GYSFTGYT | INPSNGGT | TRRNYGYYFDY | QSLANSYGNTY | GIS | LQGTHQPLT |
| | (878) | (788) | (1144) | (853) | (854) | (921) |
| SCT-Ga320 | GFTFTDYY | IRNKANGYTT | ARDGGY | DHINNW | GAT | QQYWSIPFT |
| | (986) | (1001) | (988) | (989) | (715) | (1002) |
| SCT-Ga321 | GFSLSTSGIG | IWWDDDN | AQSGGITLMDY | QSIVHSDGNTY | KVS | FQGSHVPGT |
| | (873) | (874) | (875) | (876) | (745) | (1040) |
| SCT-Ga322 | GYTFTSYW | IFPGSGIT | AREGLYAFDY | QSIRNS | YAS | QQSNSWPYT |
| | (772) | (1072) | (939) | (966) | (941) | (956) |
| SCT-Ga323 | GFPFNDYY | IRNKAKGYTT | ARDGGY | DHINNW | GAT | QQYWSIPFT |
| | (1000) | (987) | (988) | (989) | (715) | (1002) |
| SCT-Ga324 | GFPFNDYY | IRNKANGYTT | ARDGGY | DHINNW | GAT | QQYWSIPFT |
| | (1000) | (1001) | (988) | (989) | (715) | (1002) |
| SCT-Ga325 | GYTFTSFW | IYPGRGIA | SRGLGYFDV | QNIGTS | SAS | QQSNRWPYT |
| | (953) | (1145) | (1078) | (1105) | (751) | (1146) |
| SCT-Ga326 | GYTFTSYN | IYPGNGYT | AREGGWAMDY | SSVSQ | DTS | QQWSSNPST |
| | (943) | (1003) | (1004) | (1147) | (872) | (1050) |
| SCT-Ga327 | GFSLSTSGIG | IWWDDDN | AQSGGITLMDY | QSIVHSDGNTY | KVS | FQASHVPGT |
| | (873) | (874) | (875) | (876) | (745) | (1148) |
| SCT-Ga328 | GYTFTNFW | IFPGGGYT | SRSTMLTGTY | RDIKSY | YAT | LHHGESPYT |
| | (1149) | (1150) | (1151) | (1152) | (936) | (1153) |
| SCT-Ga329 | GFTFNDYY | IRNKANGYTT | ARDGGY | DHINNW | GAT | QQYWSTPFT |
| | (1029) | (1001) | (988) | (989) | (715) | (990) |
| SCT-Ga330 | GYTFTNFW | IFPGGGHT | SRSTILTGAY | QDIKSY | YAT | LHHGESPYT |
| | (1149) | (1154) | (1155) | (1156) | (936) | (1153) |
| SCT-Ga331 | GFTFTDYY | IRNKANGYTT | ARDGGY | DHINNW | GAT | QQYWSTPFT |
| | (986) | (1001) | (988) | (989) | (715) | (990) |
| SCT-Ga332 | GFTFNDYY | IRNKANGYTT | ARDGGY | DHINNW | GAT | QQYWSIPFT |
| | (1029) | (1001) | (988) | (989) | (715) | (1002) |
| SCT-Ga333 | GYAFTNYL | INPGSGGN | ARRGDKYGSAWFAY | QDVSTA | SAS | QQHYSSPRT |
| | (687) | (1157) | (1158) | (757) | (751) | (752) |
| SCT-Ga334 | GFTFNDYY | IRDKANGYTT | ARDGGY | DHINNW | GAT | QQYWSIPFT |
| | (1029) | (1063) | (988) | (989) | (715) | (1002) |
| SCT-Ga335 | GYSFTDYN | IDVYYSGT | ARSSLRWYFDL | SSVNSTY | DTS | QQWSSYPLT |
| | (868) | (869) | (974) | (946) | (872) | (843) |
| SCT-Ga336 | GYSFTGYT | IVPYNGGT | ARSLRFSFDY | QSLANSYGTTY | GIS | LQGTHQPPT |
| | (878) | (985) | (880) | (881) | (854) | (882) |
| SCT-Ga337 | GYSFTDYN | IDVYYSST | ARSLLRWYFDV | SSVSSSY | DTS | QQWSSYPLT |
| | (868) | (1159) | (870) | (871) | (872) | (843) |
| SCT-Ga338 | GYTFTSYW | IYPGSGTT | TREGLYPFDY | QSIRNN | YAS | QQSNSWPYT |
| | (772) | (1051) | (1033) | (1069) | (941) | (956) |
| SCT-Ga339 | GYTFTDYN | INSNNGGA | ARGDYYGTTYDY | DHINNW | GAT | QQYWSIPFT |
| | (1118) | (1160) | (1161) | (989) | (715) | (1002) |
| SCT-Ga340 | GYSFTGYI | IHPYNGGT | ARLLRYAMDY | SSVSY | RTS | QQYHGYPPT |
| | (850) | (1162) | (1163) | (928) | (1164) | (1165) |
| SCT-Ga341 | GYTFISYW | IYPGDGDT | ARGTVGDY | QNVVTN | SAS | QQHNSYPYT |
| | (1166) | (1167) | (1168) | (977) | (751) | (961) |
| SCT-Ga342 | GYTFTNYG | INTYTGES | ARDSFAY | KNLLHSNGITY | RMS | GQLLELPYT |
| | (862) | (863) | (864) | (865) | (866) | (867) |
| SCT-Ga343 | GYSFTDYT | IIPYNGGT | ARGLRGYFAMDY | QSLANSYGVTY | GIS | LQGTHQPWT |
| | (978) | (888) | (1042) | (1169) | (854) | (855) |
| SCT-Ga344 | GYSFTGYT | IFPYNGGT | ARSVRFSFDY | QSLANSYGNTY | GIS | LQGTHQPPT |
| | (878) | (994) | (1092) | (853) | (854) | (882) |
| SCT-Ga345 | GFAFSRYD | ISSGGSYS | ARQDYRYPYIMDY | QNINVW | KTS | QQGQSYPWT |
| | (1170) | (1171) | (923) | (790) | (1172) | (924) |
| SCT-Ga346 | GYSFTGYT | INPYNGGT | ARFRLWQGMDY | QSLANSYGNTY | GIS | LQGTHQPPT |
| | (878) | (707) | (1173) | (853) | (854) | (882) |
| SCT-Ga347 | GYTFTSYW | IFPGSDTT | TREGLYAFDY | PNIRNS | YAS | QQSNSWPYT |
| | (772) | (1174) | (1140) | (1175) | (941) | (956) |
| SCT-Ga348 | GYTFTSYW | IFPGSGTT | AREGLYAFDY | QSIGTS | FAS | QQSNSWPYT |
| | (772) | (962) | (939) | (1079) | (1176) | (956) |
| SCT-Ga349 | GYTFTSYW | IFPHSGIT | AREGLYAFDY | QSIRNS | YVS | QQTNSWPYT |
| | (772) | (1177) | (939) | (966) | (1070) | (1073) |
| SCT-Ga350 | GFTFSTYA | ISSGGTYT | ARHFISTIITHFDY | QSVLYSSNQMNY | WAS | HQYLSSLT |
| | (1018) | (1019) | (1020) | (1021) | (724) | (816) |
| SCT-Ga351 | GYTFTSYN | IFPGNGFT | TREGWLSLGY | QNVVTN | SAS | QQHNSYPYT |
| | (943) | (944) | (945) | (977) | (751) | (961) |
| SCT-Ga352 | GYSFTSYW | IYPGNNAT | SREGGFGMDY | QSITNN | YAS | QQSNSWPYT |
| | (1123) | (1178) | (1179) | (1180) | (941) | (956) |
| SCT-Ga353 | GYSFTDYT | IFPYNGAT | ARGLRGFFALDY | QSLANSYGNTY | GIS | LQGTHQPWT |
| | (978) | (979) | (969) | (853) | (854) | (855) |
| SCT-Ga354 | GYSFTDYI | IIPYNGGT | ARGLRGYYTMDY | QSLANIYGNTY | GIS | LQGTHQPWT |
| | (910) | (888) | (1181) | (1182) | (854) | (855) |
| SCT-Ga355 | GYTFTTFW | IFPGRGIT | SRGLGYFDV | QSVGTS | YGF | QQSYRWPYT |
| | (1076) | (1077) | (1078) | (1183) | (1184) | (1081) |
| SCT-Ga356 | GYTFSNYW | ILPGSGSA | ARQKYGNYYYFDY | QGISNY | YTS | QHYSKLPYT |
| | (899) | (913) | (901) | (914) | (731) | (915) |
| SCT-Ga357 | GYTFTSYW | IYPGSGTT | TREGLYPFDY | QSIRNN | YVF | QQSNSWPYT |
| | (772) | (1051) | (1033) | (1069) | (1108) | (956) |
| SCT-Ga358 | GYSFTDYN | IDVYYSGT | ARSSLRWYFDL | SSVSSSD | DTS | QQWSSYPLT |
| | (868) | (869) | (974) | (1185) | (872) | (843) |
| SCT-Ga359 | GFSLSTSGIG | IWWDDDS | AQSGGITLMDY | QSIVHSNGNTY | KVS | FQGSHVPGT |
| | (873) | (1186) | (875) | (1187) | (745) | (1040) |
| SCT-Ga360 | GYSFTGYT | IFPYNGAT | ARGLRGYFAMDY | QSLANSYGNTY | GIS | LQGTHQPWT |
| | (878) | (979) | (1042) | (853) | (854) | (855) |
| SCT-Ga361 | GYTFTSYW | IFPGSGTT | TREGLYPFDY | QNIRNN | YAS | QQSNSWPYT |
| | (772) | (962) | (1033) | (1034) | (941) | (956) |
| SCT-Ga362 | GYTFTSYW | IFPGTGSA | TREGLSPFDY | QNIRNN | YAS | QQSNSWPYT |
| | (772) | (1032) | (1188) | (1034) | (941) | (956) |
| SCT-Ga363 | GYSFTDYN | IDVYYSGT | ARSSLRWYFDL | SSVSSRY | DTS | QQWSSYPLT |
| | (868) | (869) | (974) | (1189) | (872) | (843) |
| SCT-Ga364 | GYTFTSYS | IYPGNVIT | ARDGGFGTY | QNVHTN | SAS | QQYNSYPYT |
| | (1190) | (1191) | (1192) | (1193) | (751) | (1194) |
| SCT-Ga365 | GYTFTSYN | IYPGNGYT | AREGGWAMDY | SSVSY | ATS | QQWNSNPPT |
| | (943) | (1003) | (1004) | (928) | (800) | (1005) |
| SCT-Ga366 | GYSFTGYT | IFPYNGAT | TRGLRGYFALDY | QSLANSYGNTY | GIS | LQGTHLPWT |
| | (878) | (979) | (1195) | (853) | (854) | (1196) |
| SCT-Ga367 | GYSFTGYT | IFPYNSAT | ARGLRGFFALDY | QSLANSYGNTY | GIS | LQGTHQPWT |
| | (878) | (968) | (969) | (853) | (854) | (855) |
| SCT-Ga368 | GYTFTSYN | IYPGNGGT | ARDGFYPFDY | QNVGTN | SAS | QQYNSYPLT |
| | (943) | (1197) | (1198) | (960) | (751) | (1199) |
| SCT-Ga369 | GYTFTSYN | IYPGNGYT | AREGGWAMDY | SSVSS | DTS | QQWSSNPST |
| | (943) | (1003) | (1004) | (1049) | (872) | (1050) |
| SCT-Ga370 | GYTFTNYG | INTYTGES | ARDSFAH | KNLLHSNGITY | RMS | GQLLELPYT |
| | (862) | (863) | (1047) | (865) | (866) | (867) |
| SCT-Ga371 | GYSFTGYT | IFPYNSAT | ARGLRGFFALDY | QSLANSYGNTY | GIS | LQGIHQPWT |
| | (878) | (968) | (969) | (853) | (854) | (1200) |
| SCT-Ga372 | GYSITSYW | IYPGNSNT | TREGGYGMDY | QTISNN | YSS | QQSYSWPYT |
| | (1022) | (1201) | (1024) | (1025) | (1026) | (967) |
| SCT-Ga373 | GFAFSTYD | ISSGGSYT | ARQDYRYPYIMDY | QNINVW | KAS | QQGQSYPWT |
| | (971) | (814) | (923) | (790) | (791) | (924) |
| SCT-Ga374 | GYTFSNYW | ILPVSDSA | ARQKYGTYYYFDY | QGISNY | YTS | QHYSKLPYT |
| | (899) | (1088) | (1202) | (914) | (731) | (915) |
| SCT-Ga375 | GFSLSTSGIG | IWWDDDN | AQSGGITLMDY | QSIVHSDGNTY | KVS | FQGSHVPGT |
| | (873) | (874) | (875) | (876) | (745) | (1040) |
| SCT-Ga376 | GYTFTNFW | IFPGGDYT | SRSTIISGAY | QDIKSY | YAT | LHHGERPYT |
| | (1149) | (1203) | (1204) | (1156) | (936) | (1205) |
| SCT-Ga377 | GYTFTSYW | IYPGSGTT | TREGLYAFDY | PNIRNS | YAS | QQSNSWPYT |
| | (772) | (1051) | (1140) | (1175) | (941) | (956) |
| SCT-Ga378 | GYSFTGYI | INPYNGGT | TRLLRFAMDY | QSLANSYGSTY | GIS | LQGTHQPPT |
| | (850) | (707) | (1206) | (1207) | (854) | (882) |
| SCT-Ga379 | GFVFSSYD | ISSGGSYT | ARQDYRYPYTMDY | QNINVW | KAS | QQGQSYPWT |
| | (1208) | (814) | (1209) | (790) | (791) | (924) |
| SCT-Ga380 | GFPFNDYY | IRNKANGYTT | ARDGGY | DHINNW | GAT | QQYWSIPFT |
| | (1000) | (1001) | (988) | (989) | (715) | (1002) |
| SCT-Ga381 | GYSFTRYW | FYPGNSAT | SREGGYGMDH | QSIRNN | YVS | QQSNSWPYT |
| | (1210) | (1124) | (1125) | (1069) | (1070) | (956) |
| SCT-Ga382 | GYTFTDYA | IGTSNGNT | TRDYYGRSYLYFDY | SSVSSSY | STS | QQYSGYPLT |
| | (1211) | (1212) | (1213) | (871) | (1038) | (1214) |
| SCT-Ga383 | GYSFTGYT | IFPYNGGT | ARSRSFAMDY | QSLTNSYGDTY | GIS | LQGTHQPWT |
| | (878) | (994) | (1215) | (890) | (854) | (855) |
| SCT-Ga384 | GFTFSDYY | IRNKANGYTT | ARDGGY | DHINNW | GAT | QQYWSIPFT |
| | (1216) | (1001) | (988) | (989) | (715) | (1002) |
| SCT-Ga385 | GYTFTDHS | INTETGEP | ASPRNYDGAMDY | DHINNW | GAT | QQYWSTPLT |
| | (1217) | (916) | (1218) | (989) | (715) | (1219) |
| SCT-Ga386 | GFTFTSYY | IRDKANGYTT | ARDGGY | EHIYGA | GAT | QNVLSTPLT |
| | (1220) | (1063) | (988) | (1221) | (715) | (1222) |
| SCT-Ga387 | GYAFSSSW | IYPGDGDT | ASNYYYFDY | KSVSTSGYSY | LAS | QHSRELPWT |
| | (1223) | (1167) | (1224) | (1225) | (1226) | (1227) |
| SCT-Ga388 | GYTFTNYW | TNPSTGSS | AAYYRYGDGAY | DHINNW | GAS | QQYWSTPWT |
| | (932) | (1228) | (1229) | (989) | (691) | (716) |
| SCT-Ga389 | GFTFSTYV | ISSGGSYI | ARWLLGLYAMDY | QNINVW | KAS | QQGQSYPRT |
| | (1230) | (1231) | (1232) | (790) | (791) | (1233) |
| SCT-Ga390 | GFTFSDYY | IRNKANGYTT | ARDGGY | DHINNW | GAT | QQYWSTPFT |
| | (1216) | (1001) | (988) | (989) | (715) | (990) |
| SCT-Ga391 | GYAFSSYW | IYPGDGDS | ARRKDYGYDY | DHINNW | GAT | QQYWSTPFT |
| | (1234) | (1235) | (1236) | (989) | (715) | (990) |
| SCT-Ga392 | GFTFSEFY | SRNKANDYST | ARDAYGYPWFVY | QSLLYSGNQKNY | WAS | QQYYSYPWT |
| | (1237) | (1238) | (1239) | (1240) | (724) | (1241) |
| SCT-Ga393 | GFTFSNFG | ISSGSSTI | ARGRYDYLYAMDY | QDISNY | YTS | QQGNTLPFT |
| | (1242) | (1243) | (1244) | (766) | (731) | (767) |
| SCT-Ga394 | GFTFTDYY | ITNKANGYTT | ARDGGY | DHINNW | GAT | QQYWSIPFT |
| | (986) | (1245) | (988) | (989) | (715) | (1002) |
| SCT-Ga395 | GFTFSNFW | IRLNSDNYAT | HGNFYDLDY | QEISGF | AAS | LQYASYPPT |
| | (1246) | (1247) | (1248) | (1249) | (860) | (1250) |
| SCT-Ga396 | GFSFTAYT | IVPYNGGT | ARGLQRFFAMDF | QSLANSYGSTY | GIS | LQYTHQPPT |
| | (1251) | (985) | (1252) | (1207) | (854) | (1253) |
| SCT-Ga397 | GYSFTTYT | INPYNGGT | ARSRSFAMDY | QSLANSYGDTY | GIS | LQGTHQPWT |
| | (1254) | (707) | (1215) | (1255) | (854) | (855) |
| SCT-Ga398 | GYTFTNYW | TNPSTGSS | AAYYRYGDGAY | DHINNW | GAS | QQYWSTPWT |
| | (932) | (1228) | (1229) | (989) | (691) | (716) |
| SCT-Ga399 | GYSFTGYT | IIPYNGGT | ARGLRGYFAMDY | QSLTNSYGNTY | GIS | LQGTHQPWT |
| | (878) | (888) | (1042) | (905) | (854) | (855) |
| SCT-Ga400 | GYTFTDYN | IYPYNGGT | ARYYGYFDY | DHINNW | GAT | QQYWSTPLT |
| | (1118) | (1256) | (1257) | (989) | (715) | (1219) |
| SCT-Ga401 | GYSFTGYI | IVPYNGGT | ARGLQRFYAMDY | QSLANSYGNTY | GIS | LQGTHQPPT |
| | (850) | (985) | (1258) | (853) | (854) | (882) |
| SCT-Ga402 | GFTFTSYY | IRDKANGYTT | ARDGGY | DHINNW | GAT | QQYWSTPFT |
| | (1220) | (1063) | (988) | (989) | (715) | (990) |
| SCT-Ga403 | GFTFSNFW | IRLNSDNYAT | HGNFYTLDY | QEITGF | AAS | LQYASYPPT |
| | (1246) | (1247) | (1259) | (1260) | (860) | (1250) |
| SCT-Ga404 | GYTFTNYW | TNPSTGYS | AAYYRYGDGAY | DHINNW | GAT | QQYWSTPWT |
| | (932) | (1261) | (1229) | (989) | (715) | (716) |
| SCT-Ga405 | GFTFSSFG | ISSGSSTI | ARGRFDYLYAMDY | QDISNY | YTS | QQGNTLPFT |
| | (1262) | (1243) | (1263) | (766) | (731) | (767) |
| SCT-Ga406 | GYSFTGYT | INPYNGGT | ARGLRFSLEY | QSLANSYGNTY | GIS | LQGTHQPWT |
| | (878) | (707) | (1264) | (853) | (854) | (855) |
| SCT-Ga407 | GYSFTGYI | IIPYNGGT | ARGLSYRYDY | QSLANSYGNTY | EIS | LQGTHQPPT |
| | (850) | (888) | (1265) | (853) | (1093) | (882) |
| SCT-Ga408 | GYSFTGYT | IIPYNGGT | ARGLRGYFAMDY | QSLTNSYGNTY | GIS | LQGTHQPWT |
| | (878) | (888) | (1042) | (905) | (854) | (855) |
| SCT-Ga409 | GYSFTGYI | IYPYNGGT | ARLISYAMDY | QSLANSYGKTY | GIS | LQGTHQPPT |
| | (850) | (1256) | (1266) | (1267) | (854) | (882) |
| SCT-Ga410 | GYTFTDHS | INTETGEP | ASPRNYDGAMDY | DHINNW | GAT | QQYWSTPLT |
| | (1217) | (916) | (1218) | (989) | (715) | (1219) |
| SCT-Ga411 | GYTFTNYW | TNPSTGYS | AAYYRYGDGAY | DHINSW | GTT | QQYWSTPWT |
| | (932) | (1261) | (1229) | (1268) | (1269) | (716) |
| SCT-Ga412 | GYAFTDYL | INPGSGGA | ARYHYYGSGLFPY | QSLLNSGNQKNY | GAS | QNYHSYPFT |
| | (1270) | (1271) | (1272) | (1273) | (691) | (1274) |
| SCT-Ga413 | GYTFTSYV | INPYNDGT | AREIDY | DHINNW | GAS | QQYWSTPWT |
| | (1275) | (1276) | (1277) | (989) | (691) | (716) |
| SCT-Ga414 | GFTFSDYY | IRNKANCYTT | ARDGGY | DHINNW | GAT | QQYWSIPFT |
| | (1216) | (1278) | (988) | (989) | (715) | (1002) |
| SCT-Ga415 | GYTFTNYG | INTYTGEP | ARYEYFDY | TDIDDD | EGN | LQSDNMPYT |
| | (862) | (1135) | (1279) | (1280) | (1281) | (1282) |
| SCT-Ga416 | GYSITSDYA | ITYSGDT | ARRELGQGFTY | QDVSTT | SAS | QQHYSVPWT |
| | (1283) | (1284) | (1285) | (1286) | (751) | (1287) |
| SCT-Ga417 | GYTFTYYE | IDPETGGT | TRWGYKFAY | DHINNW | GAT | QQYWSTPFT |
| | (1288) | (1289) | (1290) | (989) | (715) | (990) |
| SCT-Ga418 | GYDFTDYL | INPGSGGT | ARYHYYGSNLFPY | QSLLNSGNQKNY | GAS | QNYHSYPFT |
| | (1291) | (1292) | (1293) | (1273) | (691) | (1274) |
| SCT-Ga419 | GYSFTTYT | INPYNGGT | ARSRTFAMDY | QSLANSYGNTY | GIS | LQGTHQPWT |
| | (1254) | (707) | (1294) | (853) | (854) | (855) |
| SCT-Ga420 | GFTFSSYT | ISGGGSYT | TRWLQSLYAMDY | QNINVW | KAS | QQGQSYPYT |
| | (804) | (1295) | (1296) | (790) | (791) | (803) |
| SCT-Ga421 | GYSFTGYT | INPYNGGT | ASGGLRYGFAY | QSLANSYGNTY | GIS | LQGTHQPLT |
| | (878) | (707) | (1297) | (853) | (854) | (921) |
| SCT-Ga422 | GFTFNTNA | IRSKSNNYAT | VGSTMTIFSY | SSVSSSY | TTS | QQYSGYPLT |
| | (1298) | (1299) | (1300) | (871) | (834) | (1214) |
| SCT-Ga423 | GFSLTSYG | IWADGST | ARDLGYLTTAWFAY | SSVSY | GIS | QQISSYPLT |
| | (906) | (1301) | (1302) | (928) | (854) | (1303) |
| SCT-Ga424 | GYSFTAYT | INPYNGGI | ARSRTFAMDY | QSLANSYGNTY | GIS | LQGTHQPWT |
| | (1304) | (1305) | (1294) | (853) | (854) | (855) |
| SCT-Ga425 | GYSFTDYI | IVPYNGGT | ARGLQRFYAMDY | QSLENSYGNTY | GIS | LQGTHQPPT |
| | (910) | (985) | (1258) | (1306) | (854) | (882) |
| SCT-Ga426 | GYTFTNYW | TNPSTGSS | AAYYRYGDGAY | DHINNW | GAS | QQYWSTPWT |
| | (932) | (1228) | (1229) | (989) | (691) | (716) |
| SCT-Ga427 | GFSFPGYT | INPYNGAT | TRGTLITGFAY | QSLANSYGNTY | GIS | LQGTHLPLT |
| | (1307) | (1308) | (1309) | (853) | (854) | (1310) |
| SCT-Ga428 | GFSFTGYT | INPYNGAT | ARGTLITGFAY | QSLANSYGNTY | GIS | LQGTHLPLT |
| | (1311) | (1308) | (1312) | (853) | (854) | (1310) |
| SCT-Ga429 | GYSFTGYI | INPYNGGT | ARSRSFAMDY | QSLANSYGNTF | GIS | LQGTHQPWT |
| | (850) | (707) | (1215) | (1313) | (854) | (855) |
| SCT-Ga430 | GYSFTGYT | IVPYNGGT | ARGLSFRYDY | QSLANSYGDTF | GIS | LQGTHQPPT |
| | (878) | (985) | (1314) | (1006) | (854) | (882) |
| SCT-Ga431 | GYSFTGYI | IFPYNGGT | ARGLPRFYAMDY | QSLANSYGNTY | GIS | LQGTHQPPT |
| | (850) | (994) | (1315) | (853) | (854) | (882) |
| SCT-Ga432 | GFSLTSYG | IWAGGTT | ARFPSYRESMDY | ENIYSN | AT | QHFWGTPYT |
| | (906) | (1316) | (1317) | (1318) | (1319) | (1320) |
| SCT-Ga433 | GFTFSSYT | ISSGGSYT | TRWLLSLYAMDY | QNINVW | KAS | QQGQTYPFT |
| | (804) | (814) | (1321) | (790) | (791) | (1322) |
| SCT-Ga434 | GYSFTGYI | IYPYNGGT | ARLISYAMDY | QSLANSYGKTY | GIS | LQGTHQPPT |
| | (850) | (1256) | (1266) | (1267) | (854) | (882) |
| SCT-Ga435 | GYSFTGYT | INPYNGAT | ARGLRFSLEY | QSLANSYGNTY | GIS | LQGTHQPWT |
| | (878) | (1308) | (1264) | (853) | (854) | (855) |
| SCT-Ga436 | GFNIKDTY | IDPANGNI | ARSLPGAY | SSVSY | GIS | QQRSSFPPT |
| | (1323) | (1324) | (1325) | (928) | (854) | (1326) |
| SCT-Ga437 | GFTFTNYY | IRNRPYGYTT | AAGLDY | QSLLDSDGKTY | LVS | WQGTHFPYT |
| | (1327) | (1328) | (1329) | (696) | (697) | (698) |
| SCT-Ga438 | GFTFSNFG | ISSGSSTI | ARGRYDYLYAMDY | QDISNY | YTS | QQGNTLPFT |
| | (1242) | (1243) | (1244) | (766) | (731) | (767) |
| SCT-Ga439 | GYAFSDYL | INPGSGGT | ARFHYYGSSLFPY | QSLLNSGNQKKY | GAS | QNYHRFPFT |
| | (1330) | (1292) | (1331) | (1332) | (691) | (1333) |
| SCT-Ga440 | GYSFTGYT | IVPYNGGT | ARGLQRFYAMDY | QSLANSYGNTY | GIS | LQGTHQPPT |
| | (878) | (985) | (1258) | (853) | (854) | (882) |
| SCT-Ga441 | GFTFTDYY | IRNKANGYTT | ARDGGY | DHINNW | GAT | QQYWSTPFT |
| | (986) | (1001) | (988) | (989) | (715) | (990) |
| SCT-Ga442 | GYIFTSYW | IYPGIGST | EVPIGTVDY | QTIGTW | AT | QQLYNTPLT |
| | (1334) | (1335) | (1336) | (1337) | (1319) | (1338) |
| SCT-Ga443 | GFSLSTSGMG | IWWDDVK | ARLGVYYRYDGDY | QSLLDSDGKTY | LVS | WQGTHFPQT |
| | (991) | (992) | (1339) | (696) | (697) | (1340) |
| SCT-Ga444 | GFTFIDYY | IRNKANGYTT | ARDGGY | DHINNW | GAT | QQYWSTPFT |
| | (1341) | (1001) | (988) | (989) | (715) | (990) |
| SCT-Ga445 | GFLITAYG | IWGDGST | ARYDYYGMDY | QEISGF | AAS | LQYASYPPT |
| | (1342) | (1343) | (1344) | (1249) | (860) | (1250) |
| SCT-Ga446 | GFTFTNYY | IRNKPYGYTT | AAGLDY | EDIYNR | GAT | QQYWSTPWT |
| | (1327) | (1345) | (1329) | (714) | (715) | (716) |
| SCT-Ga447 | GFTFSNYA | ISSGGIYT | ARQRTTATDYFDY | DHINNW | GAT | QQYWSTPFT |
| | (1346) | (1347) | (1348) | (989) | (715) | (990) |
| SCT-Ga448 | GYIFTSYW | IYPGTGGT | EVPIGTVDY | QTIGTW | AT | QQLYSTPLT |
| | (1334) | (1349) | (1336) | (1337) | (1319) | (1350) |
| SCT-Ga449 | GFTFTNYY | IRNKPYGYTT | AAGLDY | QSLLDSDGKTY | LVS | WQGTHFPYT |
| | (1327) | (1345) | (1329) | (696) | (697) | (698) |
| SCT-Ga450 | GYIFTSYW | IYPGTGST | EVPVGTFDY | QTIGTW | AT | QQLYSTPLT |
| | (1334) | (1351) | (1352) | (1337) | (1319) | (1350) |
| SCT-Ga451 | GYSITSDYA | ITYSGDT | ARREVGQGFTY | QDVSTT | SAS | QQHYSVPWT |
| | (1283) | (1284) | (1353) | (1286) | (751) | (1287) |
| SCT-Ga452 | GFSLTSFG | IWADGGT | ARDLGYLTTAWFAY | SSVRY | GIS | QQTSSYPLT |
| | (1354) | (1355) | (1302) | (1356) | (854) | (1357) |
| SCT-Ga453 | GYSFTGYI | IIPYNGGT | TRGLQRFYAMDY | QSLANSYGNTY | GIS | LQGTHQPPT |
| | (850) | (888) | (1358) | (853) | (854) | (882) |
| SCT-Ga454 | GYSITSDYA | IVYSGST | ARRQVGLGFAY | QDVSTA | SAS | QQHYSSPWT |
| | (1283) | (1359) | (1360) | (757) | (751) | (1361) |
| SCT-Ga455 | GYSFTGYT | IIPYTGGT | ARSRTFAMDY | QSLANSYGNTY | GIS | LQGTHQPWT |
| | (878) | (1362) | (1294) | (853) | (854) | (855) |
| SCT-Ga456 | GFTFSQFW | IRLKSNSYKT | TRLPIFDY | SSVSY | GIS | QQISSYPLT |
| | (1363) | (1364) | (1365) | (928) | (854) | (1303) |
| SCT-Ga457 | GFSLTRHG | IWPGGNT | ARSGGYGNFAPYYFDQ | QSVLYSSNQKNF | WAS | HQYLSSYT |
| | (856) | (857) | (858) | (931) | (724) | (725) |
| SCT-Ga458 | GFSLTRHG | IWPGGNT | ARSGGYGNFAPYYFDQ | QSVLYSSNQKNF | WAS | HQYLSSYT |
| | (856) | (857) | (858) | (931) | (724) | (725) |
| SCT-Ga459 | GFTFSSYT | ISSGGSYT | TRWLLSLYAMDY | QNINVW | KAS | QQGQTYPFT |
| | (804) | (814) | (1321) | (790) | (791) | (1322) |
| SCT-Ga460 | GFTFSSYT | ISSGGSYT | TRWLLSLYAMDY | QNINVW | KAS | QQGQTYPFT |
| | (804) | (814) | (1321) | (790) | (791) | (1322) |
| SCT-Ga461 | GFTFSSYT | ISSGGSYT | TRWLLSLYAMDY | QNINVW | KAS | QQGQTYPFT |
| | (804) | (814) | (1321) | (790) | (791) | (1322) |
| SCT-Ga462 | GFTFSSYT | ISSGGSYT | TRWLLSLYAMDY | QNINVW | KAS | QQGQTYPFT |
| | (804) | (814) | (1321) | (790) | (791) | (1322) |
| SCT-Ga463 | GFAFSSFD | ISSGGSYT | TRQDYRYPYIMDY | QNINVW | KAS | QQGQSYPWT |
| | (1058) | (814) | (1059) | (790) | (791) | (924) |
| SCT-Ga464 | GFAFSSFD | ISSGGSYT | TRQDYRYPYIMDY | QNINVW | KAS | QQGQSYPWT |
| | (1058) | (814) | (1059) | (790) | (791) | (924) |
| SCT-Ga465 | GFAFSSFD | ISSGGSYT | TRQDYRYPYIMDY | QNINVW | KAS | QQGQSYPWT |
| | (1058) | (814) | (1059) | (790) | (791) | (924) |
| SCT-Ga466 | GFAFSSFD | ISSGGSYT | TRQDYRYPYIMDY | QNINVW | KAS | QQGQSYPWT |
| | (1058) | (814) | (1059) | (790) | (791) | (924) |
| SCT-Ga467 | GYAFTDYL | INPGSGGT | ARYHYYGSGLFPY | QSLLNSGNQKNY | GAS | QNYHSYPFT |
| | (1270) | (1292) | (1272) | (1273) | (691) | (1274) |
| SCT-Ga468 | GYAFTDYL | INPGSGGT | ARYHYYGSGLFPY | QSLLNSGNQKNY | GAS | QNYHSYPFT |
| | (1270) | (1292) | (1272) | (1273) | (691) | (1274) |
| SCT-Ga469 | GYAFTDYL | INPGSGGT | ARYHYYGSGLFPY | QSLLNSGNQKNY | GAS | QNYHSYPFT |
| | (1270) | (1292) | (1272) | (1273) | (691) | (1274) |
| SCT-Ga470 | GYAFTDYL | INPGSGGT | ARYHYYGSGLFPY | QSLLNSGNQKNY | GAS | QNYHSYPFT |
| | (1270) | (1292) | (1272) | (1273) | (691) | (1274) |
| SCT-Ga471 | GYTFSNYW | ILPGSGSA | ARQKYGNFYYFDF | QGIGNY | YTS | HQYSKLPYT |
| | (899) | (913) | (1043) | (1044) | (731) | (1045) |
| SCT-Ga472 | GYTFSNYW | ILPGSGSA | ARQKYGNFYYFDF | QGIGNY | YTS | HQYSKLPYT |
| | (899) | (913) | (1043) | (1044) | (731) | (1045) |
| SCT-Ga473 | GYTFSNYW | ILPGSGSA | ARQKYGNFYYFDF | QGIGNY | YTS | HQYSKLPYT |
| | (899) | (913) | (1043) | (1044) | (731) | (1045) |
| SCT-Ga474 | GYTFSNYW | ILPGSGSA | ARQKYGNFYYFDF | QGIGNY | YTS | HQYSKLPYT |
| | (899) | (913) | (1043) | (1044) | (731) | (1045) |
| SCT-Ga475 | GYAFTNYL | INPGSGGT | ARTGVYGNFLIFDY | QSLLNSGIQKNY | GAS | QNDHRYPFT |
| | (687) | (1292) | (689) | (690) | (691) | (692) |
| SCT-Ga476 | GYAFTNYL | INPGSGGT | ARTGVYGNFLIFDY | QSLLNSGIQKNY | GAS | QNDHRYPFT |
| | (687) | (1292) | (689) | (690) | (691) | (692) |
| SCT-Ga477 | GYAFTNYL | INPGSGGT | ARTGVYGNFLIFDY | QSLLNSGIQKNY | GAS | QNDHRYPFT |
| | (687) | (1292) | (689) | (690) | (691) | (692) |
| SCT-Ga478 | GYAFTNYL | INPGSGGT | ARTGVYGNFLIFDY | QSLLNSGIQKNY | GAS | QNDHRYPFT |
| | (687) | (1292) | (689) | (690) | (691) | (692) |
| SCT-Ga479 | GFSLTSYG | IWAGGT | ARFPSYRESMDY | ENIYSN | AT | QHFWGTPYT |
| | (906) | (1316) | (1317) | (1318) | (1319) | (1320) |
| SCT-Ga480 | GYAFTNYL | INPGSGGT | ARRGDKYGSAWFAY | QDVSTA | SAS | QQHYSSPRT |
| | (687) | (1292) | (1158) | (757) | (751) | (752) |
| SCT-Ga481 | GYAFTNYL | INPGSGGT | ARRGDKYGSAWFAY | QDVSTA | SAS | QQHYSSPRT |
| | (687) | (1292) | (1158) | (757) | (751) | (752) |
| SCT-Ga482 | GYAFTNYL | INPGSGGT | ARRGDKYGSAWFAY | QDVSTA | SAS | QQHYSSPRT |
| | (687) | (1292) | (1158) | (757) | (751) | (752) |
| SCT-Ga483 | GYAFTNYL | INPGSGGT | ARRGDKYGSAWFAY | QDVSTA | SAS | QQHYSSPRT |
| | (687) | (1292) | (1158) | (757) | (751) | (752) |
| SCT-Ga484 | GYAFTNYL | INPGSGGT | ARTGVYGNFLIFDY | QSLLNSGIQKNY | GAS | QNDHRYPFT |
| | (687) | (1292) | (689) | (690) | (691) | (692) |
| SCT-Ga485 | GYAFTNYL | INPGSGGT | ARTGVYGNFLIFDY | QSLLNSGIQKNY | GAS | QNDHRYPFT |
| | (687) | (1292) | (689) | (690) | (691) | (692) |
| SCT-Ga486 | GYAFTNYL | INPGSGGT | ARTGVYGNFLIFDY | QSLLNSGIQKNY | GAS | QNDHRYPFT |
| | (687) | (1292) | (689) | (690) | (691) | (692) |
| SCT-Ga487 | GYTFSNYW | ILPGSGSA | ARQKYGNFYYFDF | QGIGNY | YTS | HQYSKLPYT |
| | (899) | (913) | (1043) | (1044) | (731) | (1045) |
| SCT-Ga488 | GYTFSNYW | ILPGSGSA | ARQKYGNFYYFDF | QGIGNY | YTS | HQYSKLPYT |
| | (899) | (913) | (1043) | (1044) | (731) | (1045) |
| SCT-Ga489 | GYTFSNYW | ILPGSGSA | ARQKYGNFYYFDF | QGIGNY | YTS | HQYSKLPYT |
| | (899) | (913) | (1043) | (1044) | (731) | (1045) |
| SCT-Ga490 | GYTFSNYW | ILPGSGSA | ARQKYGNFYYFDF | QGIGNY | YTS | HQYSKLPYT |
| | (899) | (913) | (1043) | (1044) | (731) | (1045) |
| SCT-Ga491 | GYTFSNYW | ILPGSGSA | ARQKYGNFYYFDF | QGIGNY | YTS | HQYSKLPYT |
| | (899) | (913) | (1043) | (1044) | (731) | (1045) |
| SCT-Ga492 | GYTFSNYW | ILPGSGSA | ARQKYGNFYYFDF | QGIGNY | YTS | HQYSKLPYT |
| | (899) | (913) | (1043) | (1044) | (731) | (1045) |
| SCT-Ga493 | GFAFSSFD | ISSGGSYT | TRQDYRYPYIMDY | QNINVW | KAS | QQGQSYPWT |
| | (1058) | (814) | (1059) | (790) | (791) | (924) |
| SCT-Ga494 | GFAFSSFD | ISSGGSYT | TRQDYRYPYIMDY | QNINVW | KAS | QQGQSYPWT |
| | (1058) | (814) | (1059) | (790) | (791) | (924) |
| SCT-Ga495 | GFAFSSFD | ISSGGSYT | TRQDYRYPYIMDY | QNINVW | KAS | QQGQSYPWT |
| | (1058) | (814) | (1059) | (790) | (791) | (924) |
| SCT-Ga496 | GFAFSSFD | ISSGGSYT | TRQDYRYPYIMDY | QNINVW | KAS | QQGQSYPWT |
| | (1058) | (814) | (1059) | (790) | (791) | (924) |
| SCT-Ga497 | GYAFTNYL | INPGSGGT | ARRGDKYGSAWFAY | QDVSTA | SAS | QQHYSSPRT |
| | (687) | (1292) | (1158) | (757) | (751) | (752) |
| SCT-Ga498 | GYAFTNYL | INPGSGGT | ARRGDKYGSAWFAY | QDVSTA | SAS | QQHYSSPRT |
| | (687) | (1292) | (1158) | (757) | (751) | (752) |

TABLE 5

| | | SEQ | | SEQ |
|---|---|---|---|---|
| | | ID | | ID |
| mAb AA ID | V_{H} Amino Acid Sequence | NO: | V_{L} Amino Acid Sequence | NO: |
| SCT-Ga001 | QVQLQQSGAELVRPGTSVKVSCKASGYAFTNYL | 4 | DIVLTQSPSSLSVAAGEKVTLSCKSSQSLLNSG | 380 |
| | IEWVKQRPGQGLEWIGVINPGSGGSKYNEKFKG | | IQKNYLAWYQQKPGQPPKLLIYGASTRESGVPD | |
| | KTTLTADKSSSTAYMQLSSLTSEDSAVYFCART | | RFTGSGSGTDFTLTISSVQAEDLAVYYCQNDHR | |
| | GVYGNFLIFDYWGQGTTLTVSS | | YPFTFGGGTKLEIK | |
| SCT-Ga002 | DVQLQVSGPALVKPSQTVSLTCTVTGYSITNGN | 5 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga003 | DVQLQESGPALVKPSQTVSLTCTVTGYSITNGN | 6 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga004 | DVQLQESGPALVKPSQTVSLTCTVTGYSITNGN | 7 | DVVMTQTPLTLSVAIGQPASISCKSSQSLLDSD | 382 |
| | HWWNWIRQVSGSKLEWIGYITSSGRTDSNPSLK | | GKTYLNWLLQRPGQSPKRLIYLVSKLDSGVPNR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga005 | DVQLQESGPALVKPSQTVSLTCTVTGYSITNGN | 8 | DVVMTQTPLTLSVTIGQPASISCKSSQSLLDSD | 381 |
| | HWWMWTRQVSGSKLEWIGYITSSGRTDSNPSLK | | GKTYLNWLLQRPGQSPKRLIYLVSKLDSGVPDR | |
| | SRISITRDTSKNQLFLQLNSVTTEEIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga006 | DVQLQESGPALVKPSQTVSLTCTVTGYSITNGN | 9 | DVVMTQTPLTLSVTIGQPASISCKSSQSLLDSD | 381 |
| | HWWNWTRQVSGSKLEWIGYITSSGRTDSNPSLK | | GKTYLNWLLQRPGQSPKRLIYLVSKLDSGVPDR | |
| | SRISITRDTSKNQLFLQLTSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga007 | EVKLVESGGGLVQPGGSLSLSCAASGFTFTDHY | 10 | DIKMTQSPSSIYAYLGERVTITCKASQDINSYL | 383 |
| | MSWVRQPPGKALEWLGFIRNKVNGYTTEYSASV | | SWFQQKPGKSPKTLIYRANRLLDGVPSRFSGSG | |
| | KGRFSISRDNSQSILYLQMNALRAEDSATYYCA | | SGQDYSLTISSLEYEDMGIYYCLQYDELYTFGG | |
| | RYIPYYDPFDYWGQGTTLTVSS | | GTKLEIK | |
| SCT-Ga008 | DVQLQESGPALVKPSQTVSLTCTVTGYSITNGN | 11 | DVVMTQTPLTLSVTIGQPASISCKSSQSLLDSD | 381 |
| | HWWMWTRQVSGSKLEWIGYITSSGRTDSNPSLK | | GKTYLNWLLQRPGQSPKRLIYLVSKLDSGVPDR | |
| | SRISITRDTSKNQFFLQLNSVPAEDIATYYCSR | | FTGSGSGTDFTLKISRVEAEDLGVYYCWQGTHF | |
| | EGGWLPPVWGTGTTVTVSS | | PYTFGGGTKLEIK | |
| SCT-Ga009 | EVQLQQSGPVLVKPGASVKMSCKASGYTFTDYY | 12 | DVVMTQTPLTLSVTFGQPASISCKSSQSLLDSD | 384 |
| | MNWVKQSHGKSLEWTGVTNPYNGGTSYNQKFKG | | GKTYLNWLLQRPGQSPKRLIYLVSKLDSGVPDR | |
| | KATLTVDKSSSTAYMELNSLTSEDSAVYYCARE | | FTGSGSGTDFTLKISRVEAEDLGVYYCWQGTHF | |
| | GTDWGQGTTLTVSS | | PWTFGGGTKLEIK | |
| SCT-Ga010 | DVQLQESGPALVKPSQTVSLTCTVTGYSITNGN | 13 | DVVMTQTPLTLSVTIGQPASISCKSSQSLLDSD | 385 |
| | HWWNWIRQVSGGKLEWIGYITSSGRTDSNPSLK | | GKTYLNWLLQRPGQSPKRLIYLVSKLDSGVPNR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga011 | DVQLQESGPALVKPSQTVSLTCTVTGYSITNGN | 14 | DVVMTQTPLTLSVTIGQPASISCKSSQSLLDSD | 381 |
| | HWWNWIRQVSGSKLEWIGYISSSGRIDSNPSLK | | GKTYLNWLLQRPGQSPKRLIYLVSKLDSGVPDR | |
| | SRISITRDTSKNQIFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga012 | EVQLVESGGDLVKPGGSLKLSCAASGFTFSNYG | 15 | DIQMTQSSSSFSVSLGDRVTITCKASEDIYNRL | 386 |
| | MSWVRQTPDKRLEWVATISTGGSYTYYPDSVKG | | AWYQQKPGNAPRLLISGATSLETGVPSRFSGSG | |
| | RFTISRDNAKNTLYLQMSSLKSEDTAMYYCARP | | SGKDYTLSIISLQTEDVGTYYCQQYWSTPWTFG | |
| | HYYGSSSGNYFDYWGQGTTLTVSS | | GGTKLEIK | |
| SCT-Ga013 | DVQLQASGPALVKPSQTVSLTCTVTGYSITHGN | 16 | DVVMTQTPLTLSVTIGQPASISCKSSQSLLDSD | 381 |
| | HWWNWIRQVSGSKLEWIGYISSSGRIDSNPSLK | | GKTYLNWLLQRPGQSPKRLIYLVSKLDSGVPDR | |
| | SRISITRDTSKNKLFLQLNSVTSEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga014 | DVQLQESGPALVKPSQTVSLTCTVTGYSITNGN | 17 | DVVMTQTPLTLSVTIGQPASISCKSSQSLLDSD | 381 |
| | HWWNWIRQVSGSKLEWIGYITSSGRTDSNPSLQ | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga015 | DVQLQESGPALVKPSQTVSLTCTVTGYSITNGN | 18 | DVVMTQTPLSLSITIGQPASISCKSSQSLLDSD | 387 |
| | HWWNWIRQVSGSKLEWIGYIRSSGSTDSNPSLK | | GKTYLNWLLQRPGQSPKRLIYLLSKLDSGVPDR | |
| | SRISITRDTSKNQLFLQLNSVTTEDIGTYYCAR | | FTGSGSGTDFTLKISRVEAEDLGVYYCWQGTHF | |
| | EGGWLPPVWGTGTTVTVSS | | PYTFGGGTKLEIK | |
| SCT-Ga016 | QVQLKESGPGLVAPSQSLSLTCTVSGFSLTSYV | 19 | NIMMTQSPSSLAVSAGEKVTMSCKSSQSVLYSS | 388 |
| | ISWVRQPPGKGLEWLGIIWTGGGTNFNSALKSR | | NQKNYLAWYQQKPGQSPKLLIYWASTRESGVPD | |
| | LSISKDNSKSQVFLKMNSLQTDDTARYYCARTR | | RFTGSGSGTDFTLTISSVQAEDLAVYYCHQYLS | |
| | AYYSNWGYFDVWGTGTTVTVSS | | SYTFGGGTKLEIK | |
| SCT-Ga017 | DVQLQASGPALVKPSQTVSLTCTVTGYSITNGN | 20 | DVVMTQTPLTLSVTIGQPASISCKSGQSLLDSD | 389 |
| | HWWNWIRQVSGSKLEWIGYISSSGRTDSNPSLK | | GKTYLNWLLQRPGQSPKRLIYLVSKLDSGVPDR | |
| | SRISFTRDTSKNQLFLHLNSVTTEDMATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga018 | DVQLQASGPALVKPSQTVSLTCTVTGYSITNGN | 21 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNKLFLQLNSVTNEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga019 | DVQLQESGPGLVKPSQSLSLTCSVTGYSITSGY | 22 | DIQMTQSPSSLSASLGGKVTITCKASQDINKYI | 390 |
| | YWNWIRQFPGNKLEWMGYISYDGSNNYNPSLKN | | AWYQHKPGKGPRLLIRYTSTLQPGIPSRFSGSG | |
| | RISITRDTSKNQFFLKLNSVTTEDTATYYCARG | | SGRDYSFSISNLEPEDIATYYCLQYDNLWTFGG | |
| | LYYDYDDYFDYWGQGTTLTVSS | | GTKLEIK | |
| SCT-Ga020 | DVQLQESGPALVKPSQTVSLTCTVTGYSITNGN | 23 | DVVMTQTPLTLSVTIGQPASISCKSSQSLLDSD | 381 |
| | HWWNWIRQVSGGKLEWIGYIRSSGSTDFNPSLK | | GKTYLNWLLQRPGQSPKRLIYLVSKLDSGVPDR | |
| | SRISITRDISKNELFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga021 | DVQLQESGPALVKPSQTVSLTCTVTGYSITNGN | 24 | DVVMTQTPLTLSVTIGHPASISCKSSQSLFDSD | 391 |
| | HWWNWIRQVSGSKLEWIGYITSSGRIDSNPSLK | | GKTYLNWLLQRPGQSPKRLIYLVSKLDSGVPDR | |
| | SRISITRDTSKNQLFLQLNSVTTEDIGTYYCAR | | FTGSGSGTDFTLKISRVEAEDLGVYYCWQGTHF | |
| | EGGWLPPVWGTGTTVTVSS | | PYTFGGGTKLEIK | |
| SCT-Ga022 | DVQLQASGPALVKPSQTVSLTCTVTGYSITNGN | 20 | DVVMTQTPLTLSVTIGQPASISCKSSQSLLDSD | 381 |
| | HWWNWIRQVSGSKLEWIGYISSSGRTDSNPSLK | | GKTYLNWLLQRPGQSPKRLIYLVSKLDSGVPDR | |
| | SRISFTRDTSKNQLFLHLNSVTTEDMATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga023 | DVQLQESGPALVKPSQTVSLTCTVTGYSITNGN | 25 | DVVMTQTPLTLSVTIGQPASISCKSSQSLLDSD | 381 |
| | HWWNWIRQVSGSKLEWIGYISSSGSTDSNPSLN | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga024 | DVQLQESGPALVKPSQTVSLTCTVTGYSITNGN | 26 | DVVMTQTPLTLSVTIGQPASISCKSSQSLLDSD | 392 |
| | HWWMWTRQVSGSKLEWIGYITSSGRTDSNPSLK | | GKTYLNWLLQRPGQSPKRLIYLVSKLDSGVPDR | |
| | RRISITRDTSKNQFFLQLNSVPTDDVATYYCAR | | FTGSGSGTDFTLKISRVEAEDLGVYYCWQGTHF | |
| | EGGWLPPVWGTGTTVTVSS | | PYTFGGGTRLEIK | |
| SCT-Ga025 | DVQLQESGPALVKPSQTVSLTCTVTGYSITNGN | 27 | DVVMTQTPLTLSVTIGQPASISCKSSQSLLDSD | 381 |
| | HWWNWIRQVSGGKLEWIGYIRSSGSTDSNPSLK | | GKTYLNWLLQRPGQSPKRLIYLVSKLDSGVPDR | |
| | SRISITRDISKNELFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga026 | DVQLQESGPALVKPSQTVSLTCTVTGYSITNGN | 28 | DVVMTQTPLTLSVTIGHPASISCKSTQSLLDID | 393 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIVTYYCTR | | FTGSGSGTDFTLKISRVEAEDLGVYYCWQGTHF | |
| | EGGWLPPVWGTGTTVTVSS | | PYTFGGGTKLEIK | |
| SCT-Ga027 | QIQLVQSGPDLKKPGETVKISCKASGYTFTTYG | 29 | DVVMTQTPLSLPVSLGDQASFSCRSSQSLVHSN | 394 |
| | MNWVKQAPGKGLKWMGWINTYSGVPKYTDDFKG | | GNTYLHWYLQKPGQSPKLLIYKISNRFSGVPDR | |
| | RFAFSLETSASTAYLQINNLKNEDTATYFCARY | | FSGSGSGTDFTLKISRVEAEDLGVYFCSQSTHV | |
| | DYDVGFAYWGQGTLVTVSA | | PAFGGGTKLEIK | |
| SCT-Ga028 | DIQLQESGPALVKPSQTVSLTCTVTGYSITNGN | 30 | DVVMTQTPLTLSVTIGQPASISCKSSQSLLDSD | 395 |
| | HWWNWIRQVSGSKLEWIGYISSSGRTDSNPSLK | | GKTYLNWLLQRPGQSPKRLIYLVSKLDSGVPDR | |
| | SRISITRDTSKNKLFLQLNSVTTEDIATYYCAR | | FTGSGSGTDFTLKISRVEADDLGIYYCWQGTHF | |
| | EGGWLPPVWGTGTTVTVSS | | PYTFGGGTKLEIK | |
| SCT-Ga029 | QIQLVQSGPELKKPGETVKISCKASGYTFTTYG | 31 | DVVMTQTPLSLPVSLGDQASISCRSSQSLVHSN | 396 |
| | MNWVKQAPGKGLKWMGWINTFSRVPTYADDFKG | | GNTYLHWYLQKPGQSPKLLIYKVSNRFSGVPDR | |
| | RFAFSLETSASTAYLQINNLTNEDTATYFCARY | | FSGSGSGTDFTLKISRVEAEDLGVYFCSQSTHV | |
| | DYDVGFAYWGQGTLVTVSA | | PTFGGGTKLEIK | |
| SCT-Ga030 | DVQLQESGPALVKPSQTVSLTCTVTGYSITNGN | 32 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCSR | | FTGSGSGTDFTLKISRVEAEDLGVYYCWQGTHF | |
| | EGGWLPPVWGTGTTVTVSS | | PYTFGGGTKLEIK | |
| SCT-Ga031 | DVQLQESGPGLVKPSQTVFLTCTVTGISITTGN | 33 | DIVMTQSHKFMSPSVGDRVSITCRASQDVGTAV | 397 |
| | YKWSWIRQFPGNKLEWIGYIYYSGTITYNPSLT | | AWYQQKPGQSPKLLFYSASYRYTGVPDRFTGGG | |
| | SRTTITRDTPKNQFFLEMNSLTAEDTATYYCAR | | SGTDFTFTISSVQAEDLAVYYCQQHYSSPRTFG | |
| | ETVLRVFDYWGQGTTLTVSS | | GGTKVEIK | |
| SCT-Ga032 | DVQLQASGPALVKPYQTVSLTCTVTGYSITNGH | 34 | DVVMTQTPLTLSVTIGQPASISCKSSQSLLDSD | 381 |
| | HWWNWIRQVSGSKLEWIGYISSSGSTDNNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga033 | DVQLQESGPALVKPSQTVSLTCTVTGYSITNGN | 35 | DVVMTQTPLTLSVTIGQPASISCKSSQSLLDSD | 398 |
| | HWWNWIRQVSGSKLEWIGYITSSGRTDSNPSLR | | GKTYLNWLLQRPGQSPKRLIYLVSKLDSGVPDR | |
| | SRISITRDTSKNKLFLQLNSVTNEEKATYYCAR | | FTGSGSGTDFTLKISRVEAEDLGLYYCWQGTHF | |
| | EGGWLPPVWGTGTTVTVSS | | PYTFGGGTKLEIK | |
| SCT-Ga034 | DVQLQESGPALVKPSQTVSLTCTVTGYSITNGN | 36 | DVVMTQTPLTLSVTIGQPASISCRSSQSLLDSD | 399 |
| | HWWNWVRQVSGSKLEWIGYISSSGRIDSNPSLE | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga035 | DVQLQESGPALVKPSQTVSLTCTVTGYSITNGN | 37 | DVVMTQTPLTLSVTIGQPASISCKSSQSLLDSD | 400 |
| | HWWNWIRQVSGGKLEWIGYIRSSGSTDRNPSLK | | GKTYLNWLLLRPGQSPKRLIYLVSKLDSGVPDR | |
| | SRISITRDTSKNQLFLQLNSVTNEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga036 | DVQLQESGPALVKPSQTVSLTCTVTGYSITNGN | 38 | DVWTQTPLTLSVTIGQPASISCKSSQSLLDSD | 401 |
| | HWWNWVRQVSGRKLEWIGYITSSGRTDSNPSLK | | GKTYLNWLLQRPGQSPKRLIYLVSKLDSGVPDR | |
| | SRISITRDTSKNHLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga037 | QVQLQQSDAELVKPGASVKISCKVSGYTFTDHT | 39 | DIVMTQSHKFMSTSVGDRVSITCKASQDVSTAV | 402 |
| | IHWMKQRPEQGLEWIGYIYPRDGSTKYNEKFKG | | AWYQQKPGQSPKLLIYSASYRYTGVPDRFTGSG | |
| | KATLTADKSSSTAYMQLNSLTSEDSAVYFCARR | | SGTDFTFTISSVQAEDLAVYYCQQHYSTPYTFG | |
| | WASGMDYWGQGTSVTVSS | | GGTKLEIK | |
| SCT-Ga038 | QVQLQQSGAELVRPGTSVKVSCKASGYAFTNYL | 40 | DIVLTQSPSSLSVAAGEKVTLSCKSSQSLLNSG | 380 |
| | IEWIKQRPGQGLEWIGVINPGSGGSNCNEKFKA | | IQKNYLAWYQQKPGQPPKLLIYGASTRESGVPD | |
| | KTTLTADKSSSTAYMQLSSLTSEDSAVYFCART | | RFTGSGSGTDFTLTISSVQAEDLAVYYCQNDHR | |
| | GVYGNFLIFDYWGQGTTLTVSS | | YPFTFGGGTKLEIK | |
| SCT-Ga039 | DVQLQASGPALVKPSQTVSLTCTVTGYSITNGN | 41 | DVVMTQTPLTLSVIIGQPASISCKSGQSLLDSD | 403 |
| | HWWNWIRQVSGSKLEWIGYISSSGRTDSNPSLK | | GKTYLNWLLQRPGQSPKRLIYLVSKLDSGVPDR | |
| | SRISFTRDTSKNQLFLLLNSVTTEDMATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga040 | DVQLQESGPALVKPSQTVSLTCTVTGYSITNGN | 42 | DVVMTQTPLTLSVTIGQPASISCKSSQSLLDSD | 398 |
| | HWWNWIRQVSGSKLEWIGYITSSGRTDSNPSLR | | GKTYLNWLLQRPGQSPKRLIYLVSKLDSGVPDR | |
| | SRISITRDISKNKLFLQLNSVTNEEIATYYCAR | | FTGSGSGTDFTLKISRVEAEDLGLYYCWQGTHF | |
| | EGGWLPPVWGTGTTVTVSS | | PYTFGGGTKLEIK | |
| SCT-Ga041 | DVQLQESGPALVKPSQTVSLTCTVTGYSITNGN | 43 | DVVMTQTPLTLSATIGESASIYCKSSQSLLDSD | 404 |
| | HWWNWIRQVSGSKLEWIGYISSSGRTDSNPSLK | | GKTYLNWLLQRPGQSPKRLIYLVSKLDSGVPDR | |
| | SRISITRDTSKNQLFLQLNSVTTEDVATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga042 | DVQLQESGPALVKPSQTVSLTCTVTGYSITNGN | 6 | DVVMTQTPLTLSVTIGQPASISCRSSQSLLDSD | 405 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISGVEAEDLGAYYCWQGTHF | |
| | EGGWLPPVWGTGTTVTVSS | | PYTFGGGTKLEIK | |
| SCT-Ga043 | DVQLQESGPALVKPSQTVSLTCTVTGYSITNGN | 27 | DVVMTQTPLTLSVTIGQPASIFCKSSQSLLDRD | 406 |
| | HWWNWIRQVSGGKLEWIGYIRSSGSTDSNPSLK | | GKTYLNWLLQRPGQSPKRLIYLVSKLDSGVPDR | |
| | SRISITRDISKNELFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga044 | DVQLQESGPALVKPSQTVSLTCTVTGYSITNGN | 23 | DVVMTQTPLTLSVAIGQPASISCKSSQSLLDSD | 382 |
| | HWWNWIRQVSGGKLEWIGYIRSSGSTDFNPSLK | | GKTYLNWLLQRPGQSPKRLIYLVSKLDSGVPNR | |
| | SRISITRDISKNELFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga045 | DVQLQASGPALVKPSQTVSLTCTVTGYSITNGN | 20 | DVVMTQTPLTLSVTIGQPASIFCKSSQSLLDRD | 406 |
| | HWWNWIRQVSGSKLEWIGYISSSGRTDSNPSLK | | GKTYLNWLLQRPGQSPKRLIYLVSKLDSGVPDR | |
| | SRISFTRDTSKNQLFLHLNSVTTEDMATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga046 | DVQLQESGPALVKPSQTVSLTCSVTGYSITNGN | 44 | DVVMTQTPLTLSVTIGQPASISCKSSQSLLDSD | 398 |
| | HWWNWIRQVSGSKLEWIGYITSSGRTDSNPSLR | | GKTYLNWLLQRPGQSPKRLIYLVSKLDSGVPDR | |
| | SRISITRDTSKNKLFLQLNSVTNEDIATYYCAR | | FTGSGSGTDFTLKISRVEAEDLGLYYCWQGTHF | |
| | EGGWLPPVWGTGTTVTVSS | | PYTFGGGTKLEIK | |
| SCT-Ga047 | DVQLQASGPALVKPSQTVSLTCTVTGYSITNGN | 45 | DVVMTQTPLTLSVTIGQPASISCKSSQSLLDSD | 381 |
| | HWWNWIRQVSGSKLEWIGYIRSSGSTDSNPSLK | | GKTYLNWLLQRPGQSPKRLIYLVSKLDSGVPDR | |
| | SRISITRDTSKNKLFLQLKSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga048 | DVQLQESGPALVKPSQTVSLTCTVTGYSITSGN | 46 | DVVMTQTPLTLSVTIGQPASISCRSSQSLLDSD | 399 |
| | HWWNWVRQVSGSKLEWIGYISSSGRIDSNPSLE | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga049 | DVQLQESGPGLVKPSQTVFLTCTVTGISITTGN | 47 | DIVMTQSHKFMSTLLGDRVSITCKASQDVSTAV | 407 |
| | YKWSWIRQFPGNKLEWIGYIYYSGTITYNPSLA | | AWYQQKPGQSPKLLIYSASYRYTGVPDRFTGSG | |
| | SRTTITRDTPKNQFFLELSSLTAEDTASYYCAR | | SGTDFTFTISSVQAEDLAVYYCQQHYSSPRTFG | |
| | ETVLRVFDYWGQGTTLTVSS | | GGTKVEIK | |
| SCT-Ga050 | DVQLQASGPALVKPSQTVSLTCTVTGYSITNGN | 21 | DVVMTQTPLTLSVAIGQPASISCKSSQSLLDSD | 382 |
| | HWWNWIRQVSGSKLEWIGYITSSGRTDSNPSLK | | GKTYLNWLLQRPGQSPKRLIYLVSKLDSGVPNR | |
| | SRISITRDTSKNKLFLQLNSVTNEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga051 | QVQLQQSGPELVKPGASVKISCKASGYTFTDYY | 48 | DVVMTQTPLTLSVTIGQPASISCKSSQSLLDSD | 408 |
| | INWVKQRPGQGLEWIGWIFPGSGSTYYNEKFKG | | GKTYLNWLLQRPGQSPKRLIYLVSKLDSGVPDR | |
| | KATLTVDKSSSTTYMLLSSLTSEDSAVYFCARF | | FTGSGSGTDFTLKISRVEAEDLGVYYCWQGTHF | |
| | TVWGTGTTVTVSS | | PHTFGGGTKLEIK | |
| SCT-Ga052 | QVQLKQSGAELVKPGASVKLSCKASGYTFTDYY | 49 | DIQMTQTTSSLSASLGDRVTISCRASQDISNYL | 409 |
| | INWVKQRPGQGLEWIARIYPGSGNTYYNEKFKG | | NWYQQKPDGTVKLLIYYTSRLHSGVPSRFSGSG | |
| | KATLTAEKSSSTAYMQLSSLTSEDSAVYFCARV | | SGTDYSLTISNLEQEDIATYFCQQGNTLPFTFG | |
| | TYYGGYFDYWGQGTTLTVSS | | SGTKLEIK | |
| SCT-Ga053 | DVQLQESGPALVKPSQTVSLTCTVTGYSITNGN | 50 | DVVMTQTPLTLSVTIGQPASISCKSSQSLLDSD | 410 |
| | HWWNWIRQVSGSKLEWIGYISSSGRTDSNPSLK | | GKTYLNWLLQRPGQSPKRLIYLVSKLDSGVPDR | |
| | SRISITRDTSKNQLFLQLNSVTTENIATYYCAR | | FTGSGSGTDFTLKISRVEAEDLGVYYCWQGTHF | |
| | EGGWLPPVWGTGTTVTVSS | | PYTFGGGTKLEIQ | |
| SCT-Ga054 | DVQLQASGPALVKPYQTVSLTCTVTGYSITNGN | 51 | DVVMTQTPLTLSVTIGQAASFSCKSSQSLLDSD | 411 |
| | HWWNWIRQVSGSKLEWIGYIRSSGSTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga055 | QVQLQQSGAELMKPGASVKLSCKAIGYILSGYW | 52 | DIVMTQSHKFMSTSVGDRVSITCKASQDVSKAV | 412 |
| | IEWVKQRPGHGLEWIGEIFPGSGSTNYNEKFKG | | AWYQQKPGQSPKVVIYWASTRHTGVPDRFTGSG | |
| | KATFTADTSSNTAYIQLSSLTTEDSAIYYCARI | | SGTDYTLTISSVQAEDLALYYCQQHYSTPWTFG | |
| | RITTVPYFDFWGQGTTLTVSS | | GGTKLEIK | |
| SCT-Ga056 | QVQLQQPGTELVKPGASVKLSCKTSGYTFTSYW | 53 | DIQMTQSPASLSASVGETVTITCRASVNIHNYL | 413 |
| | MHWVKQRPGQGLEWIGNINPSNGVTNYNEKFKS | | AWYQQKQGKSPQLLVYNAKTLADGVPSRFSGSG | |
| | KATLTVDKSSSTAYMQLSSLTSEDSAVYYCARD | | SGTQSSLKINSLQPEDFGSYYCQHFWSTPYTFG | |
| | YDYFDYWGQGTTLTVSS | | GGTKLEIK | |
| SCT-Ga057 | DVQLQESGPGLVKPSQSLSLTCSVTGYSITSAY | 54 | DIVMTQSQKFMSTSVGDRVSVTCKASQNVGSNV | 414 |
| | YWNWIRQFPGNKLEWMGYISYDGTNNYNPSLKD | | AWYQQKPGQSPKVLIYSASYRYSGVPDRFTGSG | |
| | RISITRDASKNQFFLRLNSVTTEDTATYYCGRG | | SGTDFTLTISNVQSEDLTNYFCQQYNNYPFTFG | |
| | RPFVPYWGQGTLVTVSA | | SGTKLDIK | |
| SCT-Ga058 | DVQLQESGPALVKPSQTVSLTCTVTGYSITNGN | 55 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNKLFLQLNSVTNEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga059 | QVQLQQSDAELVKPGASVKISCKVSGYTFTDHT | 56 | DIVMTQSHKFMSTSVGDRVSITCKASQDVFTAV | 415 |
| | IHWMIQRPEQGLEWIGYIYPRDGSTKYNEKFKG | | AWYQQKPGQSPKLLIYSASYRFTGVPDRFTGSG | |
| | KATLTADKSSSTAYMQLNSLTSEDSAVYFCARR | | SGTDFTFTISSVQAEDLAVYYCQQHYSTPLTFG | |
| | ALPYFDCWGQGTTLTVSS | | AGTKLELR | |
| SCT-Ga060 | DVQLQASGPALVKPSQTVSLTCTVTGYSITNGN | 57 | DVVMTQTPLTLSVTIGQPASISCKSSQSLLDSD | 381 |
| | HWWNWIRQISGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga061 | DVQLQESGPALVKPSQTVSLTCTVTGYSITNGN | 58 | DVVMTQTPLTLSVTIGQPASISCKSSQSLLDSD | 381 |
| | HWWNWIRQVSGNKLEWIGYISSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga062 | DVQLQESGPGLVKPSQTVFLTCTVTGISITTGN | 33 | DIVMTQSHKFMSTSVGDRVSITCKASQDVSTAV | 416 |
| | YKWSWIRQFPGNKLEWIGYIYYSGTITYNPSLT | | AWYQQKPGQSPKLLIYSASYRYTGVPDRFTGSG | |
| | SRTTITRDTPKNQFFLEMNSLTAEDTATYYCAR | | SGTDFTFTISSVQAEDLAVYYCQQHYSSPRTFG | |
| | ETVLRVFDYWGQGTTLTVSS | | GGTKVEIK | |
| SCT-Ga063 | QVQLQQSGAELVRPGTSVKVSCKASGYAFTNYL | 59 | DIVLTQSPSSLSVAAGEKVTLSCKSSQSLLNSG | 417 |
| | IEWVKQRPGQGLEWIGVINPGSGGSNYNEKFKG | | VQKNYLAWYQQKPGRPPKLLIYGASTRESGVPD | |
| | KTTLTADKSSSTAYMQLSSLTSEDSAVYFCART | | RFTGSGSGTDFTLTISSVQAEDLAIYYCQNDHR | |
| | GVYGNFLIFDYWGQGTTLTVSS | | YPFTFGGGTKLEIK | |
| SCT-Ga064 | DVQLQESGPALVKPSQTVSLTCTVTGYSITNGN | 60 | DVVMTQTPLTLSVTIGQPASISCKSSQSLLDSD | 381 |
| | HWWNWIRQVSGNKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga065 | QVQLQQSGAELVRPGTSVKVSCKASGYAFTNYL | 61 | DIVLTQSPSSLRVAAGEKVTLSCKSSQSLLNSG | 418 |
| | IEWVKQRPGQGLEWIGVINPGSGGSNYNEKFKG | | IQKNYLAWYQQKPGQPPKLLIYGASTRESGVPD | |
| | KTTLTADKSSSTAYMQLSSLTSEDSAVYFCART | | RFTGSGSGTDFTLTISSVQAEDLAVYYCQNDHR | |
| | GVYANFLIFDYWGQGTTLTVSS | | YPFTFGGGTKLEIK | |
| SCT-Ga066 | DVQLQASGPALVKPSQTVSLTCTVTGYSITNGN | 20 | DVAMTQTPLTLSVTIGQPASISCKSGQSLLDSD | 419 |
| | HWWNWIRQVSGSKLEWIGYISSSGRTDSNPSLK | | GKTYLNWLLQRPGQSPKRLIYLVSKLDSGVPDR | |
| | SRISFTRDTSKNQLFLHLNSVTTEDMATYYCAR | | FTGSGSGTDFTLTISRVEAEDLGVYYCWQGTHF | |
| | EGGWLPPVWGTGTTVTVSS | | PYTFGGGTKLEIK | |
| SCT-Ga067 | DVQLQESGPALVKPSQTVSLTCTVTGYSITNGN | 62 | DVWTQTPLTLSVTIGQPASISCKSSQSLLDSD | 401 |
| | HWWNWVRQVSGRKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga068 | DVQLQESGPALVKPSQTVSLTCTVTGYSITNGN | 7 | DVVMTQTPLTLSVTIGQPASISCKSSQSLLDSD | 385 |
| | HWWNWIRQVSGSKLEWIGYITSSGRTDSNPSLK | | GKTYLNWLLQRPGQSPKRLIYLVSKLDSGVPNR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga069 | DVQLQESGPALVKPSQTVSLTCTVTGYSITNGN | 62 | DVWTQTPLTLSVTTGQPASISCKSSQSLLDSD | 420 |
| | HWWNWVRQVSGRKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga070 | DVQLQVSGPALVMPSQTVSLTCTVTGYSITNGN | 63 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYFCAR | | FTGSGSGTDFTLKISRVEAEDLGVYYCWQGTHF | |
| | EGGWLPPVWGTGTTVTVSS | | PYTFGGGTKLEIK | |
| SCT-Ga071 | DVQLQESGPALVKPSQTVSLTCTVTGYSITNGN | 62 | DVVMTQTPLTLSVTLGQPASISCKSSQSLLDSD | 421 |
| | HWWNWVRQVSGRKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGAGTDFTLKISRVEAEDLGVYYCWQGTHF | |
| | EGGWLPPVWGTGTTVTVSS | | PYTFGGGTKLEIK | |
| SCT-Ga072 | DVQLQESGPALVKPSQTVSLTCTVTGYSITNGN | 64 | DVVMTQTPLTLSVTIGQPASISCKSSQSLLDSD | 381 |
| | HWWMWTRQVSGSKLEWIGYITSSGRTDSNPSLK | | GKTYLNWLLQRPGQSPKRLIYLVSKLDSGVPDR | |
| | SRISITRDTSKNQLFLQLNSVTTEYIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga073 | DVQLQESGPALVKPSQTVSLTCTVTGYSITNGN | 7 | DVVMTQTPLTLSVTIGQPASISCKSSQSLLDSD | 422 |
| | HWWNWIRQVSGSKLEWIGYITSSGRTDSNPSLK | | GKTYLNWLLQRPGQSPKRLIYLLSKLDSGVPNR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga074 | QVQLQQPGTELVKPGASVKLSCKASGYTFTSYW | 65 | DIQMNQSPSSLSASLGDTITITCHASQNINVWL | 423 |
| | MHWVKQRPGQGLEWIGNINPSNGGTNYNEKFKT | | SWYQQKPGNIPKLLIYKASNLHTGVPSRFSGSG | |
| | KATLTVDRSSSTAYMQLSRLTSEDSAVYYCARR | | SGTGFTLTISSLQPEDIATYYCQQGQSYPLTFG | |
| | TTVLAGPYFDYWGQGTILTVSS | | SGTKLEIK | |
| SCT-Ga075 | DVQLQASGPALVKPSQTVSLTCTVTGYSITNGN | 66 | DVVMTQTPLTLSVTIGQPASISCKSSQSLLDSD | 424 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFSLKISRVEAEDLGVYFCWQGTHF | |
| | EGGWLPPVWGTGTTVTVSS | | PYTFGGGTKLEIK | |
| SCT-Ga076 | DVQLQASGPALVKPSQTVSLTCTVTGYSITNGN | 67 | DVVMTQSPLTLSVTIGQPASISCKSSQSLLDSD | 425 |
| | HWWNWIRQVSGSKLEWIGYITSSGRTDSNPSLK | | GKTYLNWLLQRPGQSPKRLIYLVSKLDSGVPNR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKVEIK | |
| SCT-Ga077 | DVQLQESGPALVKPSQTVSLTCTVTGYSITNGN | 55 | DVVMTQTPLTLSVTIGQPASISCKSSQSLLDSD | 426 |
| | HWWNWIRQVSGSKLEWIGYITSSGRTDSNPSLK | | GQTYLNWLLQRPGQSPKRLIYLVSKLDSGVPNR | |
| | SRISITRDTSKNKLFLQLNSVTNEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga078 | DVQLQESGPALVKPSQTVSLTCTVTGYSITNGN | 68 | DVVMTQTPLTLSVTIGQPASISCKSSQSLLDSD | 381 |
| | HWWNWIRQVSGGKLEWIGYIRSSGSTDSNPSLK | | GKTYLNWLLQRPGQSPKRLIYLVSKLDSGVPDR | |
| | SRISITRDISKNELFLQLNSVTTEDIATYFCTR | | FTGSGSGTDFTLKISRVEAEDLGVYYCWQGTHF | |
| | EGGWLPPVWGTGTTVTVSS | | PYTFGGGTKLEIK | |
| SCT-Ga079 | DVQLQASGPALVKPYQTVSLTCTVTGYSITNGN | 69 | DVVMTQTPLTLSVTIGQPASISCKSSQSLLDSD | 381 |
| | HWWNWIRQVSGSKLEWIGFIRSSGSTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGRLPPVWGTGTTVTVSS | | PYTFGGGTKLEIK | |
| SCT-Ga080 | DVQLQESGPALVKPSQTVSLTCTVTGYSITNGN | 62 | DVVMTQTPLTLSVTIGQPASISCKSSQSLLDSD | 381 |
| | HWWNWVRQVSGRKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga081 | DVQLQESGPALVKPSQTVSLTCTVTGYSISNGN | 70 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga082 | DVQLQESGPALVKPSQTVSLTCTVTGYSITNGN | 71 | DVVMTQTPLTLSVTIGQPASISCKSSQSLLDSD | 381 |
| | HWWNWVRQVSGSKLEWIGYISSSGRTDNNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTSVTVSS | | PYTFGGGTKLEIK | |
| SCT-Ga083 | DVQLQESGPALVKPSQTVSLTCTVTGYSITNGN | 36 | DVVMTQTPLTLSVTIGQPASISCKSSQSLLDSD | 381 |
| | HWWNWVRQVSGSKLEWIGYISSSGRIDSNPSLE | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga084 | DVQLQASGPALVKPSQTVSLTCTVTGYSITNGN | 72 | DVVMTQTPLTLSVTIGQPASISCKSSQSLLDSD | 385 |
| | HWWNWIRQVSGSKLEWIGYITSSGRTDSNPSLK | | GKTYLNWLLQRPGQSPKRLIYLVSKLDSGVPNR | |
| | SRISITRDTSKNQFFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga085 | DVQLQESGPALVKPSQTVSLTCTVTGYSITNGN | 73 | DVVMTQTPLTLSVTIGQPASISCKSSQSLLDSD | 381 |
| | HWWNWIRQVSGSKLEWIGYISSSGRIDSNPSLK | | GKTYLNWLLQRPGQSPKRLIYLVSKLDSGVPDR | |
| | SRISITRDTSKNQLFLQLNSVTTEDIGTYYCAR | | FTGSGSGTDFTLKISRVEAEDLGVYYCWQGTHF | |
| | EGGWLPPVWGTGTTVTVSS | | PYTFGGGTKLEIK | |
| SCT-Ga086 | DVQLQESGPALVKPSQTVSLTCTVTGYSITNGN | 74 | DVVMTQTPLTLSVTIGQPASISCKSSQSLLDSD | 381 |
| | HWWNWIRQVSGSKLEWIGYITSSGRTDSNPSLR | | GKTYLNWLLQRPGQSPKRLIYLVSKLDSGVPDR | |
| | SRISITRDTSKNQLLLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga087 | EVMLVESGGDLVKPGGSLKLSCAASGFTFSRYI | 75 | DIQMTQSPSSLSASLGERVSLTCRASQDIGSSL | 427 |
| | MSWVRQTPEKRLEWVATISGGGGNTYYPDSVKG | | NWLQQEPDGTIQRLIYATSSLDSGVPKRFSGSR | |
| | RFTISRDNAKNTLYLQVSSLRSEDTALYYCARW | | SGSDYSLTISSLESEDFVDYYCLQYASSPWTFG | |
| | LPSYYAMDYWGQGTSVTVSS | | GGTKLEIK | |
| SCT-Ga056 | QVQLQQPGTELVKPGASVKLSCKTSGYTFTSYW | 53 | DIQMTQSPASLSASVGETVTITCRASVNIHNYL | 413 |
| | MHWVKQRPGQGLEWIGNINPSNGVTNYNEKFKS | | AWYQQKQGKSPQLLVYNAKTLADGVPSRFSGSG | |
| | KATLTVDKSSSTAYMQLSSLTSEDSAVYYCARD | | SGTQSSLKINSLQPEDFGSYYCQHFWSTPYTFG | |
| | YDYFDYWGQGTTLTVSS | | GGTKLEIK | |
| SCT-Ga057 | DVQLQESGPGLVKPSQSLSLTCSVTGYSITSAY | 54 | DIVMTQSQKFMSTSVGDRVSVTCKASQNVGSNV | 414 |
| | YWNWIRQFPGNKLEWMGYISYDGTNNYNPSLKD | | AWYQQKPGQSPKVLIYSASYRYSGVPDRFTGSG | |
| | RISITRDASKNQFFLRLNSVTTEDTATYYCGRG | | SGTDFTLTISNVQSEDLTNYFCQQYNNYPFTFG | |
| | RPFVPYWGQGTLVTVSA | | SGTKLDIK | |
| SCT-Ga058 | DVQLQESGPALVKPSQTVSLTCTVTGYSITNGN | 55 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNKLFLQLNSVTNEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga059 | QVQLQQSDAELVKPGASVKISCKVSGYTFTDHT | 56 | DIVMTQSHKFMSTSVGDRVSITCKASQDVFTAV | 415 |
| | IHWMIQRPEQGLEWIGYIYPRDGSTKYNEKFKG | | AWYQQKPGQSPKLLIYSASYRFTGVPDRFTGSG | |
| | KATLTADKSSSTAYMQLNSLTSEDSAVYFCARR | | SGTDFTFTISSVQAEDLAVYYCQQHYSTPLTFG | |
| | ALPYFDCWGQGTTLTVSS | | AGTKLELR | |
| SCT-Ga060 | DVQLQASGPALVKPSQTVSLTCTVTGYSITNGN | 57 | DVVMTQTPLTLSVTIGQPASISCKSSQSLLDSD | 381 |
| | HWWNWIRQISGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga061 | DVQLQESGPALVKPSQTVSLTCTVTGYSITNGN | 58 | DVVMTQTPLTLSVTIGQPASISCKSSQSLLDSD | 381 |
| | HWWNWIRQVSGNKLEWIGYISSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga062 | DVQLQESGPGLVKPSQTVFLTCTVTGISITTGN | 33 | DIVMTQSHKFMSTSVGDRVSITCKASQDVSTAV | 416 |
| | YKWSWIRQFPGNKLEWIGYIYYSGTITYNPSLT | | AWYQQKPGQSPKLLIYSASYRYTGVPDRFTGSG | |
| | SRTTITRDTPKNQFFLEMNSLTAEDTATYYCAR | | SGTDFTFTISSVQAEDLAVYYCQQHYSSPRTFG | |
| | ETVLRVFDYWGQGTTLTVSS | | GGTKVEIK | |
| SCT-Ga063 | QVQLQQSGAELVRPGTSVKVSCKASGYAFTNYL | 59 | DIVLTQSPSSLSVAAGEKVTLSCKSSQSLLNSG | 417 |
| | IEWVKQRPGQGLEWIGVINPGSGGSNYNEKFKG | | VQKNYLAWYQQKPGRPPKLLIYGASTRESGVPD | |
| | KTTLTADKSSSTAYMQLSSLTSEDSAVYFCART | | RFTGSGSGTDFTLTISSVQAEDLAIYYCQNDHR | |
| | GVYGNFLIFDYWGQGTTLTVSS | | YPFTFGGGTKLEIK | |
| SCT-Ga064 | DVQLQESGPALVKPSQTVSLTCTVTGYSITNGN | 60 | DVVMTQTPLTLSVTIGQPASISCKSSQSLLDSD | 381 |
| | HWWNWIRQVSGNKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga065 | QVQLQQSGAELVRPGTSVKVSCKASGYAFTNYL | 61 | DIVLTQSPSSLRVAAGEKVTLSCKSSQSLLNSG | 418 |
| | IEWVKQRPGQGLEWIGVINPGSGGSNYNEKFKG | | IQKNYLAWYQQKPGQPPKLLIYGASTRESGVPD | |
| | KTTLTADKSSSTAYMQLSSLTSEDSAVYFCART | | RFTGSGSGTDFTLTISSVQAEDLAVYYCQNDHR | |
| | GVYANFLIFDYWGQGTTLTVSS | | YPFTFGGGTKLEIK | |
| SCT-Ga066 | DVQLQASGPALVKPSQTVSLTCTVTGYSITNGN | 20 | DVAMTQTPLTLSVTIGQPASISCKSGQSLLDSD | 419 |
| | HWWNWIRQVSGSKLEWIGYISSSGRTDSNPSLK | | GKTYLNWLLQRPGQSPKRLIYLVSKLDSGVPDR | |
| | SRISFTRDTSKNQLFLHLNSVTTEDMATYYCAR | | FTGSGSGTDFTLTISRVEAEDLGVYYCWQGTHF | |
| | EGGWLPPVWGTGTTVTVSS | | PYTFGGGTKLEIK | |
| SCT-Ga067 | DVQLQESGPALVKPSQTVSLTCTVTGYSITNGN | 62 | DVVVTQTPLTLSVTIGQPASISCKSSQSLLDSD | 401 |
| | HWWNWVRQVSGRKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga068 | DVQLQESGPALVKPSQTVSLTCTVTGYSITNGN | 7 | DVVMTQTPLTLSVTIGQPASISCKSSQSLLDSD | 385 |
| | HWWNWIRQVSGSKLEWIGYITSSGRTDSNPSLK | | GKTYLNWLLQRPGQSPKRLIYLVSKLDSGVPNR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga069 | DVQLQESGPALVKPSQTVSLTCTVTGYSITNGN | 62 | DVVVTQTPLTLSVTTGQPASISCKSSQSLLDSD | 420 |
| | HWWNWVRQVSGRKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga070 | DVQLQVSGPALVMPSQTVSLTCTVTGYSITNGN | 63 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYFCAR | | FTGSGSGTDFTLKISRVEAEDLGVYYCWQGTHF | |
| | EGGWLPPVWGTGTTVTVSS | | PYTFGGGTKLEIK | |
| SCT-Ga071 | DVQLQESGPALVKPSQTVSLTCTVTGYSITNGN | 62 | DVVMTQTPLTLSVTLGQPASISCKSSQSLLDSD | 421 |
| | HWWNWVRQVSGRKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGAGTDFTLKISRVEAEDLGVYYCWQGTHF | |
| | EGGWLPPVWGTGTTVTVSS | | PYTFGGGTKLEIK | |
| SCT-Ga072 | DVQLQESGPALVKPSQTVSLTCTVTGYSITNGN | 64 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEYIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga073 | DVQLQESGPALVKPSQTVSLTCTVTGYSITNGN | 7 | DVVMTQTPLTLSVTIGQPASISCKSSQSLLDSD | 422 |
| | HWWNWIRQVSGSKLEWIGYITSSGRTDSNPSLK | | GKTYLNWLLQRPGQSPKRLIYLLSKLDSGVPNR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga074 | QVQLQQPGTELVKPGASVKLSCKASGYTFTSYW | 65 | DIQMNQSPSSLSASLGDTITITCHASQNINVWL | 423 |
| | MHWVKQRPGQGLEWIGNINPSNGGTNYNEKFKT | | SWYQQKPGNIPKLLIYKASNLHTGVPSRFSGSG | |
| | KATLTVDRSSSTAYMQLSRLTSEDSAVYYCARR | | SGTGFTLTISSLQPEDIATYYCQQGQSYPLTFG | |
| | TTVLAGPYFDYWGQGTILTVSS | | SGTKLEIK | |
| SCT-Ga075 | DVQLQASGPALVKPSQTVSLTCTVTGYSITNGN | 66 | DVVMTQTPLTLSVTIGQPASISCKSSQSLLDSD | 424 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFSLKISRVEAEDLGVYFCWQGTHF | |
| | EGGWLPPVWGTGTTVTVSS | | PYTFGGGTKLEIK | |
| SCT-Ga076 | DVQLQASGPALVKPSQTVSLTCTVTGYSITNGN | 67 | DVVMTQSPLTLSVTIGQPASISCKSSQSLLDSD | 425 |
| | HWWNWIRQVSGSKLEWIGYITSSGRTDSNPSLK | | GKTYLNWLLQRPGQSPKRLIYLVSKLDSGVPNR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKVEIK | |
| SCT-Ga077 | DVQLQESGPALVKPSQTVSLTCTVTGYSITNGN | 55 | DVVMTQTPLTLSVTIGQPASISCKSSQSLLDSD | 426 |
| | HWWNWIRQVSGSKLEWIGYITSSGRTDSNPSLK | | GQTYLNWLLQRPGQSPKRLIYLVSKLDSGVPNR | |
| | SRISITRDTSKNKLFLQLNSVTNEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga078 | DVQLQESGPALVKPSQTVSLTCTVTGYSITNGN | 68 | DVVMTQTPLTLSVTIGQPASISCKSSQSLLDSD | 381 |
| | HWWNWIRQVSGGKLEWIGYIRSSGSTDSNPSLK | | GKTYLNWLLQRPGQSPKRLIYLVSKLDSGVPDR | |
| | SRISITRDISKNELFLQLNSVTTEDIATYFCTR | | FTGSGSGTDFTLKISRVEAEDLGVYYCWQGTHF | |
| | EGGWLPPVWGTGTTVTVSS | | PYTFGGGTKLEIK | |
| SCT-Ga079 | DVQLQASGPALVKPYQTVSLTCTVTGYSITNGN | 69 | DVVMTQTPLTLSVTIGQPASISCKSSQSLLDSD | 381 |
| | HWWNWIRQVSGSKLEWIGFIRSSGSTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGRLPPVWGTGTTVTVSS | | PYTFGGGTKLEIK | |
| SCT-Ga080 | DVQLQESGPALVKPSQTVSLTCTVTGYSITNGN | 62 | DVVMTQTPLTLSVTIGQPASISCKSSQSLLDSD | 381 |
| | HWWNWVRQVSGRKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga081 | DVQLQESGPALVKPSQTVSLTCTVTGYSISNGN | 70 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga082 | DVQLQESGPALVKPSQTVSLTCTVTGYSITNGN | 71 | DVVMTQTPLTLSVTIGQPASISCKSSQSLLDSD | 381 |
| | HWWNWVRQVSGSKLEWIGYISSSGRTDNNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTSVTVSS | | PYTFGGGTKLEIK | |
| SCT-Ga083 | DVQLQESGPALVKPSQTVSLTCTVTGYSITNGN | 36 | DVVMTQTPLTLSVTIGQPASISCKSSQSLLDSD | 381 |
| | HWWNWVRQVSGSKLEWIGYISSSGRIDSNPSLE | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga084 | DVQLQASGPALVKPSQTVSLTCTVTGYSITNGN | 72 | DVVMTQTPLTLSVTIGQPASISCKSSQSLLDSD | 385 |
| | HWWNWIRQVSGSKLEWIGYITSSGRTDSNPSLK | | GKTYLNWLLQRPGQSPKRLIYLVSKLDSGVPNR | |
| | SRISITRDTSKNQFFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga085 | DVQLQESGPALVKPSQTVSLTCTVTGYSITNGN | 73 | DVVMTQTPLTLSVTIGQPASISCKSSQSLLDSD | 381 |
| | HWWNWIRQVSGSKLEWIGYISSSGRIDSNPSLK | | GKTYLNWLLQRPGQSPKRLIYLVSKLDSGVPDR | |
| | SRISITRDTSKNQLFLQLNSVTTEDIGTYYCAR | | FTGSGSGTDFTLKISRVEAEDLGVYYCWQGTHF | |
| | EGGWLPPVWGTGTTVTVSS | | PYTFGGGTKLEIK | |
| SCT-Ga086 | DVQLQESGPALVKPSQTVSLTCTVTGYSITNGN | 74 | DVVMTQTPLTLSVTIGQPASISCKSSQSLLDSD | 381 |
| | HWWNWIRQVSGSKLEWIGYITSSGRTDSNPSLR | | GKTYLNWLLQRPGQSPKRLIYLVSKLDSGVPDR | |
| | SRISITRDTSKNQLLLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga087 | EVMLVESGGDLVKPGGSLKLSCAASGFTFSRYI | 75 | DIQMTQSPSSLSASLGERVSLTCRASQDIGSSL | 427 |
| | MSWVRQTPEKRLEWVATISGGGGNTYYPDSVKG | | NWLQQEPDGTIQRLIYATSSLDSGVPKRFSGSR | |
| | RFTISRDNAKNTLYLQVSSLRSEDTALYYCARW | | SGSDYSLTISSLESEDFVDYYCLQYASSPWTFG | |
| | LPSYYAMDYWGQGTSVTVSS | | GGTKLEIK | |
| SCT-Ga088 | EVMLVESGGDLVKPGGSLKLSCAASGFTFSRYI | 75 | DIQMNQSPSSLSASLGDTITITCHASQNINIWL | 428 |
| | MSWVRQTPEKRLEWVATISGGGGNTYYPDSVKG | | NWYQQKPGNIPKLLIYKASNLHTGVPSRFSGSG | |
| | RFTISRDNAKNTLYLQVSSLRSEDTALYYCARW | | SGTGFTLIISSLQPEDIATYYCQQGQSYPYTFG | |
| | LPSYYAMDYWGQGTSVTVSS | | GGTKLEIK | |
| SCT-Ga089 | DVQLQASGPALVKPSQTVSLTCTVTGYSITNGN | 76 | DVVMTQTPLTLSVTIGQPASISCKSSQSLLDSD | 385 |
| | HWWNWIRQVSGSKLEWIGYITSSGRTDSNPSLK | | GKTYLNWLLQRPGQSPKRLIYLVSKLDSGVPNR | |
| | SRISITRDTFKNQFFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga090 | DVQLQVSGPALVMPSQTVSLTCTVTGYSITNGN | 77 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga091 | DVQLQASGPALVKPSQTVSLTCTVTGYSITNGN | 78 | DVVMTQTPLTLSVTIGQPASISCKSSQSLLDSD | 381 |
| | HWWNWIRQVSGSKLEWIGYIRSSGSTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga092 | EVMLVESGGGLVKPGGSLKLSCAASGFTFSSYT | 79 | DIQMTQSPSSLSASLGERVSLTCRASQDIGSSL | 429 |
| | MSWVRQTPEKRLEWVATISGGGGNTYYPDSVKG | | NWLQQEPDGTIKRLIYATSSLDSGVPKRFSGSR | |
| | RFTISRDNAKNTLYLQMSSLRSEDTALYYCARW | | SGSDYSLTISSLESEDFVDYYCLQYVSCPWTFG | |
| | LLTYYAMDYWGQGTSVTVSS | | GGTKVEIK | |
| SCT-Ga093 | QVQLQQSGPELVKPGASVRISCKASGYTFTDCY | 80 | DIQMNQSPSSLSASLGDTITITCHASQNINVWL | 430 |
| | INWVKLRPGQGLEWIGWIFPGSDFTYYNEKFKG | | SWYQQKPGNIPKLLIYKASNLHTGVPSRFRGSG | |
| | KATLTVDTSSSTAYMLFSSLTSEDSAVYFCARY | | SGTGFTLSISSLQPEDIATYYCQQGQSYPLTFG | |
| | GLGHYFDYWGQGTTLTVSS | | GGTKLEIK | |
| SCT-Ga094 | DVQLQASGPALVKPSQTVSLTCTVTGYSITNGN | 67 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga095 | QIQLQQSGPELVKSGASVKISCKASGYTFTAYC | 81 | DIQMNQSPSSLSASLGDTITITCHASQNINVWL | 431 |
| | INWVKQRPGQGLEWIGWIFPGSGSTNYNEKFKG | | SWYQQKPGNIPKLLIYKASNLHTGVPSRFSGSG | |
| | KATYTVDKSSSTAYMLLSSLTSEDSAVYFCARY | | SGTGFTLTISSLQPEDIATYYCQQGQSYPLTFG | |
| | GLGHYFDYWGQGTTLTVSS | | GGTKLEIK | |
| SCT-Ga096 | DVQLQESGPALVKPSQTVSLTCTVTGYSITNGN | 82 | DVVMTQTPLTLSVTFGQPASISCKSSQSLLDSD | 432 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYFCSR | | FTGSGSGTDFTLKISRVEAEDLGVYYCWQGTHF | |
| | EGGWFPPVWGTGTTVTVSS | | PYTFGGGTKLEIK | |
| SCT-Ga097 | DVQLQESGPALVKPSQTVSLTCTVTGYSITNGN | 83 | DVVMTQTPLTLSVTIGQPASISCKSSQSLLDSD | 381 |
| | HWWNWIRQVSGGKLEWIGYIRSSGSTDYNPSLK | | GKTYLNWLLQRPGQSPKRLIYLVSKLDSGVPDR | |
| | SRISITRDISKNELFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga098 | DVQLQESGPALVKPSQTVSLTCTVTGYSITNGN | 84 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCTR | | FTGSGSGTDFTLKTSRVEAEDLGVYYCWQGTHF | |
| | EGGWLPPVWGTGTTVTVSS | | PYTFGGGTKLEIK | |
| SCT-Ga099 | QVQLQQPGTELVKPGTSVKLSCKASGYTFTSYW | 85 | DIQMNQSPSSLSASLGDTITITCHASQNINVWL | 433 |
| | MHWVKQRPGQGLEWIGNINPSNGGTNYNEKFMR | | SWYQHKSGNIPKLLIYQASNLHTGVPSRFSGSG | |
| | KATLTVDKSSSTAYMQLSSLTSEDSAVYYCARR | | SGTGFTLTISSLQPEDIATYYCQQGQSYPLTFG | |
| | TTVLAGPYFDYWGQGTTLTVSS | | SGTKLEIK | |
| SCT-Ga100 | EVMLVESGGGLVKPGGSLKLSCAASGFTFSSYT | 79 | DIQMNQSPSSLSASLGDTITITCHASQNINVWL | 434 |
| | MSWVRQTPEKRLEWVATISGGGGNTYYPDSVKG | | NWYQQKPGNIPKLLIYKASNLHTGVPSRFSGSG | |
| | RFTISRDNAKNTLYLQMSSLRSEDTALYYCARW | | SGTGFTLTISSLQPEDIATYYCQQGQSYPYTFG | |
| | LLTYYAMDYWGQGTSVTVSS | | GGTKLEIK | |
| SCT-Ga101 | EVQLVESGGDLVKPGGSLKLSCAASGFTFSSYG | 86 | NIMMTQSPSSLAVSAGEKVTMSCKSSQSVLYSS | 435 |
| | MSWVRQTPDKRLEWVATISSGGSYTYYPDSVKG | | NQKNYLAWYQQKPGQSPKLLIYWASTRESGVPD | |
| | RFTISRDNAKNTLYLQMSSLQSEDTAMYYCARH | | RFTGSGSGTDFTLTISSVQAEDLAVYYCHQYLS | |
| | HYYYGSSYIFYFDVWGTGTTVTVSS | | SLTFGAGTKLELK | |
| SCT-Ga102 | EVMMVESGGGLVKPGGSLKLSCAASGFTFSSYT | 87 | DIQMNQSPSSLSASLGDTITITCHASQNINVWL | 436 |
| | MSWVRQTPEKRLEWVATISGRGGNTYYPDSVKG | | SWYQQKPGNIPKLLIYKASTLHTGVPSRFSGSG | |
| | RFTISRDNAKSTLYLQMSSLRSEDTAMYYCARW | | SGSGFTLTISSLQPEDIATYYCQQGQSYPYTFG | |
| | LLTYYAMDYWGQGTSVTVSS | | GGTKLEIK | |
| SCT-Ga103 | EVMMVESGGGLVRPGGSLKLSCAASGFTFSSYT | 88 | DIQMNQSPSSLSASLGDTITITCHASQNINIWL | 437 |
| | MSWIRQTPEKRLEWVATISGRGGNTYYPDSVKG | | SWYQQKPGNIPKLLIYKASNLHTGVPSRFSGSG | |
| | RITISRDNAKNTLYLQMSSLRSEDTAMYYCVRW | | SGTGFTLTISSLQPEDIATYYCQQGQSYPYTFG | |
| | LLTYYAMDYWGHGTSVTVSS | | GGTKLEIK | |
| SCT-Ga104 | EVMLVESGGGLVKPGGSLKLSCAASGFTFSSYT | 89 | DIQMNQSPSSLSASLGDTITITCHASQNINVWL | 438 |
| | MSWVRQTPEKRLEWAATISGGGGNTYYPDSVKG | | NWYQQKPGNTPKLLIYKASKLHTGVPSRFSGSG | |
| | RFTISRDNAKNTLYLQMSSLRSEDTALYYCARW | | SGTGFTLTISSLQPEDIATYSCQQGQSYPYTFG | |
| | LPTYYAMDYWGQGTSVTVSS | | GGTKLEIN | |
| SCT-Ga105 | QIQLVQSGPELKKPGETVKISCKASGYSFTTYG | 90 | DVVMTQTPLSLPVSLGDQASISCRSSQSLVHSN | 439 |
| | MNWVKQAPGKGLKWMGWINTYSGVPTYDDDFKG | | GNTYLHWYLQKPGQSPKLLIYKISNRFSGVPDR | |
| | RFAFSLETSASTAYLQINNLKNEDTATYFCARY | | FSGSGSGTDFTLKISRVEAEDLGVYFCSQSTHV | |
| | DYDVGFAYWGPGTLVTVSA | | PAFGGGTKLEIK | |
| SCT-Ga106 | DVQLQASGPALVKPSQTVSLTCTVTGYSITHGN | 91 | DVVMTQTPLTLSITIGQPASISCKSSQSLLDSD | 440 |
| | HWWNWIRQVSGSKLEWIGYISSSGRIDTNPSLK | | GKTYLNWLLQRPGQSPKRLIYLVSKLDSGVPDR | |
| | SPISITRDTSKNKFFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga107 | DVQLQESGPALVKPSQTVSLTCTVTGYSITNGN | 92 | DVVMTQTPLTLSVTIGQPASISCKSSQSLLDSD | 381 |
| | HWWNWIRQVSGSKLEWIGYITSSGRTDSNPSLQ | | GKTYLNWLLQRPGQSPKRLIYLVSKLDSGVPDR | |
| | SRISITRDTSKNQLFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga108 | DVQLQESGPALVKPSQTVSLTCTVTGYSITNGN | 93 | DVVMTQTPLTLSVTIGQPASISCKSSQSLLDSD | 381 |
| | HWWNWIRQVSGRKLEWIGYITSSGRIDSNPSLK | | GKTYLNWLLQRPGQSPKRLIYLVSKLDSGVPDR | |
| | SRISITRDTSKNQLFLQLNSVTSEDLGTYYCAR | | FTGSGSGTDFTLKISRVEAEDLGVYYCWQGTHF | |
| | EGGWLPPVWGTGTTVTVSS | | PYTFGGGTKLEIK | |
| SCT-Ga109 | DVQLQVSGPALVKPSQTVSLTCTVTGYSITNGN | 94 | DVVMTQTPLTLSVTIGQPASISCKSSQSLLDSD | 381 |
| | HWWNWIRQVSGSKLEWIGYISSSGRTDSNPSLQ | | GKTYLNWLLQRPGQSPKRLIYLVSKLDSGVPDR | |
| | SRISITRDTSKNQLFLQLNSVTTEDIGTYYCAR | | FTGSGSGTDFTLKISRVEAEDLGVYYCWQGTHF | |
| | EGGWLPPVWGTGTTVTVSS | | PYTFGGGTKLEIK | |
| SCT-Ga110 | DVQLQESGPALVKPSQTVSLTCTVTGYSITNGN | 62 | DVVVTQTPLTLSVTIGQSASISCKSSQSLLDSD | 441 |
| | HWWNWVRQVSGRKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga111 | DVQLQESGPALVKPSQTVSLTCTVTGYSITNGN | 95 | DVVMTQTPLTLSVTIGQPASISCKSSQSLLDSD | 398 |
| | HWWNWIRQVSGSKLEWIGYITSSGRTDSNPSLR | | GKTYLNWLLQRPGQSPKRLIYLVSKLDSGVPDR | |
| | SRISITRDISKNKLFLQMNSVTNEEIATYYCAR | | FTGSGSGTDFTLKISRVEAEDLGLYYCWQGTHF | |
| | EGGWLPPVWGTGTTVTVSS | | PYTFGGGTKLEIK | |
| SCT-Ga112 | QVQLQQSGAELVRPGTSVKVSCKASGYAFTNYL | 96 | DIVLTQSPSSLSVAAGEKVTLSCKSSQSLLNSG | 442 |
| | IEWVKQRPGQGLEWIGVINPGSGGSNYSEKFKG | | IQKNYLAWYQQKPGQPPKLLIYGASTRESGVPD | |
| | KTTLTADRSSSTAYMQLSSLTSEDSAVYFCART | | RFTGSGSGTDFTLTISSVQVEDLAVYYCQNDHR | |
| | GVYGNFLIFDYWGQGTTLTVSS | | YPFTFGGGTKLEIK | |
| SCT-Ga113 | DVQLQESGPALVKPSQTVSLTCTVTGYSITNGN | 84 | DVVMTQTPLTLSVTIGQPASMSCKSSQSLLDSD | 443 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCTR | | FTGSGSGTDFTLKISRVEAEDLGLYYCWQGTHF | |
| | EGGWLPPVWGTGTTVTVSS | | PYTFGGGTKLEIK | |
| SCT-Ga114 | DVQLQESGPALVKPSQTVSLTCTVTGYSITNGN | 97 | DVVMTQTPLTLSVTIGQPASISCKSSQSLLDSD | 381 |
| | HWWNWIRQVSGGKLEWIGYITSSGRTDSNPSLK | | GKTYLNWLLQRPGQSPKRLIYLVSKLDSGVPDR | |
| | SRISITRDTSKNHLFLQLNSVNTDDVGTYYCAR | | FTGSGSGTDFTLKTSRVEAEDLGVYYCWQGTHF | |
| | EGGWLPPVWGTGTTVTVSS | | PYTFGGGTKLEIK | |
| SCT-Ga115 | DVQLQESGPALVKPSQTVSLTCTVTGYSITNGN | 98 | DVVMTQTPLTLSVTIGQPASISCKSSQSLLDSD | 444 |
| | HWWNWIRQVSGSKLEWIGYITSSGRTDSNPSLK | | GKTYLNWLFQRPGHSPKRLIYLVSKLDSGVPNR | |
| | SRISITRDTSRNQLFLQLNSVITEDVATYFCAR | | FTGSGSGTDFTLKISRVEAEDLGVYYCWQGTHF | |
| | EGGWLPPVWGTGTTVTVSS | | PYTFGGGTKLEIK | |
| SCT-Ga116 | DVQLQESGPALVKPSQTVSLTCTVTGYSITNGN | 99 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLDSVTTEEIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga117 | DVQLQASGPALVKPSQTVSLTCTVTGYSITNGN | 100 | DVVMTQTPLTLSVTIGQPASISCKSSQSLLDSD | 381 |
| | HWWNWIRQVSGGKLEWIGYIRSSGSTDTNPSLK | | GKTYLNWLLQRPGQSPKRLIYLVSKLDSGVPDR | |
| | SRISITRDISKNELFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga118 | DVQLQESGPALVKPSQTVSLTCTVTGYSITNGN | 7 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga119 | DVQLQESGPALVKPSQTVSLTCTVTGYSITNGN | 101 | DVVMTQTPLTLSVTIGQPASISCKSSQSLLDSD | 381 |
| | HWWNWIRQVSGSKLEWIGYIRSSGRTDSNPSLK | | GKTYLNWLLQRPGQSPKRLIYLVSKLDSGVPDR | |
| | SRISITRDTSKNQLFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga120 | DVQLQESGPALVKPSQTVSLTCTVTGYSITNGN | 102 | DVVMTQTPLTLSVTIGQPASISCKSSQSLLDSD | 445 |
| | HWWNWIRQVSGRKLEWIGYITSSGRTDSNPSLR | | GKTYLNWLLQRPGQSPKRLIYLVSKLDSGVPDR | |
| | SRISITRDTSKNQLFLQLNSVTTENIATYYCAR | | FTGSGSGTDFTLKISRVEADDLGVYYCWQGTHF | |
| | EGGWLPPVWGTGTTVTVSS | | PYTFGGGTKLEIK | |
| SCT-Ga121 | DVQLQESGPALVKPSQTVSLTCTVTGYSITNGN | 27 | DVVMTQTPLTLSVTIGQPASISCKSSQSLLDGD | 446 |
| | HWWNWIRQVSGGKLEWIGYIRSSGSTDSNPSLK | | GRTYLNWLLQRPGQSPKRLIYLVSKLDSGVPDR | |
| | SRISITRDISKNELFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga122 | DVQLQESGPALVKPSQTVSLTCTVTGYSITNGN | 103 | DVVMTQTPLTLSVIIGQPASISCRSSQSLLDSD | 447 |
| | HWWNWVRQVSGSKLEWIGYITSSGRIDNNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTTLEIK | |
| SCT-Ga123 | DVQLQESGPALVKPSQTVSLTCTVTGYSITNGN | 84 | DVVMTQTPLTLSVTIGQPASISCKSSQSLLDSD | 385 |
| | HWWNWIRQVSGSKLEWIGYITSSGRTDSNPSLK | | GKTYLNWLLQRPGQSPKRLIYLVSKLDSGVPNR | |
| | SRISITRDTSKNQLFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga124 | DVQLQESGPALVKPSQTVSLTCTVTGYSITNGN | 104 | DVVMTQTPLTLSVAIGQPASISCKSSQSLLDSD | 382 |
| | HWWNWIRQVSGGKLEWIGYIRSSGSTDINPSLK | | GKTYLNWLLQRPGQSPKRLIYLVSKLDSGVPNR | |
| | SRISITRDISKNEFFLQLNSVTTEDIATYYCSR | | FTGSGSGTDFTLKISRVEAEDLGVYYCWQGTHF | |
| | EGGWLPPVWGTGTTVTVSS | | PYTFGGGTKLEIK | |
| SCT-Ga125 | DVQLQESGPALVKPSQTVSLTCTVTGYSITNGN | 7 | DVVMTQTPLTLSVAIGQPASISCKSSQSLLDSD | 448 |
| | HWWNWIRQVSGSKLEWIGYITSSGRTDSNPSLK | | GKTYLNWLLQRPGQSPKRLIYLVSKLDSGVPKR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga126 | DVQLQESGPALVKPSQTVSLTCTVTGYSITNGN | 105 | DVVMTQTPLTLSATIGESASIYCKSSQSLLDSD | 404 |
| | HWWNWIRQVSGSKLEWIGYIRSSGRTDSNPSLE | | GKTYLNWLLQRPGQSPKRLIYLVSKLDSGVPDR | |
| | SRISITRDTSKNQIFLQLNSVTTEDIATYYCTR | | FTGSGSGTDFTLKISRVEAEDLGVYYCWQGTHF | |
| | EGGRLPPVWGTGTTVTVSS | | PYTFGGGTKLEIK | |
| SCT-Ga127 | DVQLQESGPALVKPSQTVSLTCTVTGYSITNGN | 106 | DVVMTQTPLTLSVTIGQPASISCKSSQSLLDSD | 410 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIGTYYCAR | | FTGSGSGTDFTLKISRVEAEDLGVYYCWQGTHF | |
| | EGGWFPPVWGTGTTVTVSS | | PYTFGGGTKLEIQ | |
| SCT-Ga128 | DVQLQVSGPALVKPSQTVSLTCTVTGYSITNGN | 107 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGSTVTVSS | | PYTFGGGTKLEIK | |
| SCT-Ga129 | DVQLQESGPALVKPSQTVSLTCTVTGYSITTGN | 108 | DVVMTQTPLTLSVTIGQPASISCKSSQSLLDSD | 381 |
| | HWWNWIRQVSGGKLEWIGYIRSSGSTDSNPSLE | | GKTYLNWLLQRPGQSPKRLIYLVSKLDSGVPDR | |
| | SRISITRDISKNELFLQLNSVTTADMGTYYCTR | | FTGSGSGTDFTLKISRVEAEDLGVYYCWQGTHF | |
| | EGGLFPPVWGTGTTVTVSS | | PYTFGGGTKLEIK | |
| SCT-Ga130 | DVQLQASGPALVKPSQTVSLTCTVTGYSITNGN | 109 | DVVMTQTPLTLSVTIGQPASISCKSSQSLLDSD | 381 |
| | HWWNWIRQVSGSKLEWIGYITSNGRTDSNPSLK | | GKTYLNWLLQRPGQSPKRLIYLVSKLDSGVPDR | |
| | SRISITRDTSKNKLFLQLNSVTHEDIATYYCAR | | FTGSGSGTDFTLKTSRVEAEDLGVYYCWQGTHF | |
| | EGGWLPPVWGTGTTVTVSS | | PYTFGGGTKLEIK | |
| SCT-Ga131 | EVMLVESGGGLVKPGGSLKLSCAASGFTFSRYI | 110 | DIQMTQSPSSLSASLGERVSLTCRASQDIGSSL | 449 |
| | MSWVRQTPEKRLEWVATITGGGGNTYYPDSVKG | | NWLHQGPDGTIKRLIYATYSLDSSVPKRFSGSR | |
| | RFTISRDNARNTLYLQMSSLSSEDTALFYCARW | | SGSDYSLTISSLESEDFVDYCCLLYVNSPWTFG | |
| | LPTYYAMDYWGQGTSVTVSS | | GGTKLEIK | |
| SCT-Ga132 | DVQLQESGPALVKPSQTVSLTCTVTGYSITNGN | 111 | DVVMTQTPLTLSVTIGQPASISCKSSQSLLDSD | 450 |
| | HWWNWIRQVSGSKLEWIGYITSSGRTDSNPSLR | | GRTYLNWLLKRPGQSPKRLIYLVSKLDSGVPDR | |
| | SRISITRDTSKNQLFLQLNSVTTEEIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga133 | DVQLQESGPALVKPSQTVSLTCTVTGYSITNGN | 27 | DVVMTQTPLTLSVTIGQPASISCKSSQSLLDSD | 451 |
| | HWWNWIRQVSGGKLEWIGYIRSSGSTDSNPSLK | | GKTYLNWLLQRPGQSPKRLIYLVSKLDSGVPDR | |
| | SRISITRDISKNELFLQLNSVTTEDIATYYCTR | | FTGSGSGTDFTLKISRVEAEDLGVYYCWQGTHF | |
| | EGGWLPPVWGTGTTVTVSS | | PYTFGGGTKLERK | |
| SCT-Ga134 | QVQLQQSGPELVRPGASVTISCKASGYTFTDYY | 112 | DIQMNQSPSSLSASLGDTITITCHASQNINVWL | 452 |
| | INWVKQRPGQGLEWIGWIFPGSDNTYYNAKFKG | | SWYQQKPGNIPKLLIYTTSILHTGVPSRFSGSG | |
| | KATLTVDISSSTAYMLLSSLTSEDSAVYFCARY | | SGTGFALTISSLQPEDIATYYCQQGQSYPLTFG | |
| | GLGHYFDYWGQGTTLTVSS | | GGTKLEIK | |
| SCT-Ga135 | DIQLQESGPALVKPSQTVSLTCTVTGYSITNGN | 113 | DVVMTQTPLTLSVTIGQPASISCKSSQSLLDSD | 385 |
| | HWWNWIRQVSGSKLEWIGYITSSGRTDSNPSLK | | GKTYLNWLLQRPGQSPKRLIYLVSKLDSGVPNR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga136 | QVQLQQPGAELVKPGASVKMSCKASGYTFTSYW | 114 | DIVMTQSHKFMSTSVGDRVYITCKASQDVFTAV | 453 |
| | ITWVKQRPGQGLEWIGDFYPGGGSTNYNEKFKS | | AWFQQKPGQSPKLLIYSASYRYTGVPDRFTGSG | |
| | KATLTVDTSSNTAYMQLSSLTSEDSAVYYCARS | | SGTDFTFTISSVQAEDLAVYYCQQHYNTPLTFG | |
| | YSNYVRFAYWGQGTLVTVSA | | AGTKLELK | |
| SCT-Ga137 | QVQLQQSGPELVKPGASVKISCKASGHTFTDYY | 115 | EIVLTQSPALMAASPGEKVTITCSVSSSISSSN | 454 |
| | INWVKQRPGQGLEWIGWIFPGSGNTYFNEKFKG | | LHWYQQKSETSPKPWIYGTSNLASGVPIRFSGS | |
| | KATLTVDKSSSTVYMLLSSLTSEDSAVYFCAGY | | GSGTSYSLTISSMEAEDAATYYCQQWSSYPLTF | |
| | GLGHYFDNWGQGTTLTVSS | | GGGTKLEIK | |
| SCT-Ga138 | DVQLQESGPALVKPSQTVSLTCTVTGYSITNGN | 116 | DVVMTQTPLTLSVTIGQPASISCKSSQSLLDSD | 381 |
| | HWWNWIRQVSGSKLEWIGYIRSSGSTDSNPSLK | | GKTYLNWLLQRPGQSPKRLIYLVSKLDSGVPDR | |
| | SRISITRDTSKNQLFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga139 | DVHLQESGPALVKPSQTVSLTCTVTGYSITNGN | 117 | DVVMTQTPLTLSVTIGQPASISCKSSQSLLDSD | 385 |
| | HWWNWIRQVSGSKLEWIGYITSSGRTDSNPSLK | | GKTYLNWLLQRPGQSPKRLIYLVSKLDSGVPNR | |
| | SRISITRDTSKNQLFLQLNSMTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga140 | DVQLQESGPALVKPSQTVSLTCTVTGYSITNGN | 17 | DVVMTQTPLTLSVTIGQPASISCRSSQSLLDSD | 455 |
| | HWWNWIRQVSGSKLEWIGYITSSGRTDSNPSLQ | | GKTYLNWLLQRPGQSPKRLIYLVSKLDSGVPDR | |
| | SRISITRDTSKNQLFLQLNSVTTEDIATYYCAR | | FTGSGSGTDFTLNISRVEAEDLGVYYCWQGTHF | |
| | EGGWLPPVWGTGTTVTVSS | | PYTFGGGTKLEIK | |
| SCT-Ga141 | DVQLQESGPALVKPSQTVSLTCTVTGYSITNGN | 23 | DVVMTQTPLTLSVTIGQPASISCKSSQSLLDSD | 456 |
| | HWWNWIRQVSGGKLEWIGYIRSSGSTDFNPSLK | | GRTYLNWLLQRPGQSPKRLIYLVSKLDSGVPDR | |
| | SRISITRDISKNELFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga142 | DVQLQESGPALVKPSQTVSLTCTVTGYSITNGK | 118 | DVVMTQTPLTLSVTIGQPASISCKSSQSLLDSD | 456 |
| | HWWNWIRQVSGGKLEWIGYIRSSGSTDFNPSLK | | GRTYLNWLLQRPGQSPKRLIYLVSKLDSGVPDR | |
| | SRISITRDISKNELFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga143 | DVQLQESGPALVKPSQTVSLTCTVTGYSITNGN | 119 | DVVMTQTPLTLSVTIGQPASISCKSSQSLLDSD | 398 |
| | HWWNWIRQVSGSKLEWIGYITSSGRTDSNPSLR | | GKTYLNWLLQRPGQSPKRLIYLVSKLDSGVPDR | |
| | SRFSITRDTSKNKLFLQLNSVTNEDIATYYCAR | | FTGSGSGTDFTLKISRVEAEDLGLYYCWQGTHF | |
| | EGGWLPPVWGTGTTVTVSS | | PYTFGGGTKLEIK | |
| SCT-Ga144 | DIQLQESGPALVKPSQTVSLTCTVTGYSITNGN | 120 | DVVMTQTPLTLSVTIGQPASISCKSSQSLLDSD | 381 |
| | HWWNWIRQVSGSKLEWIGYISSSGRTDRNPSLK | | GKTYLNWLLQRPGQSPKRLIYLVSKLDSGVPDR | |
| | SRISITRDTSKNQVFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga145 | DVQLQVSGPALVKPSQTVSLTCTVTGYSITNGN | 121 | DVVMTQTPLTLSVTIGQPASISCKSSQSLLDSD | 381 |
| | HWWNWIRQVSGSKLEWIGYISSSGRTDSNPSLK | | GKTYLNWLLQRPGQSPKRLIYLVSKLDSGVPDR | |
| | SRISITRDTSKNQLFLQLNSVTTEDTVTYYCAR | | FTGSGSGTDFTLKISRVEAEDLGVYYCWQGTHF | |
| | EGGWLPPVWGTGTTVTVSS | | PYTFGGGTKLEIK | |
| SCT-Ga146 | DVQLQESGPALVKPSQTVSLTCTVTGYSITNGN | 84 | DVVMTQTALTLSVTIGQPASISCKSSQSLLDSD | 457 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCTR | | FTGSGSGTVFTLKTSRVEAEDLGVYYCWQGTHF | |
| | EGGWLPPVWGTGTTVTVSS | | PYTFGGGTKLEIK | |
| SCT-Ga147 | DVQLQESGPALVKPSQTVSLTCTVTGYSITNGN | 122 | DVVMTQTPLTLSVTIGQPASISCKSSQSLLDGD | 458 |
| | HWWNWIRQVSGSKLEWIGYITSSGRTDSNPSLS | | GKTYLNWLLQRPGQSPKRLIYLVSKLDSGVPDR | |
| | SRVSITRDTSKNQLFLQLNSVTTEDIATYYCTR | | FTGSGSGTDFTLKISRLEAEDLGVYYCWQGTHF | |
| | EGGWLPPVWGPGTTVTVSS | | PYTFGGGTKLEIK | |
| SCT-Ga148 | DVQLQESGPALVKPSQTVSLTCTVTGYSITNGN | 123 | DVVMTQTPLTLSVTIGQPASIFCKSSQSLLDRD | 406 |
| | HWWNWIRQVSGGKLEWIGYIRSSGSTDINPSLK | | GKTYLNWLLQRPGQSPKRLIYLVSKLDSGVPDR | |
| | SRISITRDISKNELFLQLNSVTTEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga149 | DVQLQESGPALVKPSQTVSLTCTVTGYSITNGN | 93 | DVVMTQTPLTLSVTIGQPASISCKSSQSLLDSD | 451 |
| | HWWNWIRQVSGRKLEWIGYITSSGRIDSNPSLK | | GKTYLNWLLQRPGQSPKRLIYLVSKLDSGVPDR | |
| | SRISITRDTSKNQLFLQLNSVTSEDLGTYYCAR | | FTGSGSGTDFTLKISRVEAEDLGVYYCWQGTHF | |
| | EGGWLPPVWGTGTTVTVSS | | PYTFGGGTKLERK | |
| SCT-Ga150 | DVQLQESGPALVKPSQTVSLTCTVTGYSITNGN | 58 | DVVMTQTPLTLSVTIGQPASISCKSGQSLLDSD | 389 |
| | HWWNWIRQVSGNKLEWIGYISSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga151 | DVQLQESGPALVKPSQTVSLTCTVTGYSITNGN | 124 | DVVMTQTPLTLSVTIGQPASISCKSSQSLLDSD | 381 |
| | HWWNWIRQVSGTKLEWIGYIRSSGGTDSNPSLK | | GKTYLNWLLQRPGQSPKRLIYLVSKLDSGVPDR | |
| | SRISITRDTSKNQLFLQLNSVTTEDIGTYYCSR | | FTGSGSGTDFTLKISRVEAEDLGVYYCWQGTHF | |
| | EGGWLPPVWGTGTTVTVSS | | PYTFGGGTKLEIK | |
| SCT-Ga152 | DVQLQESGPALVKPSQTVSLTCTVTGYSITNGN | 17 | DVVMTQTPLTLSVTIGQPASISCKSSQSLLDSD | 451 |
| | HWWNWIRQVSGSKLEWIGYITSSGRTDSNPSLQ | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLERK | |
| SCT-Ga153 | DVQLQESGPALVKPSQTVSLTCTVTGYSITNGN | 125 | DVVMTQTPLTLSVTIGQPASISCKSSQSLLDSD | 381 |
| | HWWNWIRQVSGGKLEWIGYIRSSGSTDSNPSLK | | GKTYLNWLLQRPGQSPKRLIYLVSKLDSGVPDR | |
| | SRISITRDISKNEIFLQLNSVNDEDIATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga154 | DVQLQESGPALVKPSQTVSLTCTVTGYSITNGN | 27 | DVVMTQTPLTLSVTIGQPASISCKSSQSLLDSD | 445 |
| | HWWNWIRQVSGGKLEWIGYIRSSGSTDSNPSLK | | GKTYLNWLLQRPGQSPKRLIYLVSKLDSGVPDR | |
| | SRISITRDISKNELFLQLNSVTTEDIATYYCTR | | FTGSGSGTDFTLKISRVEADDLGVYYCWQGTHF | |
| | EGGWLPPVWGTGTTVTVSS | | PYTFGGGTKLEIK | |
| SCT-Ga155 | DVQLQESGPALVKPSQTVSLTCTVTGYSITNGN | 126 | DVVMTQTPLTLSVTIGQPASISCKSSQSLLDSD | 381 |
| | HWWNWIRQVSGTNLEWIGYITSSGRTDSNPSLK | | GKTYLNWLLQRPGQSPKRLIYLVSKLDSGVPDR | |
| | SRISITRDTSKNQLFLQLNSVTTEDIATYYCSR | | FTGSGSGTDFTLKISRVEAEDLGVYYCWQGTHF | |
| | EGGWLPPVWGTGTTVTVSS | | PYTFGGGTKLEIK | |
| SCT-Ga156 | DVQLQESGPALVKPSQTVSLTCTVTGYSITNGH | 127 | DVVMTQTPLTLSVTLGQPASISCKSSQSLLDSD | 459 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLQISRVEAADLGVYYCWQGTHF | |
| | EGGWLPPVWGTGTTVTVSS | | PYTFGGGTKLEIK | |
| SCT-Ga157 | DVQLQESGPALVKPSQTVSLTCTVTGYSITNGN | 7 | DVVMTQTPLTLSVTIGQPASISCKSSQSLLDSD | 460 |
| | HWWNWIRQVSGSKLEWIGYITSSGRTDSNPSLK | | GKTYLNWLLQRPGQSPKRLIYLVSKLDSGVPNR | |
| | SRISITRDTSKNQLFLQLNSVITEDIATYYCAR | | FTGSGSGTEFTLKISRVEAEDLGVYYCWQGTHF | |
| | EGGWLPPVWGTGTTVTVSS | | PYTFGGGTKLEIK | |
| SCT-Ga158 | DVQLQESGPALVKPSQTVSLTCTVTGYSITNGN | 7 | DVVMTQTPLTLSVTIGQPASISCKSSQSLLDSD | 398 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVITEDIATYYCAR | | FTGSGSGTDFTLKISRVEAEDLGLYYCWQGTHF | |
| | EGGWLPPVWGTGTTVTVSS | | PYTFGGGTKLEIK | |
| SCT-Ga159 | DVQLQASGPALVKPSQTVSLTCTVTDYSITNGN | 128 | DVVMTQTPLTLSVTIGQPASISCRSSQSLLDGD | 461 |
| | HWWNWVRQVSGSKLEWIGYISSSGRIDSNPSLK | | GKTYLNWLLQRPGQSPKRLIYLVSKLDSGVPDR | |
| | SRISITRDTSKNQLFLQLTSVTTEDIATYYCAR | | FTGSGSGTDFTLKISRVEAEDLGVYYCWQGTHF | |
| | EGGLLPPVWGTGTTVTVSS | | PYTFGGGTKLEIK | |
| SCT-Ga160 | DVQLQESGPALVKPSQTVSLTCTVTGYSITNGN | 129 | DVVMTQTPLTLSVTIGQPASISCKSSQSLLDSD | 381 |
| | HWWNWIRQVAGSKLEWIGYISSSGRIDSNPSLK | | GKTYLNWLLQRPGQSPKRLIYLVSKLDSGVPDR | |
| | SRISITRDTSKNQLFLQLNSVTTEDIGTYYCAR | | FTGSGSGTDFTLKISRVEAEDLGVYYCWQGTHF | |
| | EGGWLPPVWGTGTTVTVSS | | PYTFGGGTKLEIK | |
| SCT-Ga161 | DVQLQASGPALVKPYQTVSLTCTVTGYSITNGN | 130 | DVVMTQTPLTLSVTIGQPASISCKSSQSLLDSD | 462 |
| | HWWNWIRQVSGSKLEWIGYITSSGRTDSNPSLR | | GKTYLNWLLQRPGQSPKRLIYLVSKLDSGVPDR | |
| | SRISITRDTSKNQLFLQLNSVTTEDIATYYCTR | | FTGSGSGTDFTLKISRLEAEDLGVYYCWQGTHF | |
| | EGGWFPPVWGTGTTVTVSS | | PYTFGGGTKLEIK | |
| SCT-Ga162 | DVQLQESGPALVKPSQTVSLTCTVTGYSITNGN | 84 | DVVMTQTPLTLSVTIGQPASISCKSSQSLLDSD | 398 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTEDIATYYCTR | | FTGSGSGTDFTLKTSRVEAEDLGLYYCWQGTHF | |
| | EGGWLPPVWGTGTTVTVSS | | PYTFGGGTKLEIK | |
| SCT-Ga163 | DVQLQESGPALVKPSQTVSLTCTVTGYSITNGN | 131 | DVVMTQTPLTLSVTIGQPASISCKSSQSLLDSD | 381 |
| | HWWNWIRQVSGSKLEWIGYITSSGRTDSNPSLK | | GKTYLNWLLQRPGQSPKRLIYLVSKLDSGVPDR | |
| | SRISITRDTSKNQLFLQLNSVTTDDVATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga164 | DVQLQESGPALVKPSQTVSLTCTVTGYSITNGN | 132 | DVVMTQTPLTLSVTIGQPASISCKSSQSLLDSD | 381 |
| | HWWNWIRQVSGGKLEWIGYIRSSGSTDSNPSLK | | GKTYLNWLLQRPGQSPKRLIYLVSKLDSGVPDR | |
| | SRISITRDISKNELFLQLNSVTIEDTATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga165 | DVQLQESGPALVKPSQTVSLTCTVTGYSITNGN | 133 | DVVMTQTPLTLSVTIGQPASISCKSSQSLLDSD | 381 |
| | HWWNWIRQVSGTKLEWIGYIRSSGSTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga166 | DVQLQASGPALVKPSQTVSLTCTVTGYSITNGN | 67 | DVVMTQTPLTLSVAIGQPASISCKSSQSLLDSD | 382 |
| | HWWNWIRQVSGSKLEWIGYITSSGRTDSNPSLK | | GKTYLNWLLQRPGQSPKRLIYLVSKLDSGVPNR | |
| | SRISITRDTSKNQLFLQLNSVITEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga167 | QIELVQSGPELKKPGETVKISCKASGYTFTTYG | 134 | DVVMTQTPLSLPVSLGDQASISCRSSQSLVHSN | 396 |
| | MNWVKQAPGKGLKWMGWINTYSGVPTYADDFKG | | GNTYLHWYLQKPGQSPKLLIYKVSNRFSGVPDR | |
| | RFAFSLETSASTAYLQINNLKNEDTATYFCARY | | FSGSGSGTDFTLKISRVEAEDLGVYFCSQSTHV | |
| | DYDVGFAYWGQGTLVTVSA | | PTFGGGTKLEIK | |
| SCT-Ga168 | DVQLQASGPALVKPYQTVSLTCTVTGYSITNGN | 51 | DVVMTQTPLTLSVTIGQPASISCKSSQSLLDSD | 381 |
| | HWWMWTRQVSGSKLEWIGYIRSSGSTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga169 | DVQLQASGPALVKPYQTVSLTCTVTGYSITNGN | 51 | DVVMTQTPFTLSVTIGQPASISCKSSQSLLDSD | 463 |
| | HWWNWIRQVSGSKLEWIGYIRSSGSTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga170 | DVQLQESGPALVKPSQTVSLTCTVTGYSITNGN | 135 | DVVMTQTPLTLSVTIGQPASISCKSSQSLLDSD | 381 |
| | HWWNWIRQVSGGKLEWIGYIRSSGSTDSNPSLK | | GKTYLNWLLQRPGQSPKRLIYLVSKLDSGVPDR | |
| | SRISITRDIYKNELFLQLNSVNTEDTATYYCTR | | FTGSGSGTDFTLKISRVEAEDLGVYYCWQGTHF | |
| | EGGWLPPVWGTGTTVTVSS | | PYTFGGGTKLEIK | |
| SCT-Ga171 | DVQLQESGPALVKPSQTVSLTCTVTGYSITNGN | 102 | DVVMTQTPLTLSVTIGQPASISCKSSQSLLDSD | 381 |
| | HWWNWIRQVSGRKLEWIGYITSSGRTDSNPSLR | | GKTYLNWLLQRPGQSPKRLIYLVSKLDSGVPDR | |
| | SRISITRDTSKNQLFLQLNSVTTENIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga172 | DVQLQESGPALVKPSQTVSLTCTVTGYSITNGN | 136 | DVVMTQTPLTLSVTIGQPASISCKSSQSLLDSD | 381 |
| | HWWNWIRQVSGSKLEWIGYISSSGRTDSNPSLK | | GKTYLNWLLQRPGQSPKRLIYLVSKLDSGVPDR | |
| | SRISITRDTSKNQLFLQLNSVTTEDIATYYCAR | | FTGSGSGTDFTLKISRVEAEDLGVYYCWQGTHF | |
| | EGGWLPPVWGTGTTVTVSS | | PYTFGGGTKLEIK | |
| SCT-Ga173 | DVQLQESGPALVKPSQTVSLTCTVTGYSITNGN | 137 | DVVMTQTPLTLSVTIGQPASISCKSSQSLLDSD | 398 |
| | HWWNWIRQVSGSKLEWIGYISSSGRTDSNPSLR | | GKTYLNWLLQRPGQSPKRLIYLVSKLDSGVPDR | |
| | SRISITRDTSKNKLFLQLNSVTNEDIATYYCAR | | FTGSGSGTDFTLKISRVEAEDLGLYYCWQGTHF | |
| | EGGWLPPVWGTGTTVTVSS | | PYTFGGGTKLEIK | |
| SCT-Ga174 | DVQLQASGPALVKPYQTVSLTCTVTGYSITNGN | 138 | DVVMTQTPLTLSVTIGQPASISCKSSQSLLDSD | 462 |
| | HWWNWIRQVSGSKLEWIGYISSSGRTDSNPSLR | | GKTYLNWLLQRPGQSPKRLIYLVSKLDSGVPDR | |
| | SRISITRDTSKNQLFLQLNSVTTEDIATYYCAR | | FTGSGSGTDFTLKISRLEAEDLGVYYCWQGTHF | |
| | EGGWLPPVWGTGTTVTVSS | | PYTFGGGTKLEIK | |
| SCT-Ga175 | EVQLQQSGPELVKPGASMKISCKASGYSFTGYI | 139 | DVVMTQTPLSLPVSFGDQVSISCRSSQSLANSY | 464 |
| | LNWVKQSHGKNLEWIGLIVPYTGGTSYNQDFQG | | GNTYLSWYLHKPGQSPQLLIYGISNRFSGVPDR | |
| | KATLTVDRSSSTAYLELLSLTSEDSAVYYCARQ | | FSGSGSGTDFTLKISTIKPEDLGMYYCLQGTHQ | |
| | LRGYFAMDYWGQGTSVTVSS | | PWTFGGGTKLEIK | |
| SCT-Ga176 | QVQLKESGPGLVAPSQSLSITCTVSGFSLTRHG | 140 | DIQMTQSPSSLSASLGERVSLTCRASQEISGYL | 465 |
| | IHWVRQPPGKGLEWLGVIWPGGNTKYNSALMSR | | SWLQQKPDGTIKRLIYAASTLDSGVPKRFSGSR | |
| | LSISKDNSKSQVFLKMNSLQTDDTAMYYCARSG | | SGSDYSLTISSLESEDFADYYCLQYASYPRTFG | |
| | GYGNFAPYYFDQWGQGTTLTVSS | | GGTKLEIK | |
| SCT-Ga177 | QIQLVQSGPELKKPGETVKISCKASGYTFTNYG | 141 | DIVMTQAAFSNPVTLGTSASISCRSSKNLLHSN | 466 |
| | MNWVKKAPGKGLKWMGWINTYTGESTYADDFKG | | GITYLYWYLQRPGQSPQVLIYRMSNLASGVPNR | |
| | RFAFSSETSASTVYLQINNLKNEDTATYFCARD | | FSGSGSGTDFTLRISRVEAEDVGVYYCGQLLEL | |
| | SFAYWGQGTLVTVSA | | PYTFGGGTKLEIK | |
| SCT-Ga178 | EVQLQQSGPELEKPGSSVKISCKASGYSFTDYN | 142 | EIVLTQSPAIMSASPGEKVTMTCSASSSVSSSY | 467 |
| | MHWVKQSNGKSLEWIGDIDVYYSGTSYNQKFKG | | FHWYQQKSGSSPRLLIYDTSNLASGVPVRFSGS | |
| | KATLTVDKSSSTAYMQLKSLTSEDSAVYFCARS | | GSGTSYSLTISRMEAEDAATYYCQQWSSYPLTF | |
| | LLRWYFDVWGAGTTVTVSS | | GAGTKLELK | |
| SCT-Ga179 | QITLKESGPGIVQPSQPFRLTCTFSGFSLSTSG | 143 | DVLMTQTPLSLPVSLGDQASISCRSSQSIVHSD | 468 |
| | IGVTWIRQPSGRGLEWLATIWWDDDNRYNPSLK | | GNTYLEWYLQKPGQSPKLLIYKVSNRFSGVPDR | |
| | SRLTVSKDTSNNQAFLKIITVETADTAIYYCAQ | | FSGSGSGTDFTLKISRVEAEDLGDYYCFQSSHV | |
| | SGGITLMDYWGQGTSVTVSS | | PGTFGGGTKLEIK | |
| SCT-Ga180 | EVQLQQSGPELVKPGASMKISCKASGYSFTGYT | 144 | DVVVTQTPLSLPVSFGDQVSISCRSSQSLANSY | 469 |
| | MNWVKQSHGKNLEWIGLIVPSNGGTSYNQKFKG | | GTTYLSWYLHKPGQSPQLLIYGISNRFSGVPDR | |
| | KATLTVDKSSSTAYMELLSLTSEDSAVYYCARS | | FSGSGSGTDFTLKISTIKPEDLGMYYCLQGTHQ | |
| | LRFSFDYWGQGTTLTVSS | | PPTFGGGTKLEIK | |
| SCT-Ga181 | QIQLVQSGPELKKPGETVKISCKASGYTFTDYS | 145 | QIVLSQSPVILSASPGEKVTMTCRASSSVSDMH | 470 |
| | MHWVKQAPGKGLKWMGWINTETGVPTYADDFKG | | WYQQKPGSSPKPWIYATSNLASGVPARFSGSGS | |
| | RFAFSLETSASTAFLQINNLKNEDTATYFCART | | GTSYSFTISRVEAEDAATYYCQQWSFNPQTFGG | |
| | DYGNPYWYFDVWGAGTTVTVSS | | GTKLEIK | |
| SCT-Ga182 | EVQLQQSGPELVKPGASVKISCKASGYSFTGYT | 146 | DVVVTQTPLSLPVSFGDQVSISCKSSQSLTNSY | 471 |
| | MNWVKQSHGKNLEWIGLIIPYNGGTSYNQKFKD | | GDTYLSWYLHKPGQSPQLLIYGISNRFSGVPDR | |
| | KASLTFDKSSSTAYMELLSLTSEDSAVYYCARG | | FSGSGSGTNFTLKISTIKPEDLGVYSCLQGTHQ | |
| | FRGYFAVDYWGQGTSVTVSS | | PWTFGGGTKLEIK | |
| SCT-Ga183 | QVQLQQPGAELVRPGVSVKLSCKASGYTFTSYW | 147 | DIQMTQSPSSLSASLGERVSLTCRASQDIGSSL | 472 |
| | MHWIKQRPEQGLERIGEIDPTNDYSNYNEKFKS | | NWLQQEPDGTIKRLIYATSSLDSGVPKRFSGSR | |
| | KATLTVDKSSSTAYMHLTSLTSEDSAVYYCARS | | SGSDYSLTISSLESEDFVDYYCLQCATSWTFGG | |
| | RGKDWYFDVWGAGTTVTVSS | | GTKLEIK | |
| SCT-Ga184 | QAYLQQSGAELVRSGASVKVSCKASGYTFTSFN | 148 | DIVMTQSQKFMSTSVGDRVSVTCKASQNVYTNV | 473 |
| | MHWVKQTPGQGLEWIGYFFPGNGATNYNQKFKG | | AWYQQKPGQSPKVLIYSASHRYSGVPDRITGSG | |
| | KATLTADTSSSTAYMQISSLTSEDSAVYFCARE | | SGTDFTLTISNVQSEDLADYFCQQHNRYPYTFG | |
| | GWLSLGYWGQGTTLTVSS | | GGTKLEIK | |
| SCT-Ga185 | QVQLQQSGGDLMKPGASVKISCKATGYTFSNYW | 149 | DIQMTQTTSSLSASLGDRVTISCSASQDISNYL | 474 |
| | IEWVKQRPGHGLEWIGEIFPGGGSAYYNEKFRG | | NWYQQKPDGTVKLLLYYTSSLHSGVPSRFSGSG | |
| | KATFTADKSSDTAYMQLSSLTSEDSAVYYCARQ | | SGTDYSLTISNLEPEDIATYYCQQYSKLPYTFG | |
| | KYGNYYYFDYWGQGTTLTVSS | | GGTNLEMK | |
| SCT-Ga186 | EVQLQQSGPELVKPGASMKISCKASGYSFTGYT | 150 | DVVVTQTPLSLPVSFGDQVSISCRSSQSLANSY | 475 |
| | MNWVKQSHGKNLEWIGLFIPYNGGTSYNQNFRG | | GNTYLSWYLHKPGQSPQLLIYGISNRFSGVPDR | |
| | KATLTVDKSSSTAYMELLSLTSEDSAVYYCARG | | FSGSGSGTDFTLKISTVKPEDLGMYYCLQGTHQ | |
| | LRGFFSMDYWGQGTSVTVSS | | PWTFGGGTKLEIK | |
| SCT-Ga187 | EVQLQQSGPELVKPGASMKTSCKASGYSFTGYT | 151 | DVVVTQTPLSLPVSFGDQVSISCRSSQSLTNSY | 476 |
| | MNWVKQSHGKNLEWIGLIIPYNGGTSYNQKFKD | | GNTYLSWYLHKPGQSPQLLIYGISNRFSGVPDR | |
| | KATLTIDKSSSTAYMELLSLTSEDSAVYYCARG | | FSGSGSGTNFTLKISTIKPEDLGMYYCLQGTHQ | |
| | FRGYFAVDYWGQGTSVTVSS | | PWTFGGGTKLEIK | |
| SCT-Ga188 | QVQLKESGPDLVAPSQSLSITCTVSGFSLTSYG | 152 | DVVMTQSPLSLPVSFGDQVSISCRSSQNLTNSY | 477 |
| | VHWVRQPPGKGLEWLVVIWSDGSTTYNSALKSR | | GNTYLSWYLHKPGQSPQLLIYGISNRFSGVPDR | |
| | LSISKDNSKSQVFLKMNSLQTDDTAMYYCARQI | | FSGSGSGTDFTLKISTIKPEDLGMYYCLQGTHQ | |
| | YYDYGWFAYWGQGTLVTVSA | | PWTFGGGTKLEIK | |
| SCT-Ga189 | EVHLQQSGPELVKPGASMKISCKASGYSFTDYI | 153 | DVVVTQTPLSLPVSFGDQVSISCRSSQSLANSY | 478 |
| | MNWVKQSHGKNLEWIGLIIPYNGGTSYNQKFKG | | GSTFLSWYLHKPGQSPQLLIYGISNRFSGVPDR | |
| | KATLTVDKSSSTAYMEFLSLTSEDSAVYYCARG | | FSGSGSGTDFTLKISTVKPEDLGMYYCLQGTHQ | |
| | LRGYYAMDYWGQGTSVTVSS | | PWTFGGGTKLEIK | |
| SCT-Ga190 | QVQLEQSGGEVMKPGASVKISCKATGYTFSNYW | 154 | DIQMTQTTSSLSASLGDRVTISCSASQGISNYL | 479 |
| | IEWVKQRPGHGPEWIGEILPGSGSAYYNEKFRG | | NWYQQKPDGTVTLLIYYTSSLHSGVPSRFSGSG | |
| | KATFTADKSSNTAYMQLSSLTSEDSAVYYCARQ | | SGTDYSLTISNLEPEDIATYYCQHYSKLPYTFG | |
| | KYGNYYYFDYWGQGTTLTVSS | | GGTKLEIK | |
| SCT-Ga191 | QIQLVQSGPELRKPGETVKISCKASGYTFTDYS | 155 | QIVLSQSPAILSASPGEKVTMTCRASSSVNYIH | 480 |
| | MHWVKQAPGKGLKWMGWINTETGEPTYADDFKG | | WYQQKPGSSPKPWIYATSNLASGVPARFSGSGS | |
| | RFAFSLETSASTAHLQINNLKNEDTATYFCTRT | | GTSYSLTISRVEAEDAATYYCQQWSFNPQTFGG | |
| | DYGNPYWYFDVWGAGTTVTVSS | | GTKLEIK | |
| SCT-Ga192 | EVQLQQSGPELVKPGASVKISCKASGYSFTGYT | 156 | DVVVTQTPLSLPVSFGDQVSISCRSSQSLTNSY | 481 |
| | MNWVKQSHGKNLEWIGLIIPYNGGTSYNQKFKD | | GDTYLSWYLHKPGQSPQLLIYGISNRFSGVPDR | |
| | KATLTIDKSSSTAYMELLSLTSEDSAVYYCARG | | FSGSGSGTNFTLKISTIKPEDLGVYYCLQGTHQ | |
| | FRGYFAVDYWGQGTSVTVSS | | PWTFGGGTKLEIK | |
| SCT-Ga193 | EVQLQQSGPELVKPGASMKISCKASGYSFTGYT | 157 | DVVVTQTPLSLPVSFGDQVSISCRSSQSLANSY | 482 |
| | MNWVKQSHGKNLEWIGLISPYNGGTSYNQKFKG | | GNTYLSWYLHKPGQSPQLLIYGISNRFSGVPDR | |
| | KATLTVDKSSSTVYMELLSLTSEDSAVYYCARS | | FSGSGSGTDFTLKISTIKPEDLGMYYCLQGTHQ | |
| | LRFSFDYWGQGTTLTVSS | | PPTFGGGTKLEIK | |
| SCT-Ga194 | EVQLQQSGPELVKPGASMKTSCKASGYSFTGYT | 158 | DVVVTQTPLSLPVSFGDQVSISCRSSQSLANSY | 483 |
| | MNWVKQSHGKNLEWIGLINPYNGGTSYNQKFKG | | GNTYLSWYLHKPGQSPQLLIYGISNRFSGVPDR | |
| | KATLTVDKSSSTAYMELLSLTSEDSAVYYCARR | | FSGSGSGTDFTLKISTIKPEDLGMYYCLQGTHQ | |
| | NYGYYFDYWGQGTTLTVSS | | PLTFGAGTKLELK | |
| SCT-Ga195 | EVKLVESGGGLVKPGGSLKLSCAASGFDFSRYD | 159 | DIQMNQSPSSLSASLGDTITTTCHASQNINVWL | 484 |
| | MSWVRQTPEKRLEWAATTSSGGSYTYQPDSVKG | | SWYQQKPGNIPKLLIYKASNLHTGVPSRFSGSG | |
| | RFTISRDNARNTLYLQMSSLRSEDTALYYCARQ | | SGTGFTLTISSLQPEDIATYYCQQGQSYPWTFG | |
| | DYRYPYTMDYWGQGTSVTVSS | | GGTTLEIK | |
| SCT-Ga196 | QVTLKESGPGILQPSQTLSLTCSFSGFSLSTYG | 160 | QIVLTQSPALMSASPGEKVTMTCSASSSVSYMY | 485 |
| | IGVGWIRQPSGKGLEWLAHIWWNDNKYYNTALK | | WYQQKPRSSPKPWIYLTSNLASGVPARFSGSGS | |
| | SRLTISKDTSNNQVFLKIASVDTAETATYYCAR | | GTSYSLTISSMEAEDAATYYCQQWSSNPPTFGG | |
| | TGSGKFYFDYWGQGTTLTVSS | | GTKLEIK | |
| SCT-Ga197 | QVQLKESGPGLVAPSQSLSITCTVSGFSLTRHG | 161 | NIMMTQSPSSLAVSAGEKVTMSCKSSQSVLYSS | 486 |
| | VHWVRQPPGKGLEWLGVTWPGGNTKFNSTLMSR | | NQKNFLAWYQQKPGQSPKLLIYWASTRESGVPD | |
| | LSISKDNSKSQVFLKMNTLQTDDTAMYYCARSG | | RFTGSGSGTDFTLTISSVQAEDLAVYYCHQYLS | |
| | GYGNFAPYYFDQWGQGTTLTVSS | | SYTFGGGTKLEIK | |
| SCT-Ga198 | QVQLQQSGAELVKPGASVKMSCKASGYTFTNYW | 162 | DIKMTQSPSSMYASLGERVTITCKASQDIKNYL | 487 |
| | MHWVKLRPGQGLEWIGVIDPSDSFPTYNQKFKG | | SWYPRKPWTSPKTLIYYATALADGVPSGFSGSG | |
| | KATLTVDTSSSTAYMQLSSLTSEDSAVYYCTRG | | SGQDYSLTISSLESDDTATYYCLHHGESPPTFG | |
| | PRGYGDYWGQGTSVTVSS | | GGTKLEIK | |
| SCT-Ga199 | QVQLQQPGSELVRPGASVKLSCKASGYTFTSYW | 163 | DWLTQSPATLSVTPGDSVSLSCRASQNIRNSL | 488 |
| | IHWVKQRHGQGLEWIGNIFPGSGTSNYDEKFNN | | HWFQQKSHESPRLLINYASQSISGIPSRFRGSG | |
| | KGTLTVDTSSSTSYMHLSSLTSEDSAVYYCARE | | SGTDFTLSINSLETEDFGMYFCLQSNSWPYTFG | |
| | GLYAFDYWGQGTTLTVSS | | GGTKLEIK | |
| SCT-Ga200 | QAYLQQSGAELVRSGASVKMSCKASGYTFTSYN | 164 | DIVLTQSPAIMAASLGQKVTMTCSASSSVNSTY | 489 |
| | MHWVKQTPGQGLEWIGKIFPGNGFTDYNQKFRD | | FHWYQQKSGASPKTMIHRSSNLASGVPAHFSGS | |
| | KATLTADTSSSTAYMQISSLTSEDSAVYFCTRE | | GSGTSDSLTISSVEADDDATYYCHQWSGYPFTF | |
| | GWLSLGYWGQGTTLTVSS | | GAGTKLELK | |
| SCT-Ga201 | EVQLQQSGPELVKTGASVKISCKASGYAFTGYY | 165 | DIVMTQSHKFMSTSVGDRVSITCKASQDVSTDV | 490 |
| | IHWVKQSHGKSLEWIGHITCYNGATNYNQNFKG | | AWYQQRPGQSPKLLTYSASYRYTGVPDRFTGSG | |
| | KATFTVDTSSSTAYMQFNSLTSEDSAVYYCARS | | SGTDFTFTINNVQAEDLAVYYCQQHYSTPYTFG | |
| | NYRPWYFDVWGAGTTVTVSS | | GGTKLEIK | |
| SCT-Ga202 | QVQLQQSGGDLMKPGASVKISCKATGYTFSNYW | 166 | DIQMTQTTSSLSASLGDRVTISCSASQDISNYL | 474 |
| | IEWVKQRPGHGLEWIGEIFPGGGSAYYNEKFRG | | NWYQQKPDGTVKLLLYYTSSLHSGVPSRFSGSG | |
| | KATFTADKSSDTAYMQLSSLTSEDSAVYFCARQ | | SGTDYSLTISNLEPEDIATYYCQQYSKLPYTFG | |
| | KYGNYYYFDYWGQGTTLTVSS | | GGTNLEMK | |
| SCT-Ga203 | QVQLQQPGSELVRPGASVKLSCKASGYTFTSFW | 167 | DIVLTQSPATLSVTPGDSVSLSCRASQSVRNNL | 491 |
| | IHWVKQRPGQGLEWIGNIFPGGGDPNYDEKFRT | | HWFQQKSHESPRLLIKYASQSFSGIPSRFSGSG | |
| | KGTLTVDTSSSTAYMHLSSLTSEDSAVYFCARE | | SGTDFTLSTNSLETEDFGMYFCQQSNSWPYTFG | |
| | GLYAFDYWGQGTTLTVSS | | GGTKLEIK | |
| SCT-Ga204 | QVQLQQSGGEVMKPGASVKMSCKASGYTFSNYW | 168 | DIQMTQTTSSLSASLGDRVTISCSASQDISNYL | 492 |
| | IEWLRQRPGHGLEWIGEIFPGSDSSYYNERFKG | | NWYQQKPDGTVKLLIYYTSTLHSGVPSRFSGSG | |
| | KATFTTDKSSDTAYMQLSSLTSEDSAVYYCARQ | | SGTDYSLTISNLEPEDIATYYCQQYSKLPYTFG | |
| | KYGNFYYFDYWGQGTTLKVSS | | GGTKLEIK | |
| SCT-Ga205 | QVQLKESGPGLVAPSQSLSITCTVSGFSLTRMG | 169 | NIMMTQSPSSLAVSAGEKVTMSCKSSQSVLYSS | 493 |
| | VHWVRQPPGKGLEWLGVIWPGGSTKYNSALMSR | | NQKNYLAWYQQKPGQSPKLLIYWASTRESGVPD | |
| | LSINKDNSKSQVFLKMNSLQTADTAMYYCARSG | | RFTGSGSGTDFTLTISNVQAEDLAVYYCHQYLS | |
| | GYGNFAPYYFDQWGQGTTLTVSS | | SYTFGGGTTLEIK | |
| SCT-Ga206 | QAYLQQSGAELVRSGASVKMSCKASGYTFTSYN | 170 | DIVMTQSQKFMSTSIGDRVSVTCKASQNVGTNV | 494 |
| | MHWVKQTPGQGLEWTGKTFPGNGFTDYNQNFRD | | AWYQQKPGQSPKALTYSASYRYSGVPDRFTGSG | |
| | KATLTADTSSSTAYMQISSLTSEDSAVYFCTRE | | SGTDFTLTISNVQSEDLADYFCQQHNSYPYTFG | |
| | GWLSLGYWGQGTTLTVSS | | GGTKLEIK | |
| SCT-Ga207 | QVQLQQPGSELVRPGASVKLSCKASGYTFTSYW | 171 | DIVMSQSPSSLAVSAGEKVTMSCKSSQSLLNSR | 495 |
| | MHWVKQRHGQGLEWIGNIFPGSGTTNYDEKFKN | | TRKNSLAWYQQKPGQSPKLLVYWASTRESGVPD | |
| | KGTLTVDTSSSTAYMHLSSLTSEDSAVYYCARE | | RFTGSGSGTDFTLTISSVQAEDLAVYYCKQSFN | |
| | GLYAFDYWGQGTTLTVSS | | LYTFGGGTKLEIK | |
| SCT-Ga208 | QVQLQQPGSELVRPGASVKLSCKASGYTFTSYW | 172 | DIVLTQSPATLSVTPGDSVSLSCRASQSIRNSL | 496 |
| | MHWVKQRHGQGLEWIGNIFPGSGTNNYDEKFKN | | HWFQQKSHESPRLLINYASQSISGIPSRFRGSG | |
| | KGTLTVDTSSSTAYMHLSSLTSEDSAVYYCARE | | SGTDFTLSINSLETEDFGMYFCQQSYSWPYTFG | |
| | GLYAFDYWGQGTTLTVSS | | GGTRLEIK | |
| SCT-Ga209 | EVQLQQSGPELVKPGASMKTSCKASGYSFTGYT | 173 | DVVMTQTPLSLPVSFGDQVSISCRSSQSLANSY | 497 |
| | MNWVKQSHGKNLEWLGLIFPYNSATSYNQKFTG | | GNTYLSWYLHKPGQSPHLLIYGISNRFSGVPDR | |
| | KATITVDKSSNTTYMELLSLTSEDSAVYYCARG | | FSGSGSGTDFTLKISTIKPEDLGMYYCLQGTHQ | |
| | LRGFFALDYWGQGTSVTVSS | | PWTFGGGTKLEIK | |
| SCT-Ga210 | QVQLQQPGSELVRPGASVKLSCKASGYTFTSYW | 174 | DIVLTQSPATLSVTPGDSVSLSCRASQNIRNSL | 498 |
| | IHWVKQRHGQGLEWIGNIFPGSGTSNYDEKFKN | | HWFQQKSHESPRLLINYASQSISGIPSRFRGSG | |
| | KGTLTVDTSSSTAYMHLSSLTSEDSAVYYCARE | | SGTDFTLSINSLETEDFGMYFCLQSNSWPYTFG | |
| | GLYAFDYWGQGTTLTVSS | | GGTKLEIK | |
| SCT-Ga211 | QVQLKESGPGLVAPSQSLSITCTVSGFSLTRHG | 175 | NIMMTQSPSSLAVSAGEKVTMSCKSSQSVLYSS | 486 |
| | VHWVRQPPGKGLEWLGVIWPGGNTKFNSTLMSR | | NQKNFLAWYQQKPGQSPKLLIYWASTRESGVPD | |
| | LSISKDNSKSQVFLKMNTLQTDDTAMYYCASSG | | RFTGSGSGTDFTLTISSVQAEDLAVYYCHQYLS | |
| | GYGNFAPYYFDQWGQGTTLTVSS | | SYTFGGGTKLEIK | |
| SCT-Ga212 | EVKLVESGGGLVKPGGSLKLSCAASGFAFSTYD | 176 | DIVMTQSPATLSVTPGDRVSLSCRASQSISDYL | 499 |
| | MSWVRQTPEKRLEWVATTSSGGSYTYYPDSVKG | | HWYQQKSHESPRLLIKYASQSISGIPSRFSGSG | |
| | RFTISRDNARNTLYLQMSSLRSEDTALYYCARQ | | SGSDFTLSINSVEPEDVGVYYCQDGNTFPPTFG | |
| | DYRYPYTMDYWGQGTSVTVSS | | GGTKLEIK | |
| SCT-Ga213 | EVQLQQSGPELEKPGSSVKISCKASGYSFTDYN | 177 | EIVLTQSPAIMSASPGEKVTVTCSAGSSVSSSY | 500 |
| | MHWVKQSNGKSLEWTGDTDVYYSGTSYNQKFKG | | LHWYQQKSGSSPRLLIYDTSKLASGVPVRFSGS | |
| | KATLTVDKSSSTTYMELKSLTSEDSAVYFCARS | | GSGTSYALTISRMEAEDAATYYCQQWSSYPLTF | |
| | SLRWYFDLWGAGTTVTVSS | | GAGTKLELK | |
| SCT-Ga214 | QVQLQQSGGEVMKPGASVKISCKATGYTFSNYW | 178 | DIQMTQTTSSLSASLGDRVTISCSASQGISNYL | 479 |
| | IEWVKQRPGHGLEWIGEILPVSGSAYYNEKFRG | | NWYQQKPDGTVTLLIYYTSSLHSGVPSRFSGSG | |
| | KATFTADKSSNTAYMQLSSLTSEDSAVYYCARQ | | SGTDYSLTISNLEPEDIATYYCQHYSKLPYTFG | |
| | KYGNYYYFDYWGQGTTLTVSS | | GGTKLEIK | |
| SCT-Ga215 | QTFLQQSGAELVRSGASVKMSCRASGYTFSSHN | 179 | DIVMTQSQKFMSTSTGDRVSVTCKASQNWTNV | 501 |
| | MHWIKQTPGQGLEWIGKIFPGNGFTDYNQKFRD | | AWYQQKPGQSPKALIYSASYRYSGVPDRFTGSG | |
| | KATLTADTSSSTAYMQIGSLTSEDSAVYFCTRE | | SGTDFTLTISNVQSEDLADYFCQQHNSYPYTFG | |
| | GWLSLGYWGLGTTLTVSS | | GGTRLEIK | |
| SCT-Ga216 | EVQLQQSGPELVKPGASMKISCKASGYSFTDYT | 180 | DVVVTQTPLSLPVSFGDQVSISCRSSQSLANSY | 502 |
| | MNWVKQSHGKNLEWLGLIFPYNGATSYNQKFKG | | GNTYLSWYLHKPGQSPHLLIYGISNRFSGVPDR | |
| | KATITVDKSSSTAYMELLSLTSEDSAVYYCARG | | FSGSGSGTDFTLKISTIEPEDLGMYYCLQGTHQ | |
| | LRGFFALDYWGQGTSVTVSS | | PWTFGGGTKLEIK | |
| SCT-Ga217 | EVQLQQSGPELVKPGASMKISCKASGYSFTGYI | 181 | DVVVTQTPLSLPVSFGDQVSISCRSSQSLANSY | 503 |
| | MNWVKQSHGKNLEWIGLIVPNNGGTSYNQKFKG | | GTTYLSWYLHKPGQSPQLLIYGISNRFSGVPDR | |
| | KATLTVDKSSNTAYMELLSLTSEDSAVYYCARS | | FSGSGSGTDFTLKISTVKPEDLGMYYCLQNTHQ | |
| | LRFSFDYWGQGTTLTVSS | | PPTFGGGTKLEIK | |
| SCT-Ga218 | QITLKESGPGTVQPSQPFRLTCTFSGFSLSTSG | 182 | DVLMTQTPLSLPVSLGDQASISCRSSQSIVYSN | 504 |
| | IGVTWIRQPSGKGLEWLATIWWDDDTRYNPSLK | | GNTYLAWYLQKPGQSPKLLIYKVSNRFSGVPDR | |
| | SRLTVSKDTSNNQAFLNILTVETADTAIYYCAQ | | FSGSGSGTDFTLKISRVEAEDLGVYYCFQGSHV | |
| | SGGITLMDYWGQGTSVTVSS | | PFTFGSGTKLEIK | |
| SCT-Ga219 | EVQLQQSGPELVKPGASMKTSCKASGYSFTGYT | 183 | DVVVTQTPLSLPVSFGDQVSISCRSSQSLANSY | 505 |
| | MNWVKQSHGKNLEWIGLIVPYNGGTSYNQKFKG | | GTTYLSWYLYKPGQSPQLLIYGISNRFSGVPDR | |
| | KATLTVDKSSSTAYMELLSLTSEDSAVYYCARS | | FSGSGSGTDFTLKISTIKPEDLGMYYCLQGTHQ | |
| | LRFSFDYWGQGTTLTVSS | | PPTFGGGTKLEIK | |
| SCT-Ga220 | EVKLVESGGGLVQPGGSLRLSCATSGFTFTDYY | 184 | DIQMTQSSSYLSVSLGGRVTITCKASDHINNWL | 506 |
| | MSWVRQPPGKALEWLALIRNKAKGYTTEYSASV | | awyqqkpgnapRLLISGATSLETGVPSRFSGSG | |
| | KGRFTISRDNSQSTLYLQMNTLRAEDSATYYCA | | SGKDYILSITSLQTEDVATYYCQQYWSTPFTFG | |
| | RDGGYWGQGTTLTVSS | | SGTKLEIK | |
| SCT-Ga221 | QVSLKESGPGILQPSQTLSLTCSFSGFSLSTSG | 185 | NIMMTQSPSSLAVSAGEKVTMSCKSSQSVLYSS | 507 |
| | MGVGWIRQPSGKGLEWLAHIWWDDVKRYNPALE | | NQKNYLAWYQQKPGQSPKLLIYWASTRESGVPD | |
| | SRLTISKDSSSSQVFLKIASVDTADTATYYCAR | | RFTGSGSGTDFTLTISSVQAEDLAVYYCHQYLS | |
| | IAVGQAWFAYWGQGTLVTVSS | | SLTFGAGTKLELR | |
| SCT-Ga222 | EVQLQQSGPELVKPGASMKISCKASGYSFTGYT | 186 | DVVVTQTPLSLPVSFGDQVSISCRSSQSLANSY | 469 |
| | MNWVKQSHGKNLEWIGLIFPYNGGTSYNQKFKG | | GTTYLSWYLHKPGQSPQLLIYGISNRFSGVPDR | |
| | KATLTVDKSSSTAYMELLSLTSEDSAVYYCARS | | FSGSGSGTDFTLKISTIKPEDLGMYYCLQGTHQ | |
| | LRFSFDYWGQGTTLTVSS | | PPTFGGGTKLEIK | |
| SCT-Ga223 | EVQLQQSGPELVKPGASMKISCKASGYSFTGYI | 181 | DVVVTQTPLSLPVSFGDQVSISCRSSQSLANSY | 508 |
| | MNWVKQSHGKNLEWTGLTVPNNGGTSYNQKFKG | | GTTYLSWYLHKPGQSPQLLIYGISNRFSGVPDR | |
| | KATLTVDKSSNTAYMELLSLTSEDSAVYYCARS | | FSGSGSGTDFTLKISTVKPEDLGMYYCLQGTHQ | |
| | LRFSFDYWGQGTTLTVSS | | PPTFGGGTKLEIK | |
| SCT-Ga224 | QVQLQQSGAELVRPGTSVKISCKPSGYIFTNYW | 187 | DIQMTQSPASLSASVGETVTITCRSSENIYSYL | 509 |
| | LGWVKQRPGHGLEWIGDIYPGGGYTNYNEKFKG | | AWYQQKQGKSPQLLVYNAKILAEGVPSRFSGSG | |
| | RATLTADTSSSTAYMQLSSLTSEDSAVYFCARS | | SGTQYSLKINSLQPEDFGSYYCQHHYGTPPTFG | |
| | RQLASYAMDYWGQGTSVTASS | | GGTNLEIK | |
| SCT-Ga225 | EVQLQQSGPEMVKPGASMKTSCKASGYSFTGYT | 188 | DVVMTQTPLSLPVSFGDQVSISCRSSQSLANSY | 510 |
| | MNWVKQSHGKNLEWLGLIFPYNSATSYNQKFTG | | GNTYLSWYLHKPGQSPHLLIYGISNRFSGVPDR | |
| | KATTTVDKSSNTTYMELLSLTSEDSAVYYCARG | | FSGSGSGTDFTLKISTIKPEDLGMYYCLQGTHQ | |
| | LRGFFALDYWGQGTSVTVSS | | PWTFGGGTKLEIR | |
| SCT-Ga226 | EVNLVESGGGLVQPGGSLRLSCATSGFPFNDYY | 189 | DIQMTQSSSYLSVSLGGRVTITCKASDHINNWL | 511 |
| | MSWVRQPPGKALEWLCLIRNKANGYTTEYSSSV | | AWYQQKPGNSPRLLISGATSLGAGVPSRFSGSG | |
| | KGRFTISRDNSQSILYLQMNTLRAEDSATYYCA | | SGKDYTLSITSLQTEDVGTYYCQQYWSIPFTFG | |
| | RDGGYWGQGTTLTVSS | | SGTKLEIK | |
| SCT-Ga227 | QAYLQQSGAELVRSGASVKMSCKASGYTFTSYN | 190 | QWLTQSPAIMSASPGEKVTMTCSASSSVSYMH | 512 |
| | LHWVKQTPGQGLEWIGYIYPGNGYTNYNQKFKG | | WYQQKSGTSPKRWIYATSKLASGVPARFSGNGS | |
| | KATLTADTSSSTAYMQISSLTSEDSAVYFCARE | | GTSYSLTISSMEAEDAATYYCQQWNSNPPTFGA | |
| | GGWAMDYWGQGTSVTVSS | | GTKLDLK | |
| SCT-Ga228 | EVQLQQSGPELVKPGASMKISCKASGYSFTGYT | 191 | DVVVTQTPLSLPVSFGDQVSISCRSSQSLANSY | 513 |
| | MNWVRQSHGKNLEWIGLINPYNGGTSYNQKFKG | | GDTFLSWYLHKPGQSPQLLIYGISNRFSGVPDR | |
| | KATLTVDKSSSTAYMELRSLTSEDSAVYYCARR | | FSGSGSGTDFTLKISTIKPEDLGMYYCLQGTHQ | |
| | NYGYYFDYWGQGTTLTVSS | | PLTFGAGTKLELK | |
| SCT-Ga229 | QITLKESGPGIVQPSQPFRLTCTFSGFSLSTSG | 192 | DVLMTQTPLSLPVSLGDQASISCRSSQSIVHSD | 514 |
| | IGVTWIRQPSGKGLEWLATIWWDDDNRYNPSLK | | GNTYLEWYLQKPGQSPKLLIYKVSNRFSGVPDR | |
| | SRLTVSKDTSNNQAFLNIITVETADTAIYYCAQ | | FSGSGSGTDFTLKISRVEAEDLGVYYCFQSSHV | |
| | SGGITLMDYWGQGTSVTVSS | | PGTFGGGTKLEIK | |
| SCT-Ga230 | EVKLVESGGGLVQPGGSLRLSCATSGFTFTDYY | 193 | DIQMTQSSSYLSVSLGGRVTITCKASDHINNWL | 515 |
| | MSWVRQPPGKALEWLALIRNKAHGYTTEYSASV | | AWYQQKPGNAPRLLISGATSLKTGVPSRFSGSG | |
| | KGRFTISKDNSQSILYLQMNTLRAEDSATYYCA | | SGKDYTLSITSLQTEDVATYYCQQYWSTPFTFG | |
| | RDGGYWGQGTTLTVSS | | SGTKLEIK | |
| SCT-Ga231 | EVQLQQSGTVLARPGASVKMSCKTSDYSFTSYW | 194 | DIVLTQSPATLSVTPGDSVSLSCRASQSIYNNL | 516 |
| | MHWVKQRPGQGLEWIGAIYPGNSAARYNQKFKG | | HWYQQNSHESPRLLIKYASQSISGIPSRFSGSG | |
| | KAKLTAVTSANTAYMELSSLTNEDSAVYYCSRE | | SGTDFTLSINSVETEDFGLYFCQQSNSWPYTFG | |
| | GGYGLDYWGQGTSVTVSS | | GGTRLEIN | |
| SCT-Ga232 | EVQLQQSGPELEKPGSSVKISCKASGYSFTDYN | 177 | EIVLTQSPAIMSASPGEKVTVTCSAGSSVSSSN | 517 |
| | MHWVKQSNGKSLEWIGDIDVYYSGTSYNQKFKG | | LHWYQQKSGSSPRLLIYDTSKLASGVPVRFSGS | |
| | KATLTVDKSSSTTYMELKSLTSEDSAVYFCARS | | GSGTSYALTISRMEAEDAATYYCQQWSSYPLTF | |
| | SLRWYFDLWGAGTTVTVSS | | GAGTKLELK | |
| SCT-Ga233 | QVQLKESGPGLVAPSQSLSITCTVSGFSLTRHG | 140 | NIMMTQSPSSLAVSAGEKVTMSCKSSQNILYSS | 518 |
| | IHWVRQPPGKGLEWLGVIWPGGNTKYNSALMSR | | NQKNYLAWYQQKPGQSPKLLIYWASTRESGVPD | |
| | LSISKDNSKSQVFLKMNSLQTDDTAMYYCARSG | | RFTGSGSGTDFTLTISSVQAEDLAVYYCHQYLS | |
| | GYGNFAPYYFDQWGQGTTLTVSS | | SYTFGGGTKLEIK | |
| SCT-Ga234 | QVQLQQSGGEVMKPGASVKISCQATGYTFSNYW | 195 | DIQMTQTTSSLSASLGDRVTISCSASQGISNYL | 519 |
| | IEWLKQRPGHGLEWTGEILPVSGSSYYNEKFRG | | NWYQQKPDGTVTLLIYYTSSLNSGVPSRFSGSG | |
| | KATFSADKSSNTAYMQLSSLTSEDSAVYYCARQ | | SGTDYSLTISNLEPEDIATYYCQHYSKLPYTFG | |
| | KYGNYYYFDYWGQGTTLTVSS | | GGTKLEMK | |
| SCT-Ga235 | QVQLQQSGGDLMKPGASVKTSCKATGYTFSNYW | 196 | DIQMTQTTSSLSASLGDRVTISCSASQGLSNYL | 520 |
| | IEWVKQRPGHGLEWIGEILPGSGSSYYNEKFKG | | HWYQQKPDGTVKLLIYYTSSLHSGVPSRFSGSG | |
| | KATFTADTSSSTAYMQLSSLTSEDSAVYYCARQ | | SGTDYSLTISNLEPEDIATYYCQQYSKLPYTFG | |
| | KYGNYYYFDFWGQGTSLTVSS | | GGTKLEIK | |
| SCT-Ga236 | QVQLQQPGSELVRPGASVKLSCKASGYTFTSYW | 171 | DIVLTQSPATLSVTPGDSVSLSCRASQSIRNSL | 521 |
| | MHWVKQRHGQGLEWTGNIFPGSGTTNYDEKFKN | | HWFQQKSHESPRLLINYASQSVSGIPSRFRGSG | |
| | KGTLTVDTSSSTAYMHLSSLTSEDSAVYYCARE | | SGTDFTLSINSLETEDFGMYFCQQSNSWPYTFG | |
| | GLYAFDYWGQGTTLTVSS | | GGTKLEIK | |
| SCT-Ga237 | EVMLVESGGGLVEPGGSLKLSCAASGFTFSTYA | 197 | NIMMTQSPSSLAVSAGEKVTMSCKSSQSVLYSS | 522 |
| | MSWVRQTPEKRLEWVATISSGGTYTYYPDSVKG | | NQMNYLAWYQQKPGQSPKVLIYWASTRESGVPD | |
| | RFTISRDNAKNTLYLQMSSLRSEDTAMYYCARH | | RFTGGGSGTDFTLTISSVQAEDLALYYCHQYLS | |
| | FTSTIITHFDYWGHGTTLTVSS | | SLTFGAGTKLELK | |
| SCT-Ga238 | EVQLQQSGTVLARPGASVKMSCKASGYSITSYW | 198 | DVVLTQSPATLSVTPGDSVSLSCRASQTISNNL | 523 |
| | MHWVKQRPGQGLEWTGGVYPGNSNTNYNQKFKG | | HWFQQKSHESPRLLIKYSSQSISGIPSRFSGSG | |
| | KAKLTAVTSASTAYMELSSLTNEDSAVYYCTRE | | SGTDFTLSINSVETEDFGMYFCQQSYSWPYTFG | |
| | GGYGMDYWGQGTSVTVSS | | GGTKLEIK | |
| SCT-Ga239 | QAYLQQSGAELVRSGASVKMSCKASGYTFTSYN | 170 | DIVMTQSQKFMSTSVGDRVSITCKASQNWTNV | 524 |
| | MHWVKQTPGQGLEWIGKIFPGNGFTDYNQNFRD | | AWYQQKPGQSPKALIYSASYRYSGVPDRFTGSG | |
| | KATLTADTSSSTAYMQISSLTSEDSAVYFCTRE | | SGTDFTLTISNVQSEDLADYFCQQHNSYPYTFG | |
| | GWLSLGYWGQGTTLTVSS | | GGTKLEIK | |
| SCT-Ga240 | QVQLQQPGSELVRPGASVKLSCKASGYTFTSYW | 199 | DIVLTQSPATLSVTPGDSVSLSCRASQNIRNSL | 525 |
| | MHWVKQRHGQGLEWIGNIFPGSGTTNYDEKFKN | | HWFQQKSHESPRLLINYASQSISGIPSRFRGSG | |
| | KGTLTVDTSSNIAYMHLSSLTSEDSAVYYCARE | | SGTDFTLTINSLETEDFGVYFCQQSNSWPYTFG | |
| | GLYAFDYWGQGTTLTVSS | | GGTKLEIK | |
| SCT-Ga241 | QTQLVQSGPELKKPGETVKTSCKASGYTFTDYS | 200 | QIVLSQSPAILSASPGEKVTMTCRASSSVNYMH | 526 |
| | MHWVKQAPGKGLKWMGWINTETGEPTYADDFKG | | WYQQKPGSSPKPWIYATSNLASGVPARFSGSGS | |
| | RFAFSLETSASTAYLQINNLKNEDTATYFCTRT | | GTSYSLTISRVEAEDAATYYCQQWNFNPQTFGG | |
| | NYGNPYWYFDVWGAGTTVTVSS | | GTKLEIK | |
| SCT-Ga242 | EVNLVESGGGLVQPGGSLRLSCATSGFTFNDYY | 201 | DIQMTQSSSYLSVSLGGRVTITCKASDHINNWL | 527 |
| | MSWVRQPPGKALEWLCLIRNKANGYTTEYSASV | | AWYQQKPGNAPRLLISGATSLGAGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga243 | EVQLQQSGPELVKPGASMKISCKASGYSFIDYI | 202 | DWLTQTPLSLPVSLGDQVSISCRSSQSLTNSY | 528 |
| | MNWVKQSHGKNLEWIGLIIPYNGGTSYNQKFKG | | GSTFLSWYLHKPGQSPQLLIYGISNRFSGVPDR | |
| | KATLTVDKSSSTASMEFLSLTSEDSAVYYCARG | | FSGSGSGTDFTLKISTVKPEDLGMYYCLQGTHQ | |
| | LRGYYAMDYWGQGTSVTVSS | | PWTFGGGTKLEIK | |
| SCT-Ga244 | QVQLQQPGSELVRPGASVKLSCKASGYTFTSYW | 203 | DIVLTQSPATLSVTPGDSVSLSCRASQNIRNNL | 529 |
| | MHWVKQRHGQGLEWIGNIFPGTGSANYDERFKN | | HWFQQKSHESPRLLIKYASQSISGIPSRFSGSG | |
| | KGTLTVDTSSSTAYMHLSSLTYEDSAVYYCTRE | | SGTDFTLSINSLETEDFGMYFCQQSNSWPYTFG | |
| | GLYPFDYWGQGTTLTVSS | | GGTKLEIK | |
| SCT-Ga245 | QVQLKQSGPGLVQPSQSLSITCTVSGFSLTSYG | 204 | QIVLTQSPAIMSASLGERVTMTCTASSSVISSY | 530 |
| | VHWVRQSPGKGLEWLGVIWSGGSTDYNAAFISR | | LHWYQQKPGSSPKLWIYSTSNLASGVPARFSGS | |
| | LSISKDNSKSQVFFKMNSLQTNDTAIYYCAREV | | GSGTSYSLTISSMEAEDAATYYCHQYHRSPLTF | |
| | RRYFDVWGAGTTVTVSS | | GAGTKLELK | |
| SCT-Ga246 | QITLKESGPGIVQPSQPFRLTCTFSGFSLSTSG | 205 | DVLMTQSPLSLPVSLGDQASISCRSSQSIVHSD | 531 |
| | IGVTWIRQPSGKGLEWLATIWWDDDNRYNPSLK | | GNTYLEWYLQKPGQSPKLLIYKVSRRFSGVPDR | |
| | SRLTVSKDTSNNQVFLNIIALETADTAMYYCAQ | | FSGSGSGTDFTLKISRVGAEDLGVYYCFQGSHV | |
| | SGGITLMDYWGQGTSVTVSS | | PGTFGGGTKLEIK | |
| SCT-Ga247 | EVQLQQSGPELVRPGGSMKISCKASGYSFTGYT | 206 | DVVMTQTPLSLPVSFGDQVSISCRSSQNLTNSY | 532 |
| | MNWVKQSHGQNLEWIGLIIPYNGGSSYNQKFQG | | GNTYLSWYLHKPGQSPQLLIYGISNRFSGVPDR | |
| | KATLTVDKSSSTAYMELLSLTSEDSAVFYCARG | | FSGSGSGTDFTLKISTIKPEDLGMYYCLQGTHQ | |
| | LRGYFAMDYWGQGTSVTVSS | | PWTFGGGTKLEIK | |
| SCT-Ga248 | QAYLQQSGAELVRSGASVKMSCKASGYTFTSYN | 207 | QIVLTQSPAIMSASPGEKVTMTCSASSSVNYMH | 533 |
| | LHWVKQTPGQGLKWIGYIYPGNGYTHYNQKFKG | | WYQQKSGTSPKRWIYDTSKLASGVPARFSGSGS | |
| | KATLTADTSSSTAYMQINSLTSEDSAVYFCARE | | GTSYSLTISSMEAEDAATYYCQQWNSNPPTFGA | |
| | GGWAMDYWGQGTSVTVSS | | GTKLELK | |
| SCT-Ga249 | QVQLQQSGGELMKPGASVKISCKATGYTFSNYW | 208 | AIQMTQTTSSLSASLGDRVTISCSASQGIGNYL | 534 |
| | IEWVKQRPGHGLEWIGEILPGSGSSYYNEKFKG | | NWYQQKPDGTVKLLIYYTSSLHSGVPSRFSGSG | |
| | RATFTADKSSNTAYMQLSSLTSEDSAVYYCARQ | | SGTDYSLTISDLEPEDIATYYCHQYSKLPYTFG | |
| | KYGNFYYFDFWGQGTTLTVSS | | GGTKLEIK | |
| SCT-Ga250 | QIQLVQSGPELKKPGETVKISCKASGYTFTNYG | 209 | DIVMTQAAFSNPVTLGTSASISCRSSKNLLHSN | 535 |
| | MNWVKQAPGKGLKWMGWISTYIGEPTCADDFKG | | GITYLYWYLQRPGQSPQLLIYRVSNLASGVPNR | |
| | RFAFSLDTSASTAYLQINNLKNEDTATYFCARD | | FSGSESGTDFTLRISRVEAEDVGVYYCGQLLEL | |
| | SFAHWGQGTLVTVSA | | PYTFGGGTKLEIK | |
| SCT-Ga251 | QAYLQQSGAELVRSGASVKMSCKASGYTFTSYN | 210 | QIVLTQSSAIMSASPGEKVTMTCSATSSVSSMH | 536 |
| | LHWVKQTPGQGLKWIGYIYPGNGYTNYNQKFKG | | WYQQKSGTSPKRWIYDTSKLASGVPARFSGSGS | |
| | KATLTADTSSSTAYMQTNSLTSEDSAVYFCARE | | GTSYSLTISSMEAEDAATYYCQQWSSNPSTFGA | |
| | GGWAMDYWGQGTSVTVSS | | GTKLELK | |
| SCT-Ga252 | QVQLQQPGSELVRPGASVKLSCKASGYTFTSFW | 211 | DIVLTQSPATLSVTPGDSVSLSCRASQSIRSSL | 537 |
| | MHWVKQRHGQGLEWTGNIYPGSGTTNFDEKFKS | | HWFQQKSHESPRLLITYASQSISGIPSRFSGSG | |
| | KVTLTVDTSSSTAFMHLSSLTSEDSAVYYCTRE | | SGTDFTLSINSLETEDFGVYFCQQSNTWPYTFG | |
| | GLYAFDQWGQGTTLTVSS | | GGTKLEIK | |
| SCT-Ga253 | QVQLQQPGSELVRPGASVKLSCKASGYTFTSYW | 171 | DIVLTQSPATLSVTPGDSVSLSCRASQSIRNSL | 538 |
| | MHWVKQRHGQGLEWIGNIFPGSGTTNYDEKFKN | | HWFQQKSHESPRLLINYASQSISGIPSRFRGSG | |
| | KGTLTVDTSSSTAYMHLSSLTSEDSAVYYCARE | | SGTDFTLSINSLETEDFGMYFCQQSNSWPYTFG | |
| | GLYAFDYWGQGTTLTVSS | | GGTKLEIK | |
| SCT-Ga254 | QVQLQQSGAELVRPGTSVKISCKASGYTFTIHW | 212 | DVVMTQTPLTLSVTIGQPASISCKSSQSLLDSD | 539 |
| | LGWVRQRPGHGLEWTGDIYPGGGYTNYNEKFKG | | GKTYLNWLLQRPGQSPKRLIYLVSKLDSGVPDR | |
| | KATLTADTSSSTAYMQFSSLTSEDSAVYFCARD | | FTGSGSGTDFTLKISRVEAEDLGVYYCWQGTHF | |
| | YGYDVYAMDYWGQGTSVTVSS | | PRTFGGGTKLEIK | |
| SCT-Ga255 | EVKLVESGGGLVKPGGSLKLSCAASGFAFSSFD | 213 | DIQMNQSPSSLSASLGDTITITCHASQNINVWL | 540 |
| | MSWVRQTPEKRLEWVAVTSSGGSYTFYPDSVKG | | SWYQQKPGNIPKLLIYKASNLHTGVPSRFSGSG | |
| | RFTISRDNARNTLYLQMSSLRSEDTALYFCTRQ | | SGAGFTLTISSLQPEDIATYYCQQGQSYPWTFG | |
| | DYRYPYTMDYWGQGTSVTVSS | | GGTKLEIK | |
| SCT-Ga256 | QVQLQQSGAELVRPGTSVKISCKPSGYIFTNFW | 214 | DIQMTQSPASLSASVGETVTITCRSSENIYSYL | 541 |
| | LGWVKQRPGHGLEWIGDIYPGGGYINYNEKFKG | | AWYQQKQGKSPQLLVYNAKILAEGVPSRFSGSG | |
| | RATLTADTSSSTAYMQLSSLTSEDSAVYFCARS | | SGTQYSLKINSLQPEDFGTYYCQHHYGTPPTFG | |
| | RQLASYAMDYWGQGTSVTASS | | GGTNLEIK | |
| SCT-Ga257 | QTTLKESGPGIVQPSQPFRLTCTFSGFSLSTSG | 182 | DVLMTQTPLSLPVSLGDQASISCRSSQNIVHSN | 542 |
| | IGVTWIRQPSGKGLEWLATIWWDDDTRYNPSLK | | GNTYLEWYLQKPGQSPKLLIYKVSNRFSGVPDR | |
| | SRLTVSKDTSNNQAFLNILTVETADTATYYCAQ | | FSGSGSGTDFTLKISRVEAEDLGVYYCFQTSHV | |
| | SGGITLMDYWGQGTSVTVSS | | PGTFGGGTKLEIK | |
| SCT-Ga258 | EVKLVESGGGLVQPGGSLRLSCATSGFTFTDYY | 215 | DIQMTQSSSYLSVSLGGRVTITCKASDHINNWL | 515 |
| | MSWVRQPPGKALEWLALTRNKAHGYTTEYSASV | | AWYQQKPGNAPRLLISGATSLKTGVPSRFSGSG | |
| | KGRFTISRDNSQSILYLQMNTLRAEDSATYYCA | | SGKDYTLSITSLQTEDVATYYCQQYWSTPFTFG | |
| | RDGGYWGQGTTLTVSS | | SGTKLEIK | |
| SCT-Ga259 | EVNLVESGGGLVQPGGSLRLSCATSGFTFNDYY | 201 | DIQMTQSPSYLSVSLGGRVTINCKASDHINNWL | 543 |
| | MSWVRQPPGKALEWLCLIRNKANGYTTEYSASV | | AWYQQKPGNAPRLLISGATSLETGVPSRFSGSG | |
| | KGRFTISRDNSQSILYLQMNTLRAEDSATYYCA | | SGKDYTLSITSLQTEDAATYYCQQYWSIPFTFG | |
| | RDGGYWGQGTTLTVSS | | SGTKLEIK | |
| SCT-Ga260 | EVNLVESGGGLVQPGGSLRLSCATSGFTFNDYY | 216 | DIQMTQSSSYLSVSLGGRVTITCKASDHINNWL | 527 |
| | MAWVRQPPGKALEWLCLIRDKANGYTTEYSASV | | AWYQQKPGNAPRLLISGATSLGAGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga261 | EVQLQQSGPELEKPGSSVKISCKASGYSFSDYN | 217 | QIVLTQSPAIMSASPGEKVTMTCSASSSVTYMY | 544 |
| | MHWVKQSNGKSLEWIGNIDVYYLSTSYNQKFKG | | WYQQKSGSSPRLLIYDTSNLASGVPVRFSGSGS | |
| | KATLTVDRSSSTAYMQLKSLTSEDSAVYFCARS | | GTSYSLTISRMEAEDAATYYCQHWSSYPLTFGA | |
| | LLRWYFDVWGAGTTVTVSS | | GTKLELK | |
| SCT-Ga262 | EVQLQQSGPELVKPGASMKISCKASGYSFTGYT | 218 | DWVTQTPLSLPVSFGDQVSISCRSSQSLANSY | 482 |
| | MNWVKQSHGKNLEWIGLIIPYNGGTSYNQKFKG | | GNTYLSWYLHKPGQSPQLLIYGISNRFSGVPDR | |
| | KATLTVDKSSSTAYMELLSLTSEDSAVYFCARS | | FSGSGSGTDFTLKISTIKPEDLGMYYCLQGTHQ | |
| | LRFSFDYWGQGTSLTVSS | | PPTFGGGTKLEIK | |
| SCT-Ga263 | QVQLQQPGSELVRPGASVKLSCKASGYTFTSYW | 219 | DIVLTQSPATLSVTPGDSVSLSCRASQSIRNNL | 545 |
| | IHWVKQRHGQGLEWIGNIFPGSGTTNYDEKFKS | | HWFQQKSHESPRLLIKYVSQSISGIPSRFSGSG | |
| | RGTLTVDTSSSTAYMHLSSLTSEDSAIYYCTRE | | SGTDFTLNINSLEAEDFGMYFCQQSISWPYTFG | |
| | GLYSFDYWGQGTTLTVSS | | GGTKLEIK | |
| SCT-Ga264 | QVQLQQPGSELVRPGASVKLSCKASGYTFTSYW | 220 | DIVLTQSPATLSVTPGDSVSLSCRASQSIRNSL | 546 |
| | MHWVKQRHGQGLEWIGNIFPGSGITNYDEKFKN | | HWFQQKSHESPRLLINYASQSMSGIPSRFRGSG | |
| | KGTLTVDTSSSTAYMHLSSLTSEDSAVYYCARE | | SGTDFTLSINSLETEDFGMYFCQQTNSWPYTFG | |
| | GLYAFDYWGQGTTLTVSS | | GGTKLEIK | |
| SCT-Ga265 | QAYLQQSGAELVRSGASVKMSCKASGYTFTSYN | 221 | DIVMTQSQKFMSTSIGDRVSVTCKASQNWTNV | 547 |
| | MHWVKQTPGQGLEWIGKIFPGNGFTDYNQRFRD | | AWYQQRPGQSPKALIYSASYRYSGVPDRFAGSG | |
| | KATLTADTSSSTAYMQISNLTSEDSAVYFCTRE | | SGTDFTLTISNVQSEDLADYFCQQHNSYPYTFG | |
| | GWLSLGYWGQGTTLTVSS | | GGTKLEIK | |
| SCT-Ga266 | EVQLQQSGPELVKPGASMKISCKASGYSFTGYT | 183 | DVWTQTPLSLPVSFGDQVSISCRSSQSLANSY | 548 |
| | MNWVKQSHGKNLEWIGLIVPYNGGTSYNQKFKG | | GTTYLSWYLHKPGQSPQLLIYGISNRFSGVPDR | |
| | KATLTVDKSSSTAYMELLSLTSEDSAVYYCARS | | FSGSGSGTDFTLRISTIKPEDLGMYYCLQGTHQ | |
| | LRFSFDYWGQGTTLTVSS | | PPTFGGGTKLEIK | |
| SCT-Ga267 | EVQLQQSGPELVKPGASMKTSCKASGYSFTGYT | 222 | DVWTQTPLSLPVSFGDQVSISCRSSQSLANSY | 469 |
| | MNWVKQSHGKNLEWIGLIVPNNGGTSYNQKFKG | | GTTYLSWYLHKPGQSPQLLIYGISNRFSGVPDR | |
| | KATLTVDKSSSTAYMELLSLTSEDSAVYYCARS | | FSGSGSGTDFTLKISTIKPEDLGMYYCLQGTHQ | |
| | LRFSFDYWGQGTTLTVSS | | PPTFGGGTKLEIK | |
| SCT-Ga268 | QIQLVQSGPELKKPGETVKISCKASGYIFTNYG | 223 | DIVMTQAAFSNPVTLGTSASISCRSSKNLLHSN | 549 |
| | MNWVKKAPGKGLKWMGWINSYTGESTYGDDFKG | | GITYLYWYLQRPGQSPQLLIYRMSNLASGVPNR | |
| | RFAFSSETSASTAYLQINNLKKEDTATYFCARD | | FSGSGSGTDFTLRISRVEAEDVGVYYCGQLLEL | |
| | SFAYWGQGTLVTVSA | | PYTFGGGTKLEIK | |
| SCT-Ga269 | QAYLQQSGAELVRSGASVKMSCKASGYTFTSYN | 224 | DIVMTQSQKFMSTSIGDRVSVTCKASQNWTNV | 550 |
| | MHWVKQTPGQGLEWIGKIFPGNGFTDYNQIFRD | | AWYQQKPGQSPKALIYSASYRYSGVPDRFTGSG | |
| | KATLTADTSSSTAYMQISSLTSEDSAVYFCTRE | | SGTDFTLTINNVQSEDLADYFCQQHNSYPYTFG | |
| | GWLSLGYWGQGTTLTVSS | | GGTKLEIK | |
| SCT-Ga270 | QVQLQQPGAELVKPGASVKMSCKASGYTFTTFW | 225 | DILLTQSPAILSVSPGERVSFSCRASQSIGTSI | 551 |
| | TNWVKQRPGQGLEWTGDTFPGRGITNYNGKFKN | | QWYQQRTMGSPRLLIKYGSESISGIPSRFSGSG | |
| | KATLTLDTSSSTAYMQLSSLTSEDSAVYYCSRG | | SGTDFTLSINSVESEDSADYYCQQSYRWPYTFG | |
| | LGYFDVWGAGTTVTVSS | | GGSKLEIK | |
| SCT-Ga271 | EVNLVESGGGLVQPGGSLRLSCATSGFTFNDYY | 226 | DIQMTQSSSYLSVSLGGRVTITCKASDHINNWL | 552 |
| | MSWVRQPPGKALEWLCLIRDKANGYTTEYSASV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga272 | QVQLQQPGSELVRPGASVKLSCKASGYTFTSYW | 227 | DIVLTQSPATLSVTPGDSVSLSCRASQNIRNNL | 529 |
| | MHWVKQRHGQGLEWIGNIFPGTGSTNYDERFKN | | HWFQQKSHESPRLLIKYASQSISGIPSRFSGSG | |
| | KGTLTVDTSSSTAYMHLSSLTYEDSAVYYCTRE | | SGTDFTLSINSLETEDFGMYFCQQSNSWPYTFG | |
| | GLYPFDYWGQGTTLTVSS | | GGTKLEIK | |
| SCT-Ga273 | EVQLQQSGPELVKPGASMKISCKASGYSFTGYT | 151 | DVWTQTPLSLPVSFGDQVSISCRSSQSLTNSY | 553 |
| | MNWVKQSHGKNLEWIGLIIPYNGGTSYNQKFKD | | GNTFLSWYLHKPGQSPQLLIYGISNRFSGVPDR | |
| | KATLTIDKSSSTAYMELLSLTSEDSAVYYCARG | | FSGSGSGTNFTLKISTIKPEDLGMYYCLQGTHQ | |
| | FRGYFAVDYWGQGTSVTVSS | | PWTFGGGTKLEIK | |
| SCT-Ga274 | EVQLQQSGPELEKPGSSVKISCKASGYSFTDYN | 228 | EIVLTQSPAIMSASPGEKVTMTCSAGSSVSSSY | 554 |
| | MHWVKQSNGKSLEWIGNIDVYYSGTSYNQKFRG | | LHWYQQKSGSSPRLLIYDTSNLASGVPVRFSGS | |
| | KATLTVDKSSSTAYMELKSLTSEDSAVYFCARS | | GSGTSYSLTISPMEAEDAATYYCQQWSSYPLTF | |
| | LLRWYFDVWGAGTTVTVSS | | GAGTKLELK | |
| SCT-Ga275 | EVMLVESGGGLVKPGGSLKLSCAASGFTFSTYA | 229 | NIMMTQSPSSLAVSAGEKVTMSCKSSQSVLYSS | 555 |
| | MSWVRQTPEKRLEWVATISSGGSYTYYPDSVKG | | NQMNYLAWYQQKPGQSPKLLIYWASTRESGVPD | |
| | RFTISRDNAKNTLYLQMSSLRSEDTAMYYCARH | | RFTGSGSGTDFTLTISSVQAEDLALYYCHQYLS | |
| | FISTIITHFDYWGHGTTLTVSS | | SLTFGAGTKLELK | |
| SCT-Ga276 | EVNLVESGGGLVQPGGSLRLSCATSGFTFNDYY | 201 | DIQMTQSSSYLSVSLGGRVTITCKASDHINNWL | 552 |
| | MSWVRQPPGKALEWLCLIRNKANGYTTEYSASV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga277 | QVQLQQSGPELVKPGASVKMSCKASGYTFTSYY | 230 | DVLMTQTPLSLPVSLGDQASISCRSSQNIVHSN | 556 |
| | IHWVKQRPGQGLEWIGWIYPGDGSTKYNEKFKG | | GNTYLEWYLQKPGQSPKLLIYKVSNRFSGVPDR | |
| | KTTLTADKSSSTAYMLLSSLTSEDSAIYFCTRD | | FSGSGSGTDFTLKISRVEAEDLGVYYCFQGSHV | |
| | GNYGDWFPYWGQGTLVTVSA | | PYTFGGGTKLEIK | |
| SCT-Ga278 | QVQLQQSGGEVMKPGASVKISCKATGYTFSNYW | 231 | DIQMTQPTSSLSASLGDRVTISCSASQGISSYL | 557 |
| | IEWVKQRPGHGLEWIGEILPVSDSAYYNEKFRG | | NWYQQKPDGTVTLLIYYTSSLHSGVPSRFSGSG | |
| | KATFTADKSSNTAYMQLSTLTSEDSAVYYCARQ | | SGTDYSLTISNLEPEDIATYYCQHYSKLPYTFG | |
| | KYGNYYYFDYWGQGTTLTVSS | | GGTKLEIK | |
| SCT-Ga279 | QVQLQQPGSELVRPGASVKLSCKASGYTFTSYW | 163 | DIVLTQSPASLAVSLGQRASISCRASESVDKYG | 558 |
| | IHWVKQRHGQGLEWIGNIFPGSGTSNYDEKFNN | | ISFMSWFQQKPGQPPKLLIYAASNQGSGVPARF | |
| | KGTLTVDTSSSTSYMHLSSLTSEDSAVYYCARE | | SGSGSGTDFSLNIHPMEEDDTAMYFCQQSKEVP | |
| | GLYAFDYWGQGTTLTVSS | | WTFGGGTKLEIK | |
| SCT-Ga232 | EVQLQQSGPELEKPGSSVKISCKASGYSFTDYN | 177 | EIVLTQSPAIMSASPGEKVTVTCSAGSSVSSSN | 517 |
| | MHWVKQSNGKSLEWIGDIDVYYSGTSYNQKFKG | | LHWYQQKSGSSPRLLIYDTSKLASGVPVRFSGS | |
| | KATLTVDKSSSTTYMELKSLTSEDSAVYFCARS | | GSGTSYALTISRMEAEDAATYYCQQWSSYPLTF | |
| | SLRWYFDLWGAGTTVTVSS | | GAGTKLELK | |
| SCT-Ga233 | QVQLKESGPGLVAPSQSLSITCTVSGFSLTRHG | 140 | NIMMTQSPSSLAVSAGEKVTMSCKSSQNILYSS | 518 |
| | IHWVRQPPGKGLEWLGVIWPGGNTKYNSALMSR | | NQKNYLAWYQQKPGQSPKLLIYWASTRESGVPD | |
| | LSISKDNSKSQVFLKMNSLQTDDTAMYYCARSG | | RFTGSGSGTDFTLTISSVQAEDLAVYYCHQYLS | |
| | GYGNFAPYYFDQWGQGTTLTVSS | | SYTFGGGTKLEIK | |
| SCT-Ga234 | QVQLQQSGGEVMKPGASVKISCQATGYTFSNYW | 195 | DIQMTQTTSSLSASLGDRVTISCSASQGISNYL | 519 |
| | IEWLKQRPGHGLEWTGEILPVSGSSYYNEKFRG | | NWYQQKPDGTVTLLIYYTSSLNSGVPSRFSGSG | |
| | KATFSADKSSNTAYMQLSSLTSEDSAVYYCARQ | | SGTDYSLTISNLEPEDIATYYCQHYSKLPYTFG | |
| | KYGNYYYFDYWGQGTTLTVSS | | GGTKLEMK | |
| SCT-Ga235 | QVQLQQSGGDLMKPGASVKTSCKATGYTFSNYW | 196 | DIQMTQTTSSLSASLGDRVTISCSASQGLSNYL | 520 |
| | IEWVKQRPGHGLEWIGEILPGSGSSYYNEKFKG | | HWYQQKPDGTVKLLIYYTSSLHSGVPSRFSGSG | |
| | KATFTADTSSSTAYMQLSSLTSEDSAVYYCARQ | | SGTDYSLTISNLEPEDIATYYCQQYSKLPYTFG | |
| | KYGNYYYFDFWGQGTSLTVSS | | GGTKLEIK | |
| SCT-Ga236 | QVQLQQPGSELVRPGASVKLSCKASGYTFTSYW | 171 | DIVLTQSPATLSVTPGDSVSLSCRASQSIRNSL | 521 |
| | MHWVKQRHGQGLEWTGNIFPGSGTTNYDEKFKN | | HWFQQKSHESPRLLINYASQSVSGIPSRFRGSG | |
| | KGTLTVDTSSSTAYMHLSSLTSEDSAVYYCARE | | SGTDFTLSINSLETEDFGMYFCQQSNSWPYTFG | |
| | GLYAFDYWGQGTTLTVSS | | GGTKLEIK | |
| SCT-Ga237 | EVMLVESGGGLVEPGGSLKLSCAASGFTFSTYA | 197 | NIMMTQSPSSLAVSAGEKVTMSCKSSQSVLYSS | 522 |
| | MSWVRQTPEKRLEWVATISSGGTYTYYPDSVKG | | NQMNYLAWYQQKPGQSPKVLIYWASTRESGVPD | |
| | RFTISRDNAKNTLYLQMSSLRSEDTAMYYCARH | | RFTGGGSGTDFTLTISSVQAEDLALYYCHQYLS | |
| | FTSTIITHFDYWGHGTTLTVSS | | SLTFGAGTKLELK | |
| SCT-Ga238 | EVQLQQSGTVLARPGASVKMSCKASGYSITSYW | 198 | DVVLTQSPATLSVTPGDSVSLSCRASQTISNNL | 523 |
| | MHWVKQRPGQGLEWTGGVYPGNSNTNYNQKFKG | | HWFQQKSHESPRLLIKYSSQSISGIPSRFSGSG | |
| | KAKLTAVTSASTAYMELSSLTNEDSAVYYCTRE | | SGTDFTLSINSVETEDFGMYFCQQSYSWPYTFG | |
| | GGYGMDYWGQGTSVTVSS | | GGTKLEIK | |
| SCT-Ga239 | QAYLQQSGAELVRSGASVKMSCKASGYTFTSYN | 170 | DIVMTQSQKFMSTSVGDRVSITCKASQNVVTNV | 524 |
| | MHWVKQTPGQGLEWIGKIFPGNGFTDYNQNFRD | | AWYQQKPGQSPKALIYSASYRYSGVPDRFTGSG | |
| | KATLTADTSSSTAYMQISSLTSEDSAVYFCTRE | | SGTDFTLTISNVQSEDLADYFCQQHNSYPYTFG | |
| | GWLSLGYWGQGTTLTVSS | | GGTKLEIK | |
| SCT-Ga240 | QVQLQQPGSELVRPGASVKLSCKASGYTFTSYW | 199 | DIVLTQSPATLSVTPGDSVSLSCRASQNIRNSL | 525 |
| | MHWVKQRHGQGLEWIGNIFPGSGTTNYDEKFKN | | HWFQQKSHESPRLLINYASQSISGIPSRFRGSG | |
| | KGTLTVDTSSNIAYMHLSSLTSEDSAVYYCARE | | SGTDFTLTINSLETEDFGVYFCQQSNSWPYTFG | |
| | GLYAFDYWGQGTTLTVSS | | GGTKLEIK | |
| SCT-Ga241 | QTQLVQSGPELKKPGETVKTSCKASGYTFTDYS | 200 | QIVLSQSPAILSASPGEKVTMTCRASSSVNYMH | 526 |
| | MHWVKQAPGKGLKWMGWINTETGEPTYADDFKG | | WYQQKPGSSPKPWIYATSNLASGVPARFSGSGS | |
| | RFAFSLETSASTAYLQINNLKNEDTATYFCTRT | | GTSYSLTISRVEAEDAATYYCQQWNFNPQTFGG | |
| | NYGNPYWYFDVWGAGTTVTVSS | | GTKLEIK | |
| SCT-Ga242 | EVNLVESGGGLVQPGGSLRLSCATSGFTFNDYY | 201 | DIQMTQSSSYLSVSLGGRVTITCKASDHINNWL | 527 |
| | MSWVRQPPGKALEWLCLIRNKANGYTTEYSASV | | AWYQQKPGNAPRLLISGATSLGAGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga243 | EVQLQQSGPELVKPGASMKISCKASGYSFIDYI | 202 | DWLTQTPLSLPVSLGDQVSISCRSSQSLTNSY | 528 |
| | MNWVKQSHGKNLEWIGLIIPYNGGTSYNQKFKG | | GSTFLSWYLHKPGQSPQLLIYGISNRFSGVPDR | |
| | KATLTVDKSSSTASMEFLSLTSEDSAVYYCARG | | FSGSGSGTDFTLKISTVKPEDLGMYYCLQGTHQ | |
| | LRGYYAMDYWGQGTSVTVSS | | PWTFGGGTKLEIK | |
| SCT-Ga244 | QVQLQQPGSELVRPGASVKLSCKASGYTFTSYW | 203 | DIVLTQSPATLSVTPGDSVSLSCRASQNIRNNL | 529 |
| | MHWVKQRHGQGLEWIGNIFPGTGSANYDERFKN | | HWFQQKSHESPRLLIKYASQSISGIPSRFSGSG | |
| | KGTLTVDTSSSTAYMHLSSLTYEDSAVYYCTRE | | SGTDFTLSINSLETEDFGMYFCQQSNSWPYTFG | |
| | GLYPFDYWGQGTTLTVSS | | GGTKLEIK | |
| SCT-Ga245 | QVQLKQSGPGLVQPSQSLSITCTVSGFSLTSYG | 204 | QIVLTQSPAIMSASLGERVTMTCTASSSVISSY | 530 |
| | VHWVRQSPGKGLEWLGVIWSGGSTDYNAAFISR | | LHWYQQKPGSSPKLWIYSTSNLASGVPARFSGS | |
| | LSISKDNSKSQVFFKMNSLQTNDTAIYYCAREV | | GSGTSYSLTISSMEAEDAATYYCHQYHRSPLTF | |
| | RRYFDVWGAGTTVTVSS | | GAGTKLELK | |
| SCT-Ga246 | QITLKESGPGIVQPSQPFRLTCTFSGFSLSTSG | 205 | DVLMTQSPLSLPVSLGDQASISCRSSQSIVHSD | 531 |
| | IGVTWIRQPSGKGLEWLATIWWDDDNRYNPSLK | | GNTYLEWYLQKPGQSPKLLIYKVSRRFSGVPDR | |
| | SRLTVSKDTSNNQVFLNIIALETADTAMYYCAQ | | FSGSGSGTDFTLKISRVGAEDLGVYYCFQGSHV | |
| | SGGITLMDYWGQGTSVTVSS | | PGTFGGGTKLEIK | |
| SCT-Ga247 | EVQLQQSGPELVRPGGSMKISCKASGYSFTGYT | 206 | DVVMTQTPLSLPVSFGDQVSISCRSSQNLTNSY | 532 |
| | MNWVKQSHGQNLEWIGLIIPYNGGSSYNQKFQG | | GNTYLSWYLHKPGQSPQLLIYGISNRFSGVPDR | |
| | KATLTVDKSSSTAYMELLSLTSEDSAVFYCARG | | FSGSGSGTDFTLKISTIKPEDLGMYYCLQGTHQ | |
| | LRGYFAMDYWGQGTSVTVSS | | PWTFGGGTKLEIK | |
| SCT-Ga248 | QAYLQQSGAELVRSGASVKMSCKASGYTFTSYN | 207 | QIVLTQSPAIMSASPGEKVTMTCSASSSVNYMH | 533 |
| | LHWVKQTPGQGLKWIGYIYPGNGYTHYNQKFKG | | WYQQKSGTSPKRWIYDTSKLASGVPARFSGSGS | |
| | KATLTADTSSSTAYMQINSLTSEDSAVYFCARE | | GTSYSLTISSMEAEDAATYYCQQWNSNPPTFGA | |
| | GGWAMDYWGQGTSVTVSS | | GTKLELK | |
| SCT-Ga249 | QVQLQQSGGELMKPGASVKISCKATGYTFSNYW | 208 | AIQMTQTTSSLSASLGDRVTISCSASQGIGNYL | 534 |
| | IEWVKQRPGHGLEWIGEILPGSGSSYYNEKFKG | | NWYQQKPDGTVKLLIYYTSSLHSGVPSRFSGSG | |
| | RATFTADKSSNTAYMQLSSLTSEDSAVYYCARQ | | SGTDYSLTISDLEPEDIATYYCHQYSKLPYTFG | |
| | KYGNFYYFDFWGQGTTLTVSS | | GGTKLEIK | |
| SCT-Ga250 | QIQLVQSGPELKKPGETVKISCKASGYTFTNYG | 209 | DIVMTQAAFSNPVTLGTSASISCRSSKNLLHSN | 535 |
| | MNWVKQAPGKGLKWMGWISTYIGEPTCADDFKG | | GITYLYWYLQRPGQSPQLLIYRVSNLASGVPNR | |
| | RFAFSLDTSASTAYLQINNLKNEDTATYFCARD | | FSGSESGTDFTLRISRVEAEDVGVYYCGQLLEL | |
| | SFAHWGQGTLVTVSA | | PYTFGGGTKLEIK | |
| SCT-Ga251 | QAYLQQSGAELVRSGASVKMSCKASGYTFTSYN | 210 | QIVLTQSSAIMSASPGEKVTMTCSATSSVSSMH | 536 |
| | LHWVKQTPGQGLKWIGYIYPGNGYTNYNQKFKG | | WYQQKSGTSPKRWIYDTSKLASGVPARFSGSGS | |
| | KATLTADTSSSTAYMQTNSLTSEDSAVYFCARE | | GTSYSLTISSMEAEDAATYYCQQWSSNPSTFGA | |
| | GGWAMDYWGQGTSVTVSS | | GTKLELK | |
| SCT-Ga252 | QVQLQQPGSELVRPGASVKLSCKASGYTFTSFW | 211 | DIVLTQSPATLSVTPGDSVSLSCRASQSIRSSL | 537 |
| | MHWVKQRHGQGLEWTGNIYPGSGTTNFDEKFKS | | HWFQQKSHESPRLLITYASQSISGIPSRFSGSG | |
| | KVTLTVDTSSSTAFMHLSSLTSEDSAVYYCTRE | | SGTDFTLSINSLETEDFGVYFCQQSNTWPYTFG | |
| | GLYAFDQWGQGTTLTVSS | | GGTKLEIK | |
| SCT-Ga253 | QVQLQQPGSELVRPGASVKLSCKASGYTFTSYW | 171 | DIVLTQSPATLSVTPGDSVSLSCRASQSIRNSL | 538 |
| | MHWVKQRHGQGLEWIGNIFPGSGTTNYDEKFKN | | HWFQQKSHESPRLLINYASQSISGIPSRFRGSG | |
| | KGTLTVDTSSSTAYMHLSSLTSEDSAVYYCARE | | SGTDFTLSINSLETEDFGMYFCQQSNSWPYTFG | |
| | GLYAFDYWGQGTTLTVSS | | GGTKLEIK | |
| SCT-Ga254 | QVQLQQSGAELVRPGTSVKISCKASGYTFTIHW | 212 | DVVMTQTPLTLSVTIGQPASISCKSSQSLLDSD | 539 |
| | LGWVRQRPGHGLEWTGDIYPGGGYTNYNEKFKG | | GKTYLNWLLQRPGQSPKRLIYLVSKLDSGVPDR | |
| | KATLTADTSSSTAYMQFSSLTSEDSAVYFCARD | | FTGSGSGTDFTLKISRVEAEDLGVYYCWQGTHF | |
| | YGYDVYAMDYWGQGTSVTVSS | | PRTFGGGTKLEIK | |
| SCT-Ga255 | EVKLVESGGGLVKPGGSLKLSCAASGFAFSSFD | 213 | DIQMNQSPSSLSASLGDTITITCHASQNINVWL | 540 |
| | MSWVRQTPEKRLEWVAVTSSGGSYTFYPDSVKG | | SWYQQKPGNIPKLLIYKASNLHTGVPSRFSGSG | |
| | RFTISRDNARNTLYLQMSSLRSEDTALYFCTRQ | | SGAGFTLTISSLQPEDIATYYCQQGQSYPWTFG | |
| | DYRYPYTMDYWGQGTSVTVSS | | GGTKLEIK | |
| SCT-Ga256 | QVQLQQSGAELVRPGTSVKISCKPSGYIFTNFW | 214 | DIQMTQSPASLSASVGETVTITCRSSENIYSYL | 541 |
| | LGWVKQRPGHGLEWIGDIYPGGGYINYNEKFKG | | AWYQQKQGKSPQLLVYNAKILAEGVPSRFSGSG | |
| | RATLTADTSSSTAYMQLSSLTSEDSAVYFCARS | | SGTQYSLKINSLQPEDFGTYYCQHHYGTPPTFG | |
| | RQLASYAMDYWGQGTSVTASS | | GGTNLEIK | |
| SCT-Ga257 | QTTLKESGPGIVQPSQPFRLTCTFSGFSLSTSG | 182 | DVLMTQTPLSLPVSLGDQASISCRSSQNIVHSN | 542 |
| | IGVTWIRQPSGKGLEWLATIWWDDDTRYNPSLK | | GNTYLEWYLQKPGQSPKLLIYKVSNRFSGVPDR | |
| | SRLTVSKDTSNNQAFLNILTVETADTATYYCAQ | | FSGSGSGTDFTLKISRVEAEDLGVYYCFQTSHV | |
| | SGGITLMDYWGQGTSVTVSS | | PGTFGGGTKLEIK | |
| SCT-Ga258 | EVKLVESGGGLVQPGGSLRLSCATSGFTFTDYY | 215 | DIQMTQSSSYLSVSLGGRVTITCKASDHINNWL | 515 |
| | MSWVRQPPGKALEWLALTRNKAHGYTTEYSASV | | AWYQQKPGNAPRLLISGATSLKTGVPSRFSGSG | |
| | KGRFTISRDNSQSILYLQMNTLRAEDSATYYCA | | SGKDYTLSITSLQTEDVATYYCQQYWSTPFTFG | |
| | RDGGYWGQGTTLTVSS | | SGTKLEIK | |
| SCT-Ga259 | EVNLVESGGGLVQPGGSLRLSCATSGFTFNDYY | 201 | DIQMTQSPSYLSVSLGGRVTINCKASDHINNWL | 543 |
| | MSWVRQPPGKALEWLCLIRNKANGYTTEYSASV | | AWYQQKPGNAPRLLISGATSLETGVPSRFSGSG | |
| | KGRFTISRDNSQSILYLQMNTLRAEDSATYYCA | | SGKDYTLSITSLQTEDAATYYCQQYWSIPFTFG | |
| | RDGGYWGQGTTLTVSS | | SGTKLEIK | |
| SCT-Ga260 | EVNLVESGGGLVQPGGSLRLSCATSGFTFNDYY | 216 | DIQMTQSSSYLSVSLGGRVTITCKASDHINNWL | 527 |
| | MAWVRQPPGKALEWLCLIRDKANGYTTEYSASV | | AWYQQKPGNAPRLLISGATSLGAGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga261 | EVQLQQSGPELEKPGSSVKISCKASGYSFSDYN | 217 | QIVLTQSPAIMSASPGEKVTMTCSASSSVTYMY | 544 |
| | MHWVKQSNGKSLEWIGNIDVYYLSTSYNQKFKG | | WYQQKSGSSPRLLIYDTSNLASGVPVRFSGSGS | |
| | KATLTVDRSSSTAYMQLKSLTSEDSAVYFCARS | | GTSYSLTISRMEAEDAATYYCQHWSSYPLTFGA | |
| | LLRWYFDVWGAGTTVTVSS | | GTKLELK | |
| SCT-Ga262 | EVQLQQSGPELVKPGASMKISCKASGYSFTGYT | 218 | DVVVTQTPLSLPVSFGDQVSISCRSSQSLANSY | 482 |
| | MNWVKQSHGKNLEWIGLIIPYNGGTSYNQKFKG | | GNTYLSWYLHKPGQSPQLLIYGISNRFSGVPDR | |
| | KATLTVDKSSSTAYMELLSLTSEDSAVYFCARS | | FSGSGSGTDFTLKISTIKPEDLGMYYCLQGTHQ | |
| | LRFSFDYWGQGTSLTVSS | | PPTFGGGTKLEIK | |
| SCT-Ga263 | QVQLQQPGSELVRPGASVKLSCKASGYTFTSYW | 219 | DIVLTQSPATLSVTPGDSVSLSCRASQSIRNNL | 545 |
| | IHWVKQRHGQGLEWIGNIFPGSGTTNYDEKFKS | | HWFQQKSHESPRLLIKYVSQSISGIPSRFSGSG | |
| | RGTLTVDTSSSTAYMHLSSLTSEDSAIYYCTRE | | SGTDFTLNINSLEAEDFGMYFCQQSISWPYTFG | |
| | GLYSFDYWGQGTTLTVSS | | GGTKLEIK | |
| SCT-Ga264 | QVQLQQPGSELVRPGASVKLSCKASGYTFTSYW | 220 | DIVLTQSPATLSVTPGDSVSLSCRASQSIRNSL | 546 |
| | MHWVKQRHGQGLEWIGNIFPGSGITNYDEKFKN | | HWFQQKSHESPRLLINYASQSMSGIPSRFRGSG | |
| | KGTLTVDTSSSTAYMHLSSLTSEDSAVYYCARE | | SGTDFTLSINSLETEDFGMYFCQQTNSWPYTFG | |
| | GLYAFDYWGQGTTLTVSS | | GGTKLEIK | |
| SCT-Ga265 | QAYLQQSGAELVRSGASVKMSCKASGYTFTSYN | 221 | DIVMTQSQKFMSTSIGDRVSVTCKASQNVVTNV | 547 |
| | MHWVKQTPGQGLEWIGKIFPGNGFTDYNQRFRD | | AWYQQRPGQSPKALIYSASYRYSGVPDRFAGSG | |
| | KATLTADTSSSTAYMQISNLTSEDSAVYFCTRE | | SGTDFTLTISNVQSEDLADYFCQQHNSYPYTFG | |
| | GWLSLGYWGQGTTLTVSS | | GGTKLEIK | |
| SCT-Ga266 | EVQLQQSGPELVKPGASMKISCKASGYSFTGYT | 183 | DVVVTQTPLSLPVSFGDQVSISCRSSQSLANSY | 548 |
| | MNWVKQSHGKNLEWIGLIVPYNGGTSYNQKFKG | | GTTYLSWYLHKPGQSPQLLIYGISNRFSGVPDR | |
| | KATLTVDKSSSTAYMELLSLTSEDSAVYYCARS | | FSGSGSGTDFTLRISTIKPEDLGMYYCLQGTHQ | |
| | LRFSFDYWGQGTTLTVSS | | PPTFGGGTKLEIK | |
| SCT-Ga267 | EVQLQQSGPELVKPGASMKTSCKASGYSFTGYT | 222 | DVVVTQTPLSLPVSFGDQVSISCRSSQSLANSY | 469 |
| | MNWVKQSHGKNLEWIGLIVPNNGGTSYNQKFKG | | GTTYLSWYLHKPGQSPQLLIYGISNRFSGVPDR | |
| | KATLTVDKSSSTAYMELLSLTSEDSAVYYCARS | | FSGSGSGTDFTLKISTIKPEDLGMYYCLQGTHQ | |
| | LRFSFDYWGQGTTLTVSS | | PPTFGGGTKLEIK | |
| SCT-Ga268 | QIQLVQSGPELKKPGETVKISCKASGYIFTNYG | 223 | DIVMTQAAFSNPVTLGTSASISCRSSKNLLHSN | 549 |
| | MNWVKKAPGKGLKWMGWINSYTGESTYGDDFKG | | GITYLYWYLQRPGQSPQLLIYRMSNLASGVPNR | |
| | RFAFSSETSASTAYLQINNLKKEDTATYFCARD | | FSGSGSGTDFTLRISRVEAEDVGVYYCGQLLEL | |
| | SFAYWGQGTLVTVSA | | PYTFGGGTKLEIK | |
| SCT-Ga269 | QAYLQQSGAELVRSGASVKMSCKASGYTFTSYN | 224 | DIVMTQSQKFMSTSIGDRVSVTCKASQNVVTNV | 550 |
| | MHWVKQTPGQGLEWIGKIFPGNGFTDYNQIFRD | | AWYQQKPGQSPKALIYSASYRYSGVPDRFTGSG | |
| | KATLTADTSSSTAYMQISSLTSEDSAVYFCTRE | | SGTDFTLTINNVQSEDLADYFCQQHNSYPYTFG | |
| | GWLSLGYWGQGTTLTVSS | | GGTKLEIK | |
| SCT-Ga270 | QVQLQQPGAELVKPGASVKMSCKASGYTFTTFW | 225 | DILLTQSPAILSVSPGERVSFSCRASQSIGTSI | 551 |
| | TNWVKQRPGQGLEWTGDTFPGRGITNYNGKFKN | | QWYQQRTMGSPRLLIKYGSESISGIPSRFSGSG | |
| | KATLTLDTSSSTAYMQLSSLTSEDSAVYYCSRG | | SGTDFTLSINSVESEDSADYYCQQSYRWPYTFG | |
| | LGYFDVWGAGTTVTVSS | | GGSKLEIK | |
| SCT-Ga271 | EVNLVESGGGLVQPGGSLRLSCATSGFTFNDYY | 226 | DIQMTQSSSYLSVSLGGRVTITCKASDHINNWL | 552 |
| | MSWVRQPPGKALEWLCLIRDKANGYTTEYSASV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga272 | QVQLQQPGSELVRPGASVKLSCKASGYTFTSYW | 227 | DIVLTQSPATLSVTPGDSVSLSCRASQNIRNNL | 529 |
| | MHWVKQRHGQGLEWIGNIFPGTGSTNYDERFKN | | HWFQQKSHESPRLLIKYASQSISGIPSRFSGSG | |
| | KGTLTVDTSSSTAYMHLSSLTYEDSAVYYCTRE | | SGTDFTLSINSLETEDFGMYFCQQSNSWPYTFG | |
| | GLYPFDYWGQGTTLTVSS | | GGTKLEIK | |
| SCT-Ga273 | EVQLQQSGPELVKPGASMKISCKASGYSFTGYT | 151 | DVVVTQTPLSLPVSFGDQVSISCRSSQSLTNSY | 553 |
| | MNWVKQSHGKNLEWIGLIIPYNGGTSYNQKFKD | | GNTFLSWYLHKPGQSPQLLIYGISNRFSGVPDR | |
| | KATLTIDKSSSTAYMELLSLTSEDSAVYYCARG | | FSGSGSGTNFTLKISTIKPEDLGMYYCLQGTHQ | |
| | FRGYFAVDYWGQGTSVTVSS | | PWTFGGGTKLEIK | |
| SCT-Ga274 | EVQLQQSGPELEKPGSSVKISCKASGYSFTDYN | 228 | EIVLTQSPAIMSASPGEKVTMTCSAGSSVSSSY | 554 |
| | MHWVKQSNGKSLEWIGNIDVYYSGTSYNQKFRG | | LHWYQQKSGSSPRLLIYDTSNLASGVPVRFSGS | |
| | KATLTVDKSSSTAYMELKSLTSEDSAVYFCARS | | GSGTSYSLTISPMEAEDAATYYCQQWSSYPLTF | |
| | LLRWYFDVWGAGTTVTVSS | | GAGTKLELK | |
| SCT-Ga275 | EVMLVESGGGLVKPGGSLKLSCAASGFTFSTYA | 229 | NIMMTQSPSSLAVSAGEKVTMSCKSSQSVLYSS | 555 |
| | MSWVRQTPEKRLEWVATISSGGSYTYYPDSVKG | | NQMNYLAWYQQKPGQSPKLLIYWASTRESGVPD | |
| | RFTISRDNAKNTLYLQMSSLRSEDTAMYYCARH | | RFTGSGSGTDFTLTISSVQAEDLALYYCHQYLS | |
| | FISTIITHFDYWGHGTTLTVSS | | SLTFGAGTKLELK | |
| SCT-Ga276 | EVNLVESGGGLVQPGGSLRLSCATSGFTFNDYY | 201 | DIQMTQSSSYLSVSLGGRVTITCKASDHINNWL | 552 |
| | MSWVRQPPGKALEWLCLIRNKANGYTTEYSASV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga277 | QVQLQQSGPELVKPGASVKMSCKASGYTFTSYY | 230 | DVLMTQTPLSLPVSLGDQASISCRSSQNIVHSN | 556 |
| | IHWVKQRPGQGLEWIGWIYPGDGSTKYNEKFKG | | GNTYLEWYLQKPGQSPKLLIYKVSNRFSGVPDR | |
| | KTTLTADKSSSTAYMLLSSLTSEDSAIYFCTRD | | FSGSGSGTDFTLKISRVEAEDLGVYYCFQGSHV | |
| | GNYGDWFPYWGQGTLVTVSA | | PYTFGGGTKLEIK | |
| SCT-Ga278 | QVQLQQSGGEVMKPGASVKISCKATGYTFSNYW | 231 | DIQMTQPTSSLSASLGDRVTISCSASQGISSYL | 557 |
| | IEWVKQRPGHGLEWIGEILPVSDSAYYNEKFRG | | NWYQQKPDGTVTLLIYYTSSLHSGVPSRFSGSG | |
| | KATFTADKSSNTAYMQLSTLTSEDSAVYYCARQ | | SGTDYSLTISNLEPEDIATYYCQHYSKLPYTFG | |
| | KYGNYYYFDYWGQGTTLTVSS | | GGTKLEIK | |
| SCT-Ga279 | QVQLQQPGSELVRPGASVKLSCKASGYTFTSYW | 163 | DIVLTQSPASLAVSLGQRASISCRASESVDKYG | 558 |
| | IHWVKQRHGQGLEWIGNIFPGSGTSNYDEKFNN | | ISFMSWFQQKPGQPPKLLIYAASNQGSGVPARF | |
| | KGTLTVDTSSSTSYMHLSSLTSEDSAVYYCARE | | SGSGSGTDFSLNIHPMEEDDTAMYFCQQSKEVP | |
| | GLYAFDYWGQGTTLTVSS | | WTFGGGTKLEIK | |
| SCT-Ga280 | EVQLQQSGPELVKPGASMKISCKASGYSFTGYT | 232 | DVVVTQTPLSLPVSFGDQVSISCRSSQSLANSY | 559 |
| | MNWVKQSHGKNLEWIGLIVPYNGGTSYNQKFKG | | GTTYLSWYLHKPGQSPQLLIYEISNRFSGVPDR | |
| | KATLTVDKSSSTAYMELLSLTSEDSAVYYCARS | | FSGSGSGTDFTLKISTIKPEDLGMYYCLQGTHQ | |
| | VRFSFDYWGQGTTLTVSS | | PPTFGGGTKLEIK | |
| SCT-Ga281 | QIQLVQSGPELQKPGETVKISCKASGYTFTNYG | 233 | DIVMTQAAFSNPVTLGTSASISCRSSKNLLHSN | 466 |
| | MNWVKKAPGKGFKWMGWINTYTGESTYADDFKG | | GITYLYWYLQRPGQSPQVLIYRMSNLASGVPNR | |
| | RFAFSSETSASTVFLQINNLKNEDTATYFCARD | | FSGSGSGTDFTLRISRVEAEDVGVYYCGQLLEL | |
| | SFAYWGQGTLVTVSA | | PYTFGGGTKLEIK | |
| SCT-Ga282 | EVQLQQSGPDLVKPGASVKISCKASGYSFTGYY | 234 | DIWTQSPASLAVSLGQRATISCRASESVDSYG | 560 |
| | MHWVKQSHGKSLEWIGRVNPNNGGTSYNQKFKD | | KSFIHWYQQKPGQPPKLLIYVASNLESGVPARF | |
| | KAVLTVDKSSSTAYMEFRSLTSEDSAVYYCASS | | SGSGSRTDFTLTIDPVEADDAATYYCQQNNEDP | |
| | PLLRLRGYAMDDWGQGTSVTVSS | | WTFGGGTKLEIK | |
| SCT-Ga283 | QVQLQQPGAELVKPGASVKMSCKASGYTFTSFW | 235 | DILLTQSPAILSVSPGERVSFSCRASQSIGTSI | 561 |
| | INWVKQRPGQGLEWIGDIYPGRGITNYNGEFKS | | QWYQQRTNGSPRLLIKYGSESISGIPSRFSGSG | |
| | KATLTLDTSSSTAYMQLSSLTSEDSAVYYCSRG | | SGTDFTLSINSVESEDIADYYCQQSYRWPYTFG | |
| | LGYFDVWGAGTTVTVSS | | GGTKLEIK | |
| SCT-Ga284 | QVQLQQPGSELVRPGVSVKLSCKASGYTFTSYW | 236 | DIVLSQSPATLSVTPGDSVSLSCRASQSIRNNL | 562 |
| | MHWVKQRHGQGLEWIGNIFPGSGTTNYHEKFKN | | HWFQQKSHESPRLLITYASQSISGIPPRFSGSG | |
| | KGTLTVDTSSSTAYMHLSSLTSEDSAVYYCSRE | | SGTDFTLSINSLETEDFGMFFCQQSNSWPYTFG | |
| | GLYPFDYWGQGTTLTVSS | | GGTKLEIK | |
| SCT-Ga285 | QAYLQQSGAELVRSGASVKMSCKASGYTFTSYN | 164 | DIVMTQSQKFMSTSVGDRVSITCKASQHVVTNV | 563 |
| | MHWVKQTPGQGLEWIGKIFPGNGFTDYNQKFRD | | AWYQQKPGQSPKALIYSASYRYSGVPDRFTGSG | |
| | KATLTADTSSSTAYMQISSLTSEDSAVYFCTRE | | SGTDFTLTISNVQSEDLADYFCQQHNSYPYTFG | |
| | GWLSLGYWGQGTTLTVSS | | GGTKLEIK | |
| SCT-Ga286 | QVQLKESGPGLVAPSQSLSITCTVSGFSLTSYD | 237 | DILLTQSPAILSVSPGERVSFSCRASQNIGTSI | 564 |
| | ISWIRQPPGKGLEWLGVIWTGGGTNYNSAFMSR | | HWYQQRTNGSPRLLIKYAFESISGIPSRFSGSG | |
| | LSISKDNSKSQVFLKMNSLQTDDTAIYYCVRST | | SGTDFTLSINSVESEDIADYYCQQSNSWPLTFG | |
| | MITHYAMDYWGQGTSVTVSS | | AGTKLELK | |
| SCT-Ga287 | EVNLVESGGGLVQPGGSLRLSCATSGFTFNDYY | 201 | DIQMTQSSSYLSVSLGGRVTITCRASDHINNWL | 565 |
| | MSWVRQPPGKALEWLCLIRNKANGYTTEYSASV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga288 | QITLKESGPGIVQPSQPFRLTCTFSGFSLSTSG | 192 | DIQMTQSSSYLSVSLGGRVTITCKASDHINNWL | 566 |
| | IGVTWIRQPSGKGLEWLATIWWDDDNRYNPSLK | | AWYQQKPGNAPRLLISGATSLKTGVPSRFSGSG | |
| | SRLTVSKDTSNNQAFLNIITVETADTAIYYCAQ | | SGKDYTLSITSLQIEDVATYYCQQYWSIPFTFG | |
| | SGGITLMDYWGQGTSVTVSS | | SGTKLEIK | |
| SCT-Ga289 | QVQLQQPGSELVRPGASVKLSCKASGYTFTSYW | 238 | DIVLTQSPATLSVTPGDSVSLSCRASQSIRNSL | 567 |
| | MHWVKQRHGQGLEWIGNIYPGSGTTNYDEKFKN | | HWFQQKSHESPRLLIKYVFQSISGIPSRFSGSG | |
| | KGTLTVDTSSSTAYIHLSSLTSEDSAVYYCTRE | | SGTDFTLSINSLETEDFGMYFCQQSNSWPYTFG | |
| | GLYPFDYWGQGTTLTVSS | | GGTKLEIK | |
| SCT-Ga290 | QAYLQQSGAELVRSGASVKMSCKASGYTFTSYN | 210 | QIVLTQSPAIMSASPGEKVTMTCSATSSVSSMH | 568 |
| | LHWVKQTPGQGLKWIGYIYPGNGYTNYNQKFKG | | WYQQKSGTSPKRWIYDTSKLASGVPGRFSGSGS | |
| | KATLTADTSSSTAYMQINSLTSEDSAVYFCARE | | GTSYSLTISSMEAEDAATYYCQQWSSNPSTFGA | |
| | GGWAMDYWGQGTSVTVSS | | GTKLELK | |
| SCT-Ga291 | QVQLQQPGSELVRPGASVKLSCKASGYTFTSYW | 239 | DIVLTQSPATLSVTPGDSVSLSCRASQSIRNSL | 569 |
| | MHWVKQRHGQGLEWIGNIFPGSGITNYDEKFKN | | HWFQQKSLESPRLLINYASQSISGIPSRFRGSG | |
| | KGTLTVDTSSNTAYMHLSSLTSDDSAVYYCARE | | SGTDFTLSINNLETEDFGMYFCQQTNSWPYTFG | |
| | GLYAFDYWGQGTTLTVSS | | GGTKLEIK | |
| SCT-Ga292 | EVQLQQSGPELEKPGSSVKISCTASGFSFTDYN | 240 | EIVLTQSPAIMSASPGEKVTVTCSAGSSVSSSN | 517 |
| | IHWVKQSNGKSLEWIGDVDIYYSGTSYNQKFKG | | LHWYQQKSGSSPRLLIYDTSKLASGVPVRFSGS | |
| | KATLTVDKSSSTTYMELKSLTSEDSAVYFCARS | | GSGTSYALTISRMEAEDAATYYCQQWSSYPLTF | |
| | SLRWYFDLWGAGTTVTVSS | | GAGTKLELK | |
| SCT-Ga293 | QVQLQQPGAELVKPGASVKMSCKASGYTFSNYW | 241 | DIVLTQSPATLSVTPGDSVSLSCRASQSIRNNL | 570 |
| | MHWVKQRPGQGLEWIGTIDPSDSYTSYNPKFKG | | HWYQQKSHESPRLLIKYASQSISGIPSRFSGSG | |
| | KATLTVDPSSSTAYMQLSSPTSEDSAVYYCTRP | | SGTNFTLSINSVETEDFGMYFCQQSYSWPYTFG | |
| | SSYFFDYWGQGTTLTVSS | | GGTKLEIN | |
| SCT-Ga294 | QAYLQQSGAELVRSGASVKMSCKASGYTFTSYN | 242 | DIVMTQSQKFMSTSVGDRVSVTCKASQNVVTNV | 571 |
| | MHWVRQTPGQGLEWIGSIYPGNDGTNYIQKFKG | | AWYQQKPGQSPKPLIYSASYRNSGVPDRFTGSG | |
| | KATLTTDTSSSTAYMQISSLTSEDSAVYFCARE | | SGTDFTLTISNVQSEDLAEYFCQQYKSYPYTFG | |
| | GWLSLDYWGQGTTLTVSS | | GGTKLEIK | |
| SCT-Ga295 | EVQLQQSGPELVKPGASMKISCKASGYSFTGNT | 243 | DVVVTQTPLSLPVSFGDQVSISCRSSQSLANSY | 572 |
| | MNWVKQSHEKNLEWIGLINPYNGGTSYNQKFKG | | GNTYLSWYLHKPGQSPQLLIYGISNRFSGVPDR | |
| | KATLTVDKSSSTAYMELLSLTSEDSAVYYCARG | | FSGSGSGTDFTLKISTIKPEDLGMYYCLQGTHQ | |
| | FRGYFAMDYWGQGTSVTVSS | | PWTFGGGTKLEIK | |
| SCT-Ga296 | QITLKESGPGIVQPSQPFRLTCTFSGFSLSTSG | 182 | DVLMTQTPLSLPVSLGDQASISCRSSQNIVHSN | 573 |
| | IGVTWIRQPSGKGLEWLATIWWDDDTRYNPSLK | | GNTYLEWYLQKPGQSPKLLIYKVSKRFSGVPDR | |
| | SRLTVSKDTSNNQAFLNILTVETADTAIYYCAQ | | FSGSGSGTDFTLKISRVEAEDLGVYYCFQTSHV | |
| | SGGTTLMDYWGQGTSVTVSS | | PGTFGGGTKLEIK | |
| SCT-Ga297 | EVQLQQSGPELVKPGASVKISCKASGYTFTDYN | 244 | DIVLTQAAFSNPVTLGTSVSISCRSSKNLLHSN | 574 |
| | MHWVKQSHGKSLEWIGYIYPYNVGTAYNQKFKG | | GITYLYWHLQRPGQSPQLLIYRVSNLASGVPNR | |
| | KATLTVDNSSSTAYMELRSLTSEDSAVYYCARK | | FSGSESGTDFTLRISRVEAEDVGVYYCAQLLEL | |
| | NPLDYWGQGTTLTVSS | | PYTFGGGTKLEIK | |
| SCT-Ga298 | QVQLQQSGGELMKPGASVKTSCKATGYTFSNCW | 245 | DIQMTQTTSSLSASLGDRVTISCSASQGISNYL | 575 |
| | IEWVKQRPGHGLEWTGEILPGSGSAYYNEKFKG | | NWYQQKPDGTVKLLIYYTSSLHSGVPSRFSGSG | |
| | KATFTADKSSNTAYMQLSSLTSEDSAVYYCARQ | | SGTDYSLTISNLEPEDIATYYCQHYSKLPYTFG | |
| | KYGNYYYFDYWGQGTTLTVSS | | GGTKLEIK | |
| SCT-Ga299 | EVQLQQSGTVLARPGASVKMFCKASGYSFTSYW | 246 | DIVLTQSPATLSVTPGDSVSLSCRASQSIRNNL | 576 |
| | MHWVKQRPGQGLEWIGAFYPGNSATNYNQKFKG | | HWYQQKSHESPRLLIKYSSQSISGIPSRFSGSG | |
| | KAKLTAVTSASTAYMELSSLTNEDSAVYYCSRE | | SGTDFTLSINSVETEDFGMYFCQQSNSWPYTFG | |
| | GGYGMDHWGQGASVTVSS | | GGTKLEIN | |
| SCT-Ga300 | QVQLQQSGPELVKPGASVKMSCKASGYTFTSYY | 230 | DIVLTQSPASLAVSLGQRATISCRASQSVSTSR | 577 |
| | IHWVKQRPGQGLEWIGWIYPGDGSTKYNEKFKG | | YSYMHWYQQKPGQSPKLLIKYASNLESGVPARF | |
| | KTTLTADKSSSTAYMLLSSLTSEDSATYFCTRD | | SGSGSGTDFTLNINPVEEEDTATYYCQHSWEIP | |
| | GNYGDWFPYWGQGTLVTVSA | | LTFGAGTKLELK | |
| SCT-Ga301 | EVKLVESGGGLVKPGGSLKLSCAASGFAFSSFD | 247 | DIQMNQSPSSLSASLGDTITITCHASQNINVWL | 540 |
| | MSWVRQTPEKRLEWVAVISSGGSYTFYPDSVKG | | SWYQQKPGNIPKLLIYKASNLHTGVPSRFSGSG | |
| | RFTISRDNARNTLYLQMSSLRSEDTALYYCARQ | | SGAGFTLTISSLQPEDIATYYCQQGQSYPWTFG | |
| | DYRYPYTMDYWGQGTSVTVSS | | GGTKLEIK | |
| SCT-Ga302 | EVQLQQSGTVLARPGASVKMFCKASGYSFTSYW | 246 | DIVLTQSPATLSVTPGDSVSLSCRASQSIRNNL | 578 |
| | MHWVKQRPGQGLEWTGAFYPGNSATNYNQKFKG | | HWYQQKSHESPRLLIKYASQSISGIPSRFSGSG | |
| | KAKLTAVTSASTAYMELSSLTNEDSAVYYCSRE | | SGTDFTLSINSVETEDFGMYFCQQSNSWPYTFG | |
| | GGYGMDHWGQGASVTVSS | | GGTKLEIN | |
| SCT-Ga303 | EVQLQQSGPELVKPGASMKISCKASGYSFTGYT | 248 | DVVVTQTPLSLPVSFGDQVSISCRSSQSLANSY | 482 |
| | MNWVKQSHGKNLEWIGLIIPYNGGTSYNQKFKG | | GNTYLSWYLHKPGQSPQLLIYGISNRFSGVPDR | |
| | KATLTVDKSSSTAYMELLSLTSEDSAVYYCARS | | FSGSGSGTDFTLKISTIKPEDLGMYYCLQGTHQ | |
| | LRFSFDYWGQGTSLTVSS | | PPTFGGGTKLEIK | |
| SCT-Ga304 | QAYLQQSGAELVRSGASVKMSCKASGYTFSNYN | 249 | QIVLTQSPEIMSASPGEKVTMTCSASSSVSHMH | 579 |
| | MHWVKQTPGQGLEWIGYIFPGNGVTTYSQKFKD | | WYQQKSGTSPKRWIYDTSKLASGVPARFSGSGS | |
| | KATLTADTSSSTAYMQISSLTSEDSAVYFCARG | | GTSYSLTISSMEAEDSATFYCQQWNNNPLTFGA | |
| | GFGGALDYWGQGTSVTVSS | | GTKLELK | |
| SCT-Ga305 | EVQLQQSGTVLARPGASVKMSCKASGYSITSYW | 198 | DWLTQSPATLSVTPGDSVSLSCRASQTISNNL | 580 |
| | MHWVKQRPGQGLEWIGGVYPGNSNTNYNQKFKG | | HWYQQKSHESPRLLIKYSSQSISGIPSRFSGSG | |
| | KAKLTAVTSASTAYMELSSLTNEDSAVYYCTRE | | SGTDFTLSINSVETEDFGMYFCQQSYSWPYTFG | |
| | GGYGMDYWGQGTSVTVSS | | GGTKLEIK | |
| SCT-Ga306 | EVQLQQSGPELVKPGASMKISCKASGYSFTGYT | 173 | DWMTQTPLSLPVSFGDQVSISCRSSQSLANSY | 581 |
| | MNWVKQSHGKNLEWLGLIFPYNSATSYNQKFTG | | GNTYLSWYLHKPGQSPHLLIYGISHRFSGVPDR | |
| | KATITVDKSSNTTYMELLSLTSEDSAVYYCARG | | FSGSGSGTDFTLKISTIKPEDLGMYYCLQGTHQ | |
| | LRGFFALDYWGQGTSVTVSS | | PWTFGGGTKLEIK | |
| SCT-Ga307 | QAYLQQSGAELVRSGASVKMSCKASGYTFSNYN | 250 | QIVLTQSPEIMSASPGEKVTMTCSASSSVSHMH | 582 |
| | MHWVKQTPGQGLEWIGYIFPGNGVTTYSQKFKD | | WYQQKSGTSPKRWIYDTSKLASGVPARFSGSGS | |
| | KATLTADTSSSTAYMQISSLTSEDSAVYFCARG | | GTSYSLTISSMEAEDSATYFCQQWNNNPLTFGA | |
| | GFGGAMDYWGQGTSVTVSS | | GTKLELK | |
| SCT-Ga308 | QITLKESGPGIVQPSQPFRLTCTFSGFSLSTSG | 251 | DVLMTQTPLSLPVSLGDQASISCRSSQSIVHSD | 583 |
| | IGVTWIRQPSGKGLEWLATIWWDDDNRYNPSLK | | GNTYLQWYLQKPGQSPKLLIYKVSRRFSGVPAR | |
| | NRLTVSKDTSNNQAFLNFITVETADTAIYYCAQ | | FSGSGSGTDFTLKISRVEAEDLGVYFCFQGSHV | |
| | SGGITLMDYWGQGTSVTVSS | | PGTFGGGTKLEII | |
| SCT-Ga309 | QITLKESGPGIVQPSQPFRLTCTFSGFSLSTSG | 252 | DVLMTQSPLSLPVSLGDQASISCRSSQSIVHSD | 531 |
| | IGVTWIRQPSGKGLEWLATIWWDDDNRYNPSLK | | GNTYLEWYLQKPGQSPKLLIYKVSRRFSGVPDR | |
| | SRLTVSKDTSNNQVFLNIIDVESADTAMYYCAQ | | FSGSGSGTDFTLKISRVGAEDLGVYYCFQGSHV | |
| | SGGITLMDYWGQGTSVTVSS | | PGTFGGGTKLEIK | |
| SCT-Ga310 | QIQLVQSGPELKKPGETVKISCKASGYTFTNFG | 253 | DIVMTQAAFSNPVTLGTSASISCRSSKNLQHNN | 584 |
| | MNWVKQTPGKGLKWMGWINTYTGEPTYADDLKG | | GITYLYWYLQRPGQSLQLLIYRVSNLASGVPNR | |
| | RFALSLETSAGTSYLQINTLRDEDTATYFCARD | | FSGSESGTDFTLRISRVEPEDVGVYYCGQLLEL | |
| | SFAHWGQGTLVTVSA | | PYTFGGGTRLEIK | |
| SCT-Ga311 | QAYLQQSGAELVRSGASVKMSCKASGYTFTSYN | 254 | QIVLTQSPATMSASPGEKVTMTCSASSSVSHMH | 585 |
| | MHWVKQTPGQGLEWIGYIYPGNGFTNYNQKFKG | | WYQQKSGTSPKRWIYDTSKLASGVPVRFSGSGS | |
| | KATLTADTSSSTAYMQISSLTSEDSAVYFCARE | | GTSYSLTISSMEAEDAATYYCQQWNSNPSTFGA | |
| | GGWAMDYWGQGTSVTVSS | | GTKLELK | |
| SCT-Ga312 | EVNLVESGGGLVQPGGSLRLSCATSGFPFNDYY | 189 | DIQMTQSSSYLSVSLGGRVTITCKASDHIHNWL | 586 |
| | MSWVRQPPGKALEWLCLIRNKANGYTTEYSSSV | | AWYQQKPGNSPRLLISGATSLEAGVPSRFSGSG | |
| | KGRFTISRDNSQSILYLQMNTLRAEDSATYYCA | | SGKDYTLSITSLQTEDVGTYYCQQYWSIPFTFG | |
| | RDGGYWGQGTTLTVSS | | SGTKLEIK | |
| SCT-Ga313 | EVQLQQSGPELVKPGASMKISCKASGYSFTGYT | 255 | DVVVTQTPLSLPVSFGDQVSISCRSSQSLANSY | 482 |
| | MNWVKQSHGKSLEWIGLIIPYNGGTSYNQKFKG | | GNTYLSWYLHKPGQSPQLLIYGISNRFSGVPDR | |
| | KATLSVDKSSSTAYMELLSLTSEDSAVYYCARS | | FSGSGSGTDFTLKISTIKPEDLGMYYCLQGTHQ | |
| | LRFSFDYWGQGTTLTVSS | | PPTFGGGTKLEIK | |
| SCT-Ga314 | QVQLQQPGSELVRPGASVKLSCKASGYTFTSYW | 256 | DIVLTQSPATLSVTPGDSVSLSCRASQSIRNSL | 587 |
| | MHWVKQRHGQGLEWIGNIFPGSGTTNYDEKFKS | | HWFQQKSHESPRLLIKYASQSMSGIPSRFSGSG | |
| | KGTLTVDTSSSTAYMHLSSLTSEDSAVYYCTRE | | SGTDFTLSINSLETEDFGMYFCQQSNSWPYTFG | |
| | GLYAFDYWGQGTTLTVSS | | GGTKLEIK | |
| SCT-Ga315 | EVKLVESGGGLVQPGGS1RLSCATSGFTFTDYY | 257 | DIQMTQSSSYLSVSLGGRVTITCKASDHINNWL | 588 |
| | MSWVRQPPGKALEWLALIRNKANGYTTEYSASV | | AWYQQKPGNAPRLLISGATSLETGVPSRFSGSG | |
| | KGRFTISRDNSQSILYLQMNTLRAEDSATYYCA | | SGKDYILSITRLQTEDVATYYCQQYWSTPFTFG | |
| | RDGGYWGQGTTLTVSS | | SGTKLEIK | |
| SCT-Ga316 | QVQLQQPGSELVRPGASVKLSCKASGYTFTSYW | 258 | DLVLTQSPATLSVTPGDSVSLSCRASQSIRNNL | 589 |
| | MHWVKQRHGQGLEWIGNIYPGGGTTNYDEKFRN | | HWFQQKSHESPRLLITYVSQSISGIPSRFSGSG | |
| | KATLTVDTSSSTAYMHLSSLTSEDSAVYYCTRE | | SGTDFTLSINSLETEDFGMYFCQQSNSWPYTFG | |
| | GLYPFDYWGQGTTLTVSS | | GGTKLEIK | |
| SCT-Ga317 | EVQLQQSGTVLARPGASVKMSCKTSGYSFTSYW | 259 | DIVLTQSPATLSVTPGDSVSLSCRASQSIYNNL | 590 |
| | MHWVKQRPGQGLEWIGGIYPGNSDTTYNQKFKG | | HWYQQKSHESPRLLIKFSSQSISGIPSRFSGSG | |
| | KAKLTAVTSASTAYMELSSLTNEDSAVYYCTRE | | SGTDFTLNINSVETEDFGMYFCQQSNSWPYTFG | |
| | GGYGMDYWGQGTSVTVSS | | GGTKLEIK | |
| SCT-Ga318 | QVQLQQPGSELVRPGASVKLSCKASGYTFTSYW | 260 | DIVLTQSPATLSVTPGDSVSLSCRASQNIRNSL | 591 |
| | IHWVKQRHGQGLEWIGNIFPGSGTSNYDERFKN | | HWFQQKSHESPRLLINYASQSISGIPSRFRGGG | |
| | MGTLTVDTSSNTAYMDLSSLTSEDSAVYYCARE | | SGTDFTLSINSLETEDFGMYFCLQSNSWPYTFG | |
| | GLYAFDYWGQGTTLTVSS | | GGTKLEIK | |
| SCT-Ga319 | QVQLQQSGPELVKPGASMKISCKASGYSFTGYT | 261 | DVVVTQTPLSLPVSFGDQVSISCRSSQSLANSY | 592 |
| | MNWVKQSHGKNLEWIGTINPSNGGTSYNQKFTG | | GNTYLSWYLHKPGQSPQLLIYGISNRFSGVPDR | |
| | KATLTVDKSSTTAYMELLSLTSEDSAVYYCTRR | | FSGSGSGTDFTLKISTIKPEDLGMYFCLQGTHQ | |
| | NYGYYFDYWGQGTTLTVSS | | PLTFGAGTKLELK | |
| SCT-Ga320 | EVKLVESGGGLVQPGGSLRLSCVTSGFTFTDYY | 262 | DIQMTQSSSYLSVSLGGRVTITCKASDHINNWL | 593 |
| | MSWVRQPPGKALEWLALIRNKANGYTTEYSASV | | AWYQQKPGNAPRLLISGATSLKTGVPSRFSGSG | |
| | KGRFTISRDNSQSILYLQMNTLRAEDSATYFCA | | SGKDYILSITSLQTEDVGTYYCQQYWSIPFTFG | |
| | RDGGYWGQGTTLTVSS | | SGTKLEIK | |
| SCT-Ga321 | QITLKESGPGIVQPSQPFRLTCTFSGFSLSTSG | 263 | DVLMTQTPLSLPVSLGDQASISCRSSQSIVHSD | 594 |
| | IGVTWIRQPSGKGLEWLATIWWDDDNRYNPSLK | | GNTYLEWYLQKPGQSPKLLIYKVSRRFSGVPDR | |
| | SRLTVSKDTSNNQAFLNILTVDTADTAIYYCAQ | | FSGSGSGTDFTLKIRRVEAEDLGVYYCFQGSHV | |
| | SGGITLMDYWGQGTSVTVSS | | PGTFGGGTKLEIK | |
| SCT-Ga322 | QVQLQQPGSE’VRPGASVKLSCKASGYTFTSYW | 220 | DIVLTQSPATLSVTPGDSVSLSCRASQSIRNSL | 595 |
| | MHWVKQRHGQGLEWIGNIFPGSGITNYDEKFKN | | HWFQQKSHESPRLLINYASQSISGIPSRFRGSG | |
| | KGTLTVDTSSSTAYMHLSSLTSEDSAVYYCARE | | SGTDFTLIINSLETEDFGMYFCQQSNSWPYTFG | |
| | GLYAFDYWGQGTTLTVSS | | GGTKLEIK | |
| SCT-Ga323 | EVNLVESGGGLVQPGGSLRLSCATSGFPFNDYY | 264 | DIQMTQSSSYLSVSLGGRVTITCKASDHINNWL | 596 |
| | MSWVRQPPGKALEWLCLIRNKAKGYTTEYSSSV | | AWYQQKPGNSPRLLISGATSLGAGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga324 | EVNLVESGGGLVQPGGSLRLSCATSGFPFNDYY | 189 | DIQMTQSSSYLSVSLGGRVTITCKASDHINNWL | 596 |
| | MSWVRQPPGKALEWLCLIRNKANGYTTEYSSSV | | AWYQQKPGNSPRLLISGATSLGAGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga325 | QVQLQQPGAEFVKPGASVRMSCKASGYTFTSFW | 265 | DILLTQSPAILSVSPGERVSFSCRASQNIGTSI | 597 |
| | INWVKQRPGQGLEWIGDIYPGRGIANYNEKFNN | | HWYQQRTSGSPRLLIKSASESISGIPSRFSGSG | |
| | KATLTLDTSSNTAYMQLSSLTSEDSAVYYCSRG | | SGTDFTLSINSVESEDIADYYCQQSNRWPYTFG | |
| | LGYFDVWGAGTTVTVSS | | GGTKLEIK | |
| SCT-Ga326 | QAYLQQSGAELVRSGASVKMSCKASGYTFTSYN | 266 | QIVLTQSPAIMSASPGEKVTMTCSASSSVSQMH | 598 |
| | MHWVKQTPGQGLEWIGYIYPGNGYTNYNQKFKD | | WYQQKSGTSPKRWIYDTSKLASGVPARFSGSGS | |
| | KATLTADTSSSTAYMQISSLTSEDSAVYFCARE | | GTSYSLTLSSMEAEDAATYYCQQWSSNPSTFGV | |
| | GGWAMDYWGQGTSVTVSS | | GTKLELK | |
| SCT-Ga327 | QITLKESGPGIVQPSQPFRLTCTFSGFSLSTSG | 267 | DVLMTQSPLSLPVSLGDQASISCRSSQSIVHSD | 599 |
| | IGVTWIRQPSGKGLEWLATIWWDDDNRYNPSLK | | GNTYLEWYLQKPGQSPKLLIYKVSRRFSGVPDR | |
| | SRLTVSKDTSNNQVFLNIIAVETADTAMYYCAQ | | FSGSGSGTDFTLEISRVGAEDLGVYYCFQASHV | |
| | SGGITLMDYWGQGTSVTVSS | | PGTFGGGTKLEIK | |
| SCT-Ga328 | QVQLQQSGAELVRPGTSVKISCKASGYTFTNFW | 268 | DIKMTQSPSSMYASLGERVTITCKASRDIKSYL | 600 |
| | LGWVKQRPGHGLEWVGDIFPGGGYTNYNEKFKG | | SWYQQKPWKSPKTLIFYATRLADGVPSRFSGSG | |
| | KATLTADTSSSTAYMQLSSLTSEDSAVYFCSRS | | SGQDFSLTISSLESDDTATYYCLHHGESPYTFG | |
| | TMLTGTYWGQGTLVTVSA | | GGTKLEIK | |
| SCT-Ga329 | EVNLVESGGGLVQPGGSLRLSCATSGFTFNDYY | 201 | DIQMTQSSSYLSVSLGGRVTITCKASDHINNWL | 515 |
| | MSWVRQPPGKALEWLCLIRNKANGYTTEYSASV | | AWYQQKPGNAPRLLISGATSLKTGVPSRFSGSG | |
| | KGRFTISRDNSQSILYLQMNTLRAEDSATYYCA | | SGKDYTLSITSLQTEDVATYYCQQYWSTPFTFG | |
| | RDGGYWGQGTTLTVSS | | SGTKLEIK | |
| SCT-Ga330 | QVQLQQSGAELVRPGTSVKISCKASGYTFTNFW | 269 | DIKMTQSPSSMYASLGERVTITCKASQDIKSYL | 601 |
| | LGWVKQRPGHGLEWIGDIFPGGGHTNYNEKFKG | | SWYQQKPWKSPKTLIFYATRLADGVPSRFSGSG | |
| | KATLTADTASSTAYMQLSSLTSEDSAVYFCSRS | | SGQDFSLTISSLESDDTATYYCLHHGESPYTFG | |
| | TILTGAYWGQGTLVTVSA | | GGTKLEIK | |
| SCT-Ga331 | EVKLVESGGGLVQPGGSLRLSCATSGFTFTDYY | 257 | DIQMTQSSSYLSVSLGGRVTITCKASDHINNWL | 506 |
| | MSWVRQPPGKALEWLALIRNKANGYTTEYSASV | | AWYQQKPGNAPRLLISGATSLETGVPSRFSGSG | |
| | KGRFTISRDNSQSILYLQMNTLRAEDSATYYCA | | SGKDYILSITSLQTEDVATYYCQQYWSTPFTFG | |
| | RDGGYWGQGTTLTVSS | | SGTKLEIK | |
| SCT-Ga332 | EVNLVESGGGLVQPGGSLRLSCATSGFTFNDYY | 201 | DIQMTQSSSYLSVSLGGRVTITCKASDHINNWL | 602 |
| | MSWVRQPPGKALEWLCLIRNKANGYTTEYSASV | | AWYQQRPGNAPRLLISGATSLGTGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIN | |
| SCT-Ga333 | QVQLQQSGAELVRPGTSVKVSCKASGYAFTNYL | 270 | DIVMTQSHKFMSTSVGDRVSITCKASQDVSTAV | 603 |
| | IEWVKQRPGQGLEWIGVINPGSGGNNYNEKFKG | | AWYQQKPGQSPKLLIYSASYRYTGVPDRFTGSG | |
| | KATLTADKSSSTAYMQLSSLTSDDSAVYFCARR | | SGTDFTFTISSVQAEDLAVYYCQQHYSSPRTFG | |
| | GDKYGSAWFAYWGQGTLVTVSA | | GGTKLEIK | |
| SCT-Ga334 | EVNLVESGGGLVQPGGSLRLSCATSGFTFNDYY | 271 | DIQMTQSSSYLSVSLGGRVTITCKASDHINNWL | 552 |
| | MSWVRQPPGKALEWLGLIRDKANGYTTEYSASV | | AWYQQKPGNAPRLLISGATSLETGVPSRFSGSG | |
| | KGRFTISRDNSQSILYLQMSTLRAEDSATYYCA | | SGKDYTLSITSLQTEDVATYYCQQYWSIPFTFG | |
| | RDGGYWGQGTTLTVSS | | SGTKLEIK | |
| SCT-Ga335 | EVQLQQSGPELEKPGSSVKISCKASGYSFTDYN | 177 | EIVLTQSPAIMSASPGEKVTVTCSAGSSVNSTY | 604 |
| | MHWVKQSNGKSLEWIGDIDVYYSGTSYNQKFKG | | LHWYQQKSGSSPRLLIYDTSKLASGVPVRFSGS | |
| | KATLTVDKSSSTTYMELKSLTSEDSAVYFCARS | | GSGTSYALTISRMEAEDAATYYCQQWSSYPLTF | |
| | SLRWYFDLWGAGTTVTVSS | | GAGTKLELK | |
| SCT-Ga336 | EVQLQQSGPELVKPGASMKISCKASGYSFTGYT | 183 | DVVVTQTPLSLPVSFGDQVSISCRSSQSLANSY | 469 |
| | MNWVKQSHGKNLEWIGLIVPYNGGTSYNQKFKG | | GTTYLSWYLHKPGQSPQLLIYGISNRFSGVPDR | |
| | KATLTVDKSSSTAYMELLSLTSEDSAVYYCARS | | FSGSGSGTDFTLKISTIKPEDLGMYYCLQGTHQ | |
| | LRFSFDYWGQGTTLTVSS | | PPTFGGGTKLEIK | |
| SCT-Ga337 | EVQLQQSGPELEKPGSSVKISCKASGYSFTDYN | 272 | EIVLTQSPATMSASPGEKVTMTCSASSSVSSSY | 605 |
| | IHWVKQSNGKSLEWIGNIDVYYSSTSYNQKFKG | | LHWYQQKSGSSPRLLIYDTSNLASGVPVRFSGS | |
| | KATLTVDKSSSTAYMQLKSLTSEDSAVYFCARS | | GSGTSYSLTISRMEAEDAATYYCQQWSSYPLTF | |
| | LLRWYFDVWGAGTTVTVSS | | GAGTKLELK | |
| SCT-Ga338 | QVQLQQPGSELVRPGASVKLSCKASGYTFTSYW | 273 | DIVLTQSPATLSVTPGDSVSLSCRASQSIRNNL | 606 |
| | MHWVKQRHGQGLEWIGNIYPGSGTTNYDEKFKN | | HWFQQKSHESPRLLITYASQSISGIPSRFSGSG | |
| | KGTLTVDTSSSTAYMHLSSLTSEDSAVYYCTRE | | SGTDFTLSINSLETEDFGIYFCQQSNSWPYTFG | |
| | GLYPFDYWGQGTTLTVSS | | GGTKLEIK | |
| SCT-Ga339 | EVLLQQSGPELVKPGASVKIPCKASGYTFTDYN | 274 | DIQMTQSSSYLSVSLGGRVTITCKASDHINNWL | 596 |
| | MDWVKQSHGKSLEWIGNINSNNGGAIYNQKFKD | | AWYQQKPGNSPRLLISGATSLGAGVPSRFSGSG | |
| | KATLTVDKSSSTAYMELRSLTSEDTAVYYCARG | | SGKDYTLSITSLQTEDVATYYCQQYWSTPFTFG | |
| | DYYGTTYDYWGQGTTLTVSS | | SGTKLEIK | |
| SCT-Ga340 | EVQLQQSGPELVKPGASMKISCKASGYSFTGYI | 275 | QIVLTQSPAIMSASPGEKVTISCSASSSVSYMY | 607 |
| | MNWVKQSHGKTLEWIGLIHPYNGGTSYNQKFKG | | WYQQKPGSSPKPWIYRTSNMASGVPARFSGSGS | |
| | KATLTVDKSSSTAYMELLSLTSEDSAVYYCARL | | GTSYSLTISSMEAEDAATYYCQQYHGYPPTFGG | |
| | LRYAMDYWGQGTSVTVSS | | GTKLEIK | |
| SCT-Ga341 | QVQLQQSGAELARPGASVKLSCKASGYTFISYW | 276 | DIVMTQSQKFMSTSIGDRVSVTCKASQNVVTNV | 608 |
| | MQWVKQRPGQGLEWIGAIYPGDGDTRYTQKFKG | | AWYQQKPGQSPKALIYSASYRYSGVPDRFTGSG | |
| | KATLTADKSSSTAYMQLSSLASEDSAVYYCARG | | SGTDFTLTISNVQSEDLADYFCQQHNSYPYTFG | |
| | TVGDYWGQGTTLTVSS | | GGTKLEIK | |
| SCT-Ga342 | QIQLVQSGPELKKPGETVKISCKASGYTFTNYG | 277 | DIVMTQAAFSNPVTLGTSASISCRSSKNLLHSN | 466 |
| | MNWVKKAPGKGLKWMVWINTYTGESTYADDFKG | | GITYLYWYLQRPGQSPQVLIYRMSNLASGVPNR | |
| | RFAFSSETSASTAYLQINNLKNEDTATYFCARD | | FSGSGSGTDFTLRISRVEAEDVGVYYCGQLLEL | |
| | SFAYWGQGTLVTVSA | | PYTFGGGTKLEIK | |
| SCT-Ga343 | EVQLQQSGPELVKPGASMKISCKASGYSFTDYT | 278 | DVVVTQTPLSLPVSFGDQVSISCRSSQSLANSY | 609 |
| | MNWVKQSHGKNLEWIGLIIPYNGGTSYNQKFKG | | GVTYLSWYLHKPGQSPQLLIYGISNRFSGVPDR | |
| | KATLTLDKSSSTAYMELLSLTSEDSAVYFCARG | | FSGSGSGTDFTLKISTIKPEDLGMYYCLQGTHQ | |
| | LRGYFAMDYWGRGTSVTVSS | | PWTFGGGTKLEIK | |
| SCT-Ga344 | EVQLQQSGPELVKPGASMKISCKASGYSFTGYT | 279 | DVVVTQTPLSLPVSFGDQVSISCTSSQSLANSY | 610 |
| | MNWVKQSHGKNLEWIGLIFPYNGGTSYNQKFKD | | GNTYLSWYLHKPGQSPQLLIYGISNRFSGVPDR | |
| | KATLTVDKSSSTAYMELLSLTSEDSAVYYCARS | | FSGSGSGTDFTLKISAIKPEDLGMYYCLQGTHQ | |
| | VRFSFDYWGQGTSLTVSS | | PPTFGGGTKLEIK | |
| SCT-Ga345 | EVKLVESGGGLVKPGGSLKLSCAASGFAFSRYD | 280 | DIQMNQSPSSLSASLGDTITITCHASQNINVWL | 611 |
| | MSWVRQTPEKRLEWVATTSSGGSYSFYPDSVKG | | SWYQQKPGNFPKLLIYKTSNLHTGVPSRFSGSG | |
| | RFTISRDNGRNTLYLQMSSLRSEDTALYYCARQ | | SGTGFTLTISNLQPEDIATYYCQQGQSYPWTFG | |
| | DYRYPYIMDYWGQGTSVTVSS | | GGTKLEIK | |
| SCT-Ga346 | EVQLQQSGPELVKPGASMKISCKASGYSFTGYT | 281 | DVVVTQTPLSLPVSFGDQVSISCRSSQSLANSY | 482 |
| | MNWVKQSHGKNLEWTGLINPYNGGTSHNQKFKG | | GNTYLSWYLHKPGQSPQLLIYGISNRFSGVPDR | |
| | KATLTVDKSSSTAYMELLSLTSEDSAVYYCARF | | FSGSGSGTDFTLKISTIKPEDLGMYYCLQGTHQ | |
| | RLWQGMDYWGQGTSVTVSS | | PPTFGGGTKLEIK | |
| SCT-Ga347 | QVQLQQPGSELVRPGASVKLSCKASGYTFTSYW | 282 | DIVLTQSPATLSVTPGDSVSLSCRASPNTRNSL | 612 |
| | MHWVKQRHGQGLEWIGNIFPGSDTTNYDEKFKS | | HWFQQKSHESPRLLISYASQSISGTPSRFSGSG | |
| | KVTLTVDTSSSTVYMHLSSLTSEDSAVYYCTRE | | SGTDFTLSINSLETEDFGMYFCQQSNSWPYTFG | |
| | GLYAFDYWGQGTTLTVSS | | GGTKLEIK | |
| SCT-Ga348 | QVQLQQPGSELVRPGASVKLSCKASGYTFTSYW | 283 | DIVLTQSPAILSVSPGERVSFSCRASQSIGTSI | 613 |
| | MHWVKQRHGQGLEWTGNIFPGSGTTNYDEKFKN | | HWYQQRTNGSPRLLIKFASESISGIPSRFSGSG | |
| | KGTLTVDTSSNTAYMHLSSLTSEDSAVYYCARE | | SGTDFTLSTNSVESEDTGDYYCQQSNSWPYTFG | |
| | GLYAFDYWGQGTTLTVSS | | GGTKLEIK | |
| SCT-Ga349 | QVQLQQPGSELLRPGASVKLSCKASGYTFTSYW | 284 | DTVLTQSPATLSVTPGDSVSLSCRASQSTRNSL | 614 |
| | MHWVKQRHGQGLEWIGNIFPHSGITNYDERFKN | | HWFQQKSHESPRLLINYVSQSISGIPSRFRGSG | |
| | KVTLTVDTSSSTAYMHLSSLTSEDSAVYYCARE | | SGTDFTLTTNSLETEDFGMYFCQQTNSWPYTFG | |
| | GLYAFDYWGQGTTLTVSS | | GGTKLEIK | |
| SCT-Ga350 | EVMLVESGGGLVKPGGSLKLSCAASGFTFSTYA | 285 | MIMMTQSPSSLAVSAGEKVTMSCKSSQSVLYSS | 615 |
| | MSWVRQTPEKRLEWVATTSSGGTYTYYPDSVKG | | NQMNYLAWYQQKPGQSPKLLIYWASTRESGVPD | |
| | RFTISRDNAKNTLYLQMSSLRSEDTAMYYCARH | | RFTGSGSGTDFTLTISSVQSEDLALYYCHQYLS | |
| | FTSTIITHFDYWGHGTTLTVSS | | SLTFGAGTKLELK | |
| SCT-Ga351 | QAYLQQSGAELVRSGASVKMSCKASGYTFTSYN | 224 | DIVMTQSQKFMSTSIGDRVSVTCKASQNVVTNV | 608 |
| | MHWVKQTPGQGLEWTGKTFPGNGFTDYNQTFRD | | AWYQQKPGQSPKALIYSASYRYSGVPDRFTGSG | |
| | KATLTADTSSSTAYMQISSLTSEDSAVYFCTRE | | SGTDFTLTISNVQSEDLADYFCQQHNSYPYTFG | |
| | GWLSLGYWGQGTTLTVSS | | GGTKLEIK | |
| SCT-Ga352 | EVQLQQSGTVLARPGASVKMSCKASGYSFTSYW | 286 | DIVLTQSPATLSVTPGDSVSLSCRASQSITNNL | 616 |
| | MHWVKQRPGQGLEWIGAIYPGNNATRYNQKFMG | | HWYQQKSHESPRLLIKYASQSISGIPSRFSGSG | |
| | KAKLTAVTSASTAYMEISSLTNEDSSVYYCSRE | | SGTDFTLSINSVETEDFGLYFCQQSNSWPYTFG | |
| | GGFGMDYWGQGTSVTVSS | | GGTKLEIN | |
| SCT-Ga353 | EVQLQQSGPELVKPGASMKTSCKASGYSFTDYT | 180 | DVVVTQTPLSLPVSFGDQVSISCRSSQSLANSY | 617 |
| | MNWVKQSHGKNLEWLGLIFPYNGATSYNQKFKG | | GNTYLSWYLHKPGQSPHLLIYGTSNRFSGVPDR | |
| | KATITVDKSSSTAYMELLSLTSEDSAVYYCARG | | FSGSGSGTDFTLKISTIKPEDLGMYYCLQGTHQ | |
| | LRGFFALDYWGQGTSVTVSS | | PWTFGGGTKLEIK | |
| SCT-Ga354 | EVQLQQSGPELVKPGASMKTSCKASGYSFTDYT | 287 | DVVVTQTPLSLPVSFGDQVSTSCRSSQSLANIY | 618 |
| | MNWVKQSHGKNLEWIGLIIPYNGGTSYNQKFKG | | GNTYLSWYLHKPGQSPQLLIYGISNRFSGVPDR | |
| | KATLTVDKSSSTAYMDLLSLTSEDSAVYYCARG | | FSGSGSGTDFTLKTSTIKPEDLGMYYCLQGTHQ | |
| | LRGYYTMDYWGHGTSVTVSS | | PWTFGGGTKLEIK | |
| SCT-Ga355 | QVQLQQPGAELVKPGASVKMSCKASGYTFTTFW | 288 | DTLLTQSPAILSVSPGERVSFSCRASQSVGTST | 619 |
| | INWVKQRPGQGLEWIGDIFPGRGITNYNGKFNN | | QWYQQRTNGSPRLLIKYGFESISGIPSRFSGSG | |
| | KATLTLDTSSSTAYMQLSSLTSEDSAVYYCSRG | | SGTDFTLSTNSVESEDSADYYCQQSYRWPYTFG | |
| | LGYFDVWGAGTTVTVSS | | GGSKLEIK | |
| SCT-Ga356 | QVQLQQSGGEVMKPGASVKISCKATGYTFSNYW | 289 | DIQMTQTTSSLSASLGDRVTISCSASQGISNYL | 620 |
| | IEWVKQRPGHGLEWIGEILPGSGSAYYNEKFRG | | NWYQQKPDGTVTLLIYYTSSLHSGVPSRFSGSG | |
| | KATFTADKSSNTAYMQLSSLTSDDSAVYYCARQ | | SGTDYSLTISNLEPEDIATYFCQHYSKLPYTFG | |
| | KYGNYYYFDYWGQGTTLTVSS | | GGTKLEIK | |
| SCT-Ga357 | QVQLQQPGSELVRPGASVKLSCKASGYTFTSYW | 238 | DIVLTQSPATLSVTPGDSVSLSCRASQSIRNNL | 621 |
| | MHWVKQRHGQGLEWTGNIYPGSGTTNYDEKFKN | | HWFQQKSHESPRLLIKYVFQSISGIPSRFSGSG | |
| | KGTLTVDTSSSTAYIHLSSLTSEDSAVYYCTRE | | SGTDFTLSINSLETEDFGMYFCQQSNSWPYTFG | |
| | GLYPFDYWGQGTTLTVSS | | GGTKLEIK | |
| SCT-Ga358 | EVQLQQSGPELEKPGSSVKISCKASGYSFTDYN | 177 | EIVLTQSPAIMSASPGEKVTVTCSAGSSVSSSD | 622 |
| | MHWVKQSNGKSLEWIGDIDVYYSGTSYNQKFKG | | LHWYQQKSGSSPRLLIYDTSKLASGVPVRFSGS | |
| | KATLTVDKSSSTTYMELKSLTSEDSAVYFCARS | | GSGTSYALTISRMEAEDAATYYCQQWSSYPLTF | |
| | SLRWYFDLWGAGTTVTVSS | | GAGTKLELK | |
| SCT-Ga359 | QITLKESGPGLVQPSQPFRLTCTFSGFSLSTSG | 290 | DVLLTQTPLSLPVSLGDQASTSCRSSQSIVHSN | 623 |
| | IGVTWIRQPSGKGLEWLATIWWDDDSRYNPSLK | | GNTYLEWYLQKPGQSPKLLIYKVSNRFSGVPDR | |
| | SRLTVSKDTSNNQAFLNIITVETADTAIYYCAQ | | FSGSGSGTDFTLKIRRVEAEDLGVYYCFQGSHV | |
| | SGGITLMDYWGQGTSVTVSS | | PGTFGGGTRLEIK | |
| SCT-Ga360 | EVQLQQSGPELVKPGASIKISCKASGYSFTGYT | 291 | DVVVTQTPLSLPVSFGDQVSISCRSSQSLANSY | 572 |
| | MNWVKQSHGKNLEWIGLIFPYNGATSYNQKFQG | | GNTYLSWYLHKPGQSPQLLIYGISNRFSGVPDR | |
| | KATLTVDKSSNTTYMELLSLTSEDSAVYYCARG | | FSGSGSGTDFTLKISTIKPEDLGMYYCLQGTHQ | |
| | LRGYFAMDYWGQGTSVTVSS | | PWTFGGGTKLEIK | |
| SCT-Ga361 | QVQLQQPGSELVRPGASVKLSCKASGYTFTSYW | 292 | DIVLTQSPATLSVTPGDSVSLSCRASQNIRNNL | 624 |
| | MHWVRQRHGQGLEWIGNIFPGSGTTNYDEKFKN | | HWFQQKSHESPRLLIKYASQSISGIPSRFSGSG | |
| | RGTLTVDTSSSTANMHLSSLTSEDSAVYYCTRE | | SGTDFTLSINSLETEDFGMFFCQQSNSWPYTFG | |
| | GLYPFDYWGQGTTLTVSS | | GGTKLEIK | |
| SCT-Ga362 | QVQLQQPGSELVRPGASVKLSCKASGYTFTSYW | 293 | DIVLTQSPATLSVTPGDSVSLSCRASQNIRNNL | 529 |
| | MHWVKQRHGQGLEWIGNIFPGTGSANYDERFKN | | HWFQQKSHESPRLLIKYASQSISGIPSRFSGSG | |
| | KGTLTVDTSSSTAYMHLSSLTYEDSAVYYCTRE | | SGTDFTLSINSLETEDFGMYFCQQSNSWPYTFG | |
| | GLSPFDYWGQGTTLTVSS | | GGTKLEIK | |
| SCT-Ga363 | EVQLQQSGPELEKPGSSVKISCKASGYSFTDYN | 177 | EIVLTQSPATMSASPGEKVTVTCSAGSSVSSRY | 625 |
| | MHWVKQSNGKSLEWIGDIDVYYSGTSYNQKFKG | | FHWYQQKSGSSPRLLIYDTSKLASGVPVRFSGS | |
| | KATLTVDKSSSTTYMELKSLTSEDSAVYFCARS | | GSGTSYALTISRMEAEDAATYYCQQWSSYPLTF | |
| | SLRWYFDLWGAGTTVTVSS | | GAGTKLELK | |
| SCT-Ga364 | QAYLQQSGAELVRSGASVKMSCKASGYTFTSYS | 294 | DIVMTQSQKFMSTSVGDRVSVTCKASQNVHTNV | 626 |
| | MHWVKQTPGQGLEWIGCIYPGNVITNYNQKFKG | | AWYQQKPGQSPKTLIYSASYRYSGVPDRFTGSG | |
| | KATLTADTSSSTAYMQISSLTSEDSAVYFCARD | | SGTDFTLTISNVQSEDLAEYFCQQYNSYPYTFG | |
| | GGFGTYWGQGTLVTVSA | | GGTKLEIK | |
| SCT-Ga365 | QAYLQQSGAELVRSGASVKMSCKASGYTFTSYN | 295 | QVVLTQSPAIMSASPGEKVTMTCSASSSVSYMH | 512 |
| | LHWVKQTPGQGLEWIGYIYPGNGYTNYNQKIKG | | WYQQKSGTSPKRWIYATSKLASGVPARFSGNGS | |
| | KATLTADTSSSTAYMQISSLTSEDSAVYFCARE | | GTSYSLTISSMEAEDAATYYCQQWNSNPPTFGA | |
| | GGWAMDYWGQGTSVTVSS | | GTKLDLK | |
| SCT-Ga366 | EVQLQQSGPELVKPGASLKISCKASGYSFTGYT | 296 | DVVVTQTPLSLPVSFGDQVSISCRSSQSLANSY | 627 |
| | MNWVKQSHGKNLEWIGLIFPYNGATSYNQKFQG | | GNTYLSWYLHKPGQSPQLLIYGISNRFSGVPDR | |
| | KATLTVDKSSNTTYMELLSLTSEDSAVYYCTRG | | FSGSGSGTDFTLTISTIKPEDLGMYYCLQGTHL | |
| | LRGYFALDYWGQGTSVTVSS | | PWTFGGGTKLEIK | |
| SCT-Ga367 | EVQLQQSGPELVKPGASMKISCKASGYSFTGYT | 173 | DWMTQTPLSLPVSFGDQVSISCRSSQSLANSY | 628 |
| | MNWVKQSHGKNLEWLGLIFPYNSATSYNQKFTG | | GNTYLSWYLYKPGQSPHLLIYGISNRFSGVPDR | |
| | KATITVDKSSNTTYMELLSLTSEDSAVYYCARG | | FSGSGSGTDFTLKISTIKPEDLGMYYCLQGTHQ | |
| | LRGFFALDYWGQGTSVTVSS | | PWTFGGGTKLEIK | |
| SCT-Ga368 | QAYLQQSGAELVRSGASVKMSCKASGYTFTSYN | 297 | DIVMTQSQKFMSTSVGDRVSVTCKASQNVGTNV | 629 |
| | MHWVKQTPGQGLEWIGYIYPGNGGTNYNQKFKG | | AWYQQKPGQSPKVLIYSASYRYSGVPDRFTGSG | |
| | KATLTADTSSNTAYMQISSLTSEDSAVYFCARD | | SGTDFTLTISNVQSEDLAEYFCQQYNSYPLTFG | |
| | GFYPFDYWGQGTTLTVSS | | AGTKLELK | |
| SCT-Ga369 | QAYLQQSGAELVRSGASVKMSCKASGYTFTSYN | 298 | QIVLTQSPVTMSASPGEKVTMTCSATSSVSSMH | 630 |
| | LHWIKQTPGQGLKWIGYIYPGNGYTNYNQKFKG | | WYQQKSGTSPKRWIYDTSKLASGVPIRFSGSGS | |
| | KATLTADTSSSTAYMQINSLTSEDSAVYFCARE | | GTSYSLTISSMEAEDAATYYCQQWSSNPSTFGA | |
| | GGWAMDYWGQGTSVTVSS | | GTKLELK | |
| SCT-Ga370 | QTQLVQSGPELQKPGETVKISCRASGYTFTNYG | 299 | DIVMTQAAFSNPVTLGTSASISCRSSKNLLHSN | 466 |
| | MNWVKKAPGKGLKWMGWINTYTGESAYADDFKG | | GITYLYWYLQRPGQSPQVLIYRMSNLASGVPNR | |
| | RFAFSSETSASTAYLQINNVKNDDTATYFCARD | | FSGSGSGTDFTLRISRVEAEDVGVYYCGQLLEL | |
| | SFAHWGQGTLVTVSA | | PYTFGGGTKLEIK | |
| SCT-Ga371 | EVQLQQSGPELVKPGVSMKISCKASGYSFTGYT | 300 | DVVMTQTPLSLPVSFGDQVSISCRSSQSLANSY | 631 |
| | MNWVKQSHGKNLEWLGLIFPYNSATSYNQKFTG | | GNTYLSWYLHKPGQSPHLLIYGISNRFSGVPDR | |
| | KATITVDKSSNTTYMELFSLTSEDSAVYYCARG | | FSGSGSGTDFTLKISTIKPEDLGMYYCLQGIHQ | |
| | LRGFFALDYWGQGTSVTVSS | | PWTFGGGTKLEIK | |
| SCT-Ga372 | EVQLQQSGTVLARPGASVKMSCKASGYSITSYW | 301 | DVVLTQSPATLSVTPGDSVSLSCRASQTISNNL | 580 |
| | MHWVKQRPGQGLEWIGGIYPGNSNTNYNQKFKG | | HWYQQKSHESPRLLIKYSSQSISGIPSRFSGSG | |
| | KAKLTAVTSASTANMELSSLTNEDSAVYYCTRE | | SGTDFTLSINSVETEDFGMYFCQQSYSWPYTFG | |
| | GGYGMDYWGQGTSVTVSS | | GGTKLEIK | |
| SCT-Ga373 | EVKLVESGGGLVKPGGSLKLSCAASGFAFSTYD | 176 | DIQMNQSPSSLSASLGDTITITCHASQNINVWL | 632 |
| | MSWVRQTPEKRLEWVATISSGGSYTYYPDSVKG | | SWYQQKPGNIPKLLIYKASTLHTGVPSRFSGSG | |
| | RFTISRDNARNTLYLQMSSLRSEDTALYYCARQ | | SGTGFTLNISSLQPEDIATYYCQQGQSYPWTFG | |
| | DYRYPYIMDYWGQGTSVTVSS | | GGTKLEIK | |
| SCT-Ga374 | QVQLQQSGGEVMKPGASVKISCKATGYTFSNYW | 302 | DIQMTQTTSSLSASLGDRVTISCSASQGISNYL | 633 |
| | IEWVKQRPGHGLEWIGEILPVSDSAYYNERCRC | | NWYQQKPDGTVTLLIYYTSTLHSGVPSRFSGSG | |
| | KATFTADESSTTAYMHLSGVASEDSAVYYCARQ | | SGTEYSLTISSLEPEDIATYYCQHYSKLPYTFG | |
| | KYGTYYYFDYWGQGTTLTVSS | | GGTKLEIK | |
| SCT-Ga375 | QITLKESGPGIVQPSQPFRLTCTFSGFSLSTSG | 267 | DVLMTQSPLSLPVSLGDQASISCRSSQSIVHSD | 531 |
| | IGVTWIRQPSGKGLEWLATIWWDDDNRYNPSLK | | GNTYLEWYLQKPGQSPKLLIYKVSRRFSGVPDR | |
| | SRLTVSKDTSNNQVFLNIIAVETADTAMYYCAQ | | FSGSGSGTDFTLKISRVGAEDLGVYYCFQGSHV | |
| | SGGITLMDYWGQGTSVTVSS | | PGTFGGGTKLEIK | |
| SCT-Ga376 | QVQLQQSGVELVRPGTSVKISCKASGYTFTNFW | 303 | DIKMTQSPSSTYASLGERVTITCKASQDIKSYL | 634 |
| | LSWVKQRPGHGLEWIGDIFPGGDYTNYNEKFKG | | SWFQQKPWKSPKTLIYYATRLADGVPSRFSGSG | |
| | KATLTADTSSSTAYIQLSSLTSEDSAVYFCSRS | | SGQDYSLTISSLESDDTATYYCLHHGERPYTFG | |
| | TIISGAYWGQGTLVTVSE | | GGTKLEIK | |
| SCT-Ga377 | QVQLQQPGSELVRPGASVKLSCKASGYTFTSYW | 304 | DIVLTQSPATLSVTPGDSVSLSCRASPNIRNSL | 612 |
| | MHWVKQRHGQGLEWIGNIYPGSGTTNYDEKFKS | | HWFQQKSHESPRLLISYASQSISGIPSRFSGSG | |
| | KVTLTVDTSSSTAYMHLSSLTSEDSAVYFCTRE | | SGTDFTLSINSLETEDFGMYFCQQSNSWPYTFG | |
| | GLYAFDYWGQGTTLTVSS | | GGTKLEIK | |
| SCT-Ga378 | EVQLQQSGPELVKPGASMKISCKASGYSFTGYI | 305 | DVVVTQTPLSLPVSFGDQVSISCRSSQSLANSY | 635 |
| | MNWVRQSHGKSLEWTGLINPYNGGTSYNQKFKG | | GSTYLSWYLHKPGQSPQLLIYGISNRFSGVPDR | |
| | KATLTVDKSSSTAYMELLSLTSEDSAVYYCTRL | | FSGSGSGTDFTLKISTIKPEDLGMYYCLQGTHQ | |
| | LRFAMDYWGQGTSVTVSS | | PPTFGGGTKLEIK | |
| SCT-Ga379 | EVNLVESGGGLVKPGGSLKLSCAASGFVFSSYD | 306 | DIQMNQSPSSLSASLGDTITITCHASQNINVWL | 636 |
| | MSWVRQTPEKRLEWVATISSGGSYTYYPDSVKG | | NWYQQKPGNIPKLLIYKASNLHTGVPLRFSGSG | |
| | RFTTSRDNARNTLYLQMSSLRSEDTALYYCARQ | | SGTGFTLTISSLQPEDIATYYCQQGQSYPWTFG | |
| | DYRYPYTMDYWGQGTSVTVSS | | GGTKLEIK | |
| SCT-Ga380 | EVNLVESGGGLVQPGGSLRLSCSTSGFPFNDYY | 307 | DIQMTQSSSYLSVSLGGRVTITCKASDHINNWL | 596 |
| | MSWVRQPPGKALEWLCLIRNKANGYTTEYSSSV | | AWYQQKPGNSPRLLISGATSLGAGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTTLTVSS | | SGTKLEIK | |
| SCT-Ga381 | EFQLQQSGTVLARPGASVKMFCKASGYSFTRYW | 308 | DIVLTQSPATLSVTPRNSVSLSCRASQSIRNNL | 637 |
| | MHWVKQRPGQGLEWIGAFYPGNSATNYNQKFKG | | HWYQQKSHGSPRLLIKYVSQSISGIPSRFSGSG | |
| | KAKLTAVTSASTAYMELNSLTNEDSAVYYCSRE | | SGTDFTLSINSVETEDFGMYFCQQSNSWPYTFG | |
| | GGYGMDHWGQGASVTVSS | | GGTKLEIN | |
| SCT-Ga382 | QVQLQQSGPEWRPGVSVKISCKGSGYTFTDYA | 309 | ENVLTQSPAIMSASPGEKVTMTCRASSSVSSSY | 638 |
| | MHWVKQSHAKSLEWIGVIGTSNGNTNYNQKFRG | | LHWYQQKSGASPKLWIYSTSNLASGVPARFSGS | |
| | KATMAVDKSSSTAYMELARLTSEDSAIYYCTRD | | GSGTSYSLTISSVEAEDAATYYCQQYSGYPLTF | |
| | YYGRSYLYFDYWGQGTTLTVSS | | GGGTKLEIK | |
| SCT-Ga383 | EVQLQQSGPELVKPGASMKISCKASGYSFTGYT | 310 | DVVVTQTPLSLPVSFGDQVSISCRSSQSLTNSY | 639 |
| | MNWVKQSHGKNLEWIGLIFPYNGGTSYNQRFMG | | GDTYLSWYLHKPGQSPQLLIYGISNRFSGVPDR | |
| | KATLTVDKSSNTAYMELLSLTSEDSAVYYCARS | | FSGSGSGTDFTLKISTIKPEDLGMYYCLQGTHQ | |
| | RSFAMDYWGQGTSVTVSS | | PWTFGGGTKLEIK | |
| SCT-Ga384 | EVKLVESGGGLVQPGGSLRLSCATSGFTFSDYY | 311 | DIQMTQSSSYLSVSLGGRVTITCKASDHINNWL | 552 |
| | MSWVRQPPGKALEWLALIRNKANGYTTEYSVSV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGILVTVSS | | SGTKLEIK | |
| SCT-Ga385 | QIQLVQSGPELKKPGETVKISCKASGYTFTDHS | 312 | DIQMTQSSSYLSVSLGGRVTITCKASDHINNWL | 640 |
| | MHWVRQAPGQVLKWMGWINTETGEPTYVDDFKG | | AWYQQKPGNAPRLLISGATSLETGVPSRFSGSG | |
| | RFAFSLEASASTAYLQINNLKNEDTATYFCASP | | SGKDYTLIITSLQTEDVATYYCQQYWSTPLTFG | |
| | RNYDGAMDYWGQGISVTVSS | | AGTKLELK | |
| SCT-Ga386 | EVKLVESGGGLVQPGGSLRLSCATSGFTFTSYY | 313 | DIQMTQSPASLSASVGETVTITCGASEHIYGAL | 641 |
| | MSWVRQPPGKALEWLALIRDKANGYTTEYSVSV | | NWYQRKQGKSPQLLIYGATNLADGMSSRFSGSG | |
| | KGRFTISRDNSQSILYLQMNTLRAEDSATYYCA | | SGRQYSLKISSLHPDDVATYYCQNVLSTPLTFG | |
| | RDGGYWGQGTLVTVSA | | AGTKLELK | |
| SCT-Ga387 | QVQLQQSGPELVKPGASVKISCKASGYAFSSSW | 314 | DIVLTQSPASLAVSLGQRATISCRASKSVSTSG | 642 |
| | MNWVKQRPGQGLEWIGRIYPGDGDTYYSGKFKD | | YSYMHWYQQKPGQPPKLLIYLASNLESGVPARF | |
| | KATLTADKSSSTAYMQLSSLTSVDSAVYFCASN | | SGSGSGTDFTLNIHPVEEEDAATYYCQHSRELP | |
| | YYYFDYWGQGTTLTVSS | | WTFGGGTKLEIK | |
| SCT-Ga388 | QVQLQQSGAELAKPGASVKMSCKASGYTFTNYW | 315 | DIQMTQSSSYLSVSLGGRVTITCKASDHINNWL | 643 |
| | MHWVKQRPGQGLEWIGYTNPSTGSSDYNQKFKD | | AWYQQKPGNAPRLLISGASRLETGVPSRFSGSG | |
| | KATLTADKSSSTAYMQLSSLTSEDSAVYYCAAY | | SGKDYTLTITSLQTEDVATYYCQQYWSTPWTFG | |
| | YRYGDGAYWGQGTQVTVSA | | GGTKLEIK | |
| SCT-Ga389 | EVMLVESGGGLVKPGGSLRLSCAASGFTFSTYV | 316 | DIQMNQSPSSLSASLGDTITITCHASQNINVWL | 644 |
| | MSWVRQTPEKRLEWVATISSGGSYIHYPDSVQG | | SWYQQKPGNITKLLIYKASNLHTGVPSRFSGSG | |
| | RFTISRDNAKNTLYLQMSSLRSEDTAMYFCARW | | SGTGFTLTISSLQPEDIATYYCQQGQSYPRTFG | |
| | LLGLYAMDYWGQGTSVTVSS | | GGTKLEIK | |
| SCT-Ga390 | EVKLVESGGGLVQPGGSLRLSCATSGFTFSDYY | 311 | DIQMTQSSSYLSVSLGGRVTITCKASDHINNWL | 645 |
| | MSWVRQPPGKALEWLALIRNKANGYTTEYSVSV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSTPFTFG | |
| | RDGGYWGQGILVTVSS | | SGTKLEIK | |
| SCT-Ga391 | QVQLQQSGAELVRPGSSLKISCKASGYAFSSYW | 317 | DIQMTQSSSYLSVSLGGRVTITCKASDHINNWL | 645 |
| | MNWVKQRPGQGLEWIGQIYPGDGDSDYNGKFKV | | AWYQQKPGNAPRLLISGATSLETGVPSRFSGSG | |
| | KATLTADKSSSTAYMQLSSLTSEDSAVYFCARR | | SGKDYTLSITSLQTEDVATYYCQQYWSTPFTFG | |
| | KDYGYDYWGQGTTLTVSS | | SGTKLEIK | |
| SCT-Ga392 | EVKLVESGGGLVQPGGSLRLSCATSGFTFSEFY | 318 | DIVMSQSPSSLTVSVGEKVTMSCKSSQSLLYSG | 646 |
| | MEWVRQPPGKRLEWIAASRNKANDYSTDYSASV | | NQKNYLAWYQQKPGQSPKLLIYWASTRESGVPD | |
| | KGRFIVSRDTSQNILYLQMTALRADDTAIYYCA | | RFTGSGSGADFTLTINNMKAEDLAIYYCQQYYS | |
| | RDAYGYPWFVYWGQGTLVTVSA | | YPWTFGGGTKLEIK | |
| SCT-Ga393 | DVQLVESGGGLVQPGGSRKLSCAASGFTFSNFG | 319 | DIQMTQTTSSLSASLGDRVTISCRSSQDISNYL | 647 |
| | MDWVRQAPEKGLEWIAYISSGSSTIYYADTVKG | | NWYQQKPDGTVKLLIYYTSRLHSGVPSRFSGSG | |
| | RFTISRDNPKNTLFLQMTSLRSEDTAMYFCARG | | SGTDYSLTISNLEQEDIATYFCQQGNTLPFTFG | |
| | RYDYLYAMDYWGQGTSVTVSS | | TGTKLELK | |
| SCT-Ga394 | EVKLVESGGGLVQPGGSLRLSCATSGFTFTDYY | 320 | DIQMTQSSSYLSVSLGGRVTITCKASDHINNWL | 552 |
| | MSWVRQPPGKALEWLALITNKANGYTTDYSVSV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGTLVTVSA | | SGTKLEIK | |
| SCT-Ga395 | EVKLEKSRGGLVKPGGSMKLSCVASGFTFSNFW | 321 | DIQMTQSPSSLSASLGERVTLTCRAGQEISGFL | 648 |
| | MNWVRQSPEKGLEWVAQIRLNSDNYATHYAESV | | SWLQQKPDGTIKRLIYAASTLDSGVPKRFSGRR | |
| | KGRFTISRDDSKSSVYLQMNSLGAEDTGIYYCH | | SGSDYSLTISSLESEDFADYYCLQYASYPPTFG | |
| | GNFYDLDYWGQGTSVTVSS | | GGTKLEIK | |
| SCT-Ga396 | EVQLQQSGPELVKPGASMKISCKASGFSFTAYT | 322 | DVVVTQTPLSLPVSFGDQVSISCRSSQSLANSY | 649 |
| | MNWVKQSQGDNLEWIGLIVPYNGGTSYNLKFKG | | GSTYLSWYLHKPGQSPQLLIYGISNRFSGVPDR | |
| | KATLTVDKSSSTAYMELLSLTSEDSAVYYCARG | | FSGSGSGTDFTLKISTIEPEDLGMYYCLQYTHQ | |
| | LQRFFAMDFWGQGTSVTVSS | | PPTFGGGTKLEIK | |
| SCT-Ga397 | EVQLQQSGPELVKPGASMKMSCKASGYSFTTYT | 323 | DVVVTQTPLSLPVSFGDQVSISCRSSQSLANSY | 650 |
| | MSWVKQSHGKNLEWIGLINPYNGGTSYNQKFKG | | GDTYLSWYLHKPGQSPQLLIYGISNRFSGVPDR | |
| | KATLTVDKSSSTAYMELLSLTSEDSAVYYCARS | | FSGSGSGTDFTLKISTIKPEDLGMYYCLQGTHQ | |
| | RSFAMDYWGQGTSVTVSS | | PWTFGGGTKLEIK | |
| SCT-Ga398 | QVQLQQSGAELAKPGASVKMSCKASGYTFTNYW | 315 | DIQMTQSSSYLSVSLGGRVTITCKASDHINNWL | 651 |
| | MHWVKQRPGQGLEWIGYTNPSTGSSDYNQKFKD | | AWYQQKPGNAPRLLISGASRLETGVPSRFSGSG | |
| | KATLTADKSSSTAYMQLSSLTSEDSAVYYCAAY | | SGRDYTLTITSLQTEDVATYYCQQYWSTPWTFG | |
| | YRYGDGAYWGQGTQVTVSA | | GGTKLEIK | |
| SCT-Ga399 | EVQLQQSGPELVKPGASMKISCKASGYSFTGYT | 324 | DVVVTQTPLSLPVSFGDQVSISCRSSQSLTNSY | 652 |
| | MNWVRQSHGKDLEWIGLIIPYNGGTSYNQKFKG | | GNTYLSWYLHKPGQSPQLLIYGISNRFSGVPDR | |
| | KATITVDKASSTAYMDLLSLTSEDSAVYYCARG | | FSGSGSGTDFTLKISTIKPEDLGMYYCLQGTHQ | |
| | LRGYFAMDYWGQGTSVTVSS | | PWTFGGGTKLEIK | |
| SCT-Ga400 | EVQLQQSGPELVKPGASVKISCKASGYTFTDYN | 325 | DIQMTQSSSYLSVSLGGRVTITCKASDHINNWL | 653 |
| | MHWVKQSHGKSLEWIGYIYPYNGGTGYNQKFKS | | AWYQQKPGNAPRLLISGATSLETGVPSRFSGSG | |
| | KAPLTVDTSSSTAYMELRSLTSEDSAVYYCARY | | SGKDYTLSITSLQTEDVATYYCQQYWSTPLTFG | |
| | YGYFDYWGQGTTLTVSS | | AGTKLELK | |
| SCT-Ga401 | EVQLQQSGPELVKPGASMKISCKASGYSFTGYI | 326 | DVVVTQTPLSLPVSFGDQVSISCRSSQSLANSY | 482 |
| | MNWVKQSHGDNLEWIGLIVPYNGGTSYNLKFKG | | GNTYLSWYLHKPGQSPQLLIYGISNRFSGVPDR | |
| | KATLTVDKSSSTAYMELLSLTSEDSAVYYCARG | | FSGSGSGTDFTLKISTIKPEDLGMYYCLQGTHQ | |
| | LQRFYAMDYWGQGTSVTVSS | | PPTFGGGTKLEIK | |
| SCT-Ga402 | EVKLVESGGGLVQPGGSLRLSCATSGFTFTSYY | 313 | DIQMTQSSSYLSVSLGGRVTITCKASDHINNWL | 645 |
| | MSWVRQPPGKALEWLALIRDKANGYTTEYSVSV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSTPFTFG | |
| | RDGGYWGQGTLVTVSA | | SGTKLEIK | |
| SCT-Ga403 | EVKLEKSRGGLVQPGGSMKISCVASGFTFSNFW | 327 | DIQMTQSPSSLSASLGEGVTLTCRAGQEITGFL | 654 |
| | MNWVRHSPEKGLEWVAQIRLNSDNYATHYAESV | | SWLQQKPDGTIKRLIYAASTLDSGVPKRFSGRR | |
| | KGRFTISRDDSKRSVYLQMNSLGAEDTGIYYCH | | SGSDYSLTISSLESEDFADYYCLQYASYPPTFG | |
| | GNFYTLDYWGQGISVTVSS | | GGTKLEIK | |
| SCT-Ga404 | QVQLQQSGAELAKPGASVKMSCKASGYTFTNYW | 328 | DIQMTQSSSYLSVSLGGRVTITCKASDHINNWL | 655 |
| | MHWVKQRPGQGLEWIGYTNPSTGYSDYNQKFKD | | AWYQQKPGNAPRLLISGATSLETGVPSRFSGSG | |
| | KATLTADKSSSTAYMQLSSLTSEDSAVYYCAAY | | SGKDYTLTITSLQTEDVATYYCQQYWSTPWTFG | |
| | YRYGDGAYWGQGTQVTVSA | | GGTKLEIK | |
| SCT-Ga405 | DVQLVESGGGLVQPGGSRKLSCAASGFTFSSFG | 329 | DIQMTQTTSSLSASLGDRVTISCRASQDISNYL | 656 |
| | MDWVRQAPEKGLEWIAYISSGSSTIYYADTVKG | | NWYQQKPDGTVKLLIYYTSRLHSGVPSRFSGSG | |
| | RFTISRDNPKNTLFLQMTSLRSEDTAMYYCARG | | SGTDFSLTISNLDQEDIATYFCQQGNTLPFTFG | |
| | RFDYLYAMDYWGQGTSVTVSS | | VGTKLELK | |
| SCT-Ga406 | EVQLQQSGPELVKPGASMKISCKASGYSFTGYT | 330 | DVVVTQTPLSLPVSFGDQVSISCRSSQSLANSY | 572 |
| | MNWVKQSHGKNLEWIGLINPYNGGTSYNQKFKG | | GNTYLSWYLHKPGQSPQLLIYGISNRFSGVPDR | |
| | KATFTVDKSSSTAYMELLSLTSEDSAVYYCARG | | FSGSGSGTDFTLKISTIKPEDLGMYYCLQGTHQ | |
| | LRFSLEYWGQGTTLTVSS | | PWTFGGGTKLEIK | |
| SCT-Ga407 | EVQLQQSGPELVKPGASVKISCKASGYSFTGYI | 331 | DVVLTQTPLSLPVSFGDQVSISCRSSQSLANSY | 657 |
| | MNWVKQSHGKNLEWIGLIIPYNGGTSYNQKFRG | | GNTYLSWYLHKPGQSPQLLIYEISNRFSGVPDR | |
| | KATLTVDKSSSTAYMDLLSLTSDDSAVYYCARG | | FSGSGSGTDFTLKISTIKPEDLGMYYCLQGTHQ | |
| | LSYRYDYWGQGTTLTVSS | | PPTFGGGTKLEIK | |
| SCT-Ga408 | EVQLQQSGPELVKPGVSMKISCKASGYSFTGYT | 332 | DVVVTQTPLSLPVSFGDQVSISCRSSQSLTNSY | 652 |
| | MNWVRQSHGKDLEWIGLIIPYNGGTSYNQKFKG | | GNTYLSWYLHKPGQSPQLLIYGISNRFSGVPDR | |
| | KATITVDKASSTAYMDLLSLTSEDSAVYYCARG | | FSGSGSGTDFTLKISTIKPEDLGMYYCLQGTHQ | |
| | LRGYFAMDYWGQGTSVTVSS | | PWTFGGGTKLEIK | |
| SCT-Ga409 | EVQLQQSGPELVKPGASMKISCKASGYSFTGYI | 333 | DVVVTQTPLSLPVSFGDQVSISCRSSQSLANSY | 658 |
| | MNWVKQSHGKNLDWTGLTYPYNGGTSYNQKFKG | | GKTYLSWYLHKPGQSPQLLIYGISHRFSGVPDR | |
| | RATLTVDKSSSTAYMELLSLTSEDSAVYYCARL | | FSGSGSGTDFTLRISTIKPEDLGMYYCLQGTHQ | |
| | ISYAMDYWGQGTSVTVSS | | PPTFGGGTKLEIK | |
| SCT-Ga410 | QIQLVQSGPELKKPGETVKISCKASGYTFTDHS | 334 | DIQMTQSSSYLSVSLGGRVTITCKASDHINNWL | 659 |
| | MHWVKQAPGKGLKWMGWINTETGEPTYVDDFKG | | AWYQQKPGNAPRLLISGATSLETGVPSRFSGSG | |
| | RFAFSLETSASTAYLQINNLKNEDTAAYFCASP | | SGKDYTLSIISLQTEDVATYYCQQYWSTPLTFG | |
| | RNYDGAMDYWGQGTSVTVSS | | AGTKLELK | |
| SCT-Ga411 | QVQLQQSGAELAKPGASVKMSCKASGYTFTNYW | 335 | DIQMTQSSSYLPVSLGGRVTITCKASDHINSWL | 660 |
| | MHWVKQRPGQGLEWIGYTNPSTGYSEYNQKFKD | | AWYQQKPGNAPRLLISGTTSLETGIPSRFSGSG | |
| | KATLTADKSSSTAYMQLSSLTSEDSAVYYCAAY | | SGKDYTLTITSLQTEDVATYYCQQYWSTPWTFG | |
| | YRYGDGAYWGQGTQVTVSA | | GGTKLEIK | |
| SCT-Ga412 | QVQLQQSGTELVRPGTSVKVSCKASGYAFTDYL | 336 | DIVMTQSPSSLSVSAGEKVTMSCKSSQSLLNSG | 661 |
| | IEWVKQRPGQGLEWTGVINPGSGGAIYNEKFKG | | NQKNYLAWYQQKPGQPPKLLIYGASTRESGVPD | |
| | KVTLTADKSSSTAYMQLSSLTSDDSAVYFCARY | | RFTGSGSGTDFTLTIGSVQAEDLAVYYCQNYHS | |
| | HYYGSGLFPYWGQGTLVTVSA | | YPFTFGSGTKLEIK | |
| SCT-Ga413 | EVQLQQSGPELVKPGASVKMSCKASGYTFTSYV | 337 | DIQMTQSSSYLSVSLGGRVTITCKASDHINNWL | 643 |
| | MHWVKQKPGQGLEWIGYINPYNDGTQYNEKFRG | | AWYQQKPGNAPRLLISGASRLETGVPSRFSGSG | |
| | KATLTSDKSSSTAYMELSSLTSEDSAVYYCARE | | SGKDYTLTITSLQTEDVATYYCQQYWSTPWTFG | |
| | IDYWGQGTTVTVSS | | GGTKLEIK | |
| SCT-Ga414 | EVKLVESGGGLVQPGGSLRLSCATSGFTFSDYY | 338 | DIQMTQSSSYLSVSLGGRVTITCKASDHINNWL | 552 |
| | MSWVRQPPGKALEWLALIRNKANCYTTDYSVSV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSIPFTFG | |
| | RDGGYWGQGILVTVSS | | SGTKLEIK | |
| SCT-Ga415 | QIQLVQSGPELKKPGETVKISCKASGYTFTNYG | 339 | ETTVTQSPASLSVATGEKVTIRCITSTDIDDDM | 662 |
| | MNWVKQAPGKGLKWMGWINTYTGEPTYADDFKG | | NWYQQKPGEPPKLLISEGNTLRPGVPSRFSSSG | |
| | RFAFSLETSASTAYLQINNLKNEDTATYFCARY | | YGTDFVFTIENTLSEDVADYYCLQSDNMPYTFG | |
| | EYFDYWGQGTTLTVSS | | GGTKLEIK | |
| SCT-Ga4i6 | DVQLQESGPGLVKPSQSLSLTCTVTGYSITSDY | 340 | DIVMTQSHKFMSTSVGDRVSITCKASQDVSTTV | 663 |
| | AWNWIRQFPGNKLDWMGYITYSGDTRYNPSLKS | | AWYQQKPGQSPKPLIYSASYRYTGVPDRFTGSG | |
| | RISITRDTSKNQFFLRLNSVTTEDTATYYCARR | | SGTDFTFTISSVQAEDLAVYYCQQHYSVPWTFG | |
| | ELGQGFTYWGQGTLVTVSA | | GGTKLEIK | |
| SCT-Ga417 | QVQLQQSGAELARPGASVTLSCKASGYTFTYYE | 341 | DIQMTQSSSYLSVSLGGRVTITCKASDHINNWL | 645 |
| | IHWVKQTPVHGLEWIGPIDPETGGTAYNQKFKG | | AWYQQKPGNAPRLLISGATSLETGVPSRFSGSG | |
| | KATLTADKSSSTAYMELRSLTSEDSAVYFCTRW | | SGKDYTLSITSLQTEDVATYYCQQYWSTPFTFG | |
| | GYKFAYWGQGTLVTVSA | | SGTKLEIK | |
| SCT-Ga418 | qvqlqqsgaelvrpgtsvkvsckasgydftdyl | 342 | DIVMTQSPSSLSVSAGEKVTMSCKSSQSLLNSG | 664 |
| | IEWVKQRPGQGLEWIGVINPGSGGTKYNEKFKG | | NQKNYLAWYQQKPGQPPKLLIYGASTRESGVPD | |
| | KATLTADKSSSTAYMQLSSLTSDDSAVYFCARY | | RFTGSGSGTDFTLTISSVQAEDLAVYYCQNYHS | |
| | HYYGSNLFPYWGQGTLVTVSA | | YPFTFGSGTKLEVK | |
| SCT-Ga419 | EVQLQQSGPELVKPGASMKMSCKASGYSFTTYT | 343 | DVVVTQTPLSLPVSFGDQVSISCRSSQSLANSY | 572 |
| | MSWVKQSHGKNLEWIGLINPYNGGTSYNQKFKG | | GNTYLSWYLHKPGQSPQLLIYGISNRFSGVPDR | |
| | KATLTFDKSSSTAYMELLSLTSEDSAVYYCARS | | FSGSGSGTDFTLKISTIKPEDLGMYYCLQGTHQ | |
| | RTFAMDYWGQGTSVTVSS | | PWTFGGGTKLEIK | |
| SCT-Ga420 | DVQLVESGGGLVKPGGSLKLSCAASGFTFSSYT | 344 | DIQMNQSPSSLSASLGDTITITCHASQNINVWL | 665 |
| | MSWVRQTPEKRLEWVATISGGGSYTYYPDSVKG | | SWYQQKPGNIPKLLIYKASNLHTGVPSRFSGSG | |
| | RFTISRDNAKNTLNLQKSSLKSEDTAMYYCTRW | | SGTGFTLTISSLQPEDIATYYCQQGQSYPYTFG | |
| | LQSLYAMDYWGQGTSVTVSS | | GGTKLEIK | |
| SCT-Ga421 | EVQLQQSGPELVKPGASMKISCKASGYSFTGYT | 345 | DVVVTQTPLSLPVSFGDQVSISCRSSQSLANSY | 483 |
| | MNWVKQSHGKNLEWIGLINPYNGGTSYNQKFKG | | GNTYLSWYLHKPGQSPQLLIYGISNRFSGVPDR | |
| | KATLTVDKSSSTAYMELLSLTSEDSAVYYCASG | | FSGSGSGTDFTLKISTIKPEDLGMYYCLQGTHQ | |
| | GLRYGFAYWGQGTTLTVSS | | PLTFGAGTKLELK | |
| SCT-Ga422 | EVQLVETGGGLVQPKGSLNLSCAASGFTFNTNA | 346 | ENVLTQSPAIMSASPGEKVTMTCRASSSVSSSY | 666 |
| | MNWVRQAPGKGLEWVARIRSKSNNYATYYADSV | | LHWYQQKSGASPKLWIYTTSKLASGVPARFSGS | |
| | KDRFTISRDDSQSMLYLQMNNLKTEDTAMYYCV | | GSGTSYSLTISSVEAEDAATYYCQQYSGYPLTF | |
| | GSTMTIFSYWGQGTLVTVSA | | GGGTKLEIK | |
| SCT-Ga423 | QVQLKESGPGLVAPSQSLSITCTVSGFSLTSYG | 347 | EILLTQSPAIIAASPGEKVTITCSASSSVSYMN | 667 |
| | VHWVRQPPGKGLEWLGVIWADGSTNYNSALMSR | | WYQQKPGSSPKIWIYGISNLASGVPARFSGSGS | |
| | LSINKDNSKSQVFLKMNSLQTDDTAMYYCARDL | | GTSFSFTINSMEAEDVATYYCQQISSYPLTFGA | |
| | GYLTTAWFAYWGQGTLVTVSA | | GTKLELK | |
| SCT-Ga424 | EVQLQQSGPELVKPGVSMKMSCKASGYSFTAYT | 348 | DVVVTQTPLSLPVSFGDQVSISCRSSQSLANSY | 572 |
| | MSWVKQSHGKSLEWIGLINPYNGGISYNQKFKD | | GNTYLSWYLHKPGQSPQLLIYGISNRFSGVPDR | |
| | KATLTVDKSSTTAYMELLSLTSEDSAVYYCARS | | FSGSGSGTDFTLKISTIKPEDLGMYYCLQGTHQ | |
| | RTFAMDYWGQGTSVTVSS | | PWTFGGGTKLEIK | |
| SCT-Ga425 | EVQLQQSGPELVKPGASMKISCKASGYSFTDYI | 349 | DVVVTQTPLSLPVSFGDQVSISCRSSQSLENSY | 668 |
| | MNWVKQSHGDNLEWIGLIVPYNGGTSYNLNFKG | | GNTYLSWYLHKPGQSPQLLIYGISNRFSGVPDR | |
| | KASLTVDKSSSTAYMELLSLTSEDSAVYYCARG | | FSGSGSGTDFTLKISTIKPEDLGMYYCLQGTHQ | |
| | LQRFYAMDYWGQGTSVTVSS | | PPTFGGGTKLEIK | |
| SCT-Ga426 | QVHLQQSGAELAKPGASVKMSCKASGYTFTNYW | 350 | DIQMTQSSSYLSVSLGGRVTITCKASDHINNWL | 643 |
| | MHWVKQRPGQGLEWIGYTNPSTGSSDYNQKFKD | | AWYQQKPGNAPRLLISGASRLETGVPSRFSGSG | |
| | KATLTADKSSSTAYMQLSSLTSEDSAVYYCAAY | | SGKDYTLTITSLQTEDVATYYCQQYWSTPWTFG | |
| | YRYGDGAYWGQGTQVTVSA | | GGTKLEIK | |
| SCT-Ga427 | EVQLQQSGPELVKPGASMKTSCKASGFSFPGYT | 351 | DVVVTQTPLSLPVSFGDQVSISCRSSQSLANSY | 669 |
| | MNWVKQSHGKNLEWIGLINPYNGATSYNQKFKG | | GNTYLSWYLHKPGQSPQLLIYGISNRFSGVPDR | |
| | KATLTVDKSSNTAYMDLLSLTSEDSAVYYCTRG | | FSGSGSGTDFTLKISTIKPEDLGLYYCLQGTHL | |
| | TLITGFAYWGQGTLVTVSA | | PLTFGAGTKLELK | |
| SCT-Ga428 | EVQLQQSGPELVKPGASMKTSCKASGFSFTGYT | 352 | DVVVTQTPLSLPVSFGDQVSISCRSSQSLANSY | 669 |
| | MNWVKQSHGKNLEWIGLINPYNGATSYNQKFKG | | GNTYLSWYLHKPGQSPQLLIYGISNRFSGVPDR | |
| | KATLTVDKSSSTAYMELLSLTSEDSAVYYCARG | | FSGSGSGTDFTLKISTIKPEDLGLYYCLQGTHL | |
| | TLITGFAYWGQGTLVTVSA | | PLTFGAGTKLELK | |
| SCT-Ga429 | EVQLQQSGPELVKPGASMKTSCKASGYSFTGYI | 353 | DVVVTQTPLSLPVSFGDQVSISCRSSQSLANSY | 670 |
| | MSWVKQSHGKNLEWIGLINPYNGGTSYNQKFKG | | GNTFLSWYLHKPGQSPQLLIYGISNRFSGVPDR | |
| | KATLTIDKSSSTAYMELLSLTSEDSAVYYCARS | | FSGSGSGTDFTLKISSIKPEDLGMYFCLQGTHQ | |
| | RSFAMDYWGQGTSVTVSS | | PWTFGGGTMLEIK | |
| SCT-Ga430 | EVQLQQSGPELVKPGTSMKISCKASGYSFTGYT | 354 | DWMTQSPLSLPVSFGDQVSISCRSSQSLANSY | 671 |
| | MNWVKQSHGKNLEWIGLIVPYNGGTSYNQKFKG | | GDTFLSWYLHKPGQSPQLLIYGISNRFSGVPDR | |
| | KATLTVDKSSSTAYMELLSLTSEDSAVYYCARG | | FSGSGSGTDFTLKISTIKPEDLGMYYCLQGTHQ | |
| | LSFRYDYWGQGTTLTVSS | | PPTFGGGTKLEIK | |
| SCT-Ga431 | EVQLQQSGPELVKPGASMKISCKASGYSFTGYI | 355 | DVVVTQTPLSLPVSFGDQVSISCRSSQSLANSY | 482 |
| | MNWVKQSHGKNLEWIGLIFPYNGGTSYNQKFKG | | GNTYLSWYLHKPGQSPQLLIYGISNRFSGVPDR | |
| | KATLTVDKSSSTAYMELLSLTSEDSAVYYCARG | | FSGSGSGTDFTLKISTIKPEDLGMYYCLQGTHQ | |
| | LPRFYAMDYWGQGTSVTVSS | | PPTFGGGTKLEIK | |
| SCT-Ga432 | QVQLKESGPGLVAPSQSLSITCTVSGFSLTSYG | 356 | DIQMTQSPASLSVSVGETVTITCRASENIYSNL | 672 |
| | VHWVRQPPGKGLEWLGVIWAGGTTNYNSALMSR | | AWYQQKQGKSPQLLVYAATNLVDGVPSRFSGSG | |
| | LSISKDNSKSQVFLKMNSLQTDDTALYYCARFP | | SGTQYSLKINSLQSEDFGSYYCQHFWGTPYTFG | |
| | SYRESMDYWGQGTSVTVSS | | GGTKLEIK | |
| SCT-Ga433 | DVKLVESGGGLVKPGGSLKLSCSASGFTFSSYT | 357 | DIQMNQSPSSLSASLGDTITITCHASQNINVWL | 673 |
| | MSWVRQTPEKRLEWVATISSGGSYTYYPDSVKG | | SWYQQKPGNISKLLIYKASNFHTGVPSRFSGSG | |
| | RFTISRDNAKNTLYLQMSSLKSEDTAMFYCTRW | | SGTNFTLTISSLQPEDIATYYCQQGQTYPFTFG | |
| | LLSLYAMDYWGQGTSVTVSS | | GGTKLEIK | |
| SCT-Ga434 | EVQLQQSGPELVKPGASMKISCKASGYSFTGYI | 358 | DVVVTQTPLSLPVSFGDQVSISCRSSQSLANSY | 658 |
| | MNWVKQSHGKNLEWIGLIYPYNGGTSYNQKFKG | | GKTYLSWYLHKPGQSPQLLIYGISHRFSGVPDR | |
| | RATLTVDKSSSTAYMELLSLTSEDSAVYYCARL | | FSGSGSGTDFTLRISTIKPEDLGMYYCLQGTHQ | |
| | ISYAMDYWGQGTSVTVSS | | PPTFGGGTKLEIK | |
| SCT-Ga435 | EVQLQQSGPELVKPGASMKISCKASGYSFTGYT | 359 | DVVVTQTPLSLPVSFGDQVSISCRSSQSLANSY | 572 |
| | MNWVKQSHGKNLEWIGLINPYNGATSYNQKFKG | | GNTYLSWYLHKPGQSPQLLIYGISNRFSGVPDR | |
| | KATFTVDKSSSTAYMELLSLTSEDSAVYYCARG | | FSGSGSGTDFTLKISTIKPEDLGMYYCLQGTHQ | |
| | LRFSLEYWGQGTTLTVSS | | PWTFGGGTKLEIK | |
| SCT-Ga436 | EVQLQQSGAELVKPGASVKLSCTASGFNIKDTY | 360 | EILLTQSPAIIAASPGEKVTITCSASSSVSYMN | 674 |
| | MHWVKQRPEQGLEWIGRIDPANGNIKFDPKFQG | | WYQQKPGSSPKIWIYGISNLASGVPARFSGSGS | |
| | KATITADTSSNTAYLQLRSLTSADTAVYFCARS | | GTSFSFTINSMEAEDVATYYCQQRSSFPPTFGG | |
| | LPGAYWGQGTLVTVSA | | GTKLEIK | |
| SCT-Ga437 | EVKLVESGGGLVQPGGSLILSCATSGFTFTNYY | 361 | DWMTQTPLTLSVTIGQPASISCKSSQSLLDSD | 381 |
| | MSWVRQPPGKALEWLGFIRNRPYGYTTEYNASV | | GKTYLNWLLQRPGQSPKRLIYLVSKLDSGVPDR | |
| | KGRFTISRDNSQSILYLQMNTLAPEDSATYYCA | | FTGSGSGTDFTLKISRVEAEDLGVYYCWQGTHF | |
| | AGLDYWGQGTTLTVSS | | PYTFGGGTKLEIK | |
| SCT-Ga438 | DVQLVESGGGLVQPGGSRKLSCAASGFTFSNFG | 319 | DIQMTQTTSSLSASLGDRVTISCRASQDISNYL | 675 |
| | MDWVRQAPEKGLEWIAYISSGSSTIYYADTVKG | | NWYQQKPDGTVKLLIYYTSRLHSGVPSRFSGSG | |
| | RFTISRDNPKNTLFLQMTSLRSEDTAMYFCARG | | SGTDYSLTISNLEQEDIATYFCQQGNTLPFTFG | |
| | RYDYLYAMDYWGQGTSVTVSS | | AGTKLELK | |
| SCT-Ga439 | QVQLQQSGAELVRPGTSVKVSCKASGYAFSDYL | 362 | DIVMTQSPSSLSVSAGEKVTMSCKSSQSLLNSG | 676 |
| | IEWVKQRPGQGLEWIGVINPGSGGTNYNEKFKD | | NQKKYLAWYQQKPGQPPKLLIYGASTRESGVPD | |
| | KATLTADKSSSIAYMQLSSLTSDDSAVYFCARF | | RFTGSGSGTDFTLTISSVQAEDLAVYYCQNYHR | |
| | HYYGSSLFPYWGPGTLVTVSA | | FPFTFGSGTKLEIK | |
| SCT-Ga440 | EVQLQQSGPELVKPGASMKISCKASGYSFTGYT | 363 | DVWTQTPLSLPVSFGDQVSISCRSSQSLANSY | 482 |
| | MNWVKQSHGDNLEWIGLIVPYNGGTSYNLKFKG | | GNTYLSWYLHKPGQSPQLLIYGISNRFSGVPDR | |
| | KATLTVDKSSSTAYMELLSLTSEDSAVYYCARG | | FSGSGSGTDFTLKISTIKPEDLGMYYCLQGTHQ | |
| | LQRFYAMDYWGQGTSVTVSS | | PPTFGGGTKLEIK | |
| SCT-Ga441 | EVKLVESGGGLVQPGGSLRLSCATSGFTFTDYY | 364 | DIQMTQSSSYLSVSLGGRVTITCKASDHINNWL | 645 |
| | MSWVRQPPGKALEWLALIRNKANGYTTEYSVSV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSTPFTFG | |
| | RDGGYWGQGTLVTVSA | | SGTKLEIK | |
| SCT-Ga442 | QVQLKQSGAELVRPGASVKLSCKTSGYIFTSYW | 365 | DIQMTQSPASQSASLGESVTITCLASQTIGTWL | 677 |
| | IHWVKQRSGQGLEWIARIYPGIGSTDYNEKFKG | | AWYQQKPGKSPQLLIYAATSLADGVPSRFSGSG | |
| | KATLTADKSSSTAYMQLSSLKSEDSAVYFCEVP | | SGTKFSFKISSLQAEDFVSYYCQQLYNTPLTFG | |
| | IGTVDYWGQGTTLTVSS | | AGTKLELK | |
| SCT-Ga443 | QVTLKESGPGILQPSQTLSLTCSFSGFSLSTSG | 366 | DWMTQTPLTLSVTIGQPASISCKSSQSLLDSD | 678 |
| | MGVGWIRQPSGKGLEWLAHIWWDDVKRYNPALK | | GKTYLNWLLQRPGQSPKRLIYLVSKLDSGVPDR | |
| | SRLTISKDASSSQVFLKTASVDTADTATYYCAR | | FTGSGSGTDFTLKISRVEAEDLGIYYCWQGTHF | |
| | LGVYYRYDGDYWGQGTTLTVSS | | PQTFGGGTKLEIK | |
| SCT-Ga444 | EVKLVESGGGLVQPGGSLRLSCATSGFTFIDYY | 367 | DIQMTQSSSYLSVSLGGRVTITCKASDHINNWL | 679 |
| | MSWVRQPPGKALEWLALIRNKANGYTTEYSVSV | | AWYQQKPGNAPRLLISGATSLETGIPSRFSGSG | |
| | KGRFTISRDNSQSILYLQMNTLRAEDSATYYCA | | SGKDYTLSITSLQTEDVATYYCQQYWSTPFTFG | |
| | RDGGYWGQGTLVTVSA | | SGTKLEIK | |
| SCT-Ga445 | QVQLKESGPGLVAPAQSLSITCTVSGFLITAYG | 368 | DIQMTQSPSSLSASLGERVSLTCRASQEISGFL | 680 |
| | VNWVRQPPGKGLEWLGMIWGDGSTDYNSALKSR | | SWLQQKPDGTIKRLIYAASTLDSGVPKRFSGTR | |
| | LSISKDSSKSQVFLKMNSLQTDDTARYYCARYD | | SGSDYSLTISSLESEDFADYYCLQYASYPPTFG | |
| | YYGMDYWGQGTSVTVSS | | GGTKLEIR | |
| SCT-Ga446 | EVNLVESGGGLVQPGGSRRLSCATSGFTFTNYY | 369 | DIQMTQSSSSFSVSLGDRVTITCKASEDIYNRL | 681 |
| | MTWVRQPPGKALEWLGFIRNKPYGYTTEYNESV | | AWYQQKPGNAPRLLISGATSLETGVPSRFSGSG | |
| | KGRFTISRDNSQSILYLQMNTLRAEDSATYYCA | | SGKDYTLSITSLQTEDVATYYCQQYWSTPWTFG | |
| | AGLDYWGQGTTLTVSS | | GGTKLEIK | |
| SCT-Ga447 | EVMLVESGGGLVKPGGSLKLSCAASGFTFSNYA | 370 | DIQMTQSSSYLSVSLGGRVTITCKASDHINNWL | 645 |
| | MSWVRQTPEKRLDWVATISSGGIYTYYPDNVKG | | AWYQQKPGNAPRLLISGATSLETGVPSRFSGSG | |
| | RFSISRDNAKNTLYLQLSSLRSEDTAMYYCARQ | | SGKDYTLSITSLQTEDVATYYCQQYWSTPFTFG | |
| | RTTATDYFDYWGQGTTLTVSS | | SGTKLEIK | |
| SCT-Ga448 | QVQLKQSGAELVRPGASVKLSCKTSGYIFTSYW | 371 | DIQMTQSPASQSASLGESVTITCLASQTIGTWL | 682 |
| | IHWVKQRSGQGLEWIARIYPGTGGTSYNEKFKG | | AWYQQKPGKSPQLLIYAATSLADGVPSRFSGSG | |
| | KATLTADKSSTTAYMQLSSLKSEDSAVYFCEVP | | SGTKFSFKISSLQAEDFVSYYCQQLYSTPLTFG | |
| | IGTVDYWGQGTTLTVSS | | AGTKLELK | |
| SCT-Ga449 | EVKLVESGGGLVQPGGSLRLSCATSGFTFTNYY | 372 | DWMTQTPLTLSVTIGQPASISCKSSQSLLDSD | 381 |
| | MSWVRQPPGKALEWLGFIRNKPYGYTTEYSASV | | GKTYLNWLLQRPGQSPKRLIYLVSKLDSGVPDR | |
| | KGRFTISRDNSQSILYLQMNTLTAEDSATYYCA | | FTGSGSGTDFTLKISRVEAEDLGVYYCWQGTHF | |
| | AGLDYWGQGTTLTVSS | | PYTFGGGTKLEIK | |
| SCT-Ga450 | QVQLKQSGAELVRPGASVKLSCKTSGYIFTSYW | 373 | DIQMTQSPASQSASLGESVTITCLASQTIGTWL | 683 |
| | IHWVKQRSGQGLEWIARIYPGTGSTYYNEKFKG | | AWYQQKPGKSPQLLIYAATSLADGVPSRFSGSG | |
| | KATLTADKSSSTAYMQLSSLKSEDSAVYFCEVP | | SGTKFSFKISSLQAEDFVSYYCQQLYSTPLTFG | |
| | VGTFDYWGQGTTLTVSS | | AGTKLELI | |
| SCT-Ga451 | DVQLQESGPGLVKPSQSLSLTCTVTGYSITSDY | 374 | DIVMTQSHKFMSTSVGDRVSITCKASQDVSTTV | 684 |
| | AWNWIRQFPGNKLDWMGYITYSGDTRYNPSLKS | | AWYQQKPGQSPKPLIYSASYRYTGVPDRFTGSG | |
| | RISITRDTSKNQFFLRLNSVTTEDTATYYCARR | | SGTDFTFTISSVQAEDLAVYYCQQHYSVPWTFG | |
| | EVGQGFTYWGQGTLVTVSA | | GGTKLEIN | |
| SCT-Ga452 | QVQLKESGPGLVAPSQSLSITCTVSGFSLTSFG | 375 | EILLTQSPAIIAASPGVKVTITCSASSSVRYMN | 685 |
| | VHWVRQPPGKGLEWLGVIWADGGTNYNSALMSR | | WYQQKPGSPPKIWIYGISNLASGVPARFSGSGS | |
| | LSISKDNSKSQVFLKMNSLQTDDTAMYYCARDL | | GTSFSFTINSMEAEDVATYYCQQTSSYPLTFGA | |
| | GYLTTAWFAYWGQGTLVTVSA | | GTKLELK | |
| SCT-Ga453 | EVQLQQSGPELVKPGASMKISCKASGYSFTGYI | 376 | DVWTQTPLSLPVSFGDQVSISCRSSQSLANSY | 482 |
| | MNWVKQSHGKNLEWIGLIIPYNGGTSYNQKFKG | | GNTYLSWYLHKPGQSPQLLIYGISNRFSGVPDR | |
| | KATLTVDKSSSTAYMELLSLTSEDSAVYYCTRG | | FSGSGSGTDFTLKISTIKPEDLGMYYCLQGTHQ | |
| | LQRFYAMDYWGQGTSVTVSS | | PPTFGGGTKLEIK | |
| SCT-Ga454 | DVQLQESGPGLVKPSQSLSLTCTVTGYSITSDY | 377 | DIVMTQSHKFMSTSVGDRVSITCKASQDVSTAV | 686 |
| | AWNWIRQFPGNKLEWMGYIVYSGSTNYNPSLKS | | AWYQQKPGQSPKLLIYSASYRYTGVPDRFTGSG | |
| | RISITRDTSKNHFFLQLNSVTAEDTATYYCARR | | SGTDFTFTINSVQAEDLAVYYCQQHYSSPWTFG | |
| | QVGLGFAYWGQGTLVTVSA | | GGTKLEIK | |
| SCT-Ga455 | EVQLQQSGPELVKPGASMKISCKASGYSFTGYT | 378 | DVWTQTPLSLPVSFGDQVSISCRSSQSLANSY | 572 |
| | MSWVKQSHGKNLEWIGLIIPYTGGTSYSQKFKG | | GNTYLSWYLHKPGQSPQLLIYGISNRFSGVPDR | |
| | KATLTVDKSSTTAYMELLSLTSEDSAVYYCARS | | FSGSGSGTDFTLKISTIKPEDLGMYYCLQGTHQ | |
| | RTFAMDYWGQGTSVTVSS | | PWTFGGGTKLEIK | |
| SCT-Ga456 | EVKLEESGGGLVQPGGSMKLSCVASGFTFSQFW | 379 | EILLTQSPAIIAASPGEKVTITCSASSSVSYMN | 667 |
| | MNWVRQSPEKGLEWVAEIRLKSNSYKTQYAESV | | WYQQKPGSSPKIWIYGISNLASGVPARFSGSGS | |
| | KGRFTISRDDSKSSVYLQMNNLRAEDTGIYYCT | | GTSFSFTINSMEAEDVATYYCQQISSYPLTFGA | |
| | RLPIFDYWGQGTTLTVSS | | GTKLELK | |
| SCT-Ga457 | QVQLKQSGPGLVQPSQSLSITCTVSGFSLTRHG | 1366 | DIQLTQSPSSLSASVGDRVTMSCKSSQSVLYSS | 1367 |
| | VHWVRQSPGKGLEWLGVIWPGGNTDYNTPFTSR | | NQKNFLAWYQQKPGKAPKLLIYWASTRESGVPS | |
| | LSINKDNSKSQVFFKMNSLQSNDTAIYYCARSG | | RFSGSGSGTDFTFTISSLQPEDIATYYCHQYLS | |
| | GYGNFAPYYFDQWGQGTLVTVSA | | SYTFGGGTKLEIK | |
| SCT-Ga458 | QVQLKQSGPGLVQPSQSLSITCTVSGFSLTRHG | 1366 | DIVMTQSPDSLAVSLGERVTMNCKSSQSVLYSS | 1368 |
| | VHWVRQSPGKGLEWLGVIWPGGNTDYNTPFTSR | | NQKNFLAWYQQKPGQSPKLLIYWASTRESGVPD | |
| | LSINKDNSKSQVFFKMNSLQSNDTAIYYCARSG | | RFSGSGSGTDFTLTISSVQAEDVAVYYCHQYLS | |
| | GYGNFAPYYFDQWGQGTLVTVSA | | SYTFGGGTKLEIK | |
| SCT-Ga459 | DVKLVESGGGLVKLGGSLKLSCAASGFTFSSYT | 1369 | AIQMTQSPSSLSASVGDRVTITCQASQNINVWL | 1370 |
| | MSWVRQTPEKRLELVAAISSGGSYTYYLDTVKG | | SWYQQKPGKAPKLLIYKASTLASGVPSRFSGSG | |
| | RFTISRDNAKNTLYLQMSSLKSEDTALFYCTRW | | SGTDFTLTISSLQPDDFATYYCQQGQTYPFTFG | |
| | LLSLYAMDYWGQGTSVTVSS | | GGTKVEIK | |
| SCT-Ga460 | DVKLVESGGGLVKLGGSLKLSCAASGFTFSSYT | 1369 | DIQMTQSPSSLSASVGDRVTITCKASQNINVWL | 1371 |
| | MSWVRQTPEKRLELVAAISSGGSYTYYLDTVKG | | NWYQQKPGKAPKLLIYKASNLQTGVPSRFSGSG | |
| | RFTISRDNAKNTLYLQMSSLKSEDTALFYCTRW | | SGTDFTFTISSLQPEDIATYYCQQGQTYPFTFG | |
| | LLSLYAMDYWGQGTSVTVSS | | QGTKVEIK | |
| SCT-Ga461 | EVQLVESGGGLVQPGGSLRLSCAASGFTFSSYT | 1372 | ATQMTQSPSSLSASVGDRVTITCQASQNINVWL | 1370 |
| | MSWVRQAPGKGLEWVATISSGGSYTYYPDSVKG | | SWYQQKPGKAPKLLIYKASTLASGVPSRFSGSG | |
| | RFTISRDNAKNTLYLQMNSLRAEDTAVYYCTRW | | SGTDFTLTISSLQPDDFATYYCQQGQTYPFTFG | |
| | LLSLYAMDYWGQGTLVTVSS | | GGTKVEIK | |
| SCT-Ga462 | EVQLVESGGGLVQPGGSLRLSCAASGFTFSSYT | 1372 | DIQMTQSPSSLSASVGDRVTITCKASQNINVWL | 1371 |
| | MSWVRQAPGKGLEWVATISSGGSYTYYPDSVKG | | NWYQQKPGKAPKLLIYKASNLQTGVPSRFSGSG | |
| | RFTISRDNAKNTLYLQMNSLRAEDTAVYYCTRW | | SGTDFTFTISSLQPEDIATYYCQQGQTYPFTFG | |
| | LLSLYAMDYWGQGTLVTVSS | | QGTKVEIK | |
| SCT-Ga463 | DVKLVESGGGLVKLGGSLKLSCAASGFAFSSFD | 1373 | AIQMTQSPSSLSASVGDRVTITCQASQNINVWL | 1374 |
| | MSWVRQTPEKRLELVAAISSGGSYTYYLDTVKG | | SWYQQKPGKAPKLLIYKASTLASGVPSRFSGSG | |
| | RFTISRDNAKNTLYLQMSSLKSEDTALFYCTRQ | | SGTDFTLTISSLQPDDFATYYCQQGQSYPWTFG | |
| | DYRYPYIMDYWGQGTSVTVSS | | GGTKVEIK | |
| SCT-Ga464 | DVKLVESGGGLVKLGGSLKLSCAASGFAFSSFD | 1373 | DIQMTQSPSSLSASVGDRVTITCKASQNINVWL | 1375 |
| | MSWVRQTPEKRLELVAAISSGGSYTYYLDTVKG | | NWYQQKPGKAPKLLIYKASNLQTGVPSRFSGSG | |
| | RFTISRDNAKNTLYLQMSSLKSEDTALFYCTRQ | | SGTDFTFTISSLQPEDIATYYCQQGQSYPWTFG | |
| | DYRYPYIMDYWGQGTSVTVSS | | QGTKVEIK | |
| SCT-Ga465 | EVQLVESGGKLLKPGGSLKLSCAASGFAFSSFD | 1376 | AIQMTQSPSSLSASVGDRVTITCQASQNINVWL | 1374 |
| | MSWFRQSPEKRLEWVAEISSGGSYTYYPDTVTG | | SWYQQKPGKAPKLLIYKASTLASGVPSRFSGSG | |
| | RFTISRDNAKNTLYLEMSSLRSEDTAMYYCTRQ | | SGTDFTLTISSLQPDDFATYYCQQGQSYPWTFG | |
| | DYRYPYIMDYWGQGTSVTVSS | | GGTKVEIK | |
| SCT-Ga466 | EVQLVESGGKLLKPGGSLKLSCAASGFAFSSFD | 1376 | DIQMTQSPSSLSASVGDRVTITCKASQNINVWL | 1375 |
| | MSWFRQSPEKRLEWVAEISSGGSYTYYPDTVTG | | NWYQQKPGKAPKLLIYKASNLQTGVPSRFSGSG | |
| | RFTISRDNAKNTLYLEMSSLRSEDTAMYYCTRQ | | SGTDFTFTISSLQPEDIATYYCQQGQSYPWTFG | |
| | DYRYPYIMDYWGQGTSVTVSS | | QGTKVEIK | |
| SCT-Ga467 | QVQLQQPGAELVKPGASVKMSCKASGYAFTDYL | 1377 | DIVMTQSPDSLAVSLGERATINCKSSQSLLNSG | 1378 |
| | MHWVKQTPGRGLEWIGAINPGSGGTSYNQKFKG | | NQKNYLAWYQQKPGQPPKLLIYGASTRESGVPD | |
| | KATLTADKSSSTAYMQLSSLTSEDSAVYYCARY | | RFSGSGSGTDFTLTISSLQAEDVAVYYCQNYHS | |
| | HYYGSGLFPYWGAGTTVTVSA | | YPFTFGGGTKLEIK | |
| SCT-Ga468 | QVQLQQPGAELVKPGASVKMSCKASGYAFTDYL | 1377 | DIVMTQSPDSLAVSLGERVTMNCKSSQSLLNSG | 1379 |
| | MHWVKQTPGRGLEWIGAINPGSGGTSYNQKFKG | | NQKNYLAWYQQKPGQSPKLLIYGASTRESGVPD | |
| | KATLTADKSSSTAYMQLSSLTSEDSAVYYCARY | | RFSGSGSGTDFTLTISSVQAEDVAVYYCQNYHS | |
| | HYYGSGLFPYWGAGTTVTVSA | | YPFTFGGGTKLEIK | |
| SCT-Ga469 | QVQLVQSGAEVKKPGASVKVSCKASGYAFTDYL | 1380 | DIVMTQSPDSLAVSLGERATINCKSSQSLLNSG | 1378 |
| | IEWVRQAPGQGLEWIGVINPGSGGTNYNEKFKG | | NQKNYLAWYQQKPGQPPKLLIYGASTRESGVPD | |
| | RATITADKSTSTAYMELSSLRSEDTAVYFCARY | | RFSGSGSGTDFTLTISSLQAEDVAVYYCQNYHS | |
| | HYYGSGLFPYWGQGTTVTVSS | | YPFTFGGGTKLEIK | |
| SCT-Ga470 | QVQLVQSGAEVKKPGASVKVSCKASGYAFTDYL | 1380 | DIVMTQSPDSLAVSLGERVTMNCKSSQSLLNSG | 1379 |
| | IEWVRQAPGQGLEWIGVINPGSGGTNYNEKFKG | | NQKNYLAWYQQKPGQSPKLLIYGASTRESGVPD | |
| | RATITADKSTSTAYMELSSLRSEDTAVYFCARY | | RFSGSGSGTDFTLTISSVQAEDVAVYYCQNYHS | |
| | HYYGSGLFPYWGQGTTVTVSS | | YPFTFGGGTKLEIK | |
| SCT-Ga471 | QVQLQQPGAELVKPGASVKMSCKASGYTFSNYW | 1381 | DIQMTQSPSSLSASVGDRVTITCRASQGIGNYL | 1382 |
| | MHWVKQTPGRGLEWIGAILPGSGSTSYNQKFKG | | AWYQQKPGKAPKLLIYYTSKIHSGVPSRFSGSG | |
| | KATLTADKSSSTAYMQLSSLTSEDSAVYYCARQ | | SGTDYTFTISSLQPEDIATYYCHQYSKLPYTFG | |
| | KYGNFYYFDFWGAGTTVTVSA | | QGTKVEIK | |
| SCT-Ga472 | QVQLQQPGAELVKPGASVKMSCKASGYTFSMYW | 1381 | DIQMTQSPSSLSASVGDRVTITCSASQGIGNYL | 1383 |
| | MHWVKQTPGRGLEWIGAILPGSGSTSYNQKFKG | | NWYQQKPGKAPKVLIYYTSSLHSGVPSRFSGSG | |
| | KATLTADKSSSTAYMQLSSLTSEDSAVYYCARQ | | SGTDFTLTISSLQPEDFATYYCHQYSKLPYTFG | |
| | KYGNFYYFDFWGAGTTVTVSA | | QGTKVEIK | |
| SCT-Ga473 | QVQLVQSGAEVKKPGASVKVSCKASGYTFSNYW | 1384 | DIQMTQSPSSLSASVGDRVTITCRASQGIGNYL | 1382 |
| | IQWVRQAPGQGLEWMGEILPGSGSTEYTENFKD | | AWYQQKPGKAPKLLIYYTSKIHSGVPSRFSGSG | |
| | RVTMTRDTSTSTVYMELSSLRSEDTAVYYCARQ | | SGTDYTFTISSLQPEDIATYYCHQYSKLPYTFG | |
| | KYGNFYYFDFWGQGTLVTVSS | | QGTKVEIK | |
| SCT-Ga474 | QVQLVQSGAEVKKPGASVKVSCKASGYTFSNYW | 1384 | DIQMTQSPSSLSASVGDRVTITCSASQGIGNYL | 1383 |
| | IQWVRQAPGQGLEWMGEILPGSGSTEYTENFKD | | NWYQQKPGKAPKVLIYYTSSLHSGVPSRFSGSG | |
| | RVTMTRDTSTSTVYMELSSLRSEDTAVYYCARQ | | SGTDFTLTISSLQPEDFATYYCHQYSKLPYTFG | |
| | KYGNFYYFDFWGQGTLVTVSS | | QGTKVEIK | |
| SCT-Ga475 | QVQLQQPGAELVKPGASVKMSCKASGYAFTNYL | 1385 | DIVMTQSPDSLAVSLGERATINCKSSQSLLNSG | 1386 |
| | MHWVKQTPGRGLEWIGAINPGSGGTSYNQKFKG | | IQKNYLAWYQQKPGQPPKLLIYGASTRESGVPD | |
| | KATLTADKSSSTAYMQLSSLTSEDSAVYYCART | | RFSGSGSGTDFTLTISSLQAEDVAVYYCQNDHR | |
| | GVYGNFLIFDYWGAGTTVTVSA | | YPFTFGGGTKLEIK | |
| SCT-Ga476 | QVQLQQPGAELVKPGASVKMSCKASGYAFTNYL | 1385 | DIVMTQSPDSLAVSLGERVTMNCKSSQSLLNSG | 1387 |
| | MHWVKQTPGRGLEWIGAINPGSGGTSYNQKFKG | | IQKNYLAWYQQKPGQSPKLLIYGASTRESGVPD | |
| | KATLTADKSSSTAYMQLSSLTSEDSAVYYCART | | RFSGSGSGTDFTLTISSVQAEDVAVYYCQNDHR | |
| | GVYGNFLIFDYWGAGTTVTVSA | | YPFTFGGGTKLEIK | |
| SCT-Ga477 | QVQLVQSGAEVKKPGASVKVSCKASGYAFTNYL | 1388 | DIVMTQSPDSLAVSLGERATINCKSSQSLLNSG | 1386 |
| | IEWVRQAPGQGLEWIGVINPGSGGTNYNEKFKG | | IQKNYLAWYQQKPGQPPKLLIYGASTRESGVPD | |
| | RATITADKSTSTAYMELSSLRSEDTAVYFCART | | RFSGSGSGTDFTLTISSLQAEDVAVYYCQNDHR | |
| | GVYGNFLIFDYWGQGTTVTVSS | | YPFTFGGGTKLEIK | |
| SCT-Ga478 | QVQLVQSGAEVKKPGASVKVSCKASGYAFTNYL | 1388 | DIVMTQSPDSLAVSLGERVTMNCKSSQSLLNSG | 1387 |
| | TEWVRQAPGQGLEWTGVTNPGSGGTNYNEKFKG | | IQKNYLAWYQQKPGQSPKLLIYGASTRESGVPD | |
| | RATITADKSTSTAYMELSSLRSEDTAVYFCART | | RFSGSGSGTDFTLTISSVQAEDVAVYYCQNDHR | |
| | GVYGNFLIFDYWGQGTTVTVSS | | YPFTFGGGTKLEIK | |
| SCT-Ga479 | QVQLKQSGPGLVQPSQSLSITCTVSGFSLTSYG | 1389 | DIQMTQSPSSLSASVGDRVTITCGASENIYSNL | 1390 |
| | VHWVRQSPGKGLEWLGVIWAGGTTDYNTPFTSR | | NWYQQKPGKAPKLLIYAATNLADGVPSRFSGSG | |
| | LSINKDNSKSQVFFKMNSLQSNDTAIYYCARFP | | SGTDFTLTISSLQPEDFATYYCQHFWGTPYTFG | |
| | SYRESMDYWGQGTLVTVSA | | QGTKVEIK | |
| SCT-Ga480 | QVQLQQPGAELVKPGASVKMSCKASGYAFTNYL | 1391 | DIQMTQSPSSLSASVGDRVTITCRASQDVSTAV | 1392 |
| | MHWVKQTPGRGLEWIGAINPGSGGTSYNQKFKG | | AWYQQKPGKAPKLLIYSASFLYSGVPSRFSGSG | |
| | KATLTADKSSSTAYMQLSSLTSEDSAVYYCARR | | SGTDFTLTISSLQPEDFATYYCQQHYSSPRTFG | |
| | GDKYGSAWFAYWGAGTTVTVSA | | QGTKVEIK | |
| SCT-Ga481 | QVQLQQPGAELVKPGASVKMSCKASGYAFTNYL | 1391 | DIVMTQSQRFMSTTVGDRVSITCKASQDVSTAV | 1393 |
| | MHWVKQTPGRGLEWIGAINPGSGGTSYNQKFKG | | AWYQQKPGQSPKLLIYSASNRYTGVPDRFTGSG | |
| | KATLTADKSSSTAYMQLSSLTSEDSAVYYCARR | | SGTDFTLTISNMQSEDLADFFCQQHYSSPRTFG | |
| | GDKYGSAWFAYWGAGTTVTVSA | | GGTKLEIK | |
| SCT-Ga482 | QVQLVQSGAEVKKPGASVKVSCKASGYAFTNYL | 1394 | DIQMTQSPSSLSASVGDRVTITCRASQDVSTAV | 1392 |
| | IEWVRQAPGQGLEWIGVINPGSGGTNYNEKFKG | | AWYQQKPGKAPKLLIYSASFLYSGVPSRFSGSG | |
| | RATITADKSTSTAYMELSSLRSEDTAVYFCARR | | SGTDFTLTISSLQPEDFATYYCQQHYSSPRTFG | |
| | GDKYGSAWFAYWGQGTTVTVSS | | QGTKVEIK | |
| SCT-Ga483 | QVQLVQSGAEVKKPGASVKVSCKASGYAFTNYL | 1394 | DIVMTQSQRFMSTTVGDRVSITCKASQDVSTAV | 1393 |
| | IEWVRQAPGQGLEWIGVINPGSGGTNYNEKFKG | | AWYQQKPGQSPKLLIYSASNRYTGVPDRFTGSG | |
| | RATITADKSTSTAYMELSSLRSEDTAVYFCARR | | SGTDFTLTISNMQSEDLADFFCQQHYSSPRTFG | |
| | GDKYGSAWFAYWGQGTTVTVSS | | GGTKLEIK | |
| SCT-Ga484 | QVQLQQSGABVKKPGASVKVSCKASGYAFTNYL | 1395 | DIVMTQSPDSLAVSLGERATINCKSSQSLLNSG | 1386 |
| | IEWVRQAPGQGLEWIGVINPGSGGSKYNEKFKG | | IQKNYLAWYQQKPGQPPKLLIYGASTRESGVPD | |
| | RATITADKSTSTAYMELSSLRSEDTAVYFCART | | RFSGSGSGTDFTLTISSLQAEDVAVYYCQNDHR | |
| | GVYGNFLIFDYWGQGTTVTVSS | | YPFTFGGGTKLEIK | |
| SCT-Ga485 | QVQLVQSGAEVKKPGASVKVSCKASGYAFTNYL | 1396 | DIVMTQSPDSLAVSLGERATINCKSSQSLLNSG | 1386 |
| | IEWVKQAPGQGLEWIGVINPGSGGSKYNEKFKG | | IQKNYLAWYQQKPGQPPKLLIYGASTRESGVPD | |
| | RATITADKSTSTAYMELSSLRSEDTAVYFCART | | RFSGSGSGTDFTLTISSLQAEDVAVYYCQNDHR | |
| | GVYGNFLIFDYWGQGTTVTVSS | | YPFTFGGGTKLEIK | |
| SCT-Ga486 | QVQLQQSGAEVKKPGASVKVSCKASGYAFTNYL | 1397 | DIVMTQSPDSLAVSLGERATINCKSSQSLLNSG | 1386 |
| | IEWVKQAPGQGLEWIGVINPGSGGSKYNEKFKG | | IQKNYLAWYQQKPGQPPKLLIYGASTRESGVPD | |
| | RATITADKSTSTAYMELSSLRSEDTAVYFCART | | RFSGSGSGTDFTLTISSLQAEDVAVYYCQNDHR | |
| | GVYGNFLIFDYWGQGTTVTVSS | | YPFTFGGGTKLEIK | |
| SCT-Ga487 | QVQLQQPGAELVKPGASVKMSCKATGYTFSNYW | 1398 | AIQMTQSPSSLSASVGDRVTITCRASQGIGNYL | 1399 |
| | IEWVKQTPGRGLEWIGEILPGSGSSYYNQKFKG | | NWYQQKPGKAPKLLIYYTSSLHSGVPSRFSGSG | |
| | KATLTADKSSSTAYMQLSSLTSEDSAVYYCARQ | | SGTDYTFTISSLQPEDIATYYCHQYSKLPYTFG | |
| | KYGNFYYFDFWGAGTTVTVSA | | QGTKVEIK | |
| SCT-Ga488 | QVQLVQSGAEVKKPGASVKVSCKASGYTFSMYW | 1400 | AIQMTQSPSSLSASVGDRVTITCRASQGTGNYL | 1399 |
| | IEWVRQAPGQGLEWIGEILPGSGSSYYNENFKD | | NWYQQKPGKAPKLLIYYTSSLHSGVPSRFSGSG | |
| | RVTMTRDTSTSTVYMELSSLRSEDTAVYYCARQ | | SGTDYTFTISSLQPEDIATYYCHQYSKLPYTFG | |
| | KYGNFYYFDFWGQGTLVTVSS | | QGTKVEIK | |
| SCT-Ga489 | QVQLVQSGAEVKKPGASVKVSCKATGYTFSNYW | 1401 | AIQMTQSPSSLSASVGDRVTITCRASQGIGNYL | 1399 |
| | IEWVRQAPGQGLEWTGEILPGSGSSYYNENFKD | | NWYQQKPGKAPKLLTYYTSSLHSGVPSRFSGSG | |
| | RVTMTRDTSTSTVYMELSSLRSEDTAVYYCARQ | | SGTDYTFTISSLQPEDIATYYCHQYSKLPYTFG | |
| | KYGNFYYFDFWGQGTLVTVSS | | QGTKVEIK | |
| SCT-Ga490 | QVQLQQPGAELVKPGASVKMSCKATGYTFSNYW | 1398 | DIQMTQSPSSLSASVGDRVTITCRASQGIGNYL | 1382 |
| | IEWVKQTPGRGLEWTGEILPGSGSSYYNQKFKG | | AWYQQKPGKAPKLLIYYTSKIHSGVPSRFSGSG | |
| | KATLTADKSSSTAYMQLSSLTSEDSAVYYCARQ | | SGTDYTFTISSLQPEDIATYYCHQYSKLPYTFG | |
| | KYGNFYYFDFWGAGTTVTVSA | | QGTKVEIK | |
| SCT-Ga491 | QVQLVQSGAEVKKPGASVKVSCKASGYTFSNYW | 1400 | DIQMTQSPSSLSASVGDRVTTTCRASQGTGNYL | 1382 |
| | IEWVRQAPGQGLEWIGEILPGSGSSYYNENFKD | | AWYQQKPGKAPKLLIYYTSKIHSGVPSRFSGSG | |
| | RVTMTRDTSTSTVYMELSSLRSEDTAVYYCARQ | | SGTDYTFTTSSLQPEDTATYYCHQYSKLPYTFG | |
| | KYGNFYYFDFWGQGTLVTVSS | | QGTKVEIK | |
| SCT-Ga492 | QVQLVQSGAEVKKPGASVKVSCKATGYTFSNYW | 1401 | DIQMTQSPSSLSASVGDRVTTTCRASQGIGNYL | 1382 |
| | IEWVRQAPGQGLEWIGEILPGSGSSYYNENFKD | | AWYQQKPGKAPKLLIYYTSKIHSGVPSRFSGSG | |
| | RVTMTRDTSTSTVYMELSSLRSEDTAVYYCARQ | | SGTDYTFTISSLQPEDTATYYCHQYSKLPYTFG | |
| | KYGNFYYFDFWGQGTLVTVSS | | QGTKVEIK | |
| SCT-Ga493 | EVKLVESGGGLVQPGGSLRLSCAASGFAFSSFD | 1402 | ATQMTQSPSSLSASVGDRVTITCQASQNINVWL | 1374 |
| | MSWVRQAPGKGLEWVSVISSGGSYTYYADSVKG | | SWYQQKPGKAPKLLTYKASTLASGVPSRFSGSG | |
| | RFTISRDNSKNTLYLQMNSLRAEDTAVYYCTRQ | | SGTDFTLTISSLQPDDFATYYCQQGQSYPWTFG | |
| | DYRYPYTMDYWGQGTLVTVSS | | GGTKVEIK | |
| SCT-Ga494 | EVKLVESGGGLVQPGGSLRLSCAASGFAFSSFD | 1402 | DIQMTQSPSSLSASVGDRVTITCHASQNINVWL | 1403 |
| | MSWVRQAPGKGLEWVSVISSGGSYTYYADSVKG | | SWYQQKPGKAPKLLTYKASTLASGVPSRFSGSG | |
| | RFTISRDNSKNTLYLQMNSLRAEDTAVYYCTRQ | | SGTDFTLTISSLQPDDFATYYCQQGQSYPWTFG | |
| | DYRYPYTMDYWGQGTLVTVSS | | GGTKVEIK | |
| SCT-Ga495 | EVKLVESGGGLVQPGGSLRLSCAASGFAFSSFD | 1404 | AIQMTQSPSSLSASVGDRVTITCQASQNINVWL | 1374 |
| | MSWVRQAPGKGLEWVAVISSGGSYTFYPDSVKG | | SWYQQKPGKAPKLLTYKASTLASGVPSRFSGSG | |
| | RFTISRDNSKNTLYLQMNSLRAEDTALYFCTRQ | | SGTDFTLTISSLQPDDFATYYCQQGQSYPWTFG | |
| | DYRYPYTMDYWGQGTLVTVSS | | GGTKVEIK | |
| SCT-Ga496 | EVKLVESGGGLVQPGGSLRLSCAASGFAFSSFD | 1404 | DIQMTQSPSSLSASVGDRVTITCHASQNINVWL | 1403 |
| | MSWVRQAPGKGLEWVAVISSGGSYTFYPDSVKG | | SWYQQKPGKAPKLLIYKASTLASGVPSRFSGSG | |
| | RFTISRDNSKNTLYLQMNSLRAEDTALYFCTRQ | | SGTDFTLTISSLQPDDFATYYCQQGQSYPWTFG | |
| | DYRYPYIMDYWGQGTLVTVSS | | GGTKVEIK | |
| SCT-Ga497 | QVQLQQSGAEVKKPGASVKVSCKASGYAFTNYL | 1405 | DIVMTQSPSSLSASVGDRVTITCKASQDVSTAV | 1406 |
| | TEWVKQAPGQGLEWIGVINPGSGGNNYNEKFKG | | AWYQQKPGKAPKLLIYSASYRYSGVPSRFSGSG | |
| | RATTTADKSTSTAYMELSSLRSEDTAVYFCARR | | SGTDFTLTISSLQPEDFATYYCQQHYSSPRTFG | |
| | GDKYGSAWFAYWGQGTTVTVSA | | QGTKVEIK | |
| SCT-Ga498 | QVQLQQSGAEVKKPGASVKVSCKASGYAFTNYL | 1405 | DIVMTQSQRFMSTTVGDRVSITCKASQDVSTAV | 1407 |
| | IEWVKQAPGQGLEWIGVINPGSGGNNYNEKFKG | | AWYQQKPGQSPKLLIYSASYRYTGVPDRFTGSG | |
| | RATITADKSTSTAYMELSSLRSEDTAVYFCARR | | SGTDFTLTISNMQSEDLAVYYCQQHYSSPRTFG | |
| | GDKYGSAWFAYWGQGTTVTVSA | | GGTKLEIK | |

The preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

### REFERENCES CITED

### Other References

Boles, K. S., Vermi, W., Facchetti, F., Fuchs, A., Wilson, T. J., Diacovo, T. G., Cella, M., and Colonna, M. (2009). A novel molecular interaction for the adhesion of follicular CD4 T cells to follicular DC. Eur. J. Immunol. 39, 695-703.
Gao, J., Zheng, Q., Xin, N., Wang, W., and Zhao, C. (2017). CD155, an onco-immunologic molecule in human tumors. Cancer Sci. 108, 1934-1938.
Gur, C., Ibrahim, Y., Isaacson, B., Yamin, R., Abed, J., Gamliel, M., Enk, J., Bar-On, Y., Stanietsky-Kaynan, N., Coppenhagen-Glazer, S., et al. (2015). Binding of the Fap2 protein of Fusobacterium nucleatum to human inhibitory receptor TIGIT protects tumors from immune cell attack. Immunity 42, 344-355.
Hampton, T. (2015). Bacteria Protect Colorectal Cancer Cells From Immune Destruction. JAMA 313, 1305.
Johnston, R. J., Comps-Agrar, L., Hackney, J., Yu, X., Huseni, M., Yang, Y., Park, S., Javinal, V., Chiu, H., Irving, B., et al. (2014). The immunoreceptor TIGIT regulates antitumor and antiviral CD8(+) T cell effector function. Cancer Cell 26, 923-937.
Johnston, R. J., Yu, X., and Grogan, J. L. (2015). The checkpoint inhibitor TIGIT limits antitumor and antiviral CD8+ T cell responses. Oncoimmunology 4, e1036214.
Joller, N., Lozano, E., Burkett, P. R., Patel, B., Xiao, S., Zhu, C., Xia, J., Tan, T. G., Sefik, E., Yajnik, V., et al. (2014). Treg cells expressing the coinhibitory molecule TIGIT selectively inhibit proinflammatory Th1 and Th17 cell responses. Immunity 40, 569-581.
Levin, S. D., Taft, D. W., Brandt, C. S., Bucher, C., Howard, E. D., Chadwick, E. M., Johnston, J., Hammond, A., Bontadelli, K., Ardourel, D., et al. (2011). Vstm3 is a member of the CD28 family and an important modulator of T-cell function. Eur. J. Immunol. 41, 902-915.
Lozano, E., Dominguez-Villar, M., Kuchroo, V., and Hafler, D. A. (2012). The TIGIT/CD226 axis regulates human T cell function. J. Immunol. Baltim. Md 1950 188, 3869-3875.
Stanietsky, N., Simic, H., Arapovic, J., Toporik, A., Levy, O., Novik, A., Levine, Z., Beiman, M., Dassa, L., Achdout, H., et al. (2009). The interaction of TIGIT with PVR and PVRL2 inhibits human NK cell cytotoxicity. Proc. Natl. Acad. Sci. U.S.A 106, 17858-17863.
Stengel, K. F., Harden-Bowles, K., Yu, X., Rouge, L., Yin, J., Comps-Agrar, L., Wiesmann, C., Bazan, J. F., Eaton, D. L., and Grogan, J. L. (2012). Structure of TIGIT immunoreceptor bound to poliovirus receptor reveals a cell-cell adhesion and signaling mechanism that requires cis-trans receptor clustering. Proc. Natl. Acad. Sci. U.S.A 109, 5399-5404.
Yu, X., Harden, K., Gonzalez, L. C., Francesco, M., Chiang, E., Irving, B., Tom, I., Ivelja, S.,
Refino, C. J., Clark, H., et al. (2009). The surface protein TIGIT suppresses T cell activation by promoting the generation of mature immunoregulatory dendritic cells. Nat. Immunol. 10, 48-57.

1. An antibody or an antigen binding fragment thereof that binds human T cell immunoreceptor with Ig and ITIM domains (TIGIT) comprising SEQ ID NO: 1, the antibody or the antigen binding fragment thereof comprising:
a HCDR1 having the amino acid sequence of SEQ ID NO:804, a HCDR2 having the amino acid sequence of SEQ ID NO:814, a HCDR3 having the amino acid sequence of SEQ ID NO:1321, a LCDR1 having the amino acid sequence of SEQ ID NO:790, a LCDR2 having the amino acid sequence of SEQ ID NO:791, and a LCDR3 having the amino acid sequence of SEQ ID NO:1322.

2. (canceled)

3. An antibody or the antigen binding fragment thereof that binds human T cell immunoreceptor with Ig and ITIM domains (TIGIT) comprising SEQ ID NO: 1, the antibody comprising:
a) a heavy chain variable domain (VH) having the amino acid sequence of SEQ ID NO: 1372, and a light chain variable domain (VL) having the amino acid sequence of SEQ ID NO:1370; or
b) a VH having the amino acid sequence of SEQ ID NO:357, and a VL having the amino acid sequence of SEQ ID NO:673.

4. The antibody or the antigen binding fragment thereof of claim 3, wherein the VH has the amino acid sequence of SEQ ID NO:1372, and the VL has the amino acid sequence of SEQ ID NO:1370.

5. The antibody or the antigen binding fragment thereof of claim 3, wherein the VH has the amino acid sequence of SEQ ID NO:357, and the VL has the amino acid sequence of SEQ ID NO:673.

6. An antibody or the antigen binding fragment thereof that binds human T cell immunoreceptor with Ig and ITIM domains (TIGIT) comprising SEQ ID NO: 1, the antibody or the antigen binding fragment thereof comprising:
a) a heavy chain comprising the amino acid sequence of SEQ ID NO: 1372 and a light chain comprising the amino acid sequence of SEQ ID NO:1370; or
b) a heavy chain comprising the amino acid sequence of SEQ ID NO: 357 and a light chain comprising the amino acid sequence of SEQ ID NO:673.

7. The antibody or the antigen binding fragment thereof of claim 6, wherein the heavy chain comprises the amino acid sequence of SEQ ID NO:1372 and the light chain comprises the amino acid sequence of SEQ ID NO:1370.

8. The antibody or the antigen binding fragment thereof of claim 6, wherein the heavy chain comprises the amino acid sequence of SEQ ID NO:357 and the light chain comprises the amino acid sequence of SEQ ID NO:673.

9. A pharmaceutical composition comprising the antibody or the antigen binding fragment thereof of claim 1.

10. A kit comprising the antibody or the antigen binding fragment thereof of claim 1.

11. The antibody or the antigen binding fragment thereof of claim 1, comprising:
a) a heavy chain variable domain (VH) comprising the amino acid sequence of SEQ ID NO: 1372 or an amino acid sequence that is at least 80% identical thereto, and a light chain variable domain (VL) having the amino acid sequence of SEQ ID NO: 1370 or an amino acid sequence that is at least 80% identical thereto; or
b) a VH having the amino acid sequence of SEQ ID NO: 357 or an amino acid sequence that is at least 80% identical thereto, and a VL having the amino acid sequence of SEQ ID NO:673 or an amino acid sequence that is at least 80% identical thereto.

12. The antibody of claim 1, which comprises a full-length immunoglobulin.

13. The antigen binding fragment thereof of claim 1, wherein the antibody fragment comprises any one of: a diabody, a single-chain antibody molecule, an Fab, an Fab′, an F(ab′)₂, an Fv, or a scFv.

14. A method of treating a TIGIT-expressing cancer in a subject, comprising administering the antibody or the antigen binding fragment thereof of claim 1 to the subject.

15. A method of treating a TIGIT-expressing cancer in a subject, comprising administering the antibody or the antigen binding fragment thereof of claim 11 to the subject.

16. A method of determining whether or not a subject is amenable to treatment with a TIGIT-specific anti-cancer therapeutic, comprising contacting the antibody or the antigen binding fragment thereof of claim 1 with a sample and determining binding of the antibody to the sample.

17. The method of claim 16, wherein the contacting comprises a method selected from: an enzyme-linked immunosorbent assays (ELISA), a Western blot, an immunohistochemistry, an immunocytochemistry, a flow cytometry and fluorescence-activated cell sorting (FACS), an immunoprecipitation, or an enzyme-linked immunospot assays (ELISPOT).
